(19) United States
(12) Patent Application Publication
(10) Pub. No.: US 2013/0030000 A1
(43) Pub. Date: Jan. 31, 2013

(54) PHARMACEUTICAL COMPOSITIONS FOR THE TREATMENT OF PAIN AND OTHER INDICATIONS

(71) Applicants: Harry R. Chobanian, Aberdeen, NJ (US); Linus S. Lin, Westfield, NJ (US); Ping Liu, Westfield, NJ (US); Marc D. Chioda, New Brunswick, NJ (US); Robert J. DeVita, Westfield, NJ (US); Ravi P. Nargund, East Brunswick, NJ (US); Yan Guo, Westfield, NJ (US); Terence G. Hamill, Lansdale, PA (US); Wenping Li, Lansdale, PA (US); Darrell Arthur Henze, Collegeville, PA (US)

(72) Inventors: Harry R. Chobanian, Aberdeen, NJ (US); Linus S. Lin, Westfield, NJ (US); Ping Liu, Westfield, NJ (US); Marc D. Chioda, New Brunswick, NJ (US); Robert J. DeVita, Westfield, NJ (US); Ravi P. Nargund, East Brunswick, NJ (US); Yan Guo, Westfield, NJ (US); Terence G. Hamill, Lansdale, PA (US); Wenping Li, Lansdale, PA (US); Darrell Arthur Henze, Collegeville, PA (US)

(21) Appl. No.: 13/574,303

(22) PCT Filed: Jan. 25, 2011
(86) PCT No.: PCT/US11/22412

§ 371 Date: Oct. 12, 2012

Related U.S. Application Data
(60) Provisional application No. 61/299,087, filed on Jan. 28, 2010.

(51) Int. Cl. A61K 31/497; A61K 31/506; A61K 31/513; A61K 31/4439; A61K 31/421; A61K 31/4245; A61K 31/4545; A61K 31/501; A61K 31/4725; A61K 31/4709; A61P 29/00; A61P 25/22; A61P 3/04; A61P 27/02; A61P 11/06; A61P 27/06; A61P 9/00; A61P 25/24; A61P 43/00; A61P 19/02; A61P 25/28; A61P 25/18; A61P 25/30; A61P 25/16; A61P 25/08; A61P 11/00; A61P 35/00; A61P 1/00; A61P 1/04; A61P 1/12; A61P 17/04; A61P 1/16; A61P 25/06; A61P 25/02; A61K 31/444

(52) U.S. Cl. 514/252.03; 514/255.05; 514/333; 514/274; 514/340; 514/376; 514/364; 514/318; 514/342; 514/309; 514/314; 514/341; 514/334

(57) ABSTRACT

The present invention is directed to a composition useful for the treatment of a FAAH mediated disease, disorder or conditions comprising a FAAH inhibitor and a second activation, comprising a selected imidazole or oxazole FAAH inhibitor and a second active agent. The compositions will be useful in the treatment of a wide range of disease, disorder, or conditions including osteoarthritis, rheumatoid arthritis, diabetic neuropathy, postherpetic neuralgia, skeletomuscular pain, and fibromyalgia, as well as acute pain, migraine, sleep disorder, Alzheimer disease, and Parkinson's disease. In another aspect the invention discloses herein is directed to compositions useful in the treatment of neuropathic and nociceptive pain, said compositions comprising etoricoxib.

### BACKGROUND OF THE INVENTION

In one aspect the invention disclosed herein is directed to compositions useful in the treatment of pain and other FAAH mediated diseases, disorders and conditions. In particular, the invention disclosed herein is directed to pharmaceutical compositions comprising selected FAAH inhibitors and a second active agent.

In another aspect the invention disclosed herein is directed to compositions useful in the treatment of neuropathic and nociceptive pain, said compositions comprising etoricoxib.

In another aspect the invention disclosed herein is directed to compositions useful in the treatment of neuropathic and nociceptive pain, said compositions comprising etoricoxib and a selected FAAH inhibitor.

Disclosed herein are compounds that inhibit the activity of fatty acid amide hydrolase (FAAH), compositions that include the compounds, and methods of their use. Compounds disclosed herein as inhibitors of fatty acid amide hydrolase (FAAH) are useful in the treatment of diseases, disorders, or conditions that would benefit from the inhibition of fatty acid amide hydrolase and increases in endogenous fatty acid amides.

Fatty acid amide hydrolase (FAAH) is an enzyme that is abundantly expressed throughout the CNS (Freund et al. Physiol. Rev. 2003; 83:1017-1066) as well as in peripheral tissues, such as, for example, in the pancreas, brain, kidney, skeletal muscle, placenta, and liver (Giang, D. K. et al., Proc. Natl. Acad. Sci. U.S.A. 1997, 94, 2238-2242; Cravatt et al. Proc. Natl. Acad. Sci. U.S.A. 2004, 101, 29, 10821-10826). FAAH hydrolyzes the fatty acid amide (FAA) family of endogenous signaling lipids. General classes of fatty acid amides include the N-acylethanolamides (NAEs) and fatty acid primary amides (FAPAs). Examples of NAEs include anandamide (AEA), palmitoylethanolamide. (PEA) and oleoylethanolamide (OEA). An example of FAPAs includes 9-Z-octadecenamide or oleamide. (McKinney M K and Cravatt B F. 2005. Annu Rev Biochem 74:411-32). Another class of fatty acid amide family of endogenous signaling lipids is N-acyl taurines that have also been shown to be elevated upon FAAH deletion or inhibition and appear to act on transient receptor potential (TRP) family of calcium channels, although the functional consequences are not yet clear (Saghatelian A, et al. Biochemistry. 2004, 43:14332-9, Saghatelian A, et al. Biochemistry, 2006, 45:9007-9015). In addition to fatty acid amides, FAAH can also hydrolyze certain fatty acid esters, such as, for example, 2-arachidonylglycerol (2-AG) another endocannabinoid (Mechoularn et al. Biochem. Pharmacol. 1995; 50:83-90; Stella et al. Nature, 1997; 388:773-778; Suguria et al. Biochem. Biophys. Res. Commun. 1995; 215:89-97).

Inhibition of FAAH is expected to lead to an increase in the level of anandamide and other fatty acid amides. This increase in fatty acid amides leads to an increase in the nociceptive threshold. Thus, inhibitors of FAAH are useful in the treatment of pain (Cravatt, B F; Lichtman, A H Current Opinion in Chemical Biology 2003, 7, 469-475). Such inhibitors are useful in the treatment of other disorders that can be treated using fatty acid amides or modulators of cannabinoid receptors, such as, for example, anxiety, sleep disorder, Alzheimer disease, and Parkinson's disease, eating disorders, metabolic disorders, cardiovascular disorders, and inflammation (Simon et al Archives of Gen. Psychiatry, 2006, 63, 824-830. Kunos, G et al. Pharmacol Rev 2006, 58,389-462). In some embodiments, FAAH inhibitor compounds may be peripherally restricted and may not substantially affect neural disorders, such as, for example, depression and anxiety. Finally, agonism of cannabinoid receptors has also been shown to reduce the progression of atherosclerosis in animal models (see Steffens et al. Nature, 2005, 434, 782-786; and Steffens et al., Curr Opin. Lipid., 2006, 17, 519-526). Thus, increasing the level of endogenous cannabinergic fatty acid amides (e.g., anandamide) is expected to effectively treat or reduce the risk of developing atherosclerosis.

Inhibition of FAAH also leads to elevation of palmitoylethanolamide which is thought to work, in part, through activation of the peroxisome proliferator-activated receptor α (PPAR-α) to regulate multiple pathways including, for example; pain perception in neuropathic and inflammatory conditions such as convulsions, neurotoxicity, spasticity and to reduce inflammation, for example, in atopic eczema and arthritis (LoVerme J et al. The nuclear receptor peroxisome proliferator-activated receptor-alpha mediates the anti-inflammatory actions of palmitoylethanolamide. Mol Pharmacol 2005, 67, 15-19; LoVerme J et al The search for the palmitoylethanolamide receptor. Life Sci 2005, 77: 1685-1698. Lambert D M et al. The palmitoylethanolamide family: a new class of anti-inflammatory agents? Curr Med Chem 2002, 9: 663-674; Eberlein B, et al. Adjuvant treatment of atopic eczema: assessment of an emollient containing N-palmitoylethanolamine (ATOPA study). J Eur Acad Dermatol Venereol. 2008, 22:73-82. Re G, et al. Palmitoylethanolamide, endocannabinoids and related cannabimimetic compounds in protection against tissue inflammation and pain: potential use in companion animals. Vet J. 2007 173:21-30.). Thus, inhibition of FAAH is useful for the treatment of various pain and inflammatory conditions, such as osteoarthritis, rheumatoid arthritis, diabetic neuropathy, postherpetic neuralgia, skeletomuscular pain, and fibromyalgia.

It is also thought that certain fatty acid amides, such as, for example, OEA, act through the peroxisome proliferator-activated receptor α (PPAR-α) to regulate diverse physiological processes, including, e.g., feeding and lipolysis. Consistent with this, human adipose tissue has been shown to bind and metabolize endocannabinoids such as anandamide and 2-arachidonylglycerol (see Spoto et al., Biochimie 2006, 88, 1889-1897; and Matias et al., J. Clin. Endocrin. & Met., 2006, 91, 3171-3180). Thus, inhibiting FAAH activity in vivo leads to reduced body fat, body weight, caloric intake, and liver triglyceride levels. However, unlike other anti-lipidemic agents that act through PPAR-α, e.g., fibrates, FAAH inhibitors do not cause adverse side effects such as rash, fatigue, headache, erectile dysfunction, and, more rarely, anemia, leukopenia, angioedema, and hepatitis (see, e.g., Muscari et al. Cardiology, 2002, 97: 115-121).

Many fatty acid amides are produced on demand and rapidly degraded by FAAH. As a result, hydrolysis by FAAH is considered to be one of the essential steps in the regulation of fatty acid amide levels in the central nervous system as well as in peripheral tissues and fluids. The broad distribution of FAAH combined with the broad array of biological effects of fatty acid amides (both endocannabinoid and non-endocannabinoid mechanisms) suggests that inhibition of FAAH leads to altered levels of fatty acid amides in many tissues and fluids and may be useful to treat many different conditions. FAAH inhibitors increase the levels of endogenous fatty acid amides. FAAH inhibitors block the degradation of endocannabinoids and increase the tissue levels of these endogenous substances. FAAH inhibitors can be used in this respect in the prevention and treatment of pathologies in which endogenous cannabinoids and or any other substrates metabolized by the FAAH enzyme are involved.

The various fatty acid ethanolamides have important and diverse physiological functions. As a result, inhibitor molecules that selectively inhibit FAAH enzymatic activity would allow a corresponding selective modulation of the cellular and extra-cellular concentrations of a FAAH substrate. FAAH inhibitors that are biologically compatible could be effective pharmaceutical compounds when formulated as therapeutic agents for any clinical indication where FAAH enzymatic inhibition is desired. In some embodiments, FAAH activity in peripheral tissues can be preferentially inhibited. In some embodiments, FAAH inhibitors that do substantially cross the blood-brain-barrier can be used to preferentially inhibit FAAH activity in peripheral tissues. In some embodiments, FAAH inhibitors that preferentially inhibit FAAH activity in peripheral tissues can minimize the effects of FAAH inhibition in the central nervous system. In some embodiments, it is preferred to inhibit FAAH activity in peripheral tissues and minimize FAAH inhibition in the central nervous system.

### SUMMARY OF THE INVENTION

The present invention is directed to a composition useful for the treatment of a FAAH mediated disease, disorder or conditions comprising a selected FAAH inhibitor and a second active agent. The compositions will be useful in the treatment of a wide range of disease, disorder or conditions including osteoarthritis, rheumatoid arthritis, diabetic neuropathy, postherpetic neuralgia, skeletomuscular pain, and fibromyalgia, as well as acute pain, migraine, sleep disorder, Alzheimer disease, and Parkinson's disease.

In another aspect the invention disclosed herein is directed to compositions useful in the treatment of neuropathic and nociceptive pain, said compositions comprising etoricoxib.

In another aspect the invention is direct to a method of using these compositions.

### BRIEF DESCRIPTION OF THE FIGURES

FIG. 1 This FIGURE describes an Isobologram of the analgesic effects of etoricoxib co-dosed with the FAAH inhibitor Compound A in three different dose ratios (3:1, 1:1, 0.1:1). The solid line is the predicted line associated with additivity of analgesic effect.

Legend: Dose ratio (Etoricoxib:Compound A) ▴=3:1, ▪=1:1, =0.3:1. (Zmix is not statistically significant from Zadd for any ratio (P.0.05)).

### DETAILED DESCRIPTION OF THE INVENTION

In one aspect the invention is directed to pharmaceutical compositions comprising:

a FAAH inhibiting compound of formula I:

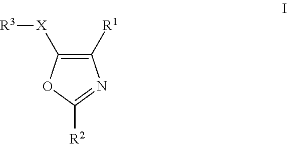

as defined hereinunder
or a FAAH inhibiting compound of formula II:

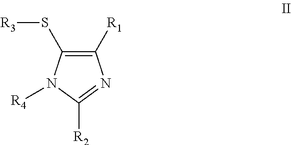

as defined hereinunder,
and a second active agent agent such as an agent.

Within this aspect there is a genus wherein the second active agent is useful for treating pain (e.g., acute pain, chronic pain, neurogenic pain, migraine; pain caused by inflammation (e.g., arthritis, osteoarthritis, spondylitis, rheumatoid arthritis, Crohn's disease and irritable bowel syndrome), and neuropathic pain), anxiety, an eating disorder (e.g., anorexia, bulimia), obesity, elevated intraocular pressure, glaucoma, a cardiovascular disorder, depression, an inflammatory disorder (allergy, respiratory inflammation, inflammation of the skin and gastrointestinal inflammation), asthma, Crohn's disease, and inflammatory bowel disease, food allergy, asthma, skin inflammation, emesis, allodynia. hyperalgesia, headache, visceral pain, dental pain, pain associated with burns, menstrual pain, dysmenhorrea, primary dysmenorrhea, rheumatoid arthritis, juvenile rheumatoid arthritis, osteoarthritis, post operative pain (e.g., associated with orthopedic surgery, gynecologic surgery, abdominal surgery, incisions, oral surgery) and back pain, epilepsy and epileptiform-iduced damage, exposure to excitotoxic neurotoxins, excitotoxicity, ischaemic brain damage, cerebral ischaema, traumatic injury (e.g. brain injury), depression, anxiety, sleep disorders, Alzheimer's disease, Parkinson's disease, Huntington's disease, amyotropic lateral sclerosis, multiple sclerosis, tourette-s syndrome, schizophrenia, glaucoma, pain, addiction, inflammation, allergic responses, eating disorders, low blood pressure, hypertension, respiratory problems, cancer (tumour growth), chemotherapy complications, asphyxia, attention deficit disorder, and gastrointestinal diseases, including nausea and vomiting, gastric ulcers, secretory diarrhea, paralytic ileus, inflammatory bowel disease, colon cancer, gastro-oesophageal reflux conditions, pruritus, fatty liver disease, and non-alcoholic steatohepatitis (NASH), and irritable bowel syndrome (IBS).

The invention is also direct to a method of treating a disease selected from acute pain, chronic pain, neurogenic pain, migraine; pain caused by inflammation, and neuropathic pain, anxiety, an eating disorder, obesity, elevated intraocular pressure, glaucoma, a cardiovascular disorder, depression, an inflammatory disorder, asthma, Crohn's disease, and inflammatory bowel disease, food allergy, asthma, skin inflammation, emesis, allodynia. hyperalgesia, headache, visceral pain, dental pain, pain associated with burns, menstrual pain, dysmenhorrea, primary dysmenorrhea, rheumatoid arthritis, juvenile rheumatoid arthritis, osteoarthritis, post operative pain, gynecologic surgery, abdominal surgery, incisions, oral surgery and back pain, epilepsy and epileptiform-iduced damage, exposure to excitotoxic neurotoxins, excitotoxicity, ischaemic brain damage, cerebral ischaema, traumatic injury, depression, anxiety, sleep disorders, Alzheimer's disease, Parkinson's disease, Huntington's disease, amyotropic lateral sclerosis, multiple sclerosis, tourette-s syndrome, schizophrenia, glaucoma, pain, addiction, inflammation, allergic responses, eating disorders, low blood pressure, hypertension, respiratory problems, cancer tumour growth, chemotherapy complications, asphyxia, attention deficit disorder, and gastrointestinal diseases, including nausea and vomiting, gastric ulcers, secretory diarrhea, paralytic ileus, inflammatory bowel disease, colon cancer, gastro-oesophageal reflux conditions, pruritus, fatty liver disease, and non-alcoholic steatohepatitis, and irritable bowel syndrome comprising: administration of a composition a compound according to formula I or II and a second active agent.

Within this genus there is a sub-genus wherein the second active agent is useful for treating osteoarthritis, rheumatoid arthritis, inflammatory pain, neuropathic and nociceptive pain, diabetic neuropathy, postherpetic neuralgia, skeletomuscular pain, and fibromyalgia, as well as acute pain, migraine, sleep disorder, Alzheimer disease, and Parkinson's disease.

Within this sub-genus there is a class wherein the second active agent is useful for treating inflammatory pain, neuropathic and nociceptive pain.

Within this class there is a sub-class wherein the second active agent is etoricoxib.

Alone or in combination with a second active agent, the FAAH inhibitors of the invention are useful in the treatment of a wide range of disorders. Among the disorders: pain (e.g., acute pain, chronic pain, neurogenic pain, migraine; pain caused by inflammation (e.g., arthritis, osteoarthritis, spondylitis, rheumatoid arthritis, Crohn's disease and irritable bowel syndrome), thalamic pain syndrome, and neuropathic pain), anxiety, an eating disorder (e.g., anorexia, bulimia), obesity, elevated intraocular pressure, glaucoma, a cardiovascular disorder, depression, an inflammatory disorder (allergy, respiratory inflammation, inflammation of the skin and gastrointestinal inflammation), asthma, Crohn's disease, and inflammatory bowel disease. Other disorders that can be treated include: food allergy, asthma, skin inflammation, emesis, allodynia. hyperalgesia, headache, visceral pain, dental pain, pain associated with burns, menstrual pain, dysmenhorrea, primary dysmenorrhea, rheumatoid arthritis, juvenile rheumatoid arthritis, osteoarthritis, post operative pain (e.g., associated with orthopedic surgery, gynecologic surgery, abdominal surgery, incisions, oral surgery) and back pain.

Alone or in combination with a second active agent, the FAAH inhibitors of the invention are expected to be useful in the treatment and/or prevention of a wide range of disorders. These FAAH inhibitors are expected to reduce one or more symptoms of one or more such disorders.

Alone or in combination with a second active agent, the FAAH inhibitors of the invention can be used to prevent and/or treat, for example, epilepsy and epileptiform-iduced damage, exposure to excitotoxic neurotoxins, excitotoxicity, ischaemic brain damage, cerebral ischaema, traumatic injury (e.g. brain injury), depression, anxiety, sleep disorders, Alzheimer's disease, Parkinson's disease, Huntington's disease, amyotropic lateral sclerosis, multiple sclerosis, tourette-s syndrome, schizophrenia, glaucoma, pain, addiction, inflammation, allergic responses, eating disorders, low blood pressure, hypertension, respiratory problems, cancer (tumour growth), chemotherapy complications, asphyxia, attention deficit disorder, and gastrointestinal diseases, including nausea and vomiting, gastric ulcers, secretory diarrhea, paralytic ileus, inflammatory bowel disease, colon cancer, gastro-oesophageal reflux conditions, pruritus, fatty liver disease, and non-alcoholic steatohepatitis (NASH). The FAAH inhibitors can also be used to treat irritable bowel syndrome (IBS), a disorder commonly associated with cramping, abdominal pain, bloating, constipation, and diarrhea. There are three major types of IBS: constipation predominant (IBS-C), diarrhea predominant (IBS-D), and alternating (IBS-A) in which constipation and diarrhea both occur.

### Glaucoma and Ocular Disorders

Alone or in combination with a second active agent, the FAAH inhibitors of the invention can be used to prevent and/or treat glaucoma and other disorders characterized by ocular hypertension.

### Sleep Disorders

Alone or in combination with a second active agent, the FAAH inhibitors of the invention can be used to prevent and/or treat a sleep disorder that affects the subject's ability to fall asleep and/or remain asleep, and/or results in unrefreshing sleep. The term “sleep disorder” includes insomnia, night terrors, bruxism, somnambulism, sleep apnea, restless leg syndrome, unrefreshing sleep, seasonal affective disorder, circadian rhythm adjustment disorders, and the like.

Insomnia is typically classed into sleep onset insomnia, where a subject takes more than 30 minutes to fall asleep; and sleep maintenance insomnia, where the subject spends more than 30 minutes awake during an expected sleep period, or for example, waking before the desired wake-up time with an inability to get back to sleep. Sleep disorders include both endogenous disorders, such as sleep apnea, and disorders related to behavioral or external environmental factors. For example, sleep disorders include a subject's difficulty in adjusting to a new circadian rhythm, for example, due to jet lag; night, extended, or irregular work shifts; and the like. A sleep disorder can also arise in a subject that has other disorders, diseases, or injuries, or in a subject being treated with other medications, where the subject as a result has difficulty falling asleep and/or remaining asleep, or experiences unrefreshing sleep. For example, the disclosed method is useful for inducing sleep in a subject having difficulty sleeping as the result of undergoing chemotherapy, or as a result of injuries, or as the result of stress or mood disorders such as depression, anxiety, and the like.

Sleep disorders include conditions recognized by one skilled in the art as sleep disorders—for example, conditions known in the art or conditions which are proposed to be sleep disorders or discovered to be sleep disorders. See, for example. Thorpy. M J International Classification of Sleep Disorders, Revised: Diagnostic and Coding Manual. American Sleep Disorders Association; Rochester, Minn. 1997; and JCD CM, International Classification of Diseases, Ninth Revision, Clinical Modification, National Center for Health Statistics. Hyattsville, Md.

Sleep disorders can be generally classed into dyssoinnias, e.g., intrinsic, extrinsic, and circadian rhythm disorders; parasomnias, e.g., arousal, sleep-wake transition, and rapid eye movement (REM) associated disorders, and other parasomnias; disorders associated with mental, neurological, and oilier medical disorders; and other sleep disorders. Intrinsic sleep disorders include, for example, psychophysiological insomnia, sleep state misperception, idiopathic insomnia, narcolepsy, recurrent hypersomnia, idiopathic hypersomnia, post-traumatic hypersomnia, obstructive sleep apnea syndrome, central sleep apnea syndrome, central alveolar hyperventilation syndrome, periodic limb movement disorder, restless legs syndrome, and the like.

Extrinsic sleep disorders include, for example, inadequate sleep hygiene, environmental sleep disorder, altitude insomnia, adjustment sleep disorder, insufficient sleep syndrome, limit-setting sleep disorder, sleep-upsilonnset association disorder, food allergy insomnia, nocturnal eating (drinking) syndrome, hypnotic-dependent sleep disorder, stiinulaiu-dependent sleep disorder, alcohol-dependent sleep disorder, toxin-induced sleep disorder, and the like.

Circadian rhythm sleep disorders include, for example, time-zone change (jet lag) syndrome, shift work sleep disorder, irregular sleep-wake pattern, delayed sleep phase syndrome, advanced sleep phase syndrome, non 24 h sleep-wake disorder, and the like.

Arousal sleep disorders include, for example, confessional arousals, sleepwalking, sleep terrors, and the like.

Sleep-wake transition disorders include, for example, rhythmic movement disorder, sleep starts, sleeptalking, nocturnal leg cramps, and the like.

REM-associated sleep disorders include, for example, nightmares, sleep paralysis, impaired sleep-related penile erections, sleep-related painful erections, REM sleep-related sinus arrest, REM sleep behavior disorders, and the like.

Other parasomnias include, for example, sleep bruxism, sleep enuresis, sleep-related abnormal swallowing syndrome, nocturnal paroxysmal dystonia, sudden unexplained nocturnal death syndrome, primary snoring, infant sleep apnea, congenital central hyperventilation syndrome, sudden infant death syndrome, benign neonatal sleep myoclonus, and the like. A “sleep disorder” may also arise in a subject that has other medical disorders, diseases, or injuries, or in a subject being treated with other medications or medical treatments, where the subject as a result has difficulty falling asleep and/or remaining asleep, or experiences unrefreshing sleep, e.g., the subject experiences sleep deprivation. For example, some subjects have difficulty sleeping after undergoing medical treatment for other conditions, e.g., chemotherapy or surgery, or as a result of pain or other effects of physical injuries.

It is well known in the art that certain medical disorders, for example, central nervous system (CNS) disorders, e.g., mental or neurological disorders, e.g. anxiety, can have a sleep disorder component, e.g., sleep deprivation. Thus, treating a sleep disorder also includes treating a sleep disorder component of other disorders, e.g., CNS disorders. Further, treating the sleep disorder component of CNS disorders can also have the beneficial effect of ameliorating other symptoms associated with the disorder. For example, in some subjects experiencing anxiety coupled with sleep deprivation, treating the sleep deprivation component also treats the anxiety component. Thus, the present invention also includes a method of treating such medical disorders.

Sleep disorders associated with mental disorders include psychoses, mood disorders anxiety disorders, panic disorder, addictions, and the like. Specific mental disorders include, for example, depression, obsessive compulsive disorder, affective neurosis/disorder, depressive neurosis/disorder, anxiety neurosis, dysthymic disorder, behavior disorder, mood disorder, schizophrenia, manic depression, delirium, alcoholism, and the like.

Sleep disorders associated with neurological disorders include, for example, cerebral degenerative disorders, dementia, parkinsonism, fatal familial insomnia, sleep related epilepsy, electrical status epileptics of sleep, sleep-related headaches, arid the like. Sleep disorders associated with other medical disorders include, fur example, sleeping sickness, nocturnal cardiac ischemia, chronic obstructive pulmonary disease, sleep-related asthma, sleep-related gastroesophageal reflux, peptic ulcer disease, fibrositis syndrome, and the like.

In some circumstances, sleep disorders are also associated with pain, e.g., neuropathic pain associated with restless leg syndrome; migraine; enhanced or exaggerated sensitivity to pain, such as hyperalgesia, causalgia and allodynia; acute pain; bum pain; atypical facial pain: neuropathic pain; back pain; complex regional pain syndromes 1 and 11; arthritic pain: sports injury pain; pain related to infection, e.g., HIV. post-polio syndrome, and post-herpetic neuralgia; phantom limb pain; labor pain; cancer pain; postchemotherapy pain; post-stroke pain, post-operative pain; neuralgia; conditions associated with visceral pain including irritable bowel syndrome, migraine and angina; and the like.

Other sleep disorders include, for example, short sleeper, long sleeper, subwakefulness syndrome, fragmentary myoclonus, sleep hyperhidrosis, menstrual associated sleep disorder, pregnancy-associated sleep disorder, terrifying hypnagogic hallucinations, sleep-related neurogenic tachypnea, sleep-related laryngospasm, sleep choking syndrome, and the like.

Insomnia is typically classed into sleep onset insomnia, where a subject takes more than 30 minutes to fall asleep; and sleep maintenance insomnia, where the subject spends more than 30 minutes awake during an expected sleep period, or, for example, waking before the desired wake-up time with difficulty or an inability to get back to sleep Some of the disclosed compounds are effective in treating sleep onset and sleep maintenance insomnias, insomnia resulting from circadian rhythm adjustment disorders, or insomnia resulting from CNS disorders. In one embodiment, a subject is treated for a circadian rhythm adjustment disorder. In another embodiment a subject is treated for insomnia resulting from a mood disorder. In other embodiments, a subject is treated for sleep apnea, somnambulism, night terrors, restless leg syndrome, sleep onset insomnia, and sleep maintenance insomnia. In other embodiments, a subject is treated for, sleep onset insomnia or sleep maintenance insomnia

Alone or in combination with a second active agent, the FAAH inhibitors of the invention can be used to for inducing, prolonging and/or enhancing sleep. This can encompass the treatment of a sleep disorder, i.e. a difficulty in achieving satisfactory sleep due to some internal or external factor, e.g. pain, stress or anxiety, misuse of stimulants or depressants, or temporary disturbance of lifestyle and it can encompass elective desires on the part of a user to achieve a particularly beneficial period of sleep. Such a desire may, for instance, arise in anticipation of important events the following day or in the near future for which a person may wish to be fully alert and refreshed.

Alone or in combination with a second active agent, the FAAH inhibitors of the invention can help achieve any of the following goals: getting to sleep, especially stage 1 sleep; staying asleep; sleeping well; waking refreshed; waking alert; faster onset to stage 1 sleep; increasing duration of sleep periods; decreasing the number and duration of awakenings; increasing total duration of sleep; increasing probability of sleeping well; reducing insomnia, especially chronic or mild-moderate insomnia; decreasing disturbances during sleeptime; and improving quality of sleep. Meeting these goals can be determined by any standard or, known subjective or objective measures, for instance the Karolinska scale, Loughborough sleep log or actimetry.

Improved sleep can assist in keeping awake; keeping alert; keeping refreshed; and performing well the next day.

The degree of refreshedness and quality of sleep may be determined by the—morning-log of the Loughborough sleep log with the highest degree of refreshedness or quality of sleep being represented as 1 and the lowest being represented as 5. Accordingly, the percentage increase in refreshedness or quality of sleep is measured in this context by the decrease in the mean refreshedness or quality of sleep.

The response of feeling extremely alert, very alert or alert can be determined, for instance, by the Karolinska 9-point scale. Other measures of sleep parameters include the sleep disturbance index (SDI) and time to sleep onset (TTSO) that can both be measured by actimetry.

The FAAH inhibitors of the invention can be used in combination with therapies currently used for the treatment of sleep disorders, e.g., Aldesleukin (Proleukin), Amantadine (Symmetrel). Baclofen (Lioresal). Bepridil (Vascor), Carisoprodol (Soma), Clonazepam (Klonopin), Diazepam (Valium), Diphenhydramine (Sominex, Nytoi), Doxylamipie (Unisom). Estazolam (ProSoni). Flurazepam (Dalmane), Gabapentin, Lorazepam (Ativan). Le vod opacarb idopa (Sinemet), Melatonin. Methylphenidate (Ritalin), Modanfinil (Provigil), Pemoline (Cylert), Pergolide, Pramipexoie, Pronietliazine (Phenergan), Quazepam (Doral), Rimantadine (Flumadine), Sibutxamuie (Meridia), Sodium oxybate, Synthetic conjugated estrogens (Cenestin), Temazepam (Restoril), Triazolam (Halcion), Zaleplon (Sonata), and Zolpidem (Ambien).

### Obesity Related Disorders

Alone or in combination with a second active agent, the FAAH inhibitors of the invention may be used to treat obesity and/or to reduce or control body weight (or fat) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). Obesity is a condition in which there is an excess of body fat. In many cases, an individual is considered obese if the individual has a body mass index (BMA) greater than or equal to 30 kg/nr or if the individual has at least one co-morbidity and a BMI greater than or equal to 27 kg/m². In certain situations, a subject at risk for obesity is an otherwise healthy subject with a BMI of 25 kg/m² to less than 30 kg/m² or a subject with at least one co-rnorbidiiy with a BMI of 25 kg/m² to less than 27 kg/in².

The increased risks associated with obesity agree thought to occur at a lower BMI in Asians. In some situations, obesity in an Asian refers to a condition whereby a subject with at least one obesity-induced or obesity-related co-morbidity that requires weight reduction or that would be improved by weight reduction, has a BMI greater than or equal to 25 kg/nr. In Asians, an obese subject sometimes refers to a subject with at least one obesity-induced or obesity-related co-morbidity that requires weight reduction or that would be improved by weight reduction, with a BMI greater than or equal to 25 kg/m². In some situations, an Asian at risk of obesity is a subject with a BMI of greater than 23 kg/m² to less than 25 kg/m².

Obesity-induced or obesity related co-morbidities include, but are not limited to diabetes, noninsulin dependent diabetes mellitus type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver, cerebral infarction, cerebral, thrombosis, transient ischemic attack, orthopedic disorders, arthritis deformans, lumbodynia, emmeniopatliy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment (of obesity and obesity-related disorders) refers to the administration of the compounds described herein to reduce or maintain the body weight of an obese subject. One outcome of treatment may be reducing the body weight of an obese subject relative to that subject's body weight immediately before the administration of the compounds described herein. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention (of obesity and obesity-related disorders) refers to the administration of the compounds described herein to reduce or maintain the body weight of a subject at risk of obesity. One outcome of prevention may be reducing the body weight of a subject at risk of obesity relative to that subject's body weight immediately before the administration of the compounds described herein. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis. Obesity-related disorders are disorders that are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrhythmias, myocardial infarction, congestive heart failure, coronary heart disease, sudden death, stroke, polycystic ovarian disease, craniopharyngioma, the Prader-Willi Syndrome, Frupsilonhhch's syndrome, GH-deficie. it subjects, normal variant short stature, Turner's syndrome, and other pathological conditions showing reduced metabolic activity or a decrease in resting energy expenditure as a percentage of total fat-free mass, e.g., children with acute lymphoblastic leukemia. The compounds described herein may be used to reduce or control body weight (or fat) or to prevent and/or treat obesity or other appetite related disorders related to the excess consumption of food, ethanol and other appetizing substances. The compounds may be used to modulate lipid metabolism, reduce body fat (e.g. via increasing fat utilization) or reduce (or suppress) appetite (e.g. via inducing satiety).

Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastroesophageal reflux, respiratory disorders, such as obesity-hypoventilatlon syndrome (Pickwickian syndrome), cardiovascular disorders, inflammation, such as systemic inflammation of the vasculature, arteriosclerosis, hypercholesterolemia, hyperuricaemia, lowerback pain, gallbladder disease, gout, and kidney cancer. The compounds described herein are also useful for reducing the risk of secondary outcomes of obesity, such as reducing the risk of left ventricular hypertrophy.

The FAAH inhibitors of the invention can be administered in combination with anti-obesity agents, including, but not limited to: 11β HSD-I (11-beta hydroxy steroid dehydrogenase type 1) inhibitors, such as BVT 3498, BVT 2733, 3-(1-adamantyl)-4-ethyl-5-(ethyl thio)-4H-1,2,4-Triazole. 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO01/90091, WO01/90090, WO01/90092 and WO02/072084; 5HT (serotonin) transporter inhibitors, such as paroxetine, fluoxetine, fenfluramine, fluvoxamine. sertraline, and imipramipie. and those disclosed in WO003/00663; 5HT antagonists such as those in WO03/037871. WO03/037887, and the like; 5HT1a modulators such as those disclosed in WO03/031439, and the like; 5HT-2 agonists; 5HT2c (serotonin receptor 2c) agonists, such as BVT933, DPCA37215, IK264, PNU 22394, WAY161503, R-1065, and YM 348 and those disclosed in U.S. Pat. No. 3,914,250 and PCT publication Nos. WO02/36596. WO02/48124, WO02/10169, WO01/66548. WO02/44152. WO02/51844, WO02/40456, and WO02/40457: 5HT6 receptor modulators, such as those in WO03/030901, WO03/035061. WO03/039547, and the like, ACC2 (acetyl-CoA carboxylase-2) inhibitors; acyl-estrogens, such as oleoyl-estrone, disclosed in del Mar-Crasa. M. et al., Obesity Research, 9:202-9 (2001) and Japanese Patent Application No. JP 2000256190; alpha-lipoic acid (alpha-LA); anorectic bicyclic compounds such as 1426 (Avepitis) and 1954 (Aventis), and the compounds disclosed in WO00/18749. WO01/32638, WO01/62746, WO01/62747. and WO03/015769; AOD9604; appetite suppressants such as those in WO03/40107; ATL-962 (Alizymc PLC); benzocaine:bcnzphetamine hydrochloride (Didrex).bladderwrack (focus vesiculosus); BKS3 (bombesin receptor subtype 3) agonists; bupropion; caffeine; CB 1 (cannabinoid-1 receptor) antagonist/inverse agonists, such as rimonabant (Acomplia; Sanofi Synthelabo). SR-147778 (Sanofi Synthelabo), BAY 65-2520 (Bayer), and SLY 319 (Solvay), and those disclosed in U.S. Pat. Nos. 4,973,587, 5,013,837, 5,081,122, 5,112,820, 5,292,736, 5,532,237, 5,624,941, 6,028,084, and 6,509,367 and WO96/33159, WO97/29079, WO98/31227. WO98/33765, WO98/37061. WO98/41519, WO98/43635, WO98/43636, WO99/02499, WO00/10967. WO00/10968. WO01/09120, WO01/58869, WO01/64632, WO01/64633. WO01/64634, WO01/70700. WO01/96330. WO02/076949, WO03/006007, WO03/007887, WO03/020217, WO03/026647, WO03/026648, WO03/027069, WO03/027076. WO03/027114, WO03/037332, WO03/040107, WO03/086940, WO03/084943 and U.S. Pat. No. 6,509,367 and EPO Application No. EP-658546; CCK agonists; CCK-A (cholecystokinin-A) agonists, such as AR-R 15849, GI 181771, JMV-180, A-71378. A-71623 and SR146131, and those described in U.S. Pat. No. 5,739,106; chitosan: chromium; CNTF (Ciliary neurotrophic factors), such as Cl-181771 (Glaxo-SmitliKline), SR146131 (Sanofi Synthelabo), butabindide, PD170292, and PD 149164 (Pfizer); CNTF derivatives, such as axokine (Regeneron), and those disclosed in PCT Application Nos. WO 94/09134, WO 98/22128, and WO 99/43813: conjugated linoleic acid; corlicotropin-releasing hormone agonists; dehydroepiandrosterone; DGAT1 (diacylglycerol acyltransferase 1) inhibitors; DGAT2 (diacylglycerol acyltransferase 2) inhibitors; dicarboxylate transporter inhibitors; diethylpropion hydrochloride (Tenuate); dipeptidyl peptidase IV (DP-IV) inhibitors, such as isoleucine thiazolidine, valine pyrrolidine, NVP-DPP728. LLAMDAF237, P93/01. TSL 225. TMC-2A/2B/2C. FE 999011. P9310/K364. VIP 0177. SDZ 274-444. sitagliptin and the DP-IV inhibitor compounds disclosed Pratley and Salsali (2007) Curr Med Res Opin. 23:919-31 and the compounds disclosed in PCT publication Nos. WO02/083128, WO02/062764, WO03/000180. WO03/000181, WO03/000250. WO03/002530. WO03/002531, WO03/002553, WO03/002593, WO03/004498, WO03/004496.WO03/017936, WO03/024942, WO03/024965, WO03/033524, WO03/037327 and EP1258476; ephedra; exendin-4 (an inhibitor of glp-1); FAS (fatty acid synthase) inhibitors, such as Cerulenin and C75; fat resorption inhibitors such as those in WO03/053451 and the like; fatty acid transporter inhibitors; fiber (psyllium, plantago. guar fiber); galanin antagonists; galega (Goat's Rue. French Lilac); garcinia cambogia; germander (teucrium chamaedrys); ghrelin antagonists, such as those disclosed in PCT Application Nos. WO 01/87335, and WO 02/08250; GLP-I (glucagon-like peptide 1) agonists (e.g. exendin-4); glp-1 (glucagon-like peptide-1); glucocorticoid antagonists; glucose transporter inhibitors; growth hormone secretagogue receptor agonists/antagonists, such as NN703. hexarelin, MK-0677. SM-5 130686, CP-424.391, L-G92.429 and L-163.255. and such as those disclosed in U.S. Pat. No. 6,358,951, U.S. Patent Application Nos. 2002/049196 and 2002/022637, and PCT Application Nos. WO 01/56592 and WO 02/32888; growth hormone secretagogues, such as those disclosed and specifically described in U.S. Pat. No. 5,536,716; H3 (histamine 113) antagonist/inverse agonists, such as thioperamide. 3-(1M-imidazol 4-gammal)propyl N-(4-pentenyl)carbamate), clobenpropit, iodophenpropit, imoproxifan, GT2394 (Gliatech), and A331440, and those disclosed in PCT publication No. WO02/15905 and O-[3-(1H-imidazol-4-yl)propanol]carbamates (Kiec-Kononowicz, K. et al., Pharmazie, 55:349-55 (2000)), piperidine-containing histamine H3 receptor antagonists (Lazewska, D. et al., Phapinazie, 56:927-32 (2001), benzophenone derivatives and related compounds (Sasse, 15 A. et al., Arch. Pharm. (Weinheim) 334:45-52 (2001)), substituted N-plienylcarbamates (Reidemeister, S. et al., Pharmazie, 55:83-6 (2000)). and proxifan derivatives (Sasse, A. et al., J. Med. Chem. 43:3335-43 (2000)) arid histamine H3 receptor modulators such as toile disclosed in WO03/024928 and WO03/024929; interleukin-6 (II.-6) and modulators thereof, as in WO03/057237, and the like; L carnitine; leptin derivatives, such as those disclosed in U.S. Pat. Nos. 5,552,524, 5,552,523, 5,552,522, 5,521,283, and PCT International Publication Nos., WO96/23513, WO96/23514, WO96/23515, WO96/23516, WO96/23517, WO96/23518, WO96/23519 and WO96/23520; leptin, including recombinant human leptin (PEG-OB, Hoffman La Roche) and recombinant methionyl human leptin (Amgen); lipase inhibitors, such as tetrahydrolipstatin (orlistat/Xenical®), Triton WR 1339, RHC80267, lipstatin, teasaponin, and didhyiumbelliferyl phosphate, FL-386, WAY-121898, Bay-N-3176, valilactone, esteracin, ebelactonc A, ebclactone B, and RHC 80267, and those disclosed in PC T Publication Nos. WO01/77094, and U.S. Pat. Nos. 4,598,089, 4,452,813, 5,512,565, 5,391,571, 5,602,151, 4,405,644, 4,189,438, and 4,242,453: lipid metabolism modulators such as maslinic acid, erythrodiol, ursolic acid uvaol, betulinic acid, betulin, and the like and compounds disclosed in WO03/011267; Mc3r (melanocortin 3 receptor) agonists; Mc4r (melanocortin 4 receptor) agonists, such as CHIR86036 (Chiron), ME-10142, ME-10145, and HS-131 (Melacure), and those disclosed in PCT Publication Nos. WO99/64002, WO00/74679, WO01/991752, WO01/25192, WO01/52880, WO01/74844, WO01/70708, WO01/70337, WO01/91752, WO02/059095, WO02/059107, WO02/059108, WO02/059117, WO02/06276, WO02/12160, WO02/11715, WO02/12178, WO02/15909, WO02/38544, WO02/068387, WO02/068388, WO02/067869, WO02/081430, WO03/06604, WO03/007949, WO03/009847, WO03/009850, WO03/013509, and WO03/031410; McSr (melanocortin 5 receptor) modulators, such as those disclosed in WO97/19952, WO00/15826, WO00/15790, US 20030092041: MCH2R (melanin concentrating hormone 2R) agonist/antagonists; melanin concentrating hormone antagonists; melanin-concentrating hormone 1 receptor (MCHR) antagonists, such as T-226296 (Takeda), SNP-7941 (Synapic), and those disclosed WO01/21169, WO01/82925, WO01/87834, WO02/051809, WO02/06245, WO02/076929, WO02/076947, WO02/04433, WO02/51809, WO02/083134, WO02/094799, WO03/004027, WO03/13574, WO03/15769, WO03/028641, WO03/035624, WO03/033476, WO03/033480 and Japanese Patent Application Nos. JP 13226269, and JP1437059; melanocortin agonists, such as Melanotan 11 or those described in WO 99/64002 and WO 00/74679; Metformin (Glucopliage®); mGluRS modulators such as those disclosed in WO03/029210, WO03/047581, WO03/048137, WO03/051315, WO03/051833, WO03/053922, WO03/059904, and the like; monoamine reuptake inhibitors, such as sibutralrnine (Meridia®/Reductil®) and salts thereof, and those compounds disclosed in U.S. Pat. Nos. 4,746,680, 4,806,570, and 5,436,272, and U.S Patent Publication No. 2002/0006964. and WO01/27068, and WO01/62341; NE (norepinephrine) transport inhibitors, such as GW 320659, despiramine, talsupram, and nomifensine; nomame herba; non-selective serotonin/nupsilonrepinephrine transport inhibitors, such as sibutramine or fenfluramine; NPY 1 antagonists, such as BIBP3226, J-115814, BIBO 3304, LY-357897, CP-671906, G1-264879A, and those disclosed in U.S. Pat. No. 6,001,836, and PCT Patent Publication Nos. WO96/14307, WO01/23387, WO99/51600, WO01/85690, WO01/85098, WO01/85173, and WO01/89528; NPY5 (neuropeptide Y Y5) antagonists, such as 152,804, GW-569180A, GW-594884A, GW-587081X, CW-548118X, FR235208, FR226928, FR240662, FR252384, 1229U91, Cl-264879A, CGP71683LAMDA, LY-377897, LY 366377, PD-160170, SR-120562LAMDA, SR-120819LAMDA, JCF-104, and H409/22 and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683, European Patent Nos. EP-0.010691, and EP-01044970 and PCT Publication Nos. WO97/19682, WO97/20820, WO97/20821, WO97/20822. WO97/20823, WO98/27063, WO00/107409, WO00/185714, WO00/185730, WO00/64880, WO00/68197, WO00/69849, WO01/09120, WO01/14376, WO01/85714, WO01/85730, WO01/07409, WO01/02379, WO01/23388, WO01/23389, WO01/44201. WO01/62737, WO01/62738, WO01/09120, WO02/20488, WO02/22592, WO02/48152, WO02/49648, WO02/051806, WO02/094789, WO03/009845, WO03/014083, WO03/022849, WO03/028726 and Norman et al., J. Med. Cliern. 43:4288-4312 (2000); opioid antagonists, such as nalmefene (Revex®), 3-methoxynaItrexone, naloxone, and naltrexone and those disclosed in WO00/21509; orexin antagonists, such as SB-334867-A and those disclosed in PCT Publication Nos. WO01/96302, WO01/68609, WO02/44172, WO02/51232, WO02/51838, WO02/089800, WO02/090355, WO03/023561, WO03/032991, and WO03/037847; PDE (phosphodiesterase) inhibitors, such as theophylline, pentoxifylline, zaprinast, sildenafil, anirinone, milrinone, cilostamide, rolipram, and cilomilast; peptide YY and fragments and variants thereof (e.g. YY₃₋₃₆ (PYY₃₋₃₆)(N. Engl. J. Med. 349:941, 2003; ikpeapge daspeelnry yaslrliylnl vtrqry) and PYY agonists such as those disclosed in WO03/026591; phendimetraxine: phentermine, phosphate transporter inhibitors; phosphodiesterase-3B (PDE3B) inhibitors; phytophapin compound 57 (CP 644.673); pyruvate; SCD-I (stearoyl-CoA desaturase-1) inhibitors; serotonin reuptake inhibitors, such as dexfenfluramine, fluoxetine, and those in U.S. Pat. No. 6,365,633, and WO01/27060, and WO01/162341; T71 (Tularik; Inc; Boulder Colo.); thyroid hoimone beta agonists, such as KB-2611 (KaroBioBMS), and those disclosed in WO02/15845 and Japanese Patent Application No. JP 2000256190; Topiramate (Topimax®): transcription factor modulators sucli as those disclosed in WO03/026576: UCP-I (uncoupling protein-1), 2, or 3 activators, such as phytanic acid, 4-((E)-2-(5,6,7,8-tetrahydro-5,5,8,8-tetramethyl-2-napthalenyl)-1-propeny-1)benzoic acid (TTNPB), retinoic acid, and those disclosed in PCT Patent Application No. WO 99/00123; β3 (beta adrenergic receptor 3) agonists, such as AD9677/TAK677 (Dainlppon/Takeda), CL-316,243, SB 418790, BRL-37344, L-796568, BMS-196085, BRL-35135LAMDA, CGP12177A, BTA-243, GW 427353, Trecadrine, Zeneca D7114, N 5984 (Nisshin Kyorin), LY-377604 (Lilly), and SR 59119lamda, and those disclosed in U.S. Pat. Nos. 5,705,515, U.S. Pat. No. 5,451,677 and PCT Publication Nos. WO94/18161, WO95/29159, WO97/46556, WO98/04526, and WO98/32753, WO01/74782, WO02/32897, WO03/014113, WO03/016276, WO03/016307, WO03/024948, WO03/024953, and WO03/037881; beta-hydroxy steroid dehydrogenase-1 inhibitors (beta-HSD-I); beta-hyclroxy-beta-methylbutyrate.

### Anxiety Related Disorders

Alone or in combination with a second active agent, the FAAH inhibitors of the invention can be used to treat anxiety disorder (including generalized anxiety disorder, panic disorder, and social anxiety disorder) and depression. Anxiety disorders are a group of psychological problems whose key features include excessive anxiety, fear, worry, avoidance, and compulsive rituals, and produce or result in inordinate morbidity, over utilization of healthcare services, and functional impairment. They are among the most prevalent psychiatric conditions in the United States and in most other countries. Anxiety disorders listed in the Diagnostic and Statistical Manual of Mental Disorders (Fourth Edition, Revised 1994, published by the American Psychiatric Association, Washington, D C. pages 393-444) include panic disorder with and without agoraphobia, agoraphobia without history of panic disorder, specific phobia, social phobia, obsessive-compulsive disorder (OCD), post-traumatic stress disorder (PTSD), acute stress disorder, generalized anxiety disorder (GAD), anxiety disorder due to a general medical condition, substance-induced anxiety disorder, specific phobia, and anxiety disorder not otherwise specified.

Obsessive compulsive disorder is characterized by recurrent and persistent ideas, thoughts, impulses or images (obsessions) that are ego-dystonic and/or repetitive, purposeful and intentional behaviors (compulsions) that are recognized by the person as excessive or unreasonable. The obsessions or compulsions cause marked distress, are time-consuming, and/or significantly interfere with social or occupational functioning.

Panic disorder is characterized by recurrent unexpected panic attacks and associated concern about having additional attacks, worry about the implications or, consequences of the attacks, and/or a significant change in behavior related to the attacks. A panic attack is defined as a discrete period of intense fear or discomfort in which four (or more) of the following symptoms develop abruptly and reach a peak within 10 minutes: (1) palpitations, pounding heart, or accelerated heart rate; (2) sweating; (3) trembling or shaking; (4) sensations of shortness of breath or smothering; (5) feeling of choking; (6) chest pain or discomfort; (7) nausea or abdominal distress; (8) feeling dizzy unsteady, lightheaded, or faint; (9) derealization (feelings of unreality) or depersonalization (being detached from oneself): (10) fear of losing control; (11) fear of dying; (12) parenthesis (numbness or tingling sensations); and (13) chills or hot flushes. Panic disorder may or may not be associated with agoraphobia, or an irrational and often disabling fear of being out in public.

Social anxiety disorder, also known as social phobia, is characterized by a marked and persistent fear of one or more social or performance situations in which the person is exposed to unfamiliar people or to possible scrutiny by others. Exposure to the feared situation almost invariably provokes anxiety, which may approach the intensity of a panic attack. The feared situations are avoided or endured with intense anxiety or distress. The avoidance, anxious anticipation, or distress in the feared situation (s) interferes significantly with the person's normal routine, occupational or academic functioning, or social activities or relationships, or there is marked distress about having the phobias. Lesser degrees of performance anxiety or shyness generally do not require psychopharmacological treatment.

Generalized anxiety disorder is characterized by excessive anxiety and worry (apprehensive expectation) that is persistent for at least 6 months and which the person finds difficult to control. It must be associated with at least 3 of the following 6 symptoms: restlessness or feeling keyed up or on edge, being easily fatigued, difficulty concentrating or mind going blank, irritability, muscle tension, and sleep disturbance.

The diagnostic criteria for this disorder are described in further detail in DSM-IV, which is incorporated herein by reference (American Psychiatric Association, 1994).

Post-traumatic stress disorder (PTSD), as defined by DSM111-R/IV, requires exposure to a traumatic event that involved actual or threatened death or serious injury, or threat to the physical integrity of self or others, and a response which involves intense fear, helplessness, or horror. Symptoms that occur as a result of exposure to the traumatic event include re-experiencing of the event in the form of intrusive thoughts, flashbacks or dreams, and intense psychological distress and physiological reactivity on exposure to cues to the event; avoidance of situations reminiscent of the traumatic event, inability to recall details of the event, and/or numbing of general responsiveness manifested as diminished interest in significant activities, estrangement from others, restricted range of affect, or sense of foreshortened future: and symptoms of autonomic arousal including hypervigilance, exaggerated startle response, sleep disturbance, impaired concentration, and irritability or outbursts of anger. A PTSD diagnosis requires that the symptoms are present for at least a month and that they cause clinically significant distress or impairment in social, occupational, or other important areas of functioning.

Alone or in combination, it is contemplated that the compounds will be effective in treating obsessions and compulsions in patients who have been diagnosed as having obsessive compulsive disorder based upon administration of appropriate tests, which may include, but are not limited to any of the following: Yale Brown Obsessive Compulsive Scale (YBOCS) (for adults), National Institute of Mental Health Global OCD Scale (NIMH GOCS), and CGl-Severity of Illness scale. It is further contemplated that the compounds will be effective in inducing improvements in certain of the factors measured in these tests, such as a reduction of several points in the YBOCS total score. It is also contemplated that the compounds of this invention will be effective in preventing relapse of obsessive-compulsive disorder.

Alone or in combination with a second active agent, the FAAH inhibitors of the invention will be effective in treating panic disorder in patients who have been diagnosed with panic disorder on the basis of frequency of occurrence of panic attacks, or by means of the CGI-Severity of Illness scale. It is further contemplated that the compounds described herein will be effective in inducing improvements in certain of the factors measured in these evaluations, such as a reduction in frequency or elimination of panic attacks an improvement in the CGI-Severity of Illness scale or a CGI Global Improvement score of 1 (very much improved), 2 (much improved) or 3 (minimally improved). It is also contemplated that the compounds of this invention will be effective in-preventing relapse of panic disorder.

Alone or in combination with a second active agent, the FAAH inhibitors of the invention will be effective in treating social anxiety disorder in patients who have been diagnosed as having social anxiety disorder based upon the administration of any of the following tests: the Liebowitz Social Anxiety Scale (LSAS), the CGI-Severity of Illness scale, the Hamilton Rating Scale for Anxiety (HAM-A), the Hamilton Rating Scale for Depression (HAM-D), the axis V Social and occupational Functioning Assessment Scale of DSM-IV, the axis II (ICD10) World-Health organization Disability Assessment, Schedule 2 (DAS-2), the Sheehan Disability Scales, the Schneier Disability Profile, the World Health Organization Quality of Life-100 (WHOQOL-100)), or other tests as described in Ballenger. J C et al. 1998, J Clin Psychiatry 59 Suppl 17:54-60., which is incorporated herein by reference. It is further contemplated that the FAAH inhibitors of the invention will be effective in inducing improvements as measured by these tests, such as the a change from baseline in the Liebowitz Social Anxiety Scale (LSAS)₁ or a CGI-Global Improvement score of 1 (very much improved), 2 (much improved) or 3 (minimally improved). It is also contemplated that the FAAH inhibitors of the invention will be effective in preventing relapse of social anxiety disorder.

Alone or in combination with a second active agent, the FAAH inhibitors of the invention will be effective in treating generalized anxiety disorder in patients who have been diagnosed as having this disorder based upon the diagnostic criteria described in DSM-IV. It is further contemplated that the compounds described herein will be effective in reducing symptoms of this disorder, such as the following: excessive worry and anxiety, difficulty controlling worry, restlessness, or feeling keyed up or on edge, being easily fatigued, difficulty concentrating or mind going blank, irritability, muscle tension, or sleep disturbance. It is also contemplated that the compounds of this invention will be effective in preventing relapse of general anxiety disorder.

Alone or in combination with a second active agent, the FAAH inhibitors of the invention will be effective in treating PTSD in patients who have been diagnosed as having PTSD based upon the administration of any of the following tests: Clinician Administered PTSD Scale Part 2 (CAPS) and the patient-rated Impact of Event Scale (IES). It is further contemplated that the compounds described herein will be effective in inducing improvements in the scores of the CAPS, IES, CCI-Severity of Illness or CGI-Global Improvement tests. It is also contemplated that the compounds of this invention will be effective in preventing relapse of PTSD.

Alone or in combination with a second active agent, the FAAH inhibitors of the invention may be used to prevent, control, or treat schizophrenia, paranoia or other related disorders of dopamine transmission.

The FAAH inhibitors of the invention can be administered in combination with anti-anxiety agents. Classes of anti-anxiety agents include: benzodiazepines (e.g. alprazolam (Xanax®), chlordiazepoxide, clonazepam, chlorazepate, diazepam, halazeparn. lupsilonrazepam, oxazeprarn, and prazepam. and pharmaceutically acceptable sails thereof); 5-HT1 A agonist or antagonist, especially 5HT1A partial agonists (e.g. the 5-HT1A receptor partial agonists buspirone, flesinoxan, gepirone and ipsapirone, and pharmaceutically acceptable salts thereof): corticotropin releasing factor (CRF) antagonists (including those described in WO 94/13643. WO 94/13644, WO 94/13661. WO 94/13676, and WO 94/13677); phenotliiazines (including promethazine. chlorpromazine. and trifluoperazine); monoamine oxidase inhibitors (MAOIs, e.g. isocarboxazid (Marplan®), phenelzine (Nardil®), tranylcypromine (Parnate®) and selegiline, and pharmaceutically acceptable salts thereof); reversible inhibitors of monoamine oxidase (RIMAs, e.g. moclobemide and pharmaceutically acceptable salts thereof); tricyclic antidepressants (TCAs, e.g. amitriptyline (Elavil®), anioxapine, clomipramine, desipramine (Norpramin®), doxepin, imiprainine (Tofrani®), maptroline, nortriptyline (Aventyl® and Pamelor®), perphenazine, protriptyline, and trimipramine (Surmentil®) and pharmaceutically acceptable salts thereof)): atypical antidepressants including bupropion, lithium, nefazodone, trazodone and viloxazine, and pharmaceutically acceptable salts thereof; and selective serotonin reuptake inhibitors (SSRIs, e.g. paroxetine (Paxil®), venlafaxine, fluvoxamine, fluoxetine (Prozac®), citalopram (Celexa®), escitaloprain, and sertraline (Zoloft®) and pharmaceutically acceptable salts thereof).

The FAAH inhibitors of the invention can also be used in a co-therapy with a second agent that has analgesic activity. Analgesics which can be used in co-therapy include, but are not limited to: NSAIDs (e.g., acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fendofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indonietliacin, indoprofen, isoxepac, isoxicam, ketoprofen, keloprupsilonfen, ketorolac, inedofenajnic acid, meclofenamic acid, niefenaniic acid, mefenamic acid, miroprofen, rpiofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam. pirprofen, pranoprofen, sudoxicam, tenoxican, sulfasalazine sulindac, suprofen, tiaprofenic acid, tiopinac, tioxaprofen, tolfenamic acid, tolmetin, tolmetin. zidometacin, zomepirac, and zomepirac), a non-narcotic analgesic such as tramadol, an opioid or narcotic analgesic (e.g., APF112, beta funaltrexamine, buprenorphine, butorphanupsilonl, codeine, cypridime, dezocine, dihydrocodeine, diphenyloxylate, enkephalin pentapeptide, fedotozinc, fentanyl, hydrocodone, hydromophihone, levorphanol, loperamide, meperidine, mepivacaine, methadone, methyl nalozone, morphine, nalbuphine, nalmefene, naloxonazine, naloxone, naltrexone, naltrindole, nor-binaltorphimlne, oxycodone, oxymorphone, pentazocine, propoxyphene, and trimebutine). NKI receptor antagonists (e.g., ezlopitant and SR-14033, SSR-241585). CCK receptor antagonists (e.g., loxiglumide), NK3 receptor antagonists (e.g., talnetant, osanetant SR-HZSOl₁ SSR-ZdISSS), norepinephrine-serotonin reuptake inhibitors (NSRI; e.g., milnacipran), vanilloid receptor agonists and antagonists, cannabinoid receptor agonists (e.g., arvanil), sialorphin, compounds or peptides that are inhibitors of neprilysin Frakefamide (H-Tyr˜D-Ala-Phe(F)-Phe-NH₂; WO 01/019849 A1), Tyr-Arg (kyotorphin), CCK receptor agonists (e.g., caerupsilonlein), conotoxin peptides, peptide analogs of thymulin, dexloxiglumide (tlie R-isomer of loxiglumide: WO 88/05774), and analgesic peptides (e.g., endomupsilonrphin-1, endomorphin-2. nocistatin, dalargin, luprupsilonn, and substance P).

In addition, certain antidepressants can be used in co-therapy either because they have analgesic activity or are otherwise beneficial to use in combination with an analgesic. Examples of such anti-depressants include: selective serotonin reuptake inhibitors (e.g., fluoxetine, paroxetine, sertraline), serotonin-norepinephrine dual uptake inhibitors, venlafaxine and nefazadone. Certain anti-convulsanls have analgesic activity and are useful in co-therapy. Such anticonvulsants include: gabapentin, carbamazepine, phenyloin, valproate, clonazepam, topiramate and lamotrigine. Such agents are considered particularly useful for treatment of neuropathic pain, e.g., treatment of trigeminal neuralgia, postherpetic neuralgia, and painful diabetic neuropathy. Additional compounds useful in co therapy include: alpha-2-adrenergic receptor agonists (e.g., tizanidine and clonidine), mexiletine. corticosteroids, compounds that block the NMDA (iN-methyl-Daspartate) receptor (e.g. dextromethorphan, ketamine, and amantadine). glycine antagonists, carisoprodol, cyclobenzaprine, various opiates, non-mu opioid antitussive (e.g. dextromethorphan, capiniphen, caramiphen and carbetapentane), opioid antitussives (e.g. codeine, hydrocodone, metaxolone. The compounds described herein can also be combined with inhalable gaseous nitric oxide (for treating pulmonary vasoconstriction or airway constriction), a thromboxane A2 receptor antagonist, a stimulant (i.e. caffeine), an H₂-antagonist (e.g. ranitidine), an antacid (e.g. aluminum or magnesium hydroxide), an antiflatulent (e.g. simethicone), a decongestant (e.g. phenylephrine, phenylpropanolamine, pseudophedrine, oxymetazoline, ephinephrine, naphazoline, xylometazoline. propylhexedrine, or levodesoxyephedrine). a prostaglandin (e.g. misoprostol, enprostil, rioprostil, oxoprostol or rosaprostol), a diuretic, a sedating or non-sedating histamine HI receptor antagonists/anlihistamines (i.e. any compound that is capable of blocking, inhibiting, reducing or otherwise interrupting the interaction between histamine and its receptor) including but not limited to: 4 astemizole, acrivastine, antazoline, astemizole, azatadine, azelasline, aslamizole, brompheniramine, brompheniramine maleate, carbinoxamine, carebastine, cetirizine, chlorpheniramine, chlorpheniramine maleate, cimetidine, clemastine, cyclizine, cyproheptadine, descarboethoxyloratadine, dexchlorpheniramine, dimethindene, diphenhydramine, diphenylpyraline, doxylamine succinate, doxylamine, ebastine, efletipizine, epinastine, famotidine, fexofenadine, hydroxyzine, hydroxyzine, ketotifen, levocabastine, levocetirizine, levocetiriziiie, loratadine, meclizine, mepyramine, mequitazine, methdilazine, mianserin, mizolastine, noberastine, norasternizole, iioraztoinizole, phenindamine, pheniramine, picumast, promethazine, pynlamine, pyrilamiiie, ianitidine, temelastine, terfenadine. trimeprazine, tripelenamine, and triprolidine; a 5HT1 agonist, such as a triptan (e.g. sumatriptan or naratriplan), an adenosine A1 agonist, an HP ligaml. a sodium channel blocker (e.g. lamotrigine), a substance P antagonist (e.g. an NK antagonist), a cannabinoid, a 5-lipoxygenase inhibitor, a leukotriene receptor antagonist/leukotriene antagonists/LTD4 antagonists (i.e, any compound that is capable of blocking, inhibiting, reducing or otherwise interrupting the interaction between leukotrienes and the Cys LT1 receptor) including but not limited to: zafirlukast, montelukast, montelukast sodium (Singulair?), pranlukast, iralukast, pobilukast. SKB-106,203 and compounds described as having LTD4 antagonizing activity described in U.S. Pat. No. 5,565,473, a DMARU (e.g. methotrexate), a neurone stabilising antiepileptic drug, a mono-aminergic uptake inhibitor (e.g. venlafaxine), a matrix metal loproteinase inhibitor, a nitric oxide, synthase (NOS) inhibitor, such as an iNOS or an nNOS inhibitor, an inhibitor of the release, or action, of tumor necrosis factor, an antibody therapy, such as a monoclonal antibody therapy, an antiviral agent, such as a nucleoside inhibitor (e.g lamivudine) or an immune system modulator (e.g. interferon), a local anaesthetic, a known FLAMDALAMDAH inhibitor (e.g., PMSF, UKB532, URB597, or BMS-I, as well as those described in those described in WO04033652, U.S. Pat. No. 6,462,054, US 2003/0092734, US 2002/0188009, US 2003/0195226, and WO04/033422), an antidepressant (e.g., VPI-013), a fatty acid amide (e.g. anandamide, N palmitoyl ethanolamine. N-oleoyl ethanoiamide, 2-arachidonoylglycerol. or oleamide). arvanil, analogs of anadamide and arvanil as described in US 20040122089, and a proton pump inhibitor (e.g., omeprazole, esomeprazole, lansoprazole, pantorazole and rabeprazole).

The FAAH inhibitors of the invention can also be used in a co-therapy with a second agent that is a cannabinoid receptor antagonist to prevent and/or treat obesity and other appetite related disorders.

### Combinations for Co-Morbid Conditions

It will be appreciated by one skilled in the art that a therapy administered in combination with the compounds described herein can be directed to the same or a different disorder target as that being targeted by the compounds described herein.

Administration of the compound described herein may be first, followed by the other therapy; or administration of the other therapy may be first or they may be administered simultaneously either in two separate compositions or combined in a single composition. The other therapy is any known in the art to treat, prevent, or reduce the symptoms of the targeted disorder, e.g., a sleep disorder, or other disorders, e.g., other CNS disorders. In addition, some embodiments of the present invention have compounds administered in combination with other known therapies for the target disorder. Furthermore, the other therapy includes any agent of benefit to the patient when administered in combination with the disclosed FAAH inhibitors.

For example, in some embodiments where the other therapy is a drug, it is administered as a separate fat ululation or in the same formulation as the compound described herein. A FAAH inhibitory compound described herein is administered in combination therapy with any one or more of commercially-available, over-the-counter or prescription medications, including, bul not limited to antimicrobial agents, fungistatic agents, germicidal agents, hormones, antipyretic agents, antidiabetic agents, bronchodilators, antidiarrheal agents, antiarrhythmic agents, coronary dilation agents, glycosides, spasmolytics, antihypertensive agents, antidepressants, antianxiety agents, other psychotherapeutic agents, corticosteroids, analgesics, contraceptives, nonsteroidal anti-inflammatory drugs, blood glucose lowering agents, cholesterol lowering agents, anticonvulsant agents, other antiepileptic agents, immunoinodulatupsilonrs, anticholinergics, sympatholytics, sympathomimetics, vasodilatory agents, anticoagulants, antiarrhythmics, prostaglandins having various pharmacologic activities, diuretics, sleep aids, antihistainiriic agents, antineoplastic agents, oncolytic agents, antiandrogens, antimalarial agents, anti leprosy agents, and various other types of drugs. See Goodman and Oilman's The Basis of Therapeutics {Eighth Edition, Pergainopi Press, Inc, USA, 1990) and The Merck Index (Eleventh Edition, iMerck AND Co., Inc., USA, 1989).

### Combinations Useful in Treatment of Diabetes

Suitable agents of use in combination with a FAAH inhibitor of the invention include antidiabetic agents such as (1) PPARGAMMA agonists such as glitazones (e.g, ciglitazone; darglitazone; englitazone; isaglitazone (MCC-555); pioglitazone; rosiglitazone; troglitazone; BRL49653; CLX-0921; 5-BTZD, and GW-0207, LG-100641, and LY-300512, and the like and compounds disclosed in PCT publication Nos. WO97/10813, WO97/27857, WO97/28115, WO97/28137, WO97/27847, WO03/000685, WO03/027112, WO03/035602, WO03/048130, WO03/055867, and the like; (2) biguanides such as buformin: metformin; and phenformin, and the like; (3) protein tyrosine phosphatase-IB (PTP-IB) inhibitors, such as ISIS 11371, and those disclosed in WO03/032916, WO03/032982, WO03/041729, WO03/055883; (4) sulfonylureas such as acetohexamide; carbutamidc: chlorpropamide; diabinese: glibenclamide; glipizide; glyburide (glibenclamide); glimepiride; gliclazide; glipentide; gliquidone; glisolamide; tolazamide; and tolbutamide, and the like: (5) meglitinides such as repaglinide, and nateglinide, and the like; (6) alpha glucoside hydrolase inhibitors such as acarbose; adiposine; camiglihose; emiglitate; miglitol; voglibose; pradimicin-Q; salbostatin; CKD-711; MDL-25,637: MDL-73,945; and MOR14, and the like; (7) alpha-amylase inhibitors such as tendamistat. trestatin, and A 1-3688, and the like: (8) insulin secreatagogues such as linogliricle: and A-4166, and the like; (9) fatty acid oxidation inhibitors, such as clomoxir. and etomoxir, and the like; (10) A2 antagonists, such as midaglizole; isagliclole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and Ultralente); Lys-Pro insulin, GLP-I (73-7) (insulinotropin); and GLP-I (7-36)-NH₂), and the like; (12) non-thiazolidinediones such as JT-501, and farglitazar (GW-2570/G1-262579), and the like; (13) PPARALPHA/gamma dual agonists such as BVT-142. CLX-0940, GW-1536, CW-1929, GW-2433, KRP-297, L-796449, LR-90, MK-0767, SB 219994, inuraglitazar and reglitazar (JTT-501) and those disclosed in WO99/1G758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976; and (14) other insulin sensitizing drugs; (15) VPAC2 receptor agonists; (16) GLK modulators, such as those disclosed in WO03/015774; (17) retinoid modulators such as those disclosed in WO03/000249; (18) GSK 3β/GSK 3 inhibitors such as 4-[2-(2-bromophenyl)-4-(4-fluorophenyl-1H-imidazol-5-yl)pyridine and those compounds disclosed in WO03/024447, WO03/037869, WO03/037877, WO03/037891, WO03/068773, EP 1295884, EP 1295885, and the like; (19) glycogen phosphorylase (HGLPa) inhibitors, such as those disclosed in WO03/037864; (20) ATP consumption promoters such as those disclosed in WO03/007990; (21) TRB3 inhibitors, (22) vanilloid receptor ligands such as those disclosed in WO03/049702, (23) hypoglycemic agents such as those disclosed in WO03/015781, WO03/040114, (24) glycogen synthase kinase 3 inhibitors such as those disclosed in WO03/035663, (25) and agents such as those disclosed in WO99/51225, and US 20030134890; and WO01/24786, WO03/059870; (26) Insulin-responsive DNA binding protein-1 (IRDBP-I) as disclosed in WO03/057827, and the like; (27) Adenosine A2 antagonists such as those disclosed in WO03/035639, WO03/035640, and the like.

### Combinations Useful in Treatment of Hyperlipidemia

Suitable agents of use in combination with a FAAH inhibitor of the invention include lipid lowering agents such as:

(1) bile acid sequestrants such as, cholestyramine, coleseveleni, colestipol, dialkylaminoalkyl derivatives of a cross-linked dextran; Colestid®; LoCholest®: and Questran®, and the like; (2) HMG-CoA reductase inhibitors such as atorvastatin, bervastatin, carvastatin, cerivastatin, crilvastatin, dalvastatin, fluvastatin, glenvastatin, itavaslatin, lovastatin, mevastatin, pilavastatin, pravastatin, rivastatin, rosuvastatin, simvastatin, sirrivastatin, and ZD-4522, and the like and compounds disclosed in WO03/033481; (3) HMG-CoA synthase inhibitors; (4) cholesterol absorption inhibitors such as stanol esters, beta-sitosterol, sterol glycosides such as tiqueside; and azetidinones such as ezetimibe, and the like; (5) acyl coenzyme A-cholesterol acyl transferase (ACAT) inhibitors such as avasimibe (Current Opinion in Investigational Drugs. 3(9):291-297 (2003)), efludmibe, KY505, SMP 797, CL-277,082 (Clin Pharmacol Ther. 48(2): 189-94 (1990)) and the like; (6) CETP inhibitors such as JTT 705 identified as in Nature, 406, (6792):203-7 (2000), torcetrapib (CP-529,414 described in US20030186952 and WO20Q00171G4), CP 532,032, BAY03-2149, SC 591, SC 795, and the like including those described in Current Opinion in Investigational Drugs. 4(3):291-297 (2003); (7) squalene synthetase inhibitors, (8) antioxidants such as murobucol, AG I-1067 and the like; (9) PPAKALPHA agonists such as beclofibrate, benzafibrate, binifibrate, ciprofibrate, clinofibrate, clofibrate, etofibrate, fenofibrate, gemcabene, and gemfibrozil, lifibrol, GW 7647, BM 170744, LY518674; and other fibric acid derivatives, such as Atromid, Lopid and Tricor and those disclosed in WO03/033456, WO03/033481, WO03/043997, WO03/048116, WO03/053974, WO03/059864, WO03/05875, and the like: (10) FXR receptor modulators such as GW 4064, SR 103912, and the like; (11) LXR receptor modulators such as GW 3965. BO1 3137, and XTCOl 79628, and those disclosed in US20030125357, WO03/045382, WO03/053352, WO03/059874, and the like; (12) lipoprotein synthesis inhibitors such as niacin; (13) renin angiotensin system inhibitors; (14) PPARDELTA partial agonists, such as those disclosed in WO03/024395; (15) bile acid reabsorption inhibitors, such as BAR1 1453, SC435, PHA384640, S8921, AZD7706, and the like; (16) PPARDELTA agonists such as GW 501516, and GW 590735, and the like, such as those disclosed in WO97/28149. WO01/79197, WO02/14291, WO02/46154, WO02/46176, WO02/076957, WO03/016291, WO03/033493; (17) triglyceride synthesis inhibitors; (18) microsomal triglyceride transport (MTI?) inhibitors, such as inplitapide, LAB687, and CP346086, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists; (25) PPAR modulators such as those disclosed in WO99/07357, WO99/1 1255, WO99/12534, WO99/15520, WO99/46232, WO00/12491, WO00/23442, WO00/236331, WO00/236332, WO00/218355, WO00/238553, WO01/25181, WO01/79150, WO02/79162, WO02/100403, WO02/102780, WO02/081428, WO03/0162G5, WO03/033453, WO03/042194, WO03/043997, WO03/066581, and the like: (26) niacin-bound chromium, as disclosed in WO03/039535; (27) substituted acid derivatives disclosed in WO03/040114; (28) apolipoprotein B inhibitors such as those disclosed in WO02/090347, WO02/28835, WO03/045921, WO03/047575; (29) Factor Xa modulators such as those disclosed in WO03/047517, WO03/047520, and WO03/048081.

### Combinations Useful in Treatment of Hypertension

Suitable agents of use in combination with FAAH inhibitor of the invention include antihypertensive agents such as: (1) diuretics, such as thiazides, including chlorthalidone, clilorthiazide, dichlorophenamide, hydroflumethiazide, indapamide, polytlnazide, and hydrochlorotliiazide, loop diuretics, such as bumetanide, ethacrynic acid, furosemide. and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, caivedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridtl, cinaldipine, clevidipine, diltiazem, efonidipine, felodipine, gallopamil, isradipine, lacidipine, lemildipine, lercanidipine, nicardipine, nifedipine, nilvadipine nimodepine, ntsoldipine, nitrendipine, manidipine, pranidipine, and verapamil, and the like; (4) angiotensin converting enzyme (LAMDACE) inhibitors such as benazepril; captopril; ceranapril; cilazapril; delapril; enalapril; enalopril; fosinoppil; imidapril; lisinopril: losinopril: moexipril; quinapril; quinaprilat; ramipril; peripidopril; perindropril; quanipril; spirapnl; tenocapril; trandolapril, and zofenopril, and the like; (5) neutral endopeptidase inhibitors such as oinapatrilat, cadoxatril and ecadotril, fosidotril, sampatrilat, LAMDAVE7688, ER4030, and the like; (6) endothelin antagonists such as tezosentan, lamda308165, and YM62899, and the like; (7) vasodilators such as hydralazine, clonidine, minoxidil, and nicotinyl alcohol, and the like; (8)-angiotensin II receptor antagonists such as aprosartan, candesartan, eprosarlan, irbesartan, losartan, olmesartapi, pratosartan, tasosartan, lelmisartan. vaisartan, and EXP-3137, FIG828K, and RNH6270, and the like; (9) alpha/beta adrenergic blockers such as nipradilol, arotinolol and amosulalol, and the like, (10) alpha 1 blockers, such as terazosin, urapidil, prazosin, bunazosin, trimazosin, doxazocin, naftopidil, Uidoramin, WHP 164, and XENOlO, and the like; (11) alpha 2 agonists such as lofexidine, tiamenidine, moxonidine, rilmenidine, and guanobenz, and the like; (12) aldosterone inhibitors, and the like; and (13) angiopoietin-2-binding agents such as those disclosed in WO03/030833.

### COX-2 and FAAH Related Therapeutic Methods

Alone or in combination with a second active agent, the FAAH inhibitors of the invention can be used to treat conditions or disorders in which it is considered desirable to reduce or eliminate COX-2 activity and/or FAAH activity and/or MAGL. Thus, they can be used in any situation in which a COX-2 inhibitor or FAAH inhibitor or MAGL inhibitor is used as well as in other situations. For example, compounds and related prodrugs can be used to treat an inflammatory disorder, including both disorders in which inflammation is considered a significant component of the disorder and those in which inflammation is considered a relatively minor component of the disorder, to treat acute and chronic pain (analgesic) and to treat fever (antipyretic). Among the inflammatory disorders that can be treated are auto immune disorders.

Disorders that can be treated include: arthritis (including rheumatoid arthritis, spondyloarthropathies, gouty arthritis, degenerative joint diseases (i.e. osteoarthritis), systemic lupus erythematosus, ankylosing spondylitis, acute painful shoulder, psoriatic, and juvenile arthritis), asthma, atherosclerosis, osteoporosis, bronchitis, tendonitis, bursitis, skin inflammation disorders (i.e. psoriasis, eczema, burns, dermatitis), enuresis, eosinophilic disease, gastrointestinal disorders (including inflammatory bowel disease, peptic ulcers, regional enteritis, diverticulitis, gastrointestinal bleeding. Crohn's disease, gastritis, irritable bowel syndrome (IBS-C, IBS-A and IBS-D) and ulcerative colitis), and disorders ameliorated by a gastroprokinetic agent (i.e. ileus, for example post-operative ileus and ileus during sepsis: gastroesophageal reflux disease (GORD, or its synonym GERD); eosinophilic esophagitis, gastroparesis such as diabetic gastroparesis: food intolerances and food allergies and other functional bowel disorders, such as nonulcerative dyspepsia (NUD) and non-cardiac chest pain (NCCP)).

Alone or in combination with a second active agent, the FAAH inhibitors of the invention can also be used in the treatment of symptoms associated with influenza or other viral infections, common cold, sprains and strains, myositis, neuralgia, synovitis, injuries such as sports injuries and those following surgical and dental procedures, coagulation disorders, kidney disease (e.g., impaired renal function), ophthalmic disorders (including glaucoma, retinitis, retinopathies, uveitis and acute injury to the eye tissue), liver diseases (i.e., inflammatory liver disease including chronic viral hepatitis B, chronic viral hepatitis C, alcoholic liver injury, primary biliary cirrhosis, autoimmune hepatitis, non-alcoholic steatohepatitis (NASH) and liver transplant rejection), and pulmonary inflammatory diseases (e.g., including asthma, allergic rhinitis, respiratory distress syndrome chronic bronchitis, and emphysema). Compositions comprising a FAAH compound described herein and related prodrugs thereof can also be used to treat, for example, inflammation associated with: vascular diseases, migraine headaches, tension headaches, periarteritis nodosa, thyroiditis, aplastic anemia, Hodgkins disease, scicrodoma, rheumatic fever, type I diabetes, myasthenia gravis, sarcoidosis, nephrotic syndrome, Behcet's syndrome, polymyositis, gingivitis, hypersensitivity, conjunctivitis, multiple sclerosis, and ischemia (e.g., myocardial ischemia), and the like. The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis, and inflammatory bowel disease). The FAAH compounds may also be useful in treating inflammation associated with trauma and non-inflammatory myalgia. The compounds can also be administered to those prior to surgery or taking anticoagulants. The compounds may reduce the risk of a thrombotic cardiovascular event which is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fagax), reversible ischemic neurologic deficits, and any similar thrombotic event in any vascular bed (splanchnic, renal, aortic, peripheral, etc.).

The FAAH inhibitors of the invention may inhibit uterus contraction caused by hormones and prostanoid-induced smooth muscle contraction. The compounds may be useful in treating premature labor, menstrual cramps, menstrual irregularity, and dysmenorrhea.

Alone or in combination with a second active agent, the FAAH inhibitors of the invention may inhibit cellular neoplastic transformations and metastatic tumor growth. The compounds described herein may be associated with reducing die number of adenomatous colorectal polyps. Thus, compounds and prodrugs may also be useful in reducing the risk of certain cancers, e.g., solid tumor cancers such as colon or colorectal cancer. The compounds and prodrugs may also be used in the treatment of prevention of all cancers including cancers of the bladder, cancers associated with overexpression of HER-2/neu cervix, skin, esophagus, head and neck, lung including non small-cell lung cancers, kidney, pancreas, prostate, gall bladder and bile duct and endometrial cancers, gastric cancers, gliomas, hepatocellular carcinomas, colonic: adenomas, mammary cancers, ovarian cancers and salivary cancers. In addition, the compounds and prodrugs may be useful in treating large intestine cancer and prostate cancer. The compounds may also be useful in cases where the patient is at risk for cancer including oral premalignant lesions, cervical intraepithelial neoplasia, chronic hepatitis, bile duct hyperplasia, atypical adenomatous hyperplasia of lung, prostatic, intraepithelial neoplasia, bladder dysplasia, actinic keratoses of skin, colorectal adenomas, gastric metaplasia, and Barrett's esophagus.

Alone or in combination with a second active agent, the FAAH inhibitors of the invention are also useful for the treatment of cognitive disorders such as dementia, particularly degenerative dementia (including senile dementia, Alzheimer's disease (and precursors thereof), Pick's disease, Huntington's chorea, Parkinson's disease and Creutzfeldt-Jakob disease), and vascular dementia (including multiinfarct dementia), as well as dementia associated with intracranial space occupying lesions, trauma, infections and related conditions (including HIV infection), metabolism, toxins, anoxia and vitamin deficiency; and mild cognitive impalianent associated with ageing, particularly Age Associated Memory Impairment.

Alone or in combination with a second active agent, the FAAH inhibitors of the invention may also prevent neuronal injury by inhibiting the generation of neuronal free radicals (and hence oxidative stress) and therefore are of use in the treatment of stroke, epilepsy; and epileptic seizures (including grand mal. petit mal. myoclonic epilepsy and partial seizures) The compounds may be useful to control or suppress seizures (including those that are chemically induced).

Alone or in combination with a second active agent, the FAAH inhibitors of the invention can be used in treatment of all varieties of pain including pain associated with a cough condition, pain associated with cancer, preoperative pain, arthritic pain and other forms of chronic pain such as post-operative pain, lumbosacral pain, musculoskeletal pain, headache, migraine, muscle ache, lower back, and neck pain, toothache and the like. The compounds are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord, or certain regions in the brain. Neuropathic pain syndromes are traditionally classified according to the disease or event that precipitated them. Neuropathic pain syndromes include: diabetic neuropathy; sciatica; back pain, non-specific lower back pain; multiple sclerosis pain; fibromyalgia; HIV-related neuropathy; neuralgia, such as post-herpetic neuralgia and trigeminal neuralgia; pain related to chronic alcoholism, hypothyroidism, uremia, or vitamin deficiencies; pain related to compression of the nerves (e.g., Carpal Tunnel Syndrome), and pain resulting from physical trauma, amputation/phantom limb pain, cancer, toxins or chronic inflammatory conditions. The symptoms of neuropathic pain are incredibly heterogeneous and are often described as spontaneous shooting and lancinating pain, or ongoing, burning pain. In addition, there is pain associated with normally non painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Alone or in combination with a second active agent, the FAAH inhibitors of the invention may also be of use in the treatment and/or prevention of cyclooxygenase-mediated proliferative disorders such as may occur in diabetic retinopathy and tumor angiogenesis. The compounds may be used to inhibit angiogenisis, such as occurs in wet macular degeneration.

Alone or in combination with a second active agent, the FAAH inhibitors of the invention may also be used for treating sexual behavior problems and/or improving sexual performance.

Alone or in combination with a second active agent, the FAAH inhibitors of the invention are useful in the prevention and/or treatment of pain, in particular acute or chronic neurogenic pain, migraine, neuropathic pains including the forms associated with herpes virus and diabetes, acute or chronic pain associated with the inflammatory diseases: arthritis, rheumatoid arthritis, osteoarthritis, spondylitis, gout, vascularis, Crohn's disease, irritable bowel syndrome and acute/sharp or chronic pains at the periphery. The compounds can also be used to prevent and/or treat emesis, dizziness, vomiting, and nausea, especially after chemotherapy, food behavioral problems/feeding disorders (i.e. eating disorders, in particular anorexias and cachexias of various natures, weight loss associated with cancer and other wasting conditions, or bulimia), neurological pathologies, psychiatric tremors (e.g., dyskinesias, dystonia, spasticity, obsessive compulsive behavior, Tourette's syndrome, all forms of depression and anxiety of any nature and origin, mood disturbances, psychoses), acute or chronic neurodegenerative diseases (e.g., Parkinson's disease. Alzheimer's disease, senile insanity, Huntington's chorea, lesions related to cerebral ischemia and cranial and medullary traumas, epilepsy, sleep disorders (sleep apnea), cardiovascular diseases (in particular hypertension, cardiac arrhythmias, arteriosclerosis, heart attacks, cardiac ischemias, renal ischemia), cancers (benign tumors of the skin, papillomas and cerebral tumors, prostate tumors, cerebral tumors (glioblastomas, medullar}′ epitheliomas, medullary blastoinas, neuroblastomas, tumors of origin, astrocytomas, astroblastomas, ependymomas, oligodendrogliomas, plexus tumor, neuroepithelioma, epiphysis tumor, ependyblastoinas, malignant meningiomas, sarcomatosis, malignant melanomas, schvvan cell cancers), disorders of the immune system (in particular autoimmune diseases including psoriasis, erythematous lupus), diseases of conjunctive or connective tissue, Sjogren's syndrome, spondylarthritis anchylosis, undifferentiated spondylarthritis undifferentiated, Behcet's disease, autoimmune hemolytic anaemias, multiple sclerosis, amyotrophic lateral sclerosis, amyloses, graft rejection, and illnesses affecting the blastocytes. allergic diseases (i.e., immediate or delayed hypersensitivity, allergic rhinitis or conjunctivitis, contact dermatitis), viral or bacterial parasitic infectious diseases (i.e. AIDS, meningitis), inflammatory diseases (in particular arthritic diseases: arthritis, rheumatoid arthritis osteoarthritis, spondylitis, gout, vascularis, Crohn's disease, irritable bowel syndrome, osteoporosis, psoriasis, ocular infections and disorders (i.e. ocular hypertension, glaucoma, wet macular degeneration), lung diseases (i.e. diseases of the respiratory tracts, bronchyospasms, cough, asthma, chronic bronchitis, chronic obstruction of the respiratory tracts, emphysema), gastrointestinal disorders (i.e. irritable bowel syndrome, intestinal inflammatory disorders, ulcers, diarrheas, acid reflux), urinary incontinence, vesical inflammation, movement disorders, psychomotor disorders, hypertension, and AIDS-related complex. The compounds can be used as a sleep aid to treat insomnia or to induce sleep. The compounds may be used to reduce or control body weight (or fai) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). The compounds may be used to prevent, control or treat schizophrenia, paranoia or other related disorders, or other disorders of dopamine transmission.

Alone or in combination with a second active agent, the FAAH inhibitors of the invention can also be used to treat anxiety (including generalized anxiety disorder, panic disorder, and social anxiety Disorder) and depression.

Alone or in combination with a second active agent, the FAAH inhibitors of the invention can also be used in the treatment of pollakiuria, for example in the treatment of urinary incontinence, uresiesthesia urgency, or overactive bladder. Pollakiuria refers to the condition characterized by the voiding or passing of small quantities of urine more frequently than normal. Interstitial cystitis, chronic prostatitis, neuropathy (for example, resulting from neurogenic bladder or cerebral infarction), lower urinary tract prostatic hypertrophy, and aging, are among the conditions associated with pollakiuria.

In one aspect the invention is directed to pharmaceutical compositions comprising:

A FAAH inhibiting compound of formula I:

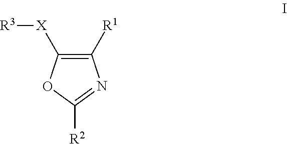

or a pharmaceutically acceptable salt thereof wherein:

### X is S or SO;

n is 0, 1 or 2;
R¹ is selected from the group consisting of:

(1) aryl, and

(2) HET¹,

wherein R¹ is optionally mono or di-substituted with substituents R⁴ and R⁵; and wherein R⁴ and R⁵ are independently selected from the group consisting of:

(a) halo,

(b) —CN,

(c) mono, di or tri-halo C₁₋₄alkyl,

(d) mono, di or tri-halo OC₁₋₄ alkyl,

(d) —OC₁₋₄ alkyl, optionally substituted with hydroxyl, halo or amino,

(e) —C₁₋₄alkyl optionally substituted with one or two substituents selected from hydroxyl, CN, —CHF₂ and —CF₃,

(f) —C₁₋₂alkyl-C₃₋₆cycloalkyl optionally substituted with hydroxy, halo or CN,

(g) —S(O)ₙC₁₋₄alkyl,

(h) —S(O)ₙNR⁶R⁷,

(i) —C(O)—NH—NR⁸R⁹,

(j) C(O)—OH,

(k) —C(O)—OC₁₋₄alkyl, optionally substituted with halo or hydroxy,

(l) —C(O)—NR¹⁰R¹¹,

(m) —C(O)—C₁₋₄alkyl optionally mono, di or tri substituted with halo,

(o) —C(NR¹²)—NR¹³R¹⁴,

(p) HET⁴,

(q) aryl,

(r) —C(O)—NH—NH—C(O)H,

(s) —CH₂—C(O)—O—C₁₋₄alkyl, whereas the CH₂ may be optionally substituted with C₁₋₄alkyl or OH

(t) —CH₂—C(O)NR¹⁵R¹⁶, whereas the CH₂ may be optionally substituted with C₁₋₄alkyl or OH, and

(u) —NR¹⁷R¹⁸,

wherein choices (p) and (q) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC₁₋₄allyl optionally substituted with hydroxyl or halo,

(7) —C(O)OH, and

(8) —C(O)O—C₁₋₃alkyl;

(9) —C(O)—NR¹⁹R²⁰,

(10) —NH₂,

(11) Oxo,

(12) ═S,

with the proviso that the substituent on choice (q) is other than oxo or
wherein R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰, are each independently selected from H and C₁₋₄alkyl,
or
R⁶ and R⁷ or R⁸ and R⁹ or R¹⁰ and R¹¹ or R¹³ and R¹⁴ or R¹⁵ and R¹⁶ or R¹⁷ and R¹⁸ or R¹⁹ and R²⁰ are joined together to form a ring with the nitrogen to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl and —S(O)nC₁₋₄alkyl;
R² is selected from the group consisting of:

(1) aryl,

(2) HET³,

(3) —CH₂-aryl,

(4) —CH₂—HET³,

(5) —C₁₋₆alkyl, and

(6) —C₃₋₆cycloalkyl,

wherein R² is optionally mono or di-substituted with substituents independently selected from the group consisting of

(a) halo,

(b) —CN,

(c) —OH,

(d) —C₁₋₄alkyl optionally substituted with hydroxy, halo or cyano,

(e) —CF₃,

(f) —OC₁₋₄alkyl optionally substituted with hydroxyl or halo,

(g) —C(O)O—C₁₋₃alkyl and

(h) —S-aryl, optionally substituted with halo, C₁₋₄alkyl or —OC₁₋₄alkyl;

R³ is selected from the group consisting of:

(1) aryl,

(2) HET⁵, and

(3) C₃₋₆cycloalkyl,
wherein R³ is optionally mono or di-substituted with substituents independently selected from the group consisting of
   (a) hydroxy,
   (b) halo,
   (c) —C₃₋₆cycloalkyl,
   (d) —OC3-5cycloalkyl,
   (e) —C₁₋₄alkyl,
   (f) —OC₁₋₄ alkyl,
   (g) —C(O)CH₃
   (h) mono, di or tri-halo C₁₋₄ alkyl,
   (i) mono, di or tri-halo —OC₁₋₄ alkyl, and
   (j) —S(O)ₙ—C₁₋₄ alkyl;
      wherein aryl is as a mono- or bi-cyclic aromatic ring system; and HET¹, HET³, HET⁴ and HET⁵ are each independently a 5 to 10-membered aromatic, partially aromatic or non-aromatic mono- or bicyclic ring, or N-oxide thereof, said containing 1 to 4 heteroatoms selected from O, S and N, and optionally substituted with 1 to 2 oxo groups;

or a FAHH inhibiting compound of formula I

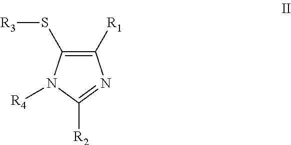

or a pharmaceutically acceptable salt thereof wherein:
n=0, 1 or 2
R₁ is selected from the group consisting of:

(1) phenyl, and

(2) HET₁,

wherein choice (1) and (2), is substituted with

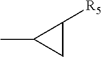

wherein R₅ is selected from the group consisting of

(a) halo,

(b) —CN,

(c) halo C₁₋₄ alkyl,

(d) —OC₁₋₄ alkyl, optionally substituted with hydroxy, halo or amino,

(e) —C₁₋₄alkyl optionally substituted with one or two substituents selected from hydroxyl, CN, —CHF₂ and —CF₃,

(f) —C₁₋₂alkyl-C₃₋₆cycloalkyl optionally substituted with hydroxy, halo or CN,

(g) —S(O)ₙC₁₋₄alkyl,

(h) —S(O)ₙNR₆R₇,

(i) —C(O)—OH,

(j) —C(O)—OC₁₋₄alkyl, optionally substituted with halo or hydroxy,

(k) —C(O)—NR₁₀R₁₁,

(l) —C(O)—C₁₋₄alkyl optionally mono, di or tri substituted with halo,

(m) HET2,

(n) aryl,

(o)—CH₂—C(O)—O—C₁₋₄alkyl, whereas the CH₂ may be optionally substituted with C₁₋₄ alkyl or OH

(t) —CH₂—C(O)NR¹⁵R¹⁶, whereas the CH₂ may be optionally substituted with C₁₋₄ alkyl or OH, and

(u) —NR₁₇R₁₈,

wherein choices (m) and (in) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)—NR₁₉R₂₀,

(9) —NH₂,

(10) Oxo,

(11) ═S,

wherein R₆, R₇, R₁₀, R₁₁, R₁₅, R₁₆, R₁₇, R₁₈, R₁₉ and R₂₀ are each independently selected from H and C₁₋₄alkyl, wherein C₁₋₄alkyl is optionally mono-, di-, or tri-substituted with halo,
or
R₆ and R₇ or R₁₀ and R₁₁ or R₁₅ and R₁₆ or R₁₇ and R₁₈ or R₁₉ and R₂₀ are joined together so that together with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄allyl, haloC₁₋₄alkyl, —C(O)—C₁₋₄alkyl and —S(O)ₙC₁₋₄alkyl;
R₂ is selected from the group consisting of:

(1) hydrogen,

(2) aryl,

(3) HET₃,

(4) —CH₂-aryl,

(5) —CH₂—HET₃,

(6) —C₁₋₆alkyl, and

(7) —C₃₋₆cycloalkyl,

wherein choice (2), (3), (4), (5), (6) and (7) is optionally mono or di-substituted with substituents independently selected from the group consisting of

(a) halo,

(b) —CN,

(c) —OH,

(d) —C₁₋₄alkyl optionally substituted with hydroxy, halo or cyano,

(e) —CF₃,

(f) —OC₁₋₄alkyl optionally substituted with hydroxyl or halo,

(g) —C(O)O—C₁₋₃alkyl;

R₃ is selected from the group consisting of
(1) aryl,
(2) HET₄, and
(3) C₃₋₆cycloalkyl,
wherein choice (1), (2) and (3) are each optionally mono or di-substituted with substituents independently selected from the group consisting of
   (a) hydroxy,
   (b) halo,
   (c) —C₃₋₆cycloalkyl,
   (d) —OC3-5cycloalkyl,
   (e) —C¹⁻⁴alkyl,
   (f) —OC₁₋₄alkyl,
   (g) —C(O)CH₃
   (h) mono, di or tri-halo C₁₋₄ alkyl,
   (i) mono, di or tri-halo —OC₁₋₄ alkyl, and
   (j) —S(O)ₙ—C₁₋₄ alkyl; and
      R₄ is selected from the group consisting of:

(1)-C₁₋₄alkyl,

(2) -haloC₁₋₄alkyl,

(3) H; and

HET₁, HET₂, HET₃ and HET₄ are each independently a 5- to 10-membered aromatic, partially aromatic or non-aromatic mono- or bicyclic ring, containing 1-4 heteroatoms selected from O, S and N, and optionally substituted with 1-2 oxo groups.

Within this aspect there is a genus wherein

R₁ is selected from the group consisting of:

(1) phenyl,

(2) pyridinyl,

(3) pyridazinyl,

(4) pyrimidinyl,

(5) pyrazinyl,

(6) thiazolyl,

(7) thienyl,

(8) pyrrolyl, and

(9) oxazolyl,

wherein choice of (1) to (9) is substituted with

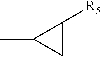

and wherein R₅, is selected from the group consisting of

(b) —CN,

(c) halo C₁₋₄alkyl,

(d) —O—C₁₋₄alkyl, optionally substituted with hydroxyl, halo or amino

(e) —C₁₋₄alkyl optionally substituted with hydroxyl or CN,

(f) —C₁₋₂alkyl-C₃₋₆cycloalkyl optionally substituted with hydroxy,

(h) —S(O)ₙC₁₋₄alkyl wherein n is 1 or 2,

(i) —S(O)₂NR₆R₇,

(j) —C(O)—NR₁₀R₁₁,

(k) HET2,

(l) aryl, and

wherein choices (k) and (l) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH,

(8) —C(O)O—C₁₋₃alkyl, and

(9) —C(O)—NR₁₉R₂₀,

wherein R₆, R₇, R₁₀, R₁₁, R₁₉ and R₂₀, are each independently selected from H and C₁₋₄alkyl, wherein the C₁₋₄alkyl is optionally momo-, di-, or tri-substituted with halo;

and a second active agent agent such as an agent selected from the group consisting of a COX-2 inhibitor (such as etoricoxib (ARCOXIA) or celecoxib (CELEBREX)); an NSAID (such as acetylsalicylic acid, salicylic acid, salicylamide, salsalate, diflunisal, gentisic acid, indomethacin, sulindac, tolmetin, diclofenac, etodolac, nabumetone, ibuprofen, fenoprofen, ketoprofen, flurbiprofen, suprofen, carprofen, naproxen, ketorolac, oxaprozin, mefenamic acid, meclofenamate sodium, piroxicam and meloxicam); an M-opioid receptor agonist (such as Tramadol); a GABA analogue (such as gabapentin), pregabalin; a PPARα agonist, a CB1 or CB2 receptor antagonist; acetaminophen; a dopamine D2 receptor antagonist; and a melanocortin receptor modulating agent.

NSAID's and COX-2 inhibitors are known to be useful as anti-inflammatory agent, anti-pyretic agents and pain relievers.

Within this aspect the is a genus of compound of formula I wherein

R¹ is selected from the group consisting of

(1) phenyl,

(2) pyridyl,

(3) pyridazinyl,

(4) pyrimidyl,

(5) pyrazinyl,

(6) thiazolyl,

(7) thienyl,

(8) pyrrolyl,

(9) oxazolyl, and

(10) oxadiazolyl;

wherein R¹ is optionally mono or di-substituted with substituents R⁴ and R⁵, wherein R⁴ and R⁵ are independently selected from the group consisting of:

(a) halo,

(b) —CN,

(c) mono, di or tri-halo C₁₋₄ alkyl,

(d) —O—C₁₋₄alkyl, optionally substituted with hydroxyl, halo or amino

(e) optionally substituted with hydroxyl or CN,

(f) —C₁₋₂alkyl-C₃₋₆cycloalkyl optionally substituted with hydroxy,

(h) —S(O)ₙC₁₋₄alkyl wherein n is 0, 1 or 2,

(i) —S(O)ₙNR⁶R⁷,

(j) —C(O)—NR¹⁰R¹¹,

(k) HET⁴,

(l) aryl, and

wherein choices (k) and (l) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC1.4alkyl optionally substituted with hydroxyl or halo,

(7) —C(O)OH,

(8) —C(O)O—C₁₋₃alkyl, and

(9) —C(O)—NR¹⁹R²⁰,

wherein R⁶, R⁷, R¹⁰, R¹¹, R¹⁹ and R²⁰ are each independently selected from H and C₁₋₄alkyl.

Within this genus there is a sub-genus of compound of formula I wherein:

R¹ is selected from the group consisting of:

(1) phenyl,

(2) pyridyl,

(3) pyrimidyl,

(4) pyrazinyl,

(5) pyridazinyl,

(6) 1,2,4-oxadiazolyl, and

(7) 1,3,4-oxadiazolyl,

optionally mono or di-substituted with substituents R⁴ and R⁵, which are independently selected from the group consisting of

(a) —C₁₋₄alkyl optionally substituted with hydroxy,

(b) —S(O)ₙC₁₋₄alkyl,

(c) —C(O)—NR¹⁰R¹¹,

(d) HET⁴, and

(e) halo,

wherein HET⁴ is optionally mono or di-substituted with substituents selected from:

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)O—C₁₋₃alkyl, and

(9) —C(O)—NR¹⁹R²⁰,

wherein R¹⁰, R¹¹, R¹⁹ and R²⁰ are each independently selected from H and C₁₋₄alkyl.

Within this aspect there is a genus of compound of formula I wherein: R² is selected from the group consisting of:

(1) aryl,

(2) HET³,

(3) —CH₂aryl, and

(4) —CH₂HET³,

wherein R² is optionally mono or di-substituted with substituents independently selected from the group consisting of

(a) halo,

(b) —CN,

(c) —OH,

(d) -Hydroxy C₁₋₄alkyl,

(e) C₁₋₄alkyl,

(f) C₁₋₄haloalkyl, and

(g) —OC₁₋₄alkyl, optionally substituted with halo or hydroxyl.

Within this genus there is a sub-genus of compound of formula I wherein: R² is selected from the group consisting of:

(1) aryl, and

(2) HET³,

wherein R² is optionally mono or di-substituted with substituents independently selected from the group consisting of

(a) halo,

(b) —CN,

(c) —OH,

(d) -hydroxy C₁₋₄alkyl,

(e) —CH₃,

(f) —CF₃, and

(g) —OCH₃.

Within this sub-genus there is a class of compound of formula I wherein: R² is selected from the group consisting of

(1) phenyl,

(2) pyridyl,

(3) pyridazinyl,

(4) pyrimidyl,

(5) pyrazinyl,

(6) thiazolyl,

(7) oxazolyl,

(8) pyrazolyl,

(9) 1,2,4-oxadiazolyl, and

(10) 1,3,4-oxadiazolyl,

wherein R² is optionally mono or di-substituted with halo, OC₁₋₄alkyl optically substituted with halogen, —C₁₋₄haloalkyl, hydroxyl and CN.

Within this aspect there is a genus of compound of formula I wherein:

R³ is selected from the group consisting of:
(1) aryl, and
(2) HET⁵,
wherein choice (1) and (2) are each optionally mono or di-substituted with substituents independently selected from the group consisting of:
   (a) halo,
   (b) —C₃₋₆cycloalkyl,
   (c) —OC₁₋₄ alkyl,
   (d) mono, di or tri-halo C₁₋₄alkyl, and
   (e) mono, di or tri-halo —OC₁₋₄alkyl.

Within this genus there is a sub-genus of compound of formula I wherein:

R³ is selected from the group consisting of

(1) phenyl,

(2) pyrimidyl,

(3) pyridyl,
wherein R³ is optionally mono or di-substituted with halo, haloC₁₋₄alkyl, or —OC₁₋₄alkyl optionally substituted with halo.

Within this aspect there is a genus of compound of formula I wherein X is S.

Within this aspect there is a genus of the Formula

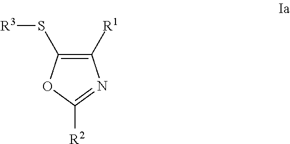

wherein
R¹ is selected from the group consisting of:

(1) phenyl,

(2) pyridyl,

(3) pyridazinyl,

(4) pyrimidyl,

(5) pyrazinyl,

(6) thiazolyl,

(7) thienyl,

(8) pyrrolyl,

(9) oxazolyl, and

(10) oxadiazole;

wherein R¹ is optionally mono or di-substituted with substituents R⁴ and R⁵, which are independently selected from the group consisting of

(a) halo,

(b) —CN,

(c) mono, di or tri-halo C₁₋₄ alkyl,

(d) —O—C₁₋₄alkyl, optionally substituted with hydroxyl, halo or amino

(e) —C₁₋₄alkyl optionally substituted with hydroxyl or CN,

(f) —C₁₋₂alkyl-C₃₋₆cycloalkyl optionally substituted with hydroxy,

(h) —S(O)ₙC₁₋₄alkyl wherein n is 0, 1 or 2,

(i) —S(O)ₙNR⁶R⁷,

(j) —C(O)—NR¹⁰R¹¹,

(k) HET⁴,

(l) aryl, and

wherein choices (k) and (l) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH,

(8) —C(O)O—C₁₋₃alkyl, and

(9) —C(O)—NR¹⁹R²⁰,

wherein R⁶, R⁷, R¹⁰, R¹¹, R¹⁹ and R²⁰, are each independently selected from H and C₁₋₄alkyl; R² is selected from the group consisting of:

(1) aryl,

(2) HET³,

(3) —C₁₋₆alkyl, and

(4) —C₃₋₆cycloalkyl,

wherein choice R² is optionally mono or di-substituted with substituents independently selected from the group consisting of

(a) halo,

(b) —CN,

(c) —OH,

(d) -hydroxy C₁₋₄alkyl,

(e) —C₁₋₄alkyl,

(f) —C₁₋₄haloalkyl, and

(g) —OC₁₋₄alkyl, optionally substituted with halo or hydroxyl; and

R³ is selected from the group consisting of:
(1) aryl, and
(2) HET⁵,
wherein choice (1) and (2) are each optionally mono or di-substituted with substituents independently selected from the group consisting of
   (a) halo,
   (b) —C₃₋₆cycloalkyl,
   (c) —C₁₋₄alkyl,
   (d) —OC₁₋₄ alkyl,
   (e) mono, di or tri-halo C₁₋₄ alkyl, and
   (f) mono, di or tri-halo —OC₁₋₄ alkyl.

Within this genus there is a sub-genus of compound of formula Ia wherein:

R¹ is selected from the group consisting of:

(1) phenyl,

(2) pyridinyl,

(3) pyrimidinyl,

(4) pyrazinyl,

(5) pyridazinyl,

(6) 1,2,4-oxadiazolyl, and

(7) 1,3,4-oxadiazolyl,

optionally mono or di-substituted with substituents R⁴ and R⁵, which are independently selected from the group consisting of

(a) —C₁₋₄optionally substituted with hydroxy,

(b) —S(O)ₙC₁₋₄alkyl,

(c) —C(O)—NR¹⁰R¹¹,

(d) HET⁴, and

(e) halo,

wherein HET⁴ is optionally mono or di-substituted with substituents selected from:

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)O—C₁₋₃alkyl, and

(9) —C(O)—NR¹⁹R²⁰,

wherein R¹⁰, R¹¹, R¹⁹ and R²⁰ are each independently selected from H and C₁₋₄alkyl.
R² is selected from the group consisting of:

(1) phenyl,

(2) pyridyl,

(3) pyridazinyl,

(4) pyrimidyl,

(5) pyrazinyl,

(6) thiazolyl,

(7) oxazolyl,

(8) pyrazolyl,

(9) 1,2,4-oxadiazolyl, and

(10) 1,3,4-oxadiazolyl,

wherein R² is optionally mono or di-substituted with halo, OC₁₋₄allyl optially substituted with halogen, —C₁₋₄haloalkyl, hydroxyl and CN; and
R³ is selected from the group consisting of:

(1) phenyl,

(2) pyrimidyl,

(3) pyridyl,
wherein R³ is optionally mono or di-substituted with halo, haloC₁₋₄alkyl, or —OC₁₋₄alkyl optionally substituted with halo.

Within this genus there is a sub-genus of the Formula

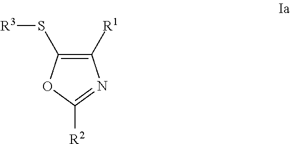

wherein:
R¹ is selected from the group consisting of:

(1) phenyl,

(2) pyridyl,

(3) pyridazinyl,

(4) pyrimidyl,

(5) pyrazinyl,

wherein R¹ is optionally mono or di-substituted with substituents R⁴ and R⁵, which are independently selected from the group consisting of

(a) halo,

(b) —CN,

(c) mono, di or tri-halo C₁₋₄ alkyl,

(d) —O—C₁₋₄alkyl, optionally substituted with hydroxyl, halo or amino

(e) —C(CH₃)₂—OH;

R² is selected from the group consisting of:

(1) phenyl,

(2) pyridyl,

(3) pyridazinyl,

(4) pyrimidyl,

(5) pyrazinyl,

(6) pyrazolyl,

wherein R² is optionally mono or di-substituted with halo, OC₁₋₄alkyl optially substituted with halogen, —C₁₋₄haloalkyl, hydroxyl and CN; and
R³ is selected from the group consisting of:

(1) phenyl,

(2) pyrimidyl,

(3) pyridyl,
wherein R³ is optionally mono or di-substituted with halo, haloC₁₋₄alkyl, or —OC₁₋₄alkyl optionally substituted with halo.

Within this sub-genus there is a class of compounds of formula Ia wherein:

R¹ is selected from the group consisting of:

(1) phenyl,

(2) pyridyl,

(3) pyrazinyl,

wherein R¹ is optionally mono or di-substituted with substituents R⁴ and R⁵, which are independently selected from the group consisting of:

(a) halo,

(b) —CN,

(c) mono, di or tri-halo C₁₋₄ alkyl,

(d) —O—C₁₋₄alkyl, optionally substituted with hydroxyl, halo or amino

(e) —C(CH₃)₂—OH;

R² is selected from the group consisting of:

(1) phenyl,

(2) pyridyl,

wherein R² is optionally mono or di-substituted with halo, OC₁₋₄alkyl optially substituted with halogen, —C₁₋₄haloallyl, hydroxyl and CN; and
R³ is selected from the group consisting of:

(1) phenyl,

(2) pyrimidyl,

(3) pyridyl,
wherein R³ is optionally mono or di-substituted with halo, haloC₁₋₄alkyl, or —OC₁₋₄alkyl optionally substituted with halo.
   Illustrating the compounds of formula I is Examples 1 to 138.

Within this aspect there is a genus of compound of formula II wherein:

R₁ is selected from the group consisting of:

(1) phenyl,

(2) pyridinyl,

(3) pyrimidinyl,

(4) pyrazinyl, and

(5) pyridazinyl,

wherein choice of (1) to (5) is substituted with

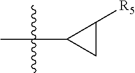

and R₅ is selected from the group consisting of

(a) —C₁₋₄alkyl optionally substituted with hydroxy,

(b) —S(O)₂C₁₋₄alkyl,

(e) —C(O)—NR₁₀R₁₁,

(d) HET₂, and

(e) halo,

wherein choice (d) is optionally mono or di-substituted with substituents selected from:

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)O—C₁₋₃alkyl, and

(9) —C(O)—NR₁₉R₂₀,

wherein R₁₀, R₁₁, R₁₉ and R₂₀ are each independently selected from H and C₁₋₄alkyl, wherein C₁₋₄alkyl is optionally mono-, di-, or tri-substituted with halo.

Within this aspect there is a genus of compounds of formula II wherein

R₂ is selected from the group consisting of:

(1) hydrogen,

(2) aryl,

(3) HET₃,

(4) —C₁₋₆alkyl, and

(5) —C₃₋₆cycloalkyl,

wherein choice (2), (3), (4) and (5) is optionally mono or di-substituted with substituents independently selected from the group consisting of

(a) halo,

(b) —CN,

(c) —OH,

(d) -hydroxy C₁₋₄alkyl,

(e) C₁₋₄alkyl,

(f) —C₁₋₄haloalkyl, and

(g) —OC₁₋₄alkyl, optionally substituted with halo or hydroxyl.

Within this genus there is a sub-genus of compounds of formula II wherein

R₂ is selected from the group consisting of:

(1) hydrogen,

(2) —C₁₋₆alkyl, and

(3) —C₃₋₆cycloalkyl,

wherein choice (2) and (3) are each optionally mono or di-substituted with substituents independently selected from the group consisting of

(a) halo,

(b) —CN,

(c) —OH,

(d) -hydroxy C₁₋₄alkyl,

(e) —CH₃,

(f) —CF₃, and

(g) —OCH₃.

Within this aspect there is a genus of compounds of formula II wherein

R₃ is selected from the group consisting of:
(1) phenyl, and
(2) HET₄,
wherein choice (1) and (2) are each optionally mono or di-substituted with substituents independently selected from the group consisting of
   (a) halo,
   (b) —C₃₋₆cycloalkyl,
   (c) —C₁₋₄ alkyl,
   (d) —OC₁₋₄alkyl,
   (e) mono, di or tri-halo C₁₋₄ alkyl, and
   (f) mono, di or tri-halo —OC₁₋₄alkyl.

Within this genus there is a sub-genus of compounds of formula II wherein

R₃ is selected from the group consisting of

(1) phenyl,

(2) pyrimidinyl,

(3) pyridinyl,

(4) pyridazinyl,

(5) pyrazinyl,
wherein choices (1), (2), (3), (4) and (5) are each optionally mono or di-substituted with halo, haloC₁₋₄alkyl, or —OC₁₋₄alkyl optionally substituted with halo.

Within this aspect there is a genus of compounds of formula II wherein

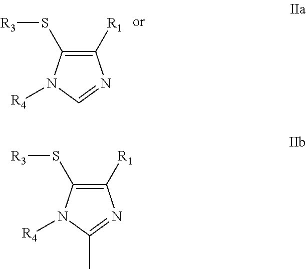

or a pharmaceutically acceptable salt thereof wherein:
R₁ is selected from the group consisting of:

(1) phenyl,

(2) pyridinyl,

(3) pyridazinyl,

(4) pyrimidinyl,

(5) pyrazinyl,

(6) thiazolyl,

(7) thienyl,

(8) pyrrolyl, and

(9) oxazolyl,

wherein choice of (1) to (9) is substituted with

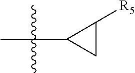

and R₅ is selected from the group consisting of

(a) —CN,

(b) halo C₁₋₄ alkyl,

(c) —O—C₁₋₄alkyl, optionally substituted with hydroxyl, halo or amino

(d) —C₁₋₄alkyl optionally substituted with hydroxyl or CN,

(e) —C₁₋₂alkyl-C₃₋₆cycloalkyl optionally substituted with hydroxy,

(g) —S(O)ₙC₁₋₄alkyl wherein n is 1 or 2,

(h) —S(O)₂NR₆R₇,

(i) —C(O)—NR₁₀R₁₁

(j) HET2,

(k) aryl, and

wherein choices (j) and (k) are each optionally mono or di-substituted with substituents selected from

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH,

(8) —C(O)O—C₁₋₃alkyl, and

(9) —C(O)—NR₁₉R₂₀,

wherein R₆, R₇, R₁₀, R₁₁, R₁₉ and R₂₀, are each independently selected from H and C₁₋₄alkyl, wherein C₁₋₄alkyl is optionally tritiated or mono-, di-, or tri-substituted with halo, or
R₂ is selected from the group consisting of:

(1) hydrogen,

(2) aryl,

(3) HET₃,

(4) —C₁₋₆alkyl, and

(5) —C₃₋₆cycloalkyl,

wherein choice (2), (3), (4) and (5) is optionally mono or di-substituted with substituents independently selected from the group consisting of

(a) halo,

(b) —CN,

(c) —OH,

(d) -hydroxy C₁₋₄alkyl,

(e) —C₁₋₄alkyl,

(f) —C₁₋₄haloalkyl, and

(g) —OC₁₋₄alkyl, optionally substituted with halo or hydroxyl; and

R₃ is selected from the group consisting of:
(1) phenyl, and
(2) HET₄,
wherein choice (1) and (2) are each optionally mono or di-substituted with substituents independently selected from the group consisting of
   (a) halo,
   (b) —C₃₋₆cycloalkyl,
   (c) —C₁₋₄ alkyl,
   (d) —OC₁₋₄alkyl,
   (e) mono, di or tri-halo C₁₋₄ alkyl, and
   (f) mono, di or tri-halo —OC₁₋₄ alkyl;
      R₄ is selected from the group consisting of:
(1) —C₁₋₄alkyl, optionally tritiated, and
(3) H;

Within this genus there is a sub-genus of compound of formula II wherein

R₁ is selected from the group consisting of:

(1) phenyl,

(2) pyridinyl,

(3) pyrimidinyl,

(4) pyrazinyl, and

(5) pyridazinyl,

wherein choice (1) to (5) is substituted with

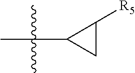

and R₅ is selected from the group consisting of

(a) —C₁₋₄alkyl optionally substituted with hydroxy,

(b) —S(O)₂C₁₋₄alkyl,

(c) —C(O)—NR₁₀R₁₁, and

(d) HET₂,

wherein choice (d) is optionally mono or di-substituted with substituents selected from:

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)O—C₁₋₃alkyl, and

(9) —C(O)—NR₁₉R₂₀,

wherein R₁₀, R₁₁, R₁₉ and R₂₀ are each independently selected from H and C₁₋₄alkyl, wherein C₁₋₄alkyl is optionally tritiated or mono-, di-, or tri-substituted with halo, or
R₂ is selected from the group consisting of:

(1) hydrogen,

(2) —C₁₋₆alkyl, and

(3) —C₃₋₆cycloalkyl,

wherein choice (2) and (3) are each optionally mono or di-substituted with substituents independently selected from the group consisting of

(a) halo,

(b) —CN,

(c) —OH,

(d) -hydroxy C₁₋₄alkyl,

(e) —CH₃,

(f) —CF₃, and

(g) —OCH₃;

R₃ is selected from the group consisting of:

(1) phenyl,

(2) pyrimidinyl,

(3) pyridinyl,

(4) pyrazinyl, and

(5) pyridazinyl,
wherein choices (1), (2), (3), (4) and (5) are each optionally mono or di-substituted with halo, haloC₁₋₄alkyl, or —OC₁₋₄alkyl optionally substituted with halo.

Within this sub-genus there is a class of compound of formula II wherein

R₁ is selected from the group consisting of:

(1) phenyl, and

(2) pyridinyl,

wherein choice (1) and (2) is substituted with

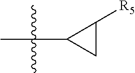

and R₅ is selected from the group consisting of

(a) —C₁₋₄alkyl optionally substituted with hydroxy,

(b) —S(O)₂C₁₋₄alkyl,

(c) —C(O)—NR₁₀R₁₁,

(d) HET₂, and

wherein choice (d) is optionally mono or di-substituted with substituents selected from:

(1) halo,

(2) —CN,

(3) —OH,

(4) —C₁₋₄alkyl optionally substituted with hydroxy, halo or cyano,

(5) —CF₃,

(6) —OC₁₋₄alkyl optionally substituted with hydroxyl or halo,

(7) —C(O)OH, and

(8) —C(O)—C₁₋₃alkyl, and

(9) —C(O)—NR₁₉R₂₀,

wherein R₁₀, R₁₁, R₁₉ and R₂₀ are each independently selected from H and C₁₋₄alkyl, wherein C₁₋₄alkyl is optionally tritiated mono-, di-, or tri-substituted with halo, or
R₂ is selected from the group consisting of:

(1) hydrogen,

(2) —C₁₋₆alkyl, and

(3) —C₃₋₆cycloalkyl,

wherein choice (2) and (3) are each optionally mono or di-substituted with substituents independently selected from the group consisting of

(a) halo,

(b) —CN,

(c) —OH,

(d) -hydroxy C₁₋₄alkyl,

(e) —CH₃,

(f) —CF₃, and

(g) —OCH₃;

R₃ is selected from the group consisting of

(1) phenyl,

(2) pyrimidinyl,

(3) pyridinyl,
wherein choices (1), (2) and (3) are each optionally mono or di-substituted with halo, haloC₁₋₄alkyl, or —OC₁₋₄alkyl optionally substituted with halo.

Illustrating the compounds of formula II are Examples 1B to 43B.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature.

Examples of isotopes suitable for inclusion in the compounds of the invention include isotopes of hydrogen such as 211 and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ²³I and ¹²⁵I, and chlorine such as ³⁶Cl.

Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The invention is described using the following definitions unless otherwise indicated.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl.

The term “alkoxy” means alkoxy groups of a straight, branched or cyclic configuration having the indicated number of carbon atoms. C₁₋₆alkoxy, for example, includes methoxy, ethoxy, propoxy, isopropoxy, and the like.

The term “alkylthio” means alkylthio groups having the indicated number of carbon atoms of a straight, branched or cyclic configuration. C₁₋₆alkylthio, for example, includes methylthio, propylthio, isopropylthio, and the like.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₃₋₆alkynyl, for example, includes propynyl, 1-methylethynyl, butynyl and the like.

The term “cycloalkyl” means mono-, bi- or ti-cyclic structures, optionally combined with linear or branched structures, the indicated number of carbon atoms. Examples of cycloalkyl groups include cyclopropyl, cyclopentyl, cycloheptyl, adamantyl, cyclododecylmethyl, 2-ethyl-1 bicyclo[4.4.0]decyl, and the like.

The term “aryl” is defined as a mono- or bi-cyclic aromatic ring system and includes, for example, phenyl, naphthyl, and the like.

The term “aralkyl” means an alkyl group as defined above of 1 to 6 carbon atoms with an aryl group as defined above substituted for one of the alkyl hydrogen atoms, for example, benzyl and the like.

The term “aryloxy” means an aryl group as defined above attached to a molecule by an oxygen atom (aryl-O) and includes, for example, phenoxy, naphthoxy and the like.

The term “aralkoxy” means an aralkyl group as defined above attached to a molecule by an oxygen atom (aralkyl-O) and includes, for example, benzyloxy, and the like.

The term “arylthio” is defined as an aryl group as defined above attached to a molecule by a sulfur atom (aryl-S) and includes, for example, thiophenoxy, thionaphthoxy and the like.

The term “aroyl” means an aryl group as defined above attached to a molecule by an carbonyl group (aryl-C(O)—) and includes, for example, benzoyl, naphthoyl and the like.

The term “aryloxy” means an aroyl group as defined above attached to a molecule by an oxygen atom (aroyl-O) and includes, for example, benzoyloxy or benzoxy, naphthoyloxy and the like.

The term “HET”, such as in “HET¹”, “HET²”, “HET³”, “HET⁴”, “HET₁”, “HET₂”, “HET₃”, “HET₄” is defined as a 5- to 10-membered aromatic, partially aromatic or non-aromatic mono- or bicyclic ring, containing 1-4 heteroatoms selected from O, S and N, and optionally substituted with 1-2 oxo groups. Where applicable, the Het group shall be defined to include the N-oxide. Preferably, “HET” is a 5- or 6-membered aromatic or non-aromatic monocyclic ring containing 1-3 heteroatoms selected from O, S and N, for example, pyridine, pyrimidine, pyridazine, furan, thiophene, thiazole, oxazole, isoxazole and the like, or HET is a 9- or 10-membered aromatic or partially aromatic bicyclic ring containing 1-3 heteroatoms selected from O, S, and N, for example, benzofuran, benzothiophene, indole, pyranopyrrole, benzopyran, quinoline, benzocyclohexyl, napthyridine and the like. “HET” also includes the following: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl. In one aspect “HET” is selected from pyridyl, pyridazinyl, pyrimidinyl, pyrazinyl, thiazolyl, thienyl, pyrrolyl, oxazolyl, and oxadiazole.

For all of the above definitions, each reference to a group is independent of all other references to the same group when referred to in the Specification. For example, if both R¹ and R² are HET, the definitions of HET are independent of each other and R¹ and R² may be different HET groups, for example furan and thiophene.

The ability of the compounds of Formula I to selectively inhibit FAAH makes them useful for treating, preventing, or reversing the progression of a variety of inflammatory and non-inflammatory diseases and conditions.

Diseases, disorders, syndromes and/or conditions, that would benefit from inhibition of FAAH enzymatic activity include, for example, Alzheimer's Disease, schizophrenia, depression, alcoholism, addiction, suicide, Parkinson's disease, Huntington's disease, stroke, emesis, miscarriage, embryo implantation, endotoxic shock, liver cirrhosis, atherosclerosis, cancer, traumatic head injury, glaucoma, and bone cement implantation syndrome.

Other diseases, disorders, syndromes and/or conditions that would benefit from inhibition of FAAH activity, include, for example, multiple sclerosis, retinitis, amyotrophic lateral sclerosis, immunodeficiency virus-induced encephalitis, attention-deficit hyperactivity disorder, pain, nociceptive pain, neuropathic pain, inflammatory pain, noninflammatory pain, painful hemorrhagic cystitis, pain associated with the herpes virus, pain associated with diabetes, peripheral neuropathic pain, central pain, thalamic pain syndrome, differentiation pain, chronic nociceptive pain, stimulus of nociceptive receptors, phantom and transient acute pain, post-operative pain, cancer pain, pain and spasticity associated with multiple sclerosis, arachnoiditis, radiculopathies, neuralgias, somatic pain, deep somatic pain, surface pain, visceral pain, acute pain, chronic pain, breakthrough pain, chronic back pain, failed back surgery syndrome, fibromyalgia, post-stroke pain, trigeminal neuralgia, sciatica, pain from radiation therapy, complex regional pain syndromes, causalgia, reflex sympathetic dystrophy, phantom limb pain, and myofascial pain.

Other diseases, disorders, syndromes and/or conditions that would benefit from inhibition of FAAH activity, include, obesity, hyperlipidemia, metabolic disorders, feeding and fasting, alteration of appetite, stress, memory, aging, hypertension, septic shock, cardiogenic shock, intestinal inflammation and motility, irritable bowel syndrome, colitis, diarrhea, ileitis, ischemia, cerebral ischemia, hepatic ischemia, myocardial infarction, cerebral excitotoxicity, seizures, febrile seizures, neurotoxicity, neuropathies, sleep, induction of sleep, prolongation of sleep, insomnia, and inflammatory diseases. Neurological and psychological disorders that would benefit from inhibition of FAAH activity include, for example, pain, depression, anxiety, generalized anxiety disorder (GAD), obsessive compulsive disorders, stress, stress urinary incontinence, attention deficit hyperactivity disorders, schizophrenia, psychosis, Parkinson's disease, muscle spasticity, epilepsy, diskenesia, seizure disorders, jet lag, and insomnia.

FAAH inhibitors can also be used in the treatment of a variety of metabolic syndromes, diseases, disorders, and/or conditions, including but not limited to, insulin resistance syndrome, diabetes, hyperlipidemia, fatty liver disease, obesity, atherosclerosis and arteriosclerosis. FAAH inhibitors are useful in the treatment of a variety of painful syndromes, diseases, disorders and/or conditions, including but not limited to those characterized by non-inflammatory pain, inflammatory pain, peripheral neuropathic pain, central pain, differentiation pain, chronic nociceptive pain, stimulus of nociceptive receptors, phantom and transient acute pain.

Inhibition of FAAH activity can also be used in the treatment of a variety of conditions involving inflammation. These conditions include, but are not limited to arthritis (such as rheumatoid arthritis, shoulder tendonitis or bursitis, gouty arthritis, and aolymyalgia rheumatica), organ-specific inflammatory diseases (such as thyroiditis, hepatitis, inflammatory bowel diseases), asthma, other autoimmune diseases (such as multiple sclerosis), chronic obstructive pulmonary disease (COPD), allergic rhinitis, and cardiovascular diseases.

In some cases, FAA H inhibitors are useful in preventing neurodegeneration or for neuroprotection.

In addition, it has been shown that when FAAH activity is reduced or absent, one of its substrates, anandamide, acts as a substrate for COX-2, which converts anandamide to prostamides (Weber et al., J Lipid, Res. 2004; 45:757). Concentrations of certain prostamides may be elevated in the presence of a FAAH inhibitor. Certain prostamides are associated with reduced intraocular pressure and ocular hypotensivity. Thus, in one embodiment, FAAH inhibitors may be useful for treating glaucoma.

In some embodiments, FAAH inhibitors can be used to treat or reduce the risk of EMDs, which include, but are not limited to, obesity, appetite disorders, overweight, cellulite, Type I and Type I1 diabetes, hyperglycemia, dyslipidemia, steatohepatitis, liver steatosis, non-alcoholic steatohepatitis, Syndrome X, insulin resistance, diabetic dyslipidemia, anorexia, bulimia, anorexia nervosa, hyperlipidemia, hypertriglyceridemia, atherosclerosis, arteriosclerosis, inflammatory disorders or conditions, Alzheimer's disease, Crohn's disease, vascular inflammation, inflammatory bowel disorders, rheumatoid arthritis, asthma, thrombosis, or cachexia.

In other embodiments, FAAH inhibitors can be used to treat or reduce the risk of insulin resistance syndrome and diabetes, i.e., both primary essential diabetes such as Type I Diabetes or Type I1 Diabetes and secondary nonessential diabetes. Administering a composition containing a therapeutically effective amount of an in vivo FAAH inhibitor reduces the severity of a symptom of diabetes or the risk of developing a symptom of diabetes, such as atherosclerosis, hypertension, hyperlipidemia, liver steatosis, nephropathy, neuropathy, retinopathy, foot ulceration, or cataracts.

In another embodiment, FAAH inhibitors can be used to treat food abuse behaviors, especially those liable to cause excess weight, e.g., bulimia, appetite for sugars or fats, and non-insulin-dependent diabetes.

In some embodiments, FAAH inhibitors can be used to treat a subject suffering from an EMD and also suffers from a depressive disorder or from an anxiety disorder. Preferably, the subject is diagnosed as suffering from the depressive or psychiatric disorder prior to administration of the FAAH inhibitor composition. Thus, a dose of a FAAH inhibitor that is therapeutically effective for both the EMD and the depressive or anxiety disorder is administered to the subject.

Preferably, the subject to be treated is human. However, the methods can also be used to treat non-human mammals. Animal models of EMDs such as those described in, e.g., U.S. Pat. No. 6,946,491 are particularly useful.

FAAH inhibitor compositions can also be used to decrease body-weight in individuals wishing to decrease their body weight for cosmetic, but not necessarily medical considerations.

It will be appreciated that when using any combination described herein, both the FAAH compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

A FAAH inhibitor composition can be administered in combination with a drug for lowering circulating cholesterol levels (e.g., statins, niacin, fibric acid derivatives, or bile acid binding resins). FAAH inhibitor compositions can also be used in combination with a weight loss drug, e.g., orlistat or an appetite suppressant such as diethylpropion, mazindol, orlistat, phendimetrazine, phentermine, or sibutramine.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition. The term “amount effective for treating” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician.

The following abbreviations have the indicated meanings:
AIBN=2,2′-azobisisobutyronitrile
B.P.=benzoyl peroxide
Bn=benzyl
CCl₄=carbon tetrachloride
D=−—O(CH₂)₃O—
DAST=diethylamine sulfur trifluoride
DCC=dicyclohexyl carbodiimide
DCI=1-(3-dimethylaminopropyl)-3-ethyl carbodiimide
DEAD=diethyl azodicarboxylate
DIBAL=diisobutyl aluminum hydride
DME=ethylene glycol dimethylether
DMAP=4-(dimethylamino)pyridine
DMF=N,N-dimethylfomamide
DMSO=dimethyl sulfoxide
Et₃N=triethylamine
LDA=lithium diisopropylamide
m-CPBA=metachloroperbenzoic acid
NBS=N-bromosuccinimide
NSAID=non-steroidal anti-inflammatory drug
PCC=pyridinium chlorochromate
PDC=pyridinium dichromate
Ph=phenyl
1,2-Ph=1,2-benzenediyl
Pyr=pyridinediyl
Qn=7-chloroquinolin-2-yl
R^{S}=—CH₂SCH₂CH₂Ph
r.t.=room temperature
rac.=racemic
THF=tetrahydrofuran
THP=tetrahydropyran-2-yl

### Alkyl Group Abbreviations

Me=methyl
Et=ethyl
n-Pr=normal propyl
i-Pr=isopropyl
n-Bu=normal butyl
i-Bu=isobutyl
s-Bu=secondary butyl
t-Bu=tertiary butyl
c-Pr=cyclopropyl
c-Bu=cyclobutyl
c-Pen=cyclopentyl
c-Hex=cyclohexyl

Some of the compounds described herein contain one or more asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that in the discussion of methods of treatment which follows, references to the compounds of Formula I are meant to also include the pharmaceutically acceptable salts.

The magnitude of prophylactic or therapeutic dose of a compound of Formula I will, of course, vary with the nature and the severity of the condition to be treated and with the particular compound of Formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for oral administration to humans may contain from about 0.5 mg to about 5 g of active agent compounded with an appropriate and convenient amount of carrier material which may vary from about 5 to about 95 percent of the total composition. Dosage unit forms will generally contain from about 1 mg to about 2 g of an active ingredient, typically 25 mg, 50 mg, 100 mg, 200 mg, 300 mg, 400 mg, 500 mg, 600 mg, 800 mg, or 1000 mg.

For the treatment of FAAH mediated diseases the compound of Formula I may be administered orally, topically, parenterally, by inhalation spray or rectally in dosage unit formulations containing conventional non-toxic pharmaceutically acceptable carriers, adjuvants and vehicles. The term parenteral as used herein includes subcutaneous, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a fond suitable for oral use, for example, as tablets, troches, lozenges, solutions, aqueous or oily suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108, 4,166,452, and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol, or sucrose. Such formulations may also contain a demulcent, a preservative, flavouring, and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. Cosolvents such as ethanol, propylene glycol, or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing a compound of Formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

### ASSAYS

The following assays illustrate the utility of the invention:

The compounds of the invention underwent pharmacological evaluations to determine their inhibitory effect on the enzyme FAAH (Fatty Acid Amide Hydrolase).

To assist in assay development stable cell lines for human, murine and rat full length FAAH were developed. Human FAAH cDNA (Accession No: NM_{—}001441.1) was purchased from Origene (Rockville, Md.). The full length FAAH was subcloned into the mammalian expression vector, peDEF.neo, using XbaI and EcoRI restriction sites and used for stable cell line generation.

| Construct | Primer | Sequence |
|---|---|---|
| Full length rodent FAAH | 1 | CAAGGTACCGCCACCATGGTGCTGAGCGAAGTGTGG |
| Full length murine FAAH | 2 | CCGGAATTCTCAAGATGGCCGCTTTTCAGG |
| Full length rat FAAH | 3 | CCGGAATTCTCACGATGGCTGCTTTTGAGG |

Murine (accession number NM_{—}010173) and Rat FAAH (accession number NM_{—}024132) was amplified by reverse transcriptase polymerase chain reaction (RT-PCR) from brain cDNA (BD Biosciences, San Jose, Calif.) using primers 1 and 2 or primers 1 and 3 respectively (see Table). The resulting PCR product was ligated into pCR4 TOPO and DNA sequence confirmed. The full length murine FAAH was subcloned into the mammalian expression vector, pcDEFneo using either EcoRI (murine) or KpnI and EcoRI (rat) restriction sites. Chinese hamster ovary cells (CHO) were transfected following manufacturers protocol (AMAXA). Forty eight hours post transfection, cells were trypsinized and transferred to 96 well plates in Iscove's DMEM media supplemented with 2 mM Glutamine, 10% fetal calf serum, 1 mg/ml geneticin and HT Supplement (0.1 mM sodium hypoxanthine, 0.016 mM thymidine) in order to isolate single clones. Following selection in geneticin, individual clones were selected and FAAH activity was assessed using a whole cell fluorescent anandamide assay, modified from Ramarao et al (2005). Following removal of tissue culture media cells were dislodged following addition of Cellstripper (Mediatech, Inc. Manassas, Va.) and transferred to 96 well black clear bottom assay plate, centrifuged at 1,000 rpm for 3 mins and media removed and replaced with assay buffer (50 mM Tris pH8.0, 1 mM EDTA, 0.1% fatty acid free BSA). The reaction was initiated by addition of fluorescent substrate, AMC Arachidonoyl Amide (Cayman Chemical, Ann Arbor, Mich.) to 1 μM and reaction allowed to proceed for 2 hours at room temperature. Release of fluorescence was monitored in a CytoFluor Multiplate Reader. Cells expressing the highest amount of FAAH activity were selected for study with FAAH inhibitors.

### Preparation of Lysate and Microsomes

CHO cells expressing FAAH were used to prepare either crude cell lysate or microsome fractions. To harvest cells, tissue culture media was decanted, the monolayer washed three times with Ca⁺⁺Mg⁺⁺ free PBS and cells recovered after 15 mM in enzyme free dissociation media (Millipore Corp, Billerica, Mass.). Cells were collected by centrifuging at 2000 rpm for 15 min. and the cell pellet re-suspended with 50 mM HEPES (pH 7.4) containing 1 mM EDTA and the protease inhibitors aprotinin (1 mg/ml) and leupeptin (100 μM). The suspension was sonicated at 4° C. and the cell lysate recovered after centrifuging at 12,000×g (14,600 rpm, SS34 rotor) for 20 min at 4° C. to form a crude pellet of cell debris, nuclei, peroxisomes, lysosomes, and mitochondria; the supernatant or cell lysate was used for FAAH enzyme assay. In some cases, microsomes fractions enriched in FAAH were prepared by centrifuging the cell lysate further at 27,000 rpm (100,000×g) in SW28 rotor for 50 minutes at 4° C. The pellet containing FAAH-enriched microsomes was re-suspend in 50 mM HEPES, (pH 7,4) 1 mM EDTA, and any remaining DNA sheared by passage of material through a 23 gauge needle and aliquots of enzyme were store at −80° C. prior to use.

### FAAH Assays

Several assays have been used to demonstrate the inhibitory activity. Enzyme activity was demonstrated in a radioenzymatic test based on measuring the product of hydrolysis (ethanolamine [³H]) of anandamide [ethanolamine 1-.sup.3H] (American Radiolabeled Chemicals; 1 mCi/ml) with FAAH (Life Sciences (1995), 56, 1999-2005 and Journal of Pharmacology and Experimented Therapeutics (1997), 283, 729-734), Analytical, Biochemistry (2003), 318, 270-5. In addition, routine assays were performed monitoring hydrolysis of arachidonoyl-7-amino-4-methylcoumarin amide (AAMCA) by following increase in fluorescence upon release of 7-amino 4-methyl coumarin (λ_{EX}=355 nm, (λ_{EM}=460 nm), Analytical, Biochemistry (2005), 343, 143-51

Assays are performed on either cell lysate or microsome fractions prepared as described or in whole cell format employing either the fluorescent substrate AAMCA (Cayman chemical, Ann Arbor, Mich.,) or ³H-anandamide ([ETHANOLAMINE-1-3H] American Radiolabeled Chemicals; 1 mCi/ml). The cell lysate or microsome assay is performed in Costar black wall, clear bottom plates by adding FAAH_CHO (whole cell, cell lysate or microsome) in assay buffer (50 mM Phosphate, pH 8.0, 1 mM EDTA, 200 mM KCl, 0.2% glycerol, 0.1% fatty acid free BSA) to each well, followed by either DMSO or compound and allowed to incubate at 22-25° C. for fifteen minutes. AAMCA substrate was used to achieve a final concentration of 1 μM and reaction allowed to proceed at room temperature for 1-3 hours. Fluorescent release as a measure of FAAH activity was monitored by reading the plate in a CytoFluor Multiplate Reader (Ex: 360/40 nM; Em: 460/40 nM). Whole cell assay is conducted with cells harvested after rinsing tissue culture flasks three times with Ca⁺⁺Mg⁺⁺ free PBS, incubating for 10 mm in Enzyme free dissociation media and centrifuging for 5 minutes at 1,000 rpm in table top centrifuge. Cells are resuspended in assay buffer at desired cell number in (4×10⁴ cells/assay in 96-well format; 1×10⁴ cells/assay in 384-well format) and assayed as described.

Alternatively, assays are performed using anandamide [ethanolamine 1-.sup.3H] (specific activity of 10 Ci/mmol) diluted with cold anandamide to achieve a final assay concentration of 1 μM anandamide (˜50,000 cpm). Enzyme (CHO cell lysate, brain or liver homogenate) is incubated in assay buffer (50 mM Phosphate, pH 8.0, 1 mM EDTA, 200 mM KCl, 0.2% glycerol, 0.1% fatty acid free BSA) with inhibitor at 25° C. for 30 minutes. The reaction was terminated by addition of 2 volumes of chloroform:methanol (1:1) and mixed by vortexing. Following a centrifugation step, 2000 rpm for 10 min. at room temperature, the aqueous phase containing the released ³H-ethanolamide was recovered and quantitated by liquid scintillation as a reflection of FAAH enzyme activity.

Ramarao M. K., et al. A fluorescence-based assay for fatty acid amide hydrolase compatible with high-throughput screening, Anal. Biochem., 343:143-51 (2005)
Wilson S. J., et l., A high-throughput-compatible assay for determining the activity of fatty acid amide hydrolase, Anal Biochem, 318:270-5 (2003).

Each of Examples 1 through 29 was tested and found to demonstrate biological activity. Results for specific Examples are provided below. Each of Examples 1 through 27 was found to have and IC50 of 3 μM or lower in these assays.

### Preparation of the Compounds of the Invention.

The compounds of the present invention can be prepared according to the procedures denoted in the following reaction Schemes and Examples or modifications thereof using readily available starting materials, reagents, and conventional procedures thereof well-known to a practitioner of ordinary skill in the art of synthetic organic chemistry. Specific definitions of variables in the Schemes are given for illustrative purposes only and are not intended to limit the procedures described.

### Preparation of the Compounds of the Invention.

The compounds of the present invention can be prepared according to the procedures denoted in the following reaction Schemes and Examples or modifications thereof. using readily available starting materials, reagents, and conventional procedures thereof well-known to a practitioner of ordinary skill in the art of synthetic organic chemistry. Specific definitions of variables in the Schemes are given for illustrative purposes only and are not intended to limit the procedures described.

### Preparation of the Compounds of the Invention.

The compounds of the present invention can be prepared according to the procedures denoted in the following reaction Schemes and Examples or modifications thereof using readily available starting materials, reagents, and conventional procedures thereof well-known to a practitioner of ordinary skill in the art of synthetic organic chemistry. Specific definitions of variables in the Schemes are given for illustrative purposes only and are not intended to limit the procedures described.

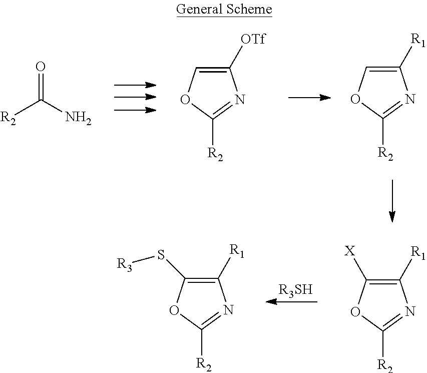

### Intermediate 1

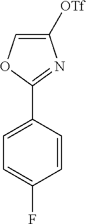 [SMILES: O=S(=O)(Oc1coc(-c2ccc(F)cc2)n1)C(F)(F)F]

### 2-(4-Fluorophenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 2

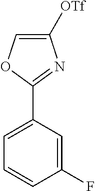 [SMILES: O=S(=O)(Oc1coc(-c2cccc(F)c2)n1)C(F)(F)F]

### 2-(3-Fluorophenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 3

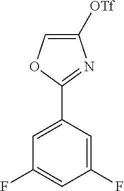 [SMILES: O=S(=O)(Oc1coc(-c2cc(F)cc(F)c2)n1)C(F)(F)F]

### 2-(3,5-Difluorophenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 4

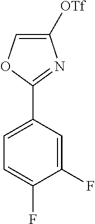 [SMILES: O=S(=O)(Oc1coc(-c2ccc(F)c(F)c2)n1)C(F)(F)F]

### 2-(3,4-Difluorophenyl)-1,3-oxazol-4-yl trifluoromethanesulfonate

The title compound was prepared using the procedure described by Langille, N. F.; Dakin, L. A.; Panek, J. S. Org. Lett, 2002, 4, 2485.

### Intermediate 5

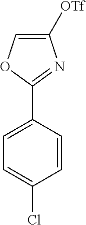 [SMILES: O=S(=O)(Oc1coc(-c2ccc(Cl)cc2)n1)C(F)(F)F]

### 2-(4-Chlorophenyl)-1,3-oxazol-4-yltrifluoromethanesulfonate

The title compound was prepared using the procedure described by Langille, N.Y.; Dakin, L. A.; Panek, J. S. Org. Lett., 2002, 4, 2485.

### Intermediate 6

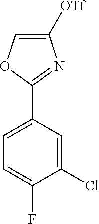 [SMILES: O=S(=O)(Oc1coc(-c2ccc(F)c(Cl)c2)n1)C(F)(F)F]

### 2-(3-Chloro-4-fluorophenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 7

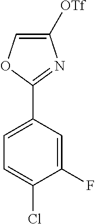 [SMILES: O=S(=O)(Oc1coc(-c2ccc(Cl)c(F)c2)n1)C(F)(F)F]

### 2-(4-Chloro-3-fluorophenyl)-1,3-oxazol-4-yltrifluoromethanesulfonate

The title compound was prepared using the procedure described by Langille, N. F.; Dakin, L. A.; Panek, J. S. Org. Lett., 2002, 4, 2485.

### Intermediate 8

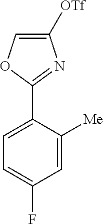 [SMILES: Cc1cc(F)ccc1-c1nc(OS(=O)(=O)C(F)(F)F)co1]

### 2-(4-Fluoro-2-methylphenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 9

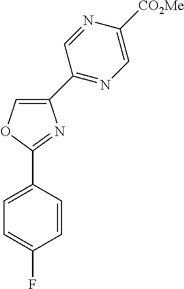 [SMILES: COC(=O)c1cnc(-c2coc(-c3ccc(F)cc3)n2)cn1]

### Methyl 5-[2-(4-fluorophenyl)-1,3-oxazol-4-yl]pyrazine-2-carboxylate

### Step A.

A solution of intermediate 1 (2.66 g, 8.55 mmol), bis-pinacolatodiboron (2.60 g, 10.3 mmol), KOAc (1.68 g, 17.1 mmol), and Pd(dppf)Cl₂ (0.70 g, 0.86 mmol) in 1,4-dioxane (25 mL) were heated to 140° C. for 30 min. Upon completion of the reaction as judged by TLC analysis, the solution was concentrated to dryness and purified on silica gel to afford the corresponding boronic acid intermediate which was taken on immediately.

Step B.

The boronic acid prepared in Step A (1.00 g, 4.80 mmol), methyl 5-chloropyrazine-2-carboxylate (1.70 g, 10.0 mmol), Pd(PPh₃)₄ (558 mg, 0.48 mmol), K₂CO₃ (2.00 g, 14.5 mmol) were dissolved in toluene (10 mL) and H₂O (1 mL) and degassed for 5 min. After which, the solution was heated in the microwave reactor to 120° C. for 30 min. Upon completion of the reaction as judged by TLC analysis, the solution was diluted with dist H₂O and extracted with EtOAc. The organic layer was removed, dried over MgSO₄, filtered and concentrated giving rise to an oil. The oil was purified on silica gel to to afford the title compound (290 mg). LC/MS: mile 300.1 (M+H).

### Intermediate 10

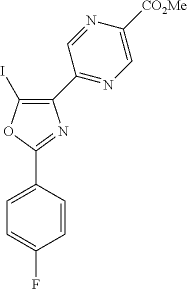 [SMILES: COC(=O)c1cnc(-c2nc(-c3ccc(F)cc3)oc2I)cn1]

### 2-Pyrazinecarboxylic acid, 5-[2-(4-fluorophenyl)-5-iodo-4-oxazolyl]-methyl ester

A solution of Intermediate 9 (1.40 g, 4.70 mmol), NIS (1.30 g, 5.60 mmol), TPA (0.40 mL) in CH₃CN (100 mL) was stirred at rt for 12 h. Upon completion of the reaction, the solution was diluted with sat aq Na₂S₂O₃ and extracted with EtOAc. The organic layer was removed, dried over MgSO₄, filtered and concentrated giving rise to an oil. The oil was purified on silica gel to afford the title compound (684 mg). LC/MS: m/e 425.9 (M+H)⁺. ¹H NMR (500 MHz, Acetone-d6): δ 4.01 (s, 3H), 7.41 (t, J=8.8 Hz, 2H), 8.20-8.25 (m, 2H), 9.28 (s, 1H), 9.39 (s, 1H).

### Intermediate 11

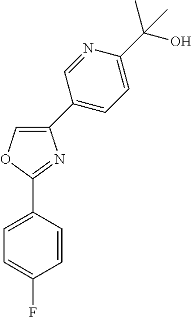 [SMILES: CC(C)(O)c1ccc(-c2coc(-c3ccc(F)cc3)n2)cn1]

### 2-{5-[2-(4-Fluorophenyl)-1,3-oxazol-4-yl]pyridin-2-yl}propan-2-ol

A solution of Intermediate 1 (60 g, 0.20 mol), bis-pinacolatodiboron (500 g, 0.25 mol), KOAc (57.0, 0.58 mol), Pd(dppf)Cl₂ (7.90 g, 9.60 mmol), and dppf (5.34 g, 9.60 mmol) in 1,4-dioxane (1.6 L) were heated to 101° C. for 3 h. Upon completion of the reaction as judged by TLC analysis, the reaction was allowed to cool to 65° C. At which point, 2-(5-bromopyridin-2-yl)propan-2-ol (62.6 g, 0.30 mol) and Pd(PPh₃)₂Cl₂ (13.6 g, 0.02 mol) were added followed by dropwise addition of aqueous Na₂CO₃ (193 mL, 0.40 mol, 2 M). The solution was heated to 91° C. for 12 h. Upon completion of the reaction as judged by LC/MS analysis, the solution was diluted with dist H₂O and extracted with EtOAc (2×). The combined organic layers were removed, dried over MgSO₄, filtered and concentrated giving rise to an oil. The oil was purified on silica gel to give afford the title compound (38.50 g). LC/MS: m/e 299.1 (M+H).

### Intermediate 12

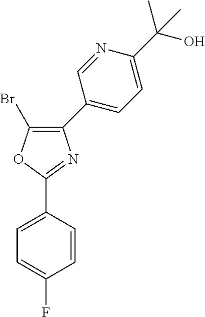 [SMILES: CC(C)(O)c1ccc(-c2nc(-c3ccc(F)cc3)oc2Br)cn1]

### 2-{5-[5-Bromo-2-(4-fluorophenyl)-1,3-oxazol-4-yl]pyridin-2-yl}propan-2-ol

A solution of Intermediate 11 (38.5 g, 0.13 mol) and NBS (28.0 g, 0.16 mol) in CH₂Cl₂ (1340 mL) was stirred at rt for 12 h. Upon completion of the reaction, the solution was diluted with sat aq NaS₂O₃ solution. The organic layer was removed, dried over MgSO₄, filtered and concentrated giving rise to an oil. The oil was purified on silica gel to afford the title compound (31.97 g). LC/MS: m/e 377.0 (M+H)⁺.

### Intermediate 13

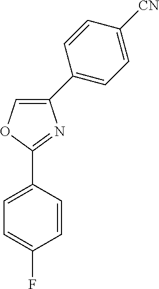 [SMILES: N#Cc1ccc(-c2coc(-c3ccc(F)cc3)n2)cc1]

### 4-[2-(4-Fluorophenyl)-1,3-oxazol-4-yl]benzonitrile

A solution of Intermediate 1 (560 mg, 1.80 mol), (4-cyanophenyl)boronic acid (291 mg, 2.00 mmol), K₂CO₃ (497 mg, 3.60 mmol) and Pd(PPh₃)₄ (104 mg, 0.09 mmol) in 1,4-dioxane (10 mL) were heated to 110° C. for 20 min. Upon completion of the reaction as judged by TLC analysis, the reaction was concentrated to dryness and purified on silica gel to afford the title compound (470 mg). LC/MS: m/e 265.2 (M+H).

### Intermediate 14

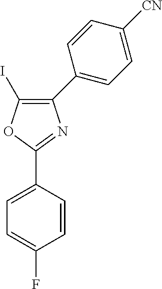 [SMILES: N#Cc1ccc(-c2nc(-c3ccc(F)cc3)oc2I)cc1]

### 4-[2-(4-Fluorophenyl)-5-iodo-1,3-oxazol-4-yl]benzonitrile

A solution of Intermediate 13 (476 mg, 1.80 mmol), NIS (608 mg, 2.70 mmol), TFA (0.14 mL) in CH₂Cl₂ (15 mL) was stirred at rt for 12 h. Upon completion of the reaction, the solution was diluted with sat aq Na₂S₂O₃ and extracted with EtOAc. The organic layer was removed, dried over MgSO₄, filtered and concentrated giving rise to an oil. The oil was purified on silica gel to afford the title compound (700 mg). LC/MS: m/e 391.1 (M+H)⁺. ¹H NMR (500 MHz, Acetone-d6): δ 7.41 (t, 2H), 7.94 (d, 2H), 8.20 (m, 2H), 8.36 (d, 2H).

### Intermediate 15

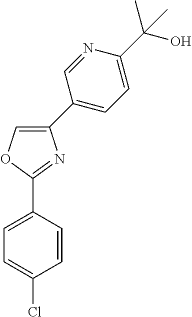 [SMILES: CC(C)(O)c1ccc(-c2coc(-c3ccc(Cl)cc3)n2)cn1]

### 2-{5-[2-(4-Chlorophenyl)-1,3-oxazol-4-yl]pyridin-2-yl}propan-2-ol

The target compound was prepared in an analogous manner to Intermediate 11 except that Intermediate 5 was coupled with 2-(5-bromopyridin-2-yl)propan-2-ol (XXX g). LC/MS: m/e 315.1 (M+H).

### Intermediate 16

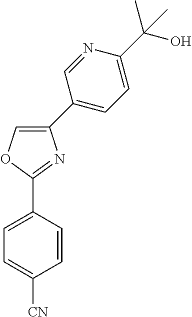 [SMILES: CC(C)(O)c1ccc(-c2coc(-c3ccc(C#N)cc3)n2)cn1]

### 4-{4-[6-(1-Hydroxy-1-methylethyl)pyridin-3-yl]-1,3-oxazol-2-yl}benzonitrile

A solution of Intermediate 15 (200 mg, 0.60 mmol), Pd₂ dba₃ (93 mg, 0.10 mmol), S-Phos (104 mg, 0.25 mmol) and Zn(CN)₂ (112 mg, 0.90 mmol) in 10 mL of 99:1 v:v DMF:H₂O were heated to 180° C. for 30 min in the microwave reactor. Upon completion of the reaction as judged by LC/MS analysis, the solution was diluted with dig H₂O and extracted with EtOAc (2×). The combined organic layers were removed, dried over MgSO₄, filtered and concentrated giving rise to an oil. The oil was purified on silica gel to afford the title compound (194 mg). LC/MS: m/e 306.1 (M+H).

### Intermediate 17

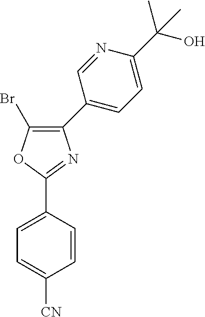 [SMILES: CC(C)(O)c1ccc(-c2nc(-c3ccc(C#N)cc3)oc2Br)cn1]

### 4-{5-Bromo-4-[6-(1-hydroxy-1-methylethyl)pyridin-3-yl]-1,3-oxazol-2-yl}benzonitrile

A solution of Intermediate 16 (476 mg, 1.80 mmol) and NBS (608 mg, 2.70 mmol) in CH₂Cl₂ (15 mL) was stirred at rt for 12 h. Upon completion of the reaction, the solution was diluted with sat aq Na₂S₂O₃ and extracted with EtOAc. The organic layer was removed, dried over MgSO₄, filtered and concentrated giving rise to an oil. The oil was purified on silica gel to afford the title compound (43.7 mg). LC/MS: m/e 384.0 (M±H)⁺.

### Example 1

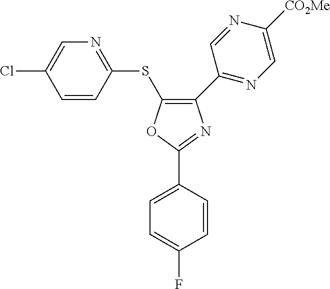 [SMILES: COC(=O)c1cnc(-c2nc(-c3ccc(F)cc3)oc2Sc2ccc(Cl)cn2)cn1]

### Methyl-5-[5-[(5-chloropyridin-2-yl)thio]-2-(4-fluorophenyl)-1,3-oxazol-4-yl]pyrazine-2-carboxylate

A solution of 5-chloropyridine-2-thiol (305 mg, 2.10 mmol) dissolved in 18 mL of NMP was treated with NaH (84 mg, 2.10 mmol). The resulting solution was stirred for 30 min at rt before Intermediate 10 (684 mg, 1.60 mmol) and CuI (306 mg, 1.60 mmol) were added to the solution. The resulting dark solution was heated to 120° C. for 2 h. After which point, the solution was poured into a rapidly stirred solution of 9:1 NH₄Cl:NH₄OH and EtOAc. Upon clarification of the organic layer, removal of the organic layer was followed by drying over MgSO₄, filtration and concentration giving rise to an oil. The oil was purified on silica gel to afford the title compound (410 mg). LC/MS: m/e 443.0 (M+H)⁺. ¹H NMR (500 MHz, Acetone-d6): δ 4.01 (s, 3H), 7.37-7.41 (m, 2H), 8.04 (m, 2H), 8.70 (s, 1H), 9.28 (d, J=1.5 Hz, 1H), 9.44 (d, J=1.0 Hz, 1H).

The compounds in Table 1 were prepared from the appropriate starting materials using the procedure for Example 1.

TABLE 1

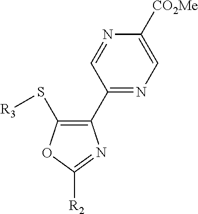

| | | | LCMS: found |
|---|---|---|---|
| Example | R₂ | R₃ | m/e (M + H) |
| 2 | 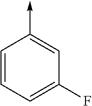 | 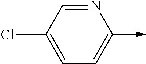 | 442.9 |

### Example 3

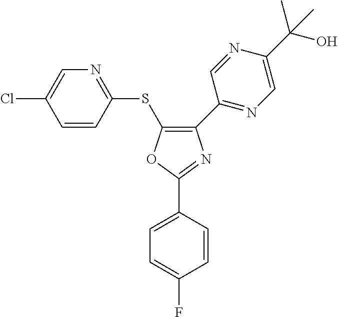 [SMILES: CC(C)(O)c1cnc(-c2nc(-c3ccc(F)cc3)oc2Sc2ccc(Cl)cn2)cn1]

### 2-{5-[5-[(5-Chloropyridin-2-yl)thio]-2-(4-fluorophenyl)-1,3-oxazol-4-yl]pyrazin-2-yl}propan-2-ol

A solution of methyl-5-[5-[(5-chloropyridin-2-yl)thio]-2-(4-fluorophenyl)-1,3-oxazol-4-yl]pyrazine-2-carboxylate (Example 1) (410 mg, 0.93 mmol) in THF (20 mL) was treated with methylmagnesium bromide (3.1 mL, 9.3 mmol, 3.0 M in THF) at it Upon completion of the reaction as judged by TLC analysis, the solution was diluted with saturated aq NH₄Cl solution and extracted with EtOAc. The organic layer was removed, dried over MgSO₄, filtered and concentrated giving rise to an oil. The oil was purified on silica gel to afford the title compound (90 mg). LC/MS: m/e 442.9 (M+H)⁺. ¹H NMR (500 MHz, Acetone-d6): δ 1.58 (s, 6H), 4.58 (s, 1H), 7.42 (m, 3H), 7.76 (dd, J=2.6, 8.8 Hz, 2H), 8.32 (m, 2H), 8.43 (d, J=2.7 Hz, 1H), 8.96 (s, 1H), 9.19 (s, 1H).

The compounds in Table 2 were prepared from the appropriate starting materials using the procedure for Example 3.

TABLE 2

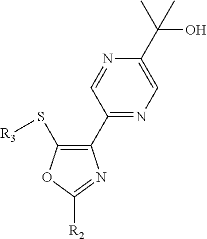

| | | | LCMS: found |
|---|---|---|---|
| Example | R₂ | R₃ | m/e (M + H) |
| 4 | 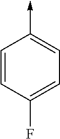 | 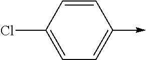 | 441.9 |
| 5 | 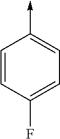 | 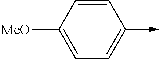 | 438.2 |
| 6 | 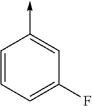 | 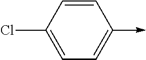 | 441.9 |
| 7 | 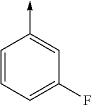 | 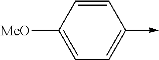 | 438.0 |
| 8 | 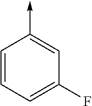 | 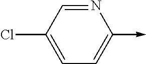 | 442.9 |

### Example 9

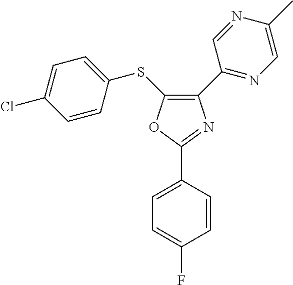 [SMILES: Cc1cnc(-c2nc(-c3ccc(F)cc3)oc2Sc2ccc(Cl)cc2)cn1]

### 2-[5-[(4-Chlorophenyl)thio]-2-(4-fluorphenyl)-1,3-oxazol-4-yl]-5-methylpyrazine

A solution of methyl-5-[5-[(4-chlorophenyl)thio]-2-(4-fluorophenyl)-1,3-oxazol-4-yl]pyrazine-2-carboxylate (Example 1) (24 mg, 0.05 mmol) in THF (5 mL) was treated with methylmagnesium bromide (0.2 mL, 0.5 mmol, 3.0 M in THF) at rt. Upon completion of the reaction as judged by TLC analysis, the solution was diluted with saturated aq NH₄Cl solution and extracted with EtOAc. The organic layer was removed, dried over MgSO₄, filtered and concentrated giving rise to an oil. The oil was purified on silica gel to afford the title compound (6.3 mg). LC/MS: m/e 397.0 (M+H)⁺. ¹H NMR (500 MHz, Acetone-d6): δ 2.55 (s, 3H), 7.46 (m, 5H), 8.06 (m, 2H), 8.50 (s, 1H), 8.55 (s, 1H), 9.10 (d, J=1.1 Hz, 1H).

The compounds in Table 3 were prepared from the appropriate starting materials using the procedure for Example 9.

TABLE 3

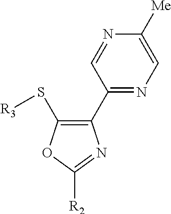

| | | | LCMS: found |
|---|---|---|---|
| Example | R₂ | R₃ | m/e (M + H) |
| 10 | 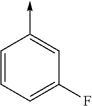 | 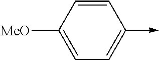 | 394.1 |
| 11 | 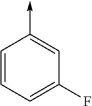 | 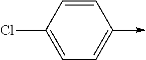 | 397.9 |

### Example 12

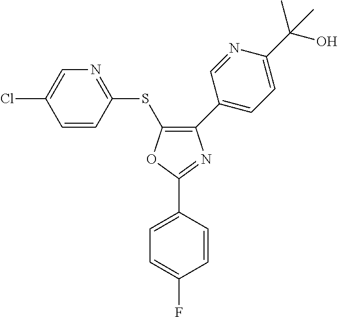 [SMILES: CC(C)(O)c1ccc(-c2nc(-c3ccc(F)cc3)oc2Sc2ccc(Cl)cn2)cn1]

### 2-{5-[5-[(5-Chloropyridin-2-yl)thio]-2-(4-fluorophenyl)-1,3-oxazol-4-yl]pyridin-2-yl}propan-2-ol

A solution of 5-chloropyridine-2-thiol (273 g, 0.20 mol) dissolved in 200 mL of NMP was treated with NaH (7.7 g, 0.20 mol). The resulting solution was stirred for 30 min at rt before Intermediate 12 (31.9 g, 0.08 mol) dissolved in 200 mL of NMP was added by addition funnel. Lastly, CuI (16.3 g, 0.08 mol) was added to the solution. The resulting dark solution was heated to 120° C. for 2 h. After which point, the solution was cooled to rt. Once at rt, the solution poured into a rapidly stirred solution of 9:1 NH₄Cl:NH₄OH and EtOAc. Upon clarification, the organic layer was removed followed by drying over MgSO₄, filtration, and concentration giving rise to an oil. The oil was purified on silica gel to afford the title compound (31.87 g). LC/MS: m/e 442.1 (M+H)⁺. ¹H NMR (500 MHz, Acetone-d6): δ 1.76 (s, 6H), 5.01 (s, 1H), 7.40 (m, 3H), 7.80 (m, 2H), 8.25 (m, 2H), 8.44 (dd, J=2.3, 8.2 Hz, 1H), 8.44 (d, J=2.3 Hz, 1H), 9.20 (d, J=1.4 Hz, 1H).

### Example 12^{a}

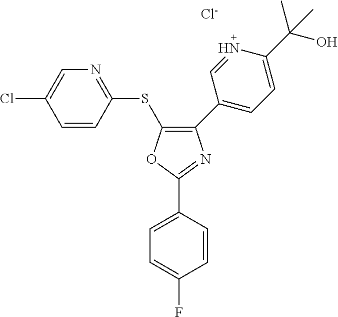 [SMILES: CC(C)(O)c1ccc(-c2nc(-c3ccc(F)cc3)oc2Sc2ccc(Cl)cn2)cn1 ; [Cl-]]

### 5-{5-[(5-Chloropyridin-2-yl)thio]-2-(4-fluorophenyl)-1,3-oxazol-4-yl]pyridin-2-yl}propan-2-ol, hydrogen chloride salt

A solution of Example 12 (138 mg, 0.31 mmol) was taken up in 7 mL of IPAC and heated to 65° C. Upon complete dissolution, HCl (78 μl, 031 mmol, 4N in dioxane) was added dropwise. The resulting slurry was maintained at 65° C. for 2 h before being allowed to cool to rt. The slurry was filtered giving rise to a white solid (100.7 mg), LC/MS: m/e 442.1 (M+H)⁺.

The compounds in Table 4 were prepared from the appropriate starting materials using the procedure for Example 12.

TABLE 4

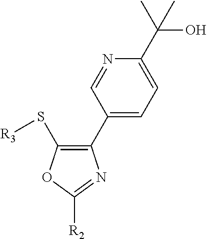

| | | | LCMS: |
|---|---|---|---|
| | | | found m/e |
| Example | R₂ | R₃ | (M + H) |
| 13 | 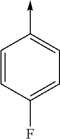 | 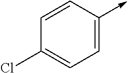 | 441.0 |
| 14 | 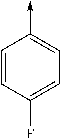 | 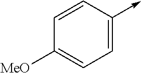 | 437.0 |
| 15 | 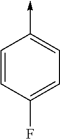 | 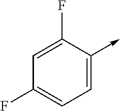 | 443.0 |
| 16 | 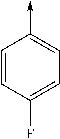 | 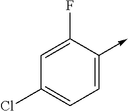 | 459.0 |
| 17 | 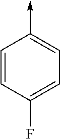 | 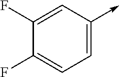 | 443.0 |
| 18 | 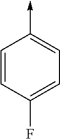 | 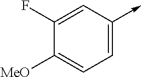 | 445.0 |
| 19 | 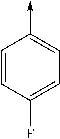 | 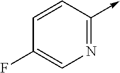 | 426.1 |
| 20 | 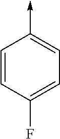 | 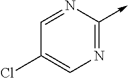 | 442.9 |
| 21 | 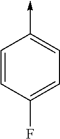 | 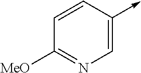 | 438.1 |
| 22 | 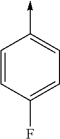 | 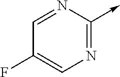 | 427.0 |
| 23 | 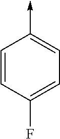 | 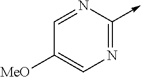 | 439.0 |
| 24 | 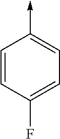 | 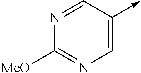 | 439.0 |
| 25 | 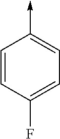 | 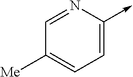 | 422.1 |
| 26 | 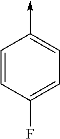 | 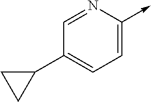 | 448.1 |
| 27 | 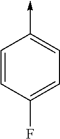 | 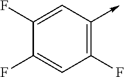 | 461.3 |
| 28 | 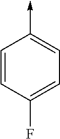 | 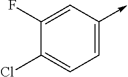 | 459.3 |
| 29 | 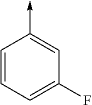 | 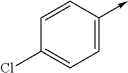 | 440.9 |
| 30 | 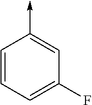 | 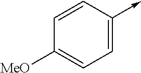 | 437.0 |
| 31 | 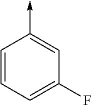 | 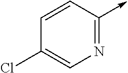 | 442.0 |
| 32 | 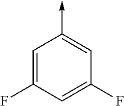 | 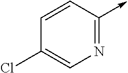 | 459.9 |
| 33 | 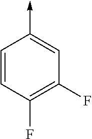 | 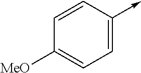 | 455.0 |
| 34 | 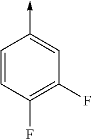 | 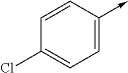 | 459.0 |
| 35 | 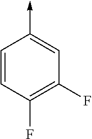 | 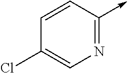 | 460.0 |
| 36 | 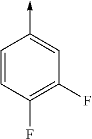 | 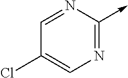 | 461.3 |
| 37 | 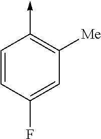 | 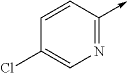 | 456.6 |
| 38 | 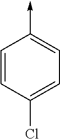 | 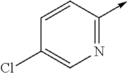 | 457.9 |
| 39 | 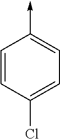 | 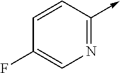 | 442.0 |
| 40 | 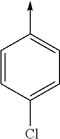 | 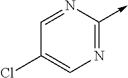 | 459.0 |
| 41 | 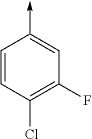 | 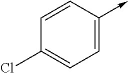 | 475.0 |
| 42 | 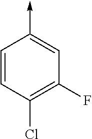 | 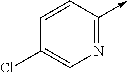 | 476.0 |
| 43 | 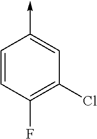 | 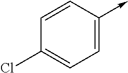 | 475.0 |
| 44 | 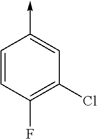 | 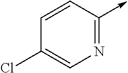 | 476.0 |

### Example 45

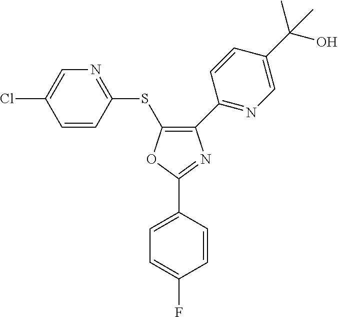 [SMILES: CC(C)(O)c1ccc(-c2nc(-c3ccc(F)cc3)oc2Sc2ccc(Cl)cn2)nc1]

### 2-{6-[5-[(5-Chloropyridin-2-yl)thio]-2-(4-fluorophenyl)-1,3-oxazol-4-yl]pyridin-3-yl}propan-2-ol

The title compound was prepared following the procedure described for Example 12, substituting 2-(5-bromopyridin-2-yl)propan-2-ol with 2-(6-bromopyridin-3-yl)propan-2-ol. The oil was purified on silica gel to afford the title compound (74 mg). LC/MS: m/e 442.0 (M+H)⁺. ¹H NMR (500 MHz, Acetone-d6): δ 1.59 (s, 6H), 4.42 (s, 1H), 7.36 (m, 3H), 7.75 (dd, J=2.6, 8.6 Hz, 1H), 8.06 (m, 2H), 8.21 (m, 2H), 8.43 (d, J=2.5 Hz, 1H), 8.77 (s, 1H).

The compounds in Table 5 were prepared from the appropriate starting materials using the procedure for Example 45.

TABLE 5

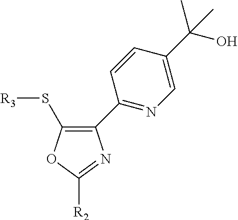

| | | | LCMS: |
|---|---|---|---|
| | | | found m/e |
| Example | R₂ | R₃ | (M + H) |
| 46 | 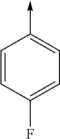 | 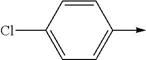 | 441.0 |

### Example 47

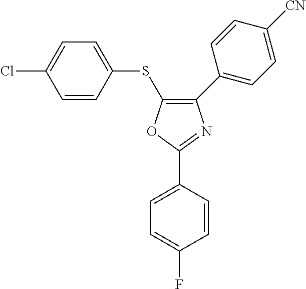 [SMILES: N#Cc1ccc(-c2nc(-c3ccc(F)cc3)oc2Sc2ccc(Cl)cc2)cc1]

### 4-[5-[(4-Chlorophenyl)thio]-2-(4-fluorophenyl)-1,3-oxazol-4-yl]benzonitrile

A solution of 4-chlorobenzenethiol (389 mg, 2.70 mmol) dissolved in 5 mL of NMP was treated with NaH (108 mg, 2.70 mmol). The resulting solution was stirred for 30 mM at rt before Intermediate 14 (700 mg, 1.80 mmol) and CuI (342 mg, 1.80 mmol) were added to the solution. The resulting dark solution was heated to 120° C. for 2 h. After which point, the solution was poured into a rapidly stirred solution of 9:1 NH₄Cl:NH₄OH and EtOAc. Upon clarification of the organic layer, removal of the organic layer was followed by drying over MgSO₄, filtration and concentration giving rise to an oil. The oil was purified on silica gel to afford the title compound. LC/MS: We 407.8 (M+H)⁺.

### Example 48

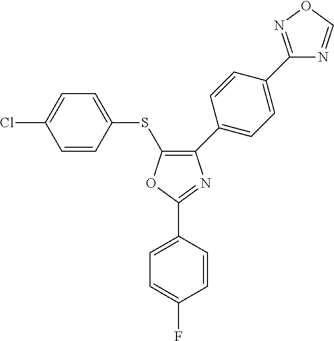 [SMILES: Fc1ccc(-c2nc(-c3ccc(-c4ncon4)cc3)c(Sc3ccc(Cl)cc3)o2)cc1]

### 3-{4-[5-[(4-Chlorophenyl)thio]-2-(4-fluorophenyl)-1,3-oxazol-4-yl]phenyl}-1,2,4-oxadiazole

To Example 47 (100 mg, 0.25 mmol) in 10 mL EtOH was added 1.0 mL of 50 wt % aqueous NH₂OH and 15 mg of K₂CO₃. The reaction was heated to 120° C. for 5 min via microwave irradiation. The reaction mixture was concentrated to dryness and the residue was dissolved in 5 mL triethylorthoformate, 10 mL EtOH and 1 mL of TFA. The reaction was heated to 100° C. for 10 min via microwave irradiation. The volatiles were removed and the residue was purified on silica gel to afford the title compound (111 mg). LC/MS: m/e 450.0 (M+H)⁺. ¹H NMR (500 MHz, Acetone-d6): δ 7.37-7.41 (m, 6H), 8.21 (m, 4H), 8.40 (m, 2H), 9.41 (s, 1H).

### Example 49

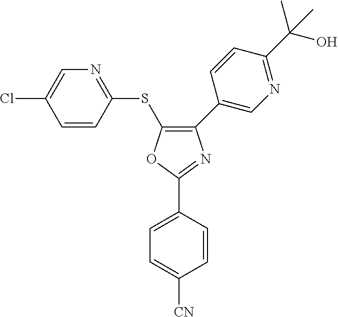 [SMILES: CC(C)(O)c1ccc(-c2nc(-c3ccc(C#N)cc3)oc2Sc2ccc(Cl)cn2)cn1]

### 4-{5-[(5-Chloropyridin-2-yl)thio]-4-[6-(1-hydroxy-1-methylethyl)pyridin-3-yl]-1,3-oxazol-2-yl}benzonitrile

The title compound was prepared following the procedure described for Example 12 using Intermediate 17 (42 mg, 0.10 mmol) and 5-chloropyridine-2-thiol (35.0 mg, 0.24 mmol). The oil was purified on silica gel to afford the title compound (44.6 mg). LC/MS: m/e 449.0 (M+H)⁺. ¹H NMR (500 MHz, Acetone-d6): δ 1.53 (s, 6H), 4.61 (s, 1H), 7.44 (d, J=8.7 Hz, 1H), 7.80 (m, 2H), 8.03 (d, J=8.5 Hz, 2H), 8.36 (d, J=8.5 Hz, 2H), 8.43 (d, J=2.5 Hz, 1H), 8.45 (t, J=2.5 Hz, 1H), 9.20 (d, J=2.1 Hz, 1H).

### Example 50

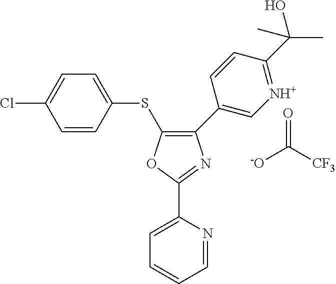

### 2-(5-{5-[(4-chlorophenyl)thio]-2-pyridin-2-yl-1,3-oxazol-4-yl}pyridin-2-yl)propan-2-ol, trifluoroacetic acid salt

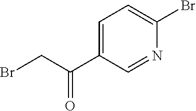 [SMILES: O=C(CBr)c1ccc(Br)nc1]

### 2-bromo-1-(6-bromopyridin-3-yl)ethanone

Step A.

To a solution of 1-(6-bromo-pyridin-3-yl)-ethanone (20.3 g, 101 mmol) and aluminum chloride (200 mg, 1.5 mmol) in chloroform (288 mL) was added bromine (5.23 mL, 101 mmol). The mixture was stirred at rt for 16 h. Upon completion of the reaction as judged by LC/MS analysis, the solution was diluted with sat aq NaHCO₃ and extracted with DCM. The organic layer was removed, dried over MgSO₄, filtered and concentrated giving rise to 31 g 2-bromo-1-(6-bromopyridin-3-yl)ethanone, which was taken on immediately. LC/MS: m/e 277.9 (M+H).

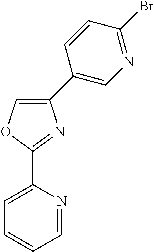 [SMILES: Brc1ccc(-c2coc(-c3ccccn3)n2)cn1]

### 2-bromo-5-(2-pyridin-2-yl-1,3-oxazole-4-yl)pyridine

Step B.

A mixture of 2-bromo-1-(6-bromopyridin-3-yl)ethanone from Step A (2.3 g, 8.25 mmol) and pyridine-2-carboxamide (1 g, 8.25 mmol) was melted at 85°. Heating was continued until the mixture reached 140° at which point the product solidified. Ice, EtOAc, and sat aq NaHCO₃ were added. The aqueous layer was then back extracted with EtOAc/THF (3:1). Pooled organics were dried over MgSO₄, filtered, concentrated, and purified on silica gel to afford 250 mg (10% yield) of 2-bromo-5-(2-pyridin-2-yl-1,3-oxazole-4-yl)pyridine. LC/MS: m/e 302.0 (M+H).

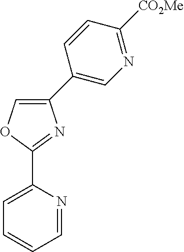 [SMILES: COC(=O)c1ccc(-c2coc(-c3ccccn3)n2)cn1]

### methyl 5-(2-pyridin-2-yl-1,3-oxazol-4-yl)pyridine-2-carboxylate

Step C.

A mixture of 2-bromo-5-(2-pyridin-2-yl-1,3-oxazole-4-yl)pyridine from Step B (250 mg, 0.827 mmol), dppf (92 mg, 0.166 mmol), Pd(OAc)₂ (19 mg, 0.0826 mmol), TEA (0.137 mL, 0.993 mmol) in MeOH (1.4 mL) and DMF (1.4 mL) was bubbled with carbon monoxide for 15 min. The mixture was then placed under a balloon filled with carbon monoxide and stirred at rt for 0.5 h before heating to 75° for 16 h. Upon completion of the reaction as judged by LCMS analysis, the solution was diluted with dist H₂O and extracted with EtOAc. The organic layer was removed, dried over MgSO₄, filtered through a pad of Celite, concentrated, and purified on silica gel giving rise to 200 mg (86% yield) of methyl 5-(2-pyridin-2-yl-1,3-oxazol-4-yl)pyridine-2-carboxylate. LCMS: m/e 282.1 (M+H).

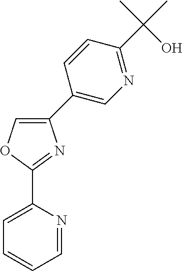 [SMILES: CC(C)(O)c1ccc(-c2coc(-c3ccccn3)n2)cn1]

### 2-[5-(2-pyridin-2-yl-1,3-oxazol-4-yl)pyridin-2-yl]propan-2-ol

Step D.

To a solution of methyl 5-(2-pyridin-2-yl-1,3-oxazol-4-yl)pyridine-2-carboxylate from Step C. (75 mg, 0.267 mmol) in THF (1 mL) at 0° was added a 3 M solution of methylmagnesium bromide in diethyl ether (0.533 mL, 1.6 mmol). The ice bath was removed and the reaction mixture was stirred for 1 h under an atmosphere of nitrogen. Upon completion of the reaction as judged by LCMS analysis, the solution was diluted with sat aq NH₄Cl and extracted with EtOAc. The organic layer was removed, dried over MgSO₄, filtered and concentrated giving rise to 2-[5-(2-pyridin-2-yl-1,3-oxazol-4-yl)pyridin-2-yl]propan-2-ol, which was taken on immediately. LC/MS: m/e 282.1 (M+H).

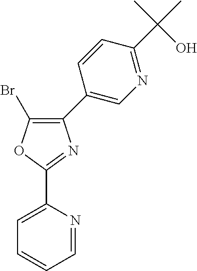 [SMILES: CC(C)(O)c1ccc(-c2nc(-c3ccccn3)oc2Br)cn1]

### 2-[5-(5-bromo-2-pyridin-2-yl-1,3-oxazol-4-yl)pyridin-2-yl]propan-2-ol

Step E.

To a solution of 2-[5-(2-pyridin-2-yl-1,3-oxazol-4-yl)pyridin-2-yl]propan-2-ol from Step D (75 mg, 0.267 mmol) in DCM (1 mL) was added NBS (62 mg, 0.347 mmol). The reaction mixture was stirred at rt for 16 h. Water was added and the mixture extracted with DCM. The organics were dried (MgSO₄), and concentrated to afford 2-[5-(5-bromo-2-pyridin-2-yl-1,3-oxazol-4-yl)pyridin-2-yl]propan-2-ol, which was used without further purification LCMS: m/z 360.0 (M+H)⁺.

Step F.

To a solution of 4-chloro thiophenol (38 mg, 0264 mmol) in NMP (0.5 mL) was added NaH (11 mg, 0.264 mmol) and stirred at rt for 0.5 h under an atmosphere of nitrogen. To the resulting sodium salt was added a solution of 2-[5-(5-bromo-2-pyridin-2-yl-1,3-oxazol-4-yl)pyridin-2-yl]propan-2-ol from Step E (38 mg, 0.105 mmol) in NMP (0.5 mL) followed by CuI (20 mg, 0.105 mmol). The mixture was then heated at 120° for 2 h under an atmosphere of nitrogen. Saturated aqueous ammonium chloride (4.5 mL) and ammonium hydroxide (0.5 mL) were added and the mixture stirred at rt for 0.5 h. The mixture was extracted 3 times with EtOAc. Combined organics were dried (MgSO₄), concentrated, and purified by reverse phase HPLC to afford 20 mg (35% yield over 3 steps) of the title compound as the TFA salt. LCMS: m/z 424.1 (M+H)⁺. ¹H NMR (500 MHz, CO(CD₃)₂: δ 9.25 (1H, s), 8.75 (1H, m), 8.50 (1H, m), 8.32 (1H, d), 8.06 (1H, m), 7.82 (1H, m), 7.60 (1H, m), 7.43 (4H, br), 1.55 (6H, s).

### Example 51

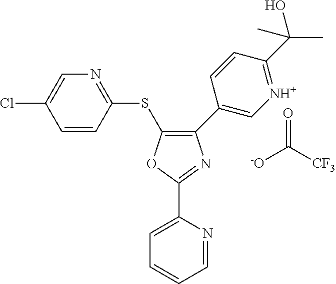

### 2-(5-{5-[(5-chloropyridin-2-1)thio]-2-pyridin-2-yl-1,3-oxazol-4-yl}pyridin-2-yl)propan-2-ol, trifluoroacetic acid salt

To a solution of 5-chloropyridine-2-thiol (38 mg, 0.264 mmol) in NMP (0.5 mL) was added Nail (11 mg, 0.264 mmol) and stirred at rt for 0.5 h under an atmosphere of nitrogen. To the resulting sodium salt was added a solution of 2-[5-(5-bromo-2-pyridin-2-yl-1,3-oxazol-4-yl)pyridin-2-yl]propan-2-ol (38 mg, 0.105 mmol) in NMP (0.5 mL) followed by CuI (20 mg, 0.105 mmol). The mixture was then heated at 120° for 2 h under an atmosphere of nitrogen. Saturated aqueous ammonium chloride (4.5 mL) and ammonium hydroxide (0.5 mL) were added and the mixture stirred at rt for 0.5 h. The mixture was extracted 3 times with EtOAc. Combined organics were dried (MgSO₄), concentrated, and purified by reverse phase HPLC to afford 18 mg (32% yield over 3 steps) the title compound as the TFA salt. LCMS: m/z 425.1 (M+H)⁺. ¹H NMR (500 MHz, CO(CD₃)₂: δ 9.22 (1H, s), 8.77 (1H, s), 8.55 (2H, br), 8.34 (1H, m), 8.07 (1H, m), 7.81 (2H, br), 7.61 (1H, m), 7.42 (111, d) 1.54 (6H, s).

### Example 52

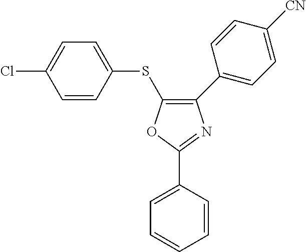 [SMILES: N#Cc1ccc(-c2nc(-c3ccccc3)oc2Sc2ccc(Cl)cc2)cc1]

### 5-[(4-chlorophenyl)thio]-4 (4-cyanophenyl)-2-phenyl-1,3-oxazole

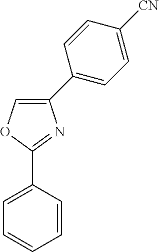 [SMILES: N#Cc1ccc(-c2coc(-c3ccccc3)n2)cc1]

### 4-(2-phenyl-1,3-oxazol-4-yl)benzonitrile

Step A.

The mixture of the 2-bromo-1-(4-cyanophenyl)ethanone (4 g, 17.85 mmol) and benzamide (5.41 g, 44.6 mmol) was heated to 135 r for 3 hours. Then the reaction mixture was cooled, and partitioned between diethyl ether and water. The aqueous layer was extracted with ether twice, and the combined organic layers were washed with 1N NaOH, 1N HCl, water, and brine, dried over MgSO₄. After concentration, the solid residue was dissolved in CHCl3. The insoluble solid was filtered through a fits funnel and discarded. The CHCl3 solution was filtered through a pad of silica and evaporate to dryness to give 2.9 g (66% yield) of 4-(2-phenyl-1,3-oxazol-4-yl)benzonitrile. LCMS: m/z 247.1 (M+H)⁺.

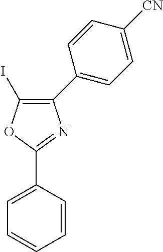 [SMILES: N#Cc1ccc(-c2nc(-c3ccccc3)oc2I)cc1]

### 4-(5-iodo-2-phenyl-1,3-oxazol-4-yl)benzonitrile

Step B.

The product of Step A (140 mg, 0.57 mmol) was dissolved in 2 mL of chloroform, to which was added NIS (282 mg, 135 mmol) and 2 drops of TFA. After stirring at rt for two days, the reaction was diluted with dichloromethane, washed with aq NaHCO₃, aq Na₂S₂O₃, water, and brine. The organic layer was dried over MgSO₄, filtered, and concentrated to give 186 mg (88% yield) of 4-(5-iodo-2-phenyl-1,3-oxazol-4-yl)benzonitrile. LCMS: m/z 373.0 (M+H)⁺.

Step C.

CuI (4.8 mg, 0.025 mmol), K₂CO₃ (138 mg, 1 mmol), the product of Step B (186 mg, 0.5 mmol), and 4-chlorobenzenethiol (72 mg, 0.5 mmol) were added to a flask, which was evacuated and backfilled with N₂ (3 cycles). 2-Propanol (2 mL) and ethylene glycol (0.056 mL, 1 mmol) were added by syringe at rt. The reaction mixture was heated at 80° C. for 18 hours. Then the reaction was diluted with EtOAc, filtered, concentrated, and the residue was subject to silica column (0-20% EtOAc in hexanes) to give the title compound. ¹H NMR (500 MHz, (CDCl₃): 8.38 (d, 2H), 8.19 (d, 2H), 7.78 (d, 2H), 7.57 (m, 3H), 7.31 (d, 2H), 7.25 (d, 2H). LCMS: m/z 389.0 (M+H)⁺.

### Example 53

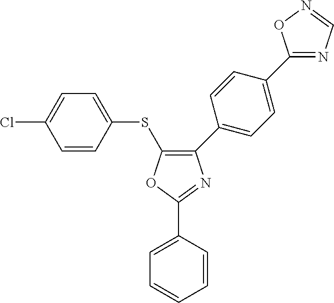 [SMILES: Clc1ccc(Sc2oc(-c3ccccc3)nc2-c2ccc(-c3ncno3)cc2)cc1]

### 3-(4-{5-[(4-Chlorophenyl)thio]-2-phenyl-1,3oxazol-4-yl}phenyl)-1,2,4oxadiazole

To 3-(4-{5-[(4-chlorophenyl)thio]-2-phenyl-1,3oxazol-4-yl}benzonitrile (30 mg, 0.075 mmol) in 2 mL EtOH was added 0.25 mL of 50% aqueous NH₂OH and catalytic amount of K₂CO₃. The reaction was heated at 120° C. for 1 h via microwave irradiation. The reaction mixture was concentrated to dryness and the residue was dissolved in 5 mL triethylorthoformate. A catalytic amount of TFA was added, and the reaction was heated at 130° C. for 3 h. The volatiles were removed and the residue was purified by reverse phase HPLC to afford 12 mg (37% yield) of the title compound.: m/z 432.1 (M+H)⁺. NMR (500 MHz, CDCl₃: δ 8.8 (1H, s), 8.39 (2H, d), 8.21 (2H, d), 8.19 (1H, m), 7.59 (4H, br), 7.24 (4H, br).

### Example 54

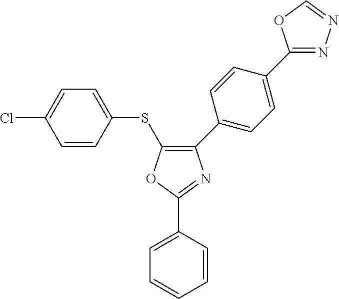 [SMILES: Clc1ccc(Sc2oc(-c3ccccc3)nc2-c2ccc(-c3nnco3)cc2)cc1]

### 2-(4-{5-[(4-Chlorophenyl)thio]-2-phenyl-1,3oxazol-4-yl}phenyl)-1,3,4oxadiazole

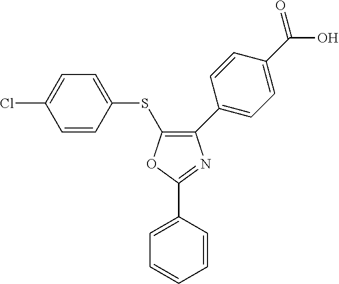 [SMILES: O=C(O)c1ccc(-c2nc(-c3ccccc3)oc2Sc2ccc(Cl)cc2)cc1]

### 4-{5-[(4-chlorophenyl)thio]-2-phenyl-1,3,-oxazol-4-yl}benzoic acid

Step A.

A solution of 3-(4-{5-[(4-chlorophenyl)thio]-2-phenyl-1,3oxazol-4-yl}benzonitrile (30 mg, 0.077 mmol) in ethanol (1 mL) and 2N NaOH (1 mL) was heated to reflux for 16 h. EtOAc was added followed by saturated aqueous ammonium chloride. The organics were dried (MgSO₄) and concentrated to afford 4-{5-[(4-chlorophenyl)thio]-2-phenyl-1,3,-oxazol-4-yl}benzoic acid, which was used with out further nitrification. LCMS: m/z 407.1 (M+H)⁺.

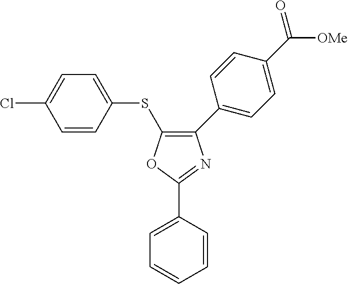 [SMILES: COC(=O)c1ccc(-c2nc(-c3ccccc3)oc2Sc2ccc(Cl)cc2)cc1]

### methyl 4-{5-[(4-chlorophenyl)thio]-2-phenyl-1,3,-oxazol-4-yl}benzoate

Step B.

4-{5-[(4-chlorophenyl)thio]-2-phenyl-1,3,-oxazol-4-yl}benzoic acid from Step A (32 mg, 0.077 mmol) was dissolved in MeOH (0.5 mL) and DCM (0.5 mL). Trimethylsilyl diazomethane (2.0 M in ether) was slowly added at 0° C. until a yellow color persisted. The volatiles were evaporated to give methyl 4-{5-[(4-chlorophenyl)thio]-2-phenyl-1,3,-oxazol-4-yl}benzoate which was used without further purification. LCMS: m/z 421.1 (M+H)⁺.

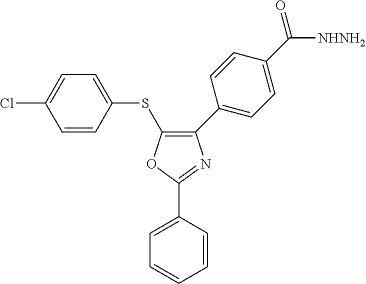 [SMILES: NNC(=O)c1ccc(-c2nc(-c3ccccc3)oc2Sc2ccc(Cl)cc2)cc1]

### 4-{5-[(4-chlorophenyl)thio]-2-phenyl-1,3,-oxazol-4-yl}benzohydrazide

Step C.

Methyl 4-{5-[(4-chlorophenyl)thio]-2-phenyl-1,3,-oxazol-4-yl}benzoate from Step B (33 mg, 0.077 mmol) was suspended in 1 mL of EtOH and 0.5 mL of anhydrous hydrazine, and heated to reflux for 2 h. EtOAc was added and washed with water 3 times. The organics were dried (MgSO₄), and concentrated to afford 4-{5-[(4-chlorophenyl)thio]-2-phenyl-1,3,-oxazol-4-yl}benzohydrazide which was used with out further purification. LCMS: m/z 421.1 (M+H)⁺.

Step D.

4-{5-[(4-chlorophenyl)thio]-2-phenyl-1,3,-oxazol-4-yl}benzohydrazide from Step C (33 mg, 0.077 mmol) was dissolved in 5 mL triethylorthoformate. A catalytic amount of TFA was added and the reaction was heated at 130° C. for 2 h. The volatiles were removed and the residue was purified by reverse phase HPLC to afford 12 mg (36% over 4 steps) of the title compound 2-(4-{5-[(4-chlorophenyl)thio]-2-phenyl-1,3oxazol-4-yl}phenyl)-1,3,4-oxadiazole. LCMS: m/z 432.1 (M+H)⁺. 1H NMR (500 MHz, CDCl₃: δ 8.55 (1H, s), 8.40 (2H, d), 8.19 (4H, br), 7.55 (3H, br), 7.30 (4H, br).

### Example 55

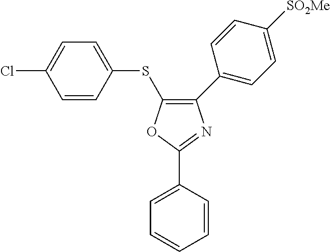 [SMILES: CS(=O)(=O)c1ccc(-c2nc(-c3ccccc3)oc2Sc2ccc(Cl)cc2)cc1]

### 5-[(4-chlorophenyl)thio]-4-[4-(methylsulfonyl)phenyl]-2-phenyl-1,3-oxazole

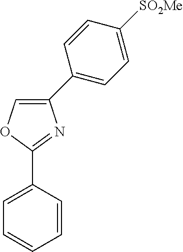 [SMILES: CS(=O)(=O)c1ccc(-c2coc(-c3ccccc3)n2)cc1]

### 4-[4-(methylsulfonyl)phenyl]-2-phenyl-1,3-oxazole

Step A.

The mixture of the 2-bromo-1-[4-(methylsulfonyl)phenyl]ethanone (2 g, 7.2 mmol) and benzamide (0.87 g, 7.2 mmol) was heated to 140˜180° C. for 4 hours. When TLC showed that the reaction had completed, the mixture was cooled, and partitioned between EtOAc and water. The aqueous layer was extracted with EtOAc twice, and the combined organic layers were washed with water and brine, dried over MgSO₄. After concentration, the residue was purified by column (eluted by PE:EA=10:1) to afford 0.6 g (yield 30%) of 4-[4-(methylsulfonyl)phenyl]-2-phenyl-1,3-oxazole.

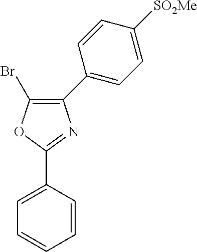 [SMILES: CS(=O)(=O)c1ccc(-c2nc(-c3ccccc3)oc2Br)cc1]

### 5-bromo-4-[4-(methylsulfonyl)phenyl]-2-phenyl-1,3-oxazole

Step B.

To a solution of Step A product (0.7 g, 2.34 mmol) in AcOH (20 ml) and CHCl₃ (30 ml) was added dropwise Br₂ (0.41 g) at rt, and the mixture was stirred for 2 hours. The reaction mixture was poured into water, and extracted with EtOAc three times. The combined organic layers were washed with aqueous NaHCO₃ and brine, dried over Na₂SO₄. After concentration, the residue was purified by column (PE:EA=4:1) to afford 0.7 g (yield 80%) of 5-bromo-4-[4-(methylsulfonyl)phenyl]-2-phenyl-1,3-oxazole.

Step C.

To a solution of Step B product (0.2 g, 0.53 mmol) and 4-chlorobenzenethiol (0.076 g, 0.53 mmol) in ethanol was added KOH (34 mg, 0.6 mmol) at rt under N₂, then the mixture was heated to reflux overnight. After cooling, the precipitate was collected by suction, and the filter cake was washed with ethanol. After drying, 200 mg (yield 80%) of the title compound was obtained. ¹H-NMR (400 MHz, DMSO)S 8.30 (d, 2H, Ar—H), 8.06 (m, 4H, Ar—H), 7.60 (m, 3 H, Ar—H), 7.40 (m, 4H, Ar—H), 326 (s, 3H, CH₃).

### Example 56

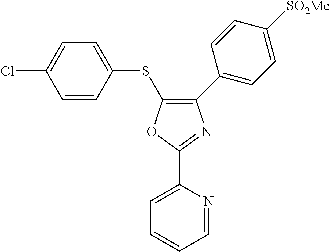 [SMILES: CS(=O)(=O)c1ccc(-c2nc(-c3ccccn3)oc2Sc2ccc(Cl)cc2)cc1]

### 2-{5-[(4-chlorophenyl)thio]-4-[4-(methylsulfonyl)phenyl]-1,3-oxazol-2-yl}pyridine

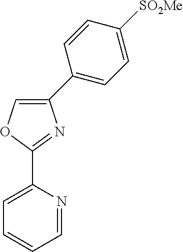 [SMILES: CS(=O)(=O)c1ccc(-c2coc(-c3ccccn3)n2)cc1]

### 2-{4-[4-(methylsulfonyl)phenyl]-1,3-oxazol-2-yl}pyridine

Step A.

The mixture of the 2-bromo-1-[4-(methylsulfonyl)phenyl]ethanone (500 mg, 1.8 mmol) and pyridine 2-carboxamide (551 mg, 4.51 mmol) was heated to 150° C. for 1 hour. Then the reaction mixture was cooled, and partitioned between ethyl acetate and water. The aqueous layer was extracted with ethyl acetate twice, and the combined organic layers were washed with water and brine, dried over MgSO₄. After concentration, the solid residue was dissolved in methanol and subject to mass-directed HPLC purification to give 21 mg of 2-{4-[4-(methylsulfonyl)phenyl]-1,3-oxazol-2-yl}pyridine. LCMS: m/z 301.0 (M+H)⁺.

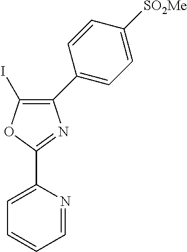 [SMILES: CS(=O)(=O)c1ccc(-c2nc(-c3ccccn3)oc2I)cc1]

### 2-{5-iodo-4-[4-(methylsulfonyl)phenyl]-1,3-oxazol-2-yl}pyridine

Step B.

The product of Step A (20 mg, 0.067 mmol) was dissolved in 1 mL of chloroform, to which was added NIS (22.5 mg, 0.1 mmol) and 1 drop of TFA. After stirring at rt for 2 hours, the reaction was diluted with dichloromethane, washed with aq NaHCO₃, aq Na₂S₂O₃, water, and brine. The organic layer was dried over MgSO₄, filtered, and concentrated to give 2-{5-iodo-4-[4-(methylsulfonyl)phenyl]-1,3-oxazol-2-yl}pyridine. LCMS: m/z 427.0 (M+H)⁺.

Step C.

CuI (2 mg, 0.01 mmol), K₂CO₃ (6.5 mg, 0.05 mmol), the product of Step B (10 mg, 0.023 mmol), and 4-chlorobenzenethiol (3.4 mg, 0.023 mmol) were added to a flask, which was evacuated and backfilled with N₂ (3 cycles). 2-Propanol (0.5 mL) and 0.01 mL of ethylene glycol were added by syringe at rt. The reaction mixture was heated at 80° C. for 18 hours. Then the reaction was diluted with acetonitrile and filtered through Celite. The filtrate was subjected to mass-directed HPLC to give the title compound. 1H NMR (500 MHz, (CDCl₃): 8.82 (broad s, 1H), 8.47 (d, 2H), 8.23 (d, 1H), 8.05 (d, 2H), 7.91 (t, 1H), 7.46 (t, 1H), 7.23 (AB quartet, 4H), 3.11 (s, 3H). LCMS: m/z 443.0 (M+H)⁺.

| | Human Lysate | Human whole cell | Rat whole cell |
|---|---|---|---|
| Example | IC50 (nM) | IC50 (nM) | IC50 (nM) |
| Ex 58 | 37 | 112 | 74 |
| Ex 59 | 20 | 67 | 40 |
| Ex 62 | 23 | 41 | 29 |
| Ex 65 | 27 | 29 | 21 |
| Ex 68 | 15 | 100 | 83 |
| Ex 71 | 10 | 30 | 14 |
| Ex 74 | 8 | 37 | 34 |
| Ex 78 | 28 | 69 | 39 |
| Ex 80 | 35 | 67 | 25 |
| Ex 90 | 46 | 1002 | 247 |
| Ex 96 | 17 | 133 | 63 |
| Ex 97 | 20 | NA | 10 |
| Ex 98 | 44 | 222 | 35 |
| Ex 100 | 161 | 337 | 39 |
| Ex 102 | 12 | 35 | 17 |
| Ex 107 | 24 | 91 | 11 |
| Ex 108 | 5 | 20 | 17 |
| Ex 111 | 11 | 64 | 24 |
| Ex 119 | 28 | 47 | 20 |
| Ex 122 | 161 | 474 | 146 |
| Ex 123 | 74 | 510 | 286 |
| Ex 124 | 11 | 98 | 16 |
| Ex 125 | 93 | 2291 | 680 |
| Ex 131 | 140 | 1119 | 782 |

### Intermediate 18

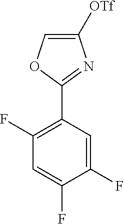 [SMILES: O=S(=O)(Oc1coc(-c2cc(F)c(F)cc2F)n1)C(F)(F)F]

### 2-(2,4,5-Trifluorophenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 19

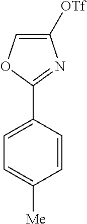 [SMILES: Cc1ccc(-c2nc(OS(=O)(=O)C(F)(F)F)co2)cc1]

### 2-(4-Methylphenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 20

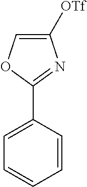 [SMILES: O=S(=O)(Oc1coc(-c2ccccc2)n1)C(F)(F)F]

### 2-Phenyl-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 21

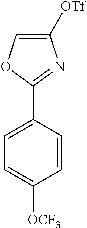 [SMILES: O=S(=O)(Oc1coc(-c2ccc(OC(F)(F)F)cc2)n1)C(F)(F)F]

### 2-[4-(Trifluoromethoxy)phenyl]-1,3-oxazol-4-yltrifluoromethanesulfonate

The title compound was prepared using the procedure described by Langille, N. F.; Dakin, L. A.; Panek, J. S. Org. Lett., 2002, 4, 2485.

### Intermediate 22

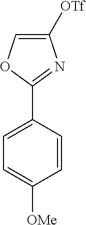 [SMILES: COc1ccc(-c2nc(OS(=O)(=O)C(F)(F)F)co2)cc1]

### 2-(4-Methoxyphenyl)-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 23

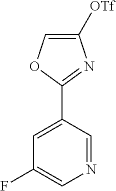 [SMILES: O=S(=O)(Oc1coc(-c2cncc(F)c2)n1)C(F)(F)F]

### 2-(5-Fluoropyridin-3-yl)-1,3-oxazol-4-yl trifluoromethanesulfonate

The title compound was prepared using the procedure described by Langille, N. F.; Dakin, L. A.; Panek, J. S. Org. Lett, 2002, 4, 2485.

### Intermediate 24

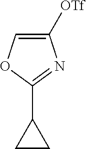 [SMILES: O=S(=O)(Oc1coc(C2CC2)n1)C(F)(F)F]

### 2-Cyclopropyl-1,3-oxazol-4-yl trifluoromethanesulfonate

The title compound was prepared using the procedure described by Langille, N. F.; Dakin, L. A.; Panek, J. S. Org. Lett., 2002, 4, 2485.

### Intermediate 25

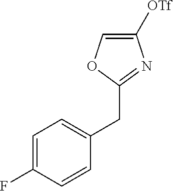 [SMILES: O=S(=O)(Oc1coc(Cc2ccc(F)cc2)n1)C(F)(F)F]

### 2-(4-Fluorobenzyl)-1,3-oxazol-4-yl trifluoromethanesulfonate

A. To a stirred solution of 4-fluorophenylacetyl chloride (2.0 g, 12.0 mmol) in 25 mL of CH₂Cl₂ was added 1.7 g (12.0 mmol) of silver cyanate. The resulting slurry was stirred for 3 h at rt. After which point, the solution was filtered through Celite and the filtrate was then taken on to the next step crude.

B. The acyl isocyanate dissolved in DCM was cooled to 0° C. and treated with TMS Diazomethane (6.9 mL, 14.0 mmol, 2.0 M solution in Et₂O). The resulting yellow solution was allowed to warm to rt and stirred for 1 h. Upon completion of the reaction as judged by TLC analysis, the solution was concentrated to dryness and purified on silica gel giving 1.3 g of oxazolidinone intermediate which was taken on directly to triflation.

C. The oxazolidinone (1.3 g, 7 mmol) was treated with Tf₂O (1.7 mL, 10.0 mmol) and TEA (2.0 mL, 14.0 mmol) at −78° C. After 1 h, the solution was diluted with sat aqueous NaCl solution and allowed to warm to rt. The organic layer was removed, dried over MgSO₄, filtered, and concentrated to dryness giving rise to an oil. The oil was purified on silica gel giving rise to the title compound (768 mg). ¹H NMR (500 MHz, Acetone-d6): δ 4.21 (s, 2H), 7.16 (m, 2H), 7.40 (m, 2H), 8.23 (s, 1H).

### Intermediate 26

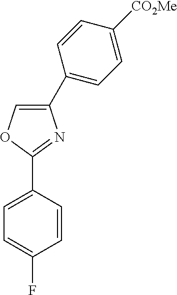 [SMILES: COC(=O)c1ccc(-c2coc(-c3ccc(F)cc3)n2)cc1]

### Methyl 4-[2-(4-fluorophenyl)-1,3-oxazol-4-yl]benzoate

A solution of Intermediate 1(3.09 g, 9.9 mmol), 4-[(methoxycarbonyl)phenyl]boronic acid (2.1 g, 12.0 mmol), Pd(dppf)Cl₂, (405 mg, 0.5 mmol), and CsF (3.0 g, 19.9 mmol) were dissolved in dioxane (150 mL) and heated to 100° C. for 12 h. Upon completion of the reaction as judged by TLC analysis, the solution was concentrated to dryness and purified on silica gel to afford the title compound (2.50 g). LC/MS: m/e 395.8 (M+H).

### Intermediate 27

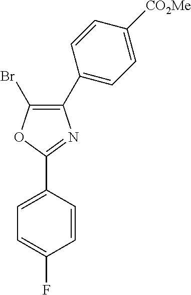 [SMILES: COC(=O)c1ccc(-c2nc(-c3ccc(F)cc3)oc2Br)cc1]

A solution of Intermediate 26 (1.06 g, 3.6 mmol) and NBS (952 mg, 5.4 mmol) in CH₂Cl₂ (50 mL) was stirred at rt for 12 h. Upon completion of the reaction, the solution was diluted with sat aq NaS₂O₃ solution. The organic layer was removed, dried over MgSO₄, filtered and concentrated giving rise to an oil. The oil was purified on silica gel to afford the title compound (1.01 g). LC/MS: m/e 375.8 (M+H)⁺.

### Intermediate 28

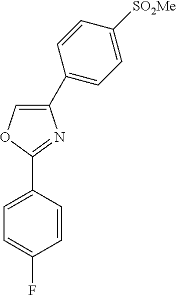 [SMILES: CS(=O)(=O)c1ccc(-c2coc(-c3ccc(F)cc3)n2)cc1]

### 2-(4-Fluorophenyl)-4-[4-(methylsulfonyl)phenyl]-1,3-oxazole

The target compound was prepared in an analogous manner to Intermediate 26 except that Intermediate 1 was coupled with [4-(methylsulfonyl)phenyl]boronic acid. LC/MS: m/e 318.1 (M+H).

### Intermediate 29

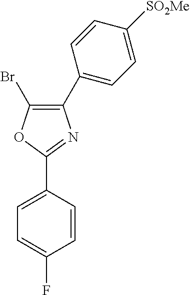 [SMILES: CS(=O)(=O)c1ccc(-c2nc(-c3ccc(F)cc3)oc2Br)cc1]

### 5-Bromo-2-(4-Fluorophenyl)-4-[4-(methylsulfonyl)phenyl]-1,3-oxazole

The target compound was prepared in an analogous manner to Intermediate 27, LC/MS: m/e 395.9 (M+H)⁺.

### Intermediate 30

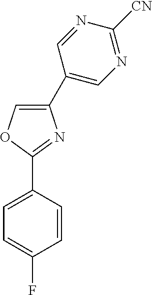 [SMILES: N#Cc1ncc(-c2coc(-c3ccc(F)cc3)n2)cn1]

### 5-[2-(4-Fluorophenyl)-1,3-oxazol-4-yl]pyrimidine-2-carbonitrile

The target compound was prepared in an analogous manner to Intermediate 11 except that Intermediate 1 was coupled with 5-bromopyrimidine-2-carbonitrile. LC/MS: m/e 267.0 (M+11)⁺.

### Intermediate 31

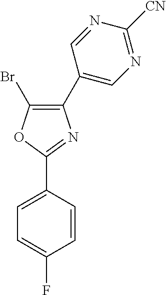 [SMILES: N#Cc1ncc(-c2nc(-c3ccc(F)cc3)oc2Br)cn1]

### 5-[5-Bromo-2-(4-fluorophenyl)-1,3-oxazol-4-yl]pyrimidine-2-carbonitrile

The target compound was prepared in an analogous manner to Intermediate 27, LC/MS: m/e 345.0 (M+H)⁺.

### Intermediate 32

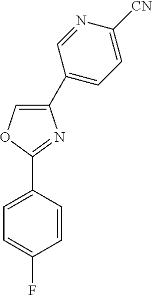 [SMILES: N#Cc1ccc(-c2coc(-c3ccc(F)cc3)n2)cn1]

### 5-[2-(4-Fluorophenyl)-1,3-oxazol-4-yl]pyridine-2-carbonitrile

The target compound was prepared in an analogous manner to Intermediate 11 except that Intermediate 1 was coupled with 5-bromopyridine-2-carbonitrile. LC/MS: m/e 266.0 (M+H).

### Intermediate 33

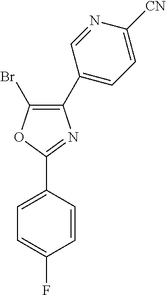 [SMILES: N#Cc1ccc(-c2nc(-c3ccc(F)cc3)oc2Br)cn1]

### 5-[5-Bromo-2-(4-fluorophenyl)-1,3-oxazol-4-yl]pyridine-2-carbonitrile

The target compound was prepared in an analogous manner to Intermediate 27, LC/MS: m/e 343.9 (M+H)⁺.

### Intermediate 34

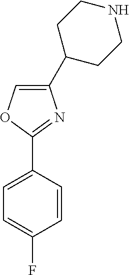 [SMILES: Fc1ccc(-c2nc(C3CCNCC3)co2)cc1]

### 4-[2-(4-Fluorophenyl)-1,3-oxazol-4-yl]piperidine

A solution of 4-fluorobenzamide (4.54 g, 32.7 mmol) and t-butyl 4-(bromoacetyl)-piperidine-1-carboxylate (5.0 g, 16.3 mmol) in DMF (40 mL) was heated at 145° C. for 16 h. Upon completion of the reaction, the solution was allowed to cool to rt and concentrated to a dark oil. The oil was purified by reverse phase HPLC to afford the title compound (760 mg). LC/MS: m/e 247.08 (M+H)⁺.

### Intermediate 35

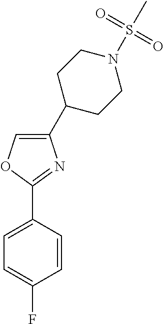 [SMILES: CS(=O)(=O)N1CCC(c2coc(-c3ccc(F)cc3)n2)CC1]

### 4-[2-(4-Fluorophenyl)-1,3-oxazol-4-yl]-1-(methylsulfonyl)piperidine

To a solution of 442-(4-fluorophenyl)-1,3-oxazol-4-ylipiperidine (220 mg, 0.90 mmol) in DCM (20 mL) was treated with DIEA (0.31 mL, 1.8 mmol) and allowed to stir at rt for 15 min. Methanesulfonyl chloride (0.2 mL, 2.7 mmol) was slowly added to the solution and the resulting mixture was stirred at rt for 2 hr. Upon completion of the reaction, DCM (20 mL) and water (40 mL) was added to the mixture and the two layers were partitioned. The organic layer was dried with MgSO₄, filtered, and concentrated. The residue was purified by reverse phase HPLC to afford the title compound (100 mg). LC/MS: m/e 325.2 (M+H)⁺.

### Intermediate 36

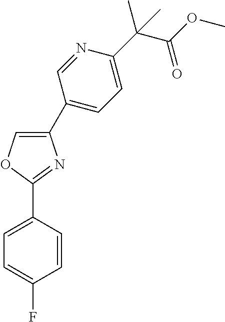 [SMILES: COC(=O)C(C)(C)c1ccc(-c2coc(-c3ccc(F)cc3)n2)cn1]

### Methyl 2-{5-[2-(4-fluorophenyl)-1,3-oxazol-4-yl]pyridin-2-yl}-2-methylpropanoate

The target compound was prepared in an analogous manner to Intermediate 11 except that Intermediate 1 was coupled with methyl 2-(5-bromopyridin-2-yl)-2-methylpropanoate (Kodanko, J. J.; Morys, A. J.; Lippard, S. J. Org. Lett. 2005, 7, 4585) LC/MS: m/e 295.4 (M+H)⁺.

### Intermediate 37

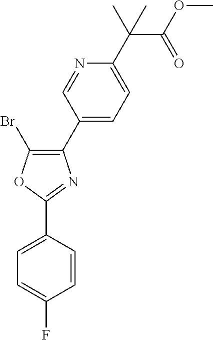 [SMILES: COC(=O)C(C)(C)c1ccc(-c2nc(-c3ccc(F)cc3)oc2Br)cn1]

### Methyl 2-{5-[5-bromo-2-(4-fluorophenyl)-1,3-oxazol-4-yl]pyridin-2-yl}-2-methyl propanoate

The target compound was prepared in an analogous manner to Intermediate 27, LC/MS: m/e 373.05 (M+H)⁺.

### Intermediate 38

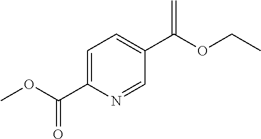

### Methyl 5-(1-ethoxyethenyl)pyridine-2-carboxylate

To a solution of methyl 5-bromopyridine-2-carboxylate (25 g, 116 mmol) in dioxane (30 mL) was added Pd(PPh₃)₄(6.7 g, 5.8 mmol) and tributyl(1-ethoxyvinyl)tin (46 g, 127.0 mmol). The resulting solution was heated to reflux under N₂ for 12 h. Upon completion of the reaction as judged by LC/MS analysis, the reaction was diluted with EtOAc, washed with KF solution (10% aqueous), filtered through Celite, dried over MgSO₄, filtered, concentrated and purified on silica gel to afford the title compound (20.4 g), LC/MS: m/e 208.1 (M+H)⁺.

### Intermediate 39

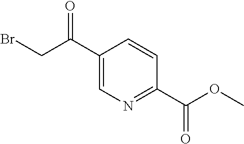

### Methyl 5-(bromoacetyl)pyridine-2-carboxylate

To a solution of Intermediate 38 (20.3 g, 98.0 mmol) in THF/H₂O (700 mL/46 mL) at rt was added NBS (15.0 g, 98.0 mmol) in one portion, The resulting solution was stirred at rt for 30 min. Upon completion of the reaction as judged by LC/MS analysis, the reaction was concentrated to dryness and purified on silica gel to afford the title compound (19.5 g). LC/MS: ink 259.9 (M+H)⁺.

### Intermediate 40

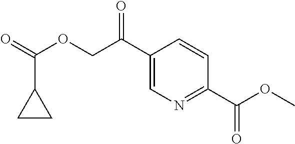 [SMILES: COC(=O)c1ccc(C(=O)COC(=O)C2CC2)cn1]

### Methyl 5-{[(cyclopropylcarbonyl)oxy]acetyl}pyridine-2-carboxylate

The mixture of cyclopropyl carboxylic acid (5.0 g, 58.1 mmol), Intermediate 39 (15.0 g, 58.1 mmol) and K₂CO₃ (9.63 g, 69.7 mmol) in DMF (50 mL) was stirred at rt for 12 h. Upon completion of the reaction as judged by LC/MS analysis, the reaction was diluted with H₂O and the resulting precipitate was filtered to afford the title compound (8.54 g), LC/MS: m/e 263.9 (M+H)⁺.

### Intermediate 41

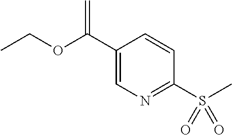

### 5-(1-Ethoxyethenyl)-2-(methylsulfonyl)pyridine

The target compound was prepared in an analogous manner to Intermediate 38 except starting with 5-bromo-2-methylsulphonylpyridine, LC/MS: m/e 228.05 (M+H)⁺.

### Intermediate 42

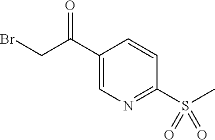

### 2-Bromo-1-[6-(methylsulfonyl)pyridin-3-yl]ethanone

The title compound was prepared in an analogous manner to Intermediate 39. LC/MS: m/e 279.76 (M+H)⁺.

### Intermediate 43

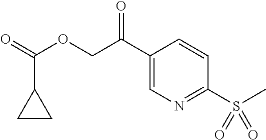 [SMILES: CS(=O)(=O)c1ccc(C(=O)COC(=O)C2CC2)cn1]

### 2-[6-(Methylsulfonyl)pyridin-3-yl]-2-oxoethyl cyclopropanecarboxylate

The title compound was prepared in an analogous manner to Intermediate 40, LC/MS: m/e 283.9 (M+H)⁺.

### Intermediate 44

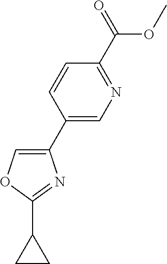 [SMILES: COC(=O)c1ccc(-c2coc(C3CC3)n2)cn1]

### Methyl 5-(2-cyclopropyl-1,3-oxazol-4-yl)pyridine-2-carboxylate

To a solution of Intermediate 40 (2.0 g, 7.6 mmol) in p-xylene (130 mL) was added acetamide (2.24 g, 38.0 mmol), and BF₃.OEt₂ (1.9 mL, 15.2 mmol). The resulting solution was heated at reflux for 72 h. After which point, the reaction was diluted with sat. NaHCO_{3 solution}, and extracted with EtOAc. The organic layer was washed with brine, dried over MgSO₄, filtered, concentrated and purified on silica gel to afford the title compound (862 mg), LC/MS: m/e 245.0 (M+H)⁺.

### Intermediate 45

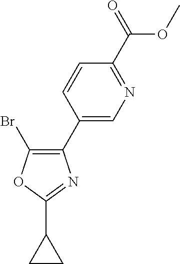 [SMILES: COC(=O)c1ccc(-c2nc(C3CC3)oc2Br)cn1]

### Methyl 5-(5-bromo-2-cyclopropyl-1,3-oxazol-4-yl)pyridine-2-carboxylate

The target compound was prepared in an analogous manner to Intermediate 27 starting with Intermediate 44, LC/MS: m/e 324.8 (M+H)⁺.

### Intermediate 46

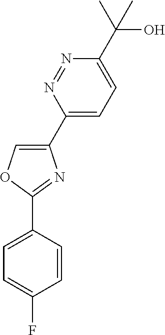 [SMILES: CC(C)(O)c1ccc(-c2coc(-c3ccc(F)cc3)n2)nn1]

### 2-{6-[2-(4-Fluorophenyl)-1,3-oxazol-4-yl]pyridazin-3-yl}propan-2-ol

The target compound was prepared in an analogous manner to Intermediate 11 except that intermediate 1 was coupled with 2-(6-chloropyridazin-3-yl) propan-2-ol. LC/MS: m/e 380.0 (M+H)⁺.

### Intermediate 47

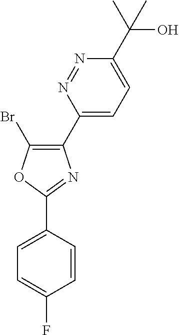 [SMILES: CC(C)(O)c1ccc(-c2nc(-c3ccc(F)cc3)oc2Br)nn1]

### 2-{6-[5-Bromo-2-(4-fluorophenyl)-1,3-oxazol-4-yl]pyridazin-3-yl}propan-2-ol

The target compound was prepared in an analogous manner to Intermediate 27. LC/MS: m/e 380.0 (M+H)⁺.

### Intermediate 48

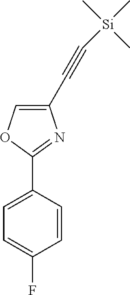 [SMILES: C[Si](C)(C)C#Cc1coc(-c2ccc(F)cc2)n1]

### 2-(4-Fluorophenyl)-4-[(trimethylsilyl)ethynyl]-1,3-oxazole

To a solution of Intermediate 1 (2.1 g, 6.8 mmol) in DMF (5 mL) was added TMS acetylene (1.9 mL, 13.6 mmol), Pd(PPh₃)Cl₂ (49 mg, 0.07 mmol), CuI (26 mg, 0.14 mmol), LiCl (433 mg, 10.2 mmol) and Diethylamine (9.2 mL, 89 mmol). The resulting solution was heated in the microwave reactor for 5 min at 120° C. After which point, the reaction was diluted with sat. NH₄Cl solution, and extracted with EtOAc. The organic layer was washed with brine, dried over MgSO₄, filtered, concentrated and purified on silica gel to afford the title compound (1.40 g), LC/MS: m/e 262.1 (M+H)⁺.

### Intermediate 49

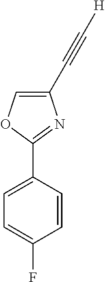 [SMILES: C#Cc1coc(-c2ccc(F)cc2)n1]

### 4-Ethynyl-2-(4-fluorophenyl)-1,3-oxazole

To a solution of Intermediate 48 (1.4 g, 5.4 mmol) in MeOH (25 mL) was added K₂CO₃ (746 mg, 5.4 mmol). The resulting solution was heated allowed to stir for 12 h. After which point, the solution was diluted with H₂O and Et₂O. The organic layer was dried over MgSO₄, filtered, concentrated to afford the title compound (1.01 g), LC/MS: m/e 188.1 (M+H)⁺.

### Intermediate 50

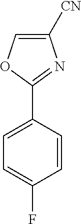 [SMILES: N#Cc1coc(-c2ccc(F)cc2)n1]

### 2-(4-Fluorophenyl)-1,3-oxazole-4-carbonitrile

To a solution of Intermediate 1 (2.1 g, 6.8 mmol) in DMF (15 mL) was added Pd(PPh₃)₄ (787 mg, 0.68 mmol), and Zn(CN)₂ (1.20 g, 10.2 mmol). The resulting solution was heated in a microwave reactor for 15 min at 120° C. After which point, the reaction was diluted with sat. NH₄Cl solution, and extracted with EtOAc. The organic layer was washed with brine, dried over MgSO₄, filtered, concentrated and purified on silica gel to afford the title compound (260 mg), LC/MS: m/e 189.2 (M+H)⁺.

### Intermediate 51

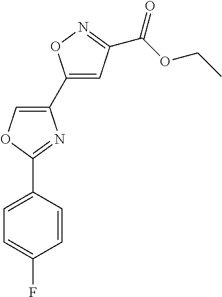 [SMILES: CCOC(=O)c1cc(-c2coc(-c3ccc(F)cc3)n2)on1]

### Ethyl 5-[2-(4-fluorophenyl)-1,3-oxazol-4-yl]isoxazole-3-carboxylate

To a stirred solution of Intermediate 49 (1.1 g, 5.9 mmol) in THF/DCM 1:1 (40 mL) was added ethyl (2Z)-chloro(hydroxyimino)ethanoate (1.3 g, 8.8 mmol) and TEA (2.4 mL, 17.6 mmol). The resulting solution was stirred for 48 h at rt. After which point, the solution was concentrated and purified on silica gel to afford the title compound (469 mg). LC/MS: m/e 303.0 (M+H)⁺.

### Intermediate 52

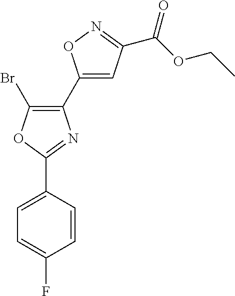 [SMILES: CCOC(=O)c1cc(-c2nc(-c3ccc(F)cc3)oc2Br)on1]

### Ethyl 5-[5-bromo-2-(4-fluorophenyl)-1,3-oxazol-4-yl]-isoxazole-3-carboxylate

The target compound was prepared in an analogous manner to Intermediate 27 starting with Intermediate 51. LC/MS: m/e 382.9 (M+H)⁺

### Intermediate 53

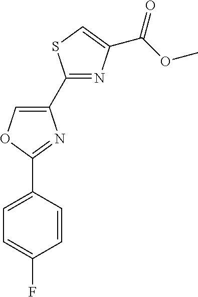 [SMILES: COC(=O)c1csc(-c2coc(-c3ccc(F)cc3)n2)n1]

### Methyl 2-[2-(4-fluorophenyl)-1,3-oxazol-4-yl]-1,3-thiazole-4-carboxylate

The target compound was prepared in an analogous manner to Intermediate 11 except that Intermediate 1 was coupled with methyl 2-bromothiazole-4-carboxylate. LC/MS: m/e 04.9 (M+H)⁺.

### Intermediate 54

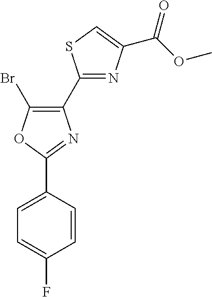 [SMILES: COC(=O)c1csc(-c2nc(-c3ccc(F)cc3)oc2Br)n1]

### Methyl 2-[5-bromo-2-(4-fluorophenyl)-1,3-oxazol-4-yl]-1,3-thiazole-4-carboxylate

The target compound was prepared in an analogous manner to Intermediate 27 starting with Intermediate 53. LC/MS: m/e 384.9 (M+H)⁺.

### Intermediate 55

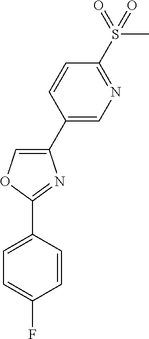 [SMILES: CS(=O)(=O)c1ccc(-c2coc(-c3ccc(F)cc3)n2)cn1]

### 5-[2-(4-Fluorophenyl)-1,3-oxazol-4-yl]-2-(methylsulfonyl)pyridine

The target compound was prepared in an analogous manner to Intermediate 11 except that Intermediate 1 was coupled with 2-bromo-5-methylsulphonylpyridine. LC/MS: m/e 318.9 (M+H)⁺.

### Intermediate 56

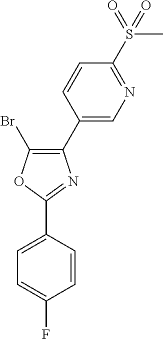 [SMILES: CS(=O)(=O)c1ccc(-c2nc(-c3ccc(F)cc3)oc2Br)cn1]

### 5-[5-Bromo-2-(4-fluorophenyl)-1,3-oxazol-4-yl]-2-(methylsulfonyl)pyridine

The target compound was prepared in an analogous manner to Intermediate 27 starting with Intermediate 55. LC/MS: in/e 398.9 (M+H)⁺.

### Intermediate 57

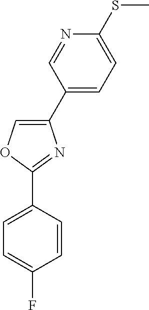 [SMILES: CSc1ccc(-c2coc(-c3ccc(F)cc3)n2)cn1]

### 5-[2-(4-Fluorophenyl)-1,3-oxazol-4-yl]-2-(methylsulfanyl)pyridine

The target compound was prepared in an analogous manner to Intermediate 11 except that Intermediate 1 was coupled with 5-bromo-2-methylthiopyridine. LC/MS: m/e 286.9 (M+H)⁺

### Intermediate 58

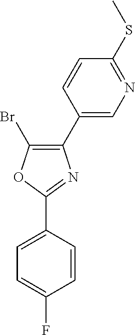 [SMILES: CSc1ccc(-c2nc(-c3ccc(F)cc3)oc2Br)cn1]

### 5-[5-Bromo-2-(4-fluorophenyl)-1,3-oxazol-4-yl]-2-(methylsulfanyl)pyridine

The target compound was prepared in an analogous manner to Intermediate 27 starting with Intermediate 57. LC/MS: m/e 366.8 (M+H)⁺

### Intermediate 59

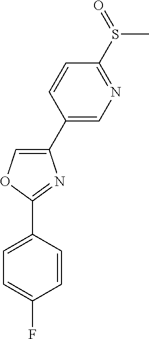 [SMILES: CS(=O)c1ccc(-c2coc(-c3ccc(F)cc3)n2)cn1]

### (R)-5-[2-(4-Fluorophenyl)-1,3-oxazol-4-yl]-2-(methylsulfinyl)pyridine and (S)-5-[2-(4-Fluorophenyl)-1,3-oxazol-4-yl]-2-(methylsulfinyl)pyridine

To a solution of Intermediate 54 (1.8 g, 63 mmol) in DCM (400 mL) at 0° C. was added a solution of mCPBA (1.4 g, 6.3 mmol) in DCM (100 mL) dropwise over 4 h. Upon complete addition, the solution was stirred for an additional 30 min. Upon completion of the reaction as judged by LC/MS analysis, the reaction was quenched with sat. NaHSO₃ solution, extracted with DCM, washed with sat. Na₂CO₃ solution, brine, dried over MgSO₄, filtered, concentrated and purified by on silica gel to afford the title compound (1.14 g), LC/MS: m/e 302.9 (M+H)⁺.

### Intermediate 60

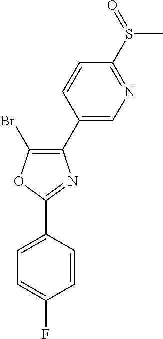 [SMILES: CS(=O)c1ccc(-c2nc(-c3ccc(F)cc3)oc2Br)cn1]

### (R)-5-[5-Bromo-2-(4-fluorophenyl)-1,3-oxazol-4-yl]-2-(methylsulfinyl)pyridine and (S)-5-[5-Bromo-2-(4-fluorophenyl)-1,3-oxazol-4-yl]-2-(methylsulfinyl)pyridine

The target compound was prepared in an analogous manner to Intermediate 27 starting with Intermediate 59. LC/MS: in/e 282.8 (M+H)⁺.

### Intermediate 61

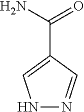

### 1H-Pyrazole-4-carboxamide

The mixture of 1H-pyrazole-4-carboxylic acid (2.0 g, 17.8 mmol) and thionyl chloride (20 mL, 168 mmol) was heated to reflux. After 4 h, the reaction mixture was concentrated, and then dried at reduced pressure for 0.5 h. The resulting residue was dissolved in CH₂Cl₂ (35 mL), cooled to 0° C. and added to a solution of ammonium hydroxide (46.8 mL, 357 mmol) in CH₂Cl₂ (20 mL). The reaction mixture was warmed to rt and stirred for 12 h. After which point, the mixture was concentrated and CH₃OH/CH₂Cl₂ (1:10, 40 ml) were added and stirred for 10 min. The solution was filtered and washed with CH₃OH/CH₂Cl₂ (1:10). The filtrate was concentrated to give the title compound (1.5 g), which was used in the next step without purification, LC/MS: m/e 112.0 (M+H)⁺.

### Intermediate 62

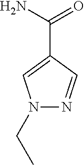

### 1-Ethyl-1H-pyrazole-4-carboxamide

To a solution of intermediate 61 (1.5 g, 13.5 mmol) in DMF (4 mL) was added powdered K₂CO₃ (5.6 g, 40.5 mmol). After 10 min, bromoethane (1.2 mL, 16.2 mmol) was added and the mixture was stirred at rt for 12 h. The reaction mixture was diluted with EtOAc, washed with water, dried over MgSO₄ and concentrated to afford the title compound as a white solid (1.0 g), which was used in the next step without purification. LC/MS: m/e 140.1 (M+H)⁺.

### Intermediate 63

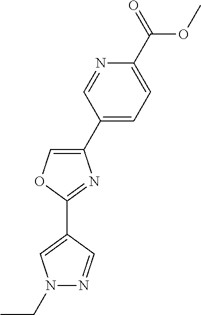 [SMILES: CCn1cc(-c2nc(-c3ccc(C(=O)OC)nc3)co2)cn1]

### Methyl 5-[2-(1-ethyl-1H-pyrazol-4-yl)-1,3-oxazol-4-yl]pyridine-2-carboxylate

To a solution of Intermediate 39 (650 mg, 2.5 mmol) in toluene (20 mL) in a sealed tube was added Intermediate 62 (876 mg, 6.3 mmol). The reaction mixture was heated to 120° C. for 12 h. The reaction mixture was then concentrated and purified on silica gel to afford the title compound as a white solid (100 mg). LC/MS: m/e 299.2 (M+H)⁺.

### Intermediate 64

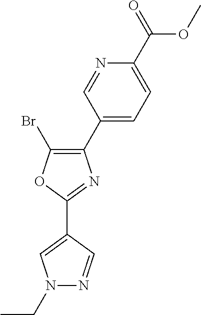 [SMILES: CCn1cc(-c2nc(-c3ccc(C(=O)OC)nc3)c(Br)o2)cn1]

### Methyl 5-[5-bromo-2-(1-ethyl-1H-pyrazol-4-yl)-1,3-oxazol-4-yl]pyridine-2-carboxylate

The target compound was prepared in an analogous manner to Intermediate 27 starting with Intermediate 63. LC/MS: m/e 379.2 (M+H)⁺.

### Intermediate 65

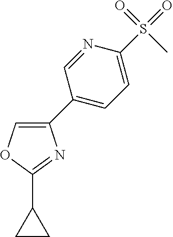 [SMILES: CS(=O)(=O)c1ccc(-c2coc(C3CC3)n2)cn1]

### 5-(2-Cyclopropyl-1,3-oxazol-4-yl)-2-(methylsulfonyl)pyridine

The target compound was prepared in an analogous manner to Intermediate 44 except starting with Intermediate 43, LC/MS: m/e 264.9 (M+H)⁺.

### Intermediate 66

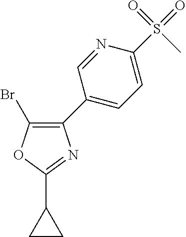 [SMILES: CS(=O)(=O)c1ccc(-c2nc(C3CC3)oc2Br)cn1]

### 5-(5-Bromo-2-cyclopropyl-1,3-oxazol-4-yl)-2-(methylsulfonyl)pyridine

The target compound was prepared in an analogues manner to Intermediate 27 starting with Intermediate 62, LC/MS: m/e 344.8 (M+H)⁺.

### Intermediate 67

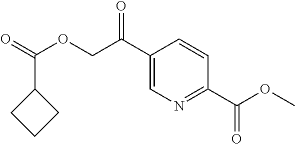 [SMILES: COC(=O)c1ccc(C(=O)COC(=O)C2CCC2)cn1]

### Meth 5-{[(cyclobutylcarbonyl)oxy]acetyl}pyridine-2-carboxylate

The target compound was prepared in an analogous manner to Intermediate 40 except that Intermediate 39 was coupled with cyclobutyl carboxylic acid. LC/MS: m/e 278.0 (M+H)⁺

### Intermediate 68

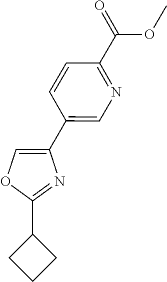 [SMILES: COC(=O)c1ccc(-c2coc(C3CCC3)n2)cn1]

### Methyl 5-(2-cyclobutyl-1,3-oxazol-4-yl)pyridine-2-carboxylate

The target compound was prepared in an analogous manner to Intermediate 44 starting with Intermediate 64. LC/MS: m/e 259.1 (M+H)⁺.

### Intermediate 69

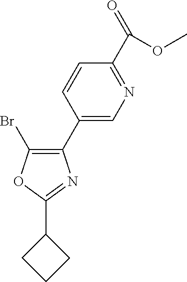 [SMILES: COC(=O)c1ccc(-c2nc(C3CCC3)oc2Br)cn1]

### Methyl 5-(5-bromo-2-cyclobutyl-1,3-oxazol-4-yl)pyridine-2-carboxylate

The target compound was prepared in an analogous manner to Intermediate 27 starting with Intermediate 68. LC/MS: m/e 338.9 (M+H)⁺

### Intermediate 70

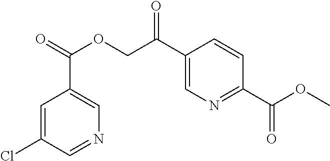 [SMILES: COC(=O)c1ccc(C(=O)COC(=O)c2cncc(Cl)c2)cn1]

### Methyl 5-({[(5-chloropyridin-3-yl)carbonyl]oxy}acetyl)pyridine-2-carboxylate

The target compound was prepared in an analogous manner to Intermediate 40 except that intermediate 39 was coupled with 5-chloropyridine-3-carboxylic acid, LC/MS: m/e 335.0 (M+H)⁺.

### Intermediate 71

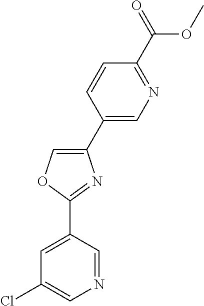 [SMILES: COC(=O)c1ccc(-c2coc(-c3cncc(Cl)c3)n2)cn1]

### Methyl 5-[2-(5-chloropyridin-3-yl)-1,3-oxazol-4-yl]pyridine-2-carboxylate

The target compound was prepared in an analogous manner to Intermediate 44 starting with Intermediate 70, LC/MS: m/e 315.9 (M+H)⁺.

### Intermediate 72

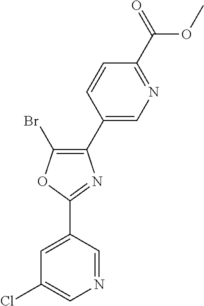 [SMILES: COC(=O)c1ccc(-c2nc(-c3cncc(Cl)c3)oc2Br)cn1]

### Methyl 5-[5-bromo-2-(5-chloropyridin-3-yl)-1,3-oxazol-4-yl]pyridine-2-carboxylate

The target compound was prepared in an analogous manner to Intermediate 27 starting with Intermediate 71. LC/MS: m/e 395.8 (M+H)⁺.

### Intermediate 73

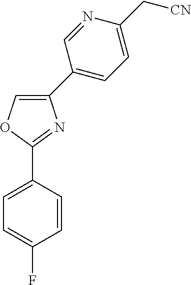 [SMILES: N#CCc1ccc(-c2coc(-c3ccc(F)cc3)n2)cn1]

### {5-[2-(4-Fluorophenyl)-1,3-oxazol-4-yl]pyridin-2-yl}acetonitrile

The target compound was prepared in an analogous manner to Intermediate 11 except that Intermediate 1 was coupled with (5-bromopyridin-2-yl)acetonitrile, LC/MS: m/e 280.0 (M+H)⁺.

### Intermediate 74

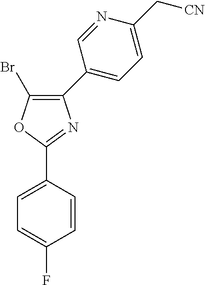 [SMILES: N#CCc1ccc(-c2nc(-c3ccc(F)cc3)oc2Br)cn1]

### {5-[5-Bromo-2-(4-fluorophenyl)-1,3-oxazol-4-yl]pyridin-2-yl}acetonitrile

The target compound was prepared in an analogous manner to Intermediate 27. LC/MS: pale 359.8 (M+H)⁺.

### Intermediate 75

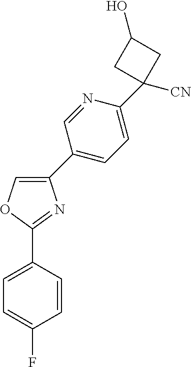 [SMILES: N#CC1(c2ccc(-c3coc(-c4ccc(F)cc4)n3)cn2)CC(O)C1]

### 1-{5-[2-(4-Fluorophenyl)-1,3-oxazol-4-yl]pyridin-2-yl}-3-hydroxycyclobu-tanecarbonitrile

To a solution of Intermediate 73 (100 mg, 0.4 mmol) in DMF (8 mL) at rt was added NaH (31.5 mg, 0.8 mmol), followed by epichlorohydrin (39.8 mg, 0.4 mmol). The resulting solution was stirred at rt for 1 h. Upon completion of the reaction as judged by TLC analysis, the reaction was quenched with H₂O, extracted with EtOAc, washed with brine, dried over MgSO₄, filtered, concentrated and purified on silica gel to afford the title compound (16 mg), LC/MS: m/e 336.1 (M+H)⁺.

### Intermediate 76

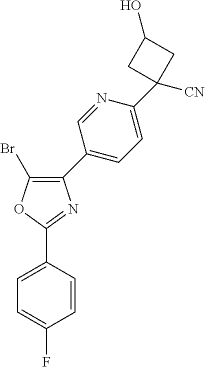 [SMILES: N#CC1(c2ccc(-c3nc(-c4ccc(F)cc4)oc3Br)cn2)CC(O)C1]

### 1-{5-[5-Bromo-2-(4-fluorophenyl)-1,3-oxazol-4-yl]pyridin-2-yl}-3-hydroxycyclobutanecarbonitrile

The target compound was prepared in an analogous manner to Intermediate 27. LC/MS: m/e 415.9 (M+H)⁺.

### Intermediate 77

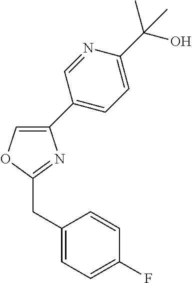 [SMILES: CC(C)(O)c1ccc(-c2coc(Cc3ccc(F)cc3)n2)cn1]

### 2-{5-[2-(4-Fluorobenzyl)-1,3-oxazol-4-yl]pyridin-2-yl}propan-2-ol

The target compound was prepared in an analogous manner to Intermediate 11 except that Intermediate 1 was replaced with Intermediate 25. LC/MS: m/e 313.1 (M+H)⁺.

### Intermediate 78

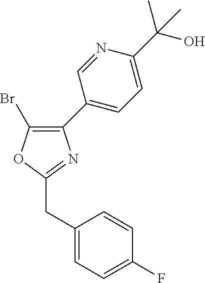 [SMILES: CC(C)(O)c1ccc(-c2nc(Cc3ccc(F)cc3)oc2Br)cn1]

### 2-{5-[5-Bromo-2-(4-fluorobenzyl)-1,3-oxazol-4-yl]pyridin-2-yl}propan-2-ol

The target compound was prepared in an analogous manner to intermediate 27. LC/MS: In/e 393.0 (M+H)⁺.

### Intermediate 79

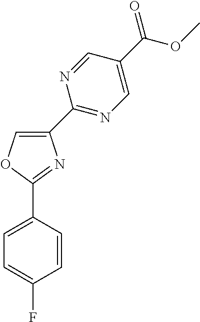 [SMILES: COC(=O)c1cnc(-c2coc(-c3ccc(F)cc3)n2)nc1]

### Methyl 2-[2-(4-fluorophenyl)-1,3-oxazol-4-yl]pyrimidine-5-carboxylate

The target compound was prepared in an analogous manner to Intermediate 11 except that Intermediate 1 was coupled with methyl 2-chloropyrimidine-5-carboxylate. LC/MS: m/e 300.1 (M+H)⁺.

### Intermediate 80

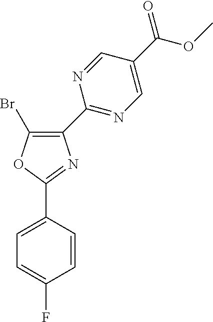 [SMILES: COC(=O)c1cnc(-c2nc(-c3ccc(F)cc3)oc2Br)nc1]

### Methyl 2-[5-bromo-2-(4-fluorophenyl)-1,3-oxazol-4-yl]pyrimidine-5-carboxylate

The target compound was prepared in an analogous manner to Intermediate 27 starting with Intermediate 79, LC/MS: m/e 377.9 (M+H)⁺.

### Intermediate 81

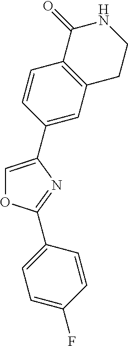 [SMILES: O=C1NCCc2cc(-c3coc(-c4ccc(F)cc4)n3)ccc21]

### 6-[2-(4-Fluorophenyl)-1,3-oxazol-4-yl]-3,4-dihydroisoquinolin-1(2H)-one

The target compound was prepared in an analogous manner to Intermediate 11 except that Intermediate 1 was coupled with 6-bromo-3,4-dihydroisoquinolin-1(2H)-one (Bioorg. Med. Chem. Lett., 2006, 16, 2584), LC/MS: m/e 309.3 (M+H)⁺.

### Intermediate 82

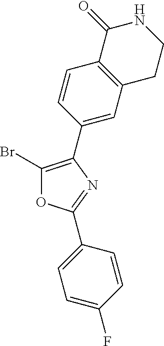 [SMILES: O=C1NCCc2cc(-c3nc(-c4ccc(F)cc4)oc3Br)ccc21]

### 6-[5-Bromo-2-(4-fluorophenyl)-1,3-oxazol-4-yl]-3,4-dihydroisoquinolin-1-(2H)-one

The target compound was prepared in an analogous manner to Intermediate 27 starting with Intermediate 81, LC/MS: m/e 388.9 (M+H)⁺.

### Intermediate 83

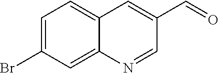 [SMILES: O=Cc1cnc2cc(Br)ccc2c1]

### 7-Bromoquinoline-3-carbaldehyde

The title compound was prepared using the procedure described by Sato, I.; Nakao, T.; Sugie, R.; Kawasaki, T.; Soai, K. Synthesis 2004, 9, 1419.

### Intermediate 84

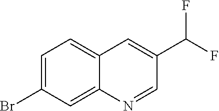 [SMILES: FC(F)c1cnc2cc(Br)ccc2c1]

### 7-Bromo-3-(difluoromethyl)quinoline

Dissolved the Intermediate 83 (72 mg, 0.30 mmol) in CH₂Cl₂ (1 mL) and added a solution of Deoxo-Fluor (0.096 mL, 0.519 mmol) in CH₂Cl₂ (1 ml) followed by EtOH (0.004 mL, 0.069 mmol). Stirred overnight at rt. Diluted with CH₂Cl₂ and added sat'd. NaHCO₃. Extracted with CH₂Cl₂ (3×), dried over MgSO₄, filtered, evaporated and dried under high vac at it Light yellow oil. Purified by prep TLC (SiO₂, 20×20 cm, 1000 microns, 1 plate; hexane-EtOAc, 9:1) to afford title compound (59 mg), LC/MS:[M+H]⁺ m/e 258, 260 (M+H)⁺.

### Intermediate 85

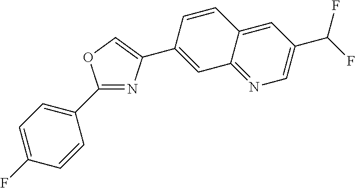 [SMILES: Fc1ccc(-c2nc(-c3ccc4cc(C(F)F)cnc4c3)co2)cc1]

### 3-(Difluoromethyl)-7-[2-(4-fluorophenyl)-1,3-oxazol-4-yl]quinoline

The target compound was prepared in an analogous manner to Intermediate 11 except that Intermediate 1 was coupled with 7-Bromo-3-(difluoromethyl)quinoline. LC/MS: m/e 341.5.

### Intermediate 86

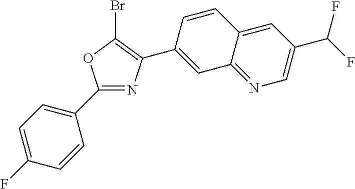 [SMILES: Fc1ccc(-c2nc(-c3ccc4cc(C(F)F)cnc4c3)c(Br)o2)cc1]

### 7-[5-Bromo-2-(4-fluorophenyl)-1,3-oxazol-4-yl]-3-(difluoromethyl)quinoline

The target compound was prepared in an analogous manner to Intermediate 27 starting with Intermediate 85 LC/MS: m/e 421 (M+H)⁺.

### Intermediate 87

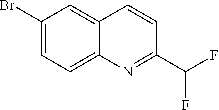 [SMILES: FC(F)c1ccc2cc(Br)ccc2n1]

### 6-Bromo-2-(difluoromethyl)quinoline

Suspended 6-bromoquinoline-2-carbaldehyde (472 mg, 2 mmol) in CH₂Cl₂ (2 mL) and added a solution of Deoxo-Fluor (0.627 mL, 3.4 mmol) in CH₂Cl₂ (2 mL) followed by EtOH (0.023 mL, 0.4 mmol). Stirred for 48 hrs at rt. Diluted with CH₂Cl₂ and added sat'd. NaHCO₃. Extracted with CH₂Cl₂ (3×), washed extracts with brine (1×), dried over MgSO₄, filtered, evaporated and dried under high vac at rt. The light brown solids were dissolved in a small amount of CH₂Cl₂-MeOH and stirred with a small amount of silica gel for 15 min. Filtered, evaporated and dried under high vac at rt to afford the title compound (491 mg), LC/MS: ink 258, 260 (M+H)⁺.

### Intermediate 88

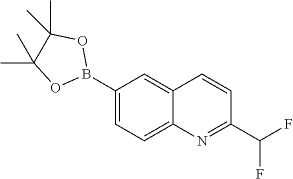 [SMILES: CC1(C)OB(c2ccc3nc(C(F)F)ccc3c2)OC1(C)C]

### 2-(Difluoromethyl)-6-(4,4,5,5-tetramethyl-1,3,2-dioxaborolan-2-yl)quinoline

Mixed the Intermediate 87 (504 mg, 1.953 mmol), bis(pinacolato)diboron (506 mg, 1.992 mmol), PdCl₂(dppf) (43 mg, 0.059 mmol) and KOAc (575 mg, 5.86 mmol) with DMSO (4.0 mL) in a sealed vial. Degassed by bubbling in N₂ gas and then blanketing vessel with N₂ and sealed with Teflon stopper. Heated to 80° C. Heated and stirred overnight. Cooled to rt after 16 hrs. Diluted with water and extracted with EtOAc (3×), washed with brine (1×), dried over MgSO₄, decolorized with charcoal, filtered, evaporated and dried under high vac at rt to afford the title compound (788 mg). LC/MS: m/e 306 (M+H)⁺.

### Intermediate 89

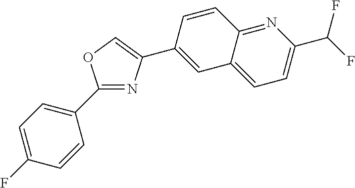 [SMILES: Fc1ccc(-c2nc(-c3ccc4nc(C(F)F)ccc4c3)co2)cc1]

### 2-(Difluoromethyl)-6-[2-(4-fluorophenyl)-1,3-oxazol-4-yl]quinoline

Dissolved 2-(4-fluorophenyl)-1,3-oxazol-4-yltrifluoromethanesulfonate (185 mg, 0.593 mmol) and Intermediate 88 (263 mg, 0.652 mmol) in DMF (3.2 mL) and added PdCl₂(dppf) (13 mg, 0.018 mmol) followed by Na₂CO₃ (314 mg, 2.97 mmol) and water (0.72 mL) in a sealed tube. The flask was sealed with a Teflon stopper and heated at 90° C. After 5 h the reaction was cooled to rt, diluted with water and extracted with CH₂Cl₂ (3×). Washed extracts with brine (1×), dried over MgSO₄, decolorized with charcoal and filtered through filtered. Evaporated filtrate to dryness and dried under high vac at rt. The brown solids were purified by prep TLC (SiO₂, 20×20 cm, 1000 microns, 3 plates; hexane-EtOAc, 3:1) to afford the title compound (109 mg). LC/MS: m/e 341 (M+H)⁺.

### Intermediate 90

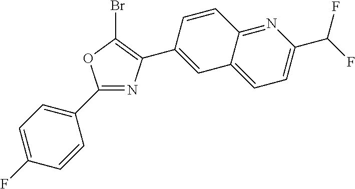 [SMILES: Fc1ccc(-c2nc(-c3ccc4nc(C(F)F)ccc4c3)c(Br)o2)cc1]

### 6-[5-Bromo-2-(4-fluorophenyl)-1,3-oxazol-4-yl]-2-(difluoromethyl)quinoline

The target compound was prepared in an analogous manner to Intermediate 27 starting with Intermediate 89, LC/MS: m/e 421 (M+H)⁺.

### Intermediate 91

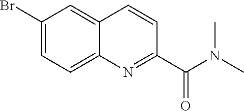 [SMILES: CN(C)C(=O)c1ccc2cc(Br)ccc2n1]

### 6-Bromo-N,N-dimethylquinoline-2-carboxamide

Suspended the 6-bromoquinoline-2-carboxylic acid (1.0 g, 3.93 mmol) in CH₂Cl₂ (20 mL), added DMF (0.91 mL, 11.78 mmol) and cooled in an ice bath. Added oxalyl chloride (0.688 mL, 7.86 mmol) dropwise over a few min. Warmed to rt and stirred for 1 hr then bubbled in dimethylamine gas for several min. The dark amber mixture was stirred at rt overnight. In am, the solution was diluted with water and extracted with CH₂Cl₂ (3×). Washed extracts with brine (1×), dried over MgSO₄, decolorized with charcoal, filtered, evaporated and dried under high vac, rt to afford the title compound (990 mg), LC/MS: m/e 279, 281 (M+H)⁺.

### Intermediate 92

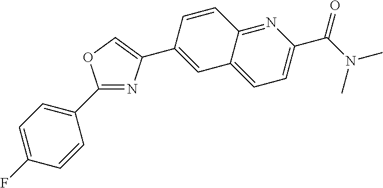 [SMILES: CN(C)C(=O)c1ccc2cc(-c3coc(-c4ccc(F)cc4)n3)ccc2n1]

### 6-[2-(4-Fluorophenyl)-1,3-oxazol-4-yl]-N,N-dimethylquinoline-2-carboxamide

The target compound was prepared in an analogous manner to Intermediate 11 except that Intermediate 1 was coupled with Intermediate 91, LC/MS: m/e 362.4 (M+H)⁺.

### Intermediate 93

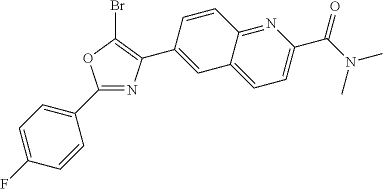 [SMILES: CN(C)C(=O)c1ccc2cc(-c3nc(-c4ccc(F)cc4)oc3Br)ccc2n1]

### 6-[5-Bromo-2-(4-fluorophenyl)-1,3-oxazol-4-yl]-N,N-dimethylquinoline-2-carboxamide

The target compound was prepared in an analogous manner to Intermediate 27 starting with Intermediate 92. LC/MS: m/e 442.1 (M+H)⁺.

### Intermediate 94

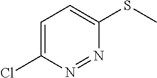

### 3-Chloro-6-(methylsulfanyl)pyridazine

Dissolved 2,5-dichloropyridazine (8.7 g, 58.4 mmol) in DMF (30 mL) and added a solution of CH₃SNa (4.1 g, 58.5 mmol) in DMF (60 mL) over 15 min. Mild exotherm which was controlled by use of a cold water bath. Stirred at rt for 12 h. Evaporated much of the DMF (˜50 mL) then diluted with a large volume of water when solid precipitates. Stirred at rt for 2 h then filtered the white solids and washed with water. Dissolved the solid in CH₂Cl₂, separated out of the water and dried over MgSO₄. Filtered, evaporated and dried under high vac at rt to afford the title compound (5.77 g), LC/MS: m/e 161 (M+H)⁺.

### Intermediate 95

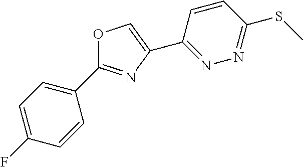 [SMILES: CSc1ccc(-c2coc(-c3ccc(F)cc3)n2)nn1]

### 3-[2-(4-Fluorophenyl)-1,3-oxazol-4-yl]-6-(methylsulfanyl)pyridazine

The target compound was prepared in an analogous manner to Intermediate 11 except that Intermediate 1 was coupled with Intermediate 94, LC/MS: m/e 288 (M+H)⁺.

### Intermediate 96

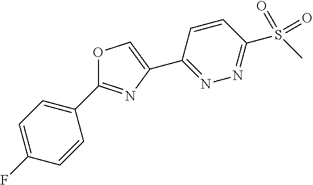 [SMILES: CS(=O)(=O)c1ccc(-c2coc(-c3ccc(F)cc3)n2)nn1]

### 3-[2-(4-Fluorophenyl)-1,3-oxazol-4-yl]-6-(methylsulfonyl)pyridazine

Intermediate 95 (135 mg, 0.47 mmol) in MeOH (25.0 mL) was treated with a solution of oxone (867 mg, 1.41 mmol) in water (5.0 mL) dropwise and stirred at rt. The solution was then evaporated to dryness, extracted with CH₂Cl₂ (3×). The combined organic extracts were dried over MgSO₄, filtered and evaporated to afford the title compound (134 mg). LC/MS: m/e 320 (M+H)⁺.

### Intermediate 97

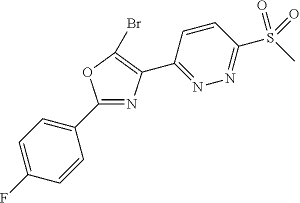 [SMILES: CS(=O)(=O)c1ccc(-c2nc(-c3ccc(F)cc3)oc2Br)nn1]

### 3-[5-Bromo-2-(4-fluorophenyl)-1,3-oxazol-4-yl]-6-(methylsulfonyl)pyridazine

The target compound was prepared in an analogous manner to Intermediate 27 starting with Intermediate 96, LC/MS: m/e 399.7 (M+H)⁺.

### Intermediate 98

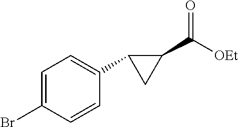 [SMILES: CCOC(=O)[C@H]1C[C@@H]1c1ccc(Br)cc1]

### Ethyl (1S,2S)-2-(4-bromophenyl)cyclopropanecarboxylate

To a 1-neck, 1-L round bottom flask equipped with a magnetic stirrer was added 265 mL methyl tert-butyl ether. The flask was evacuated and flushed with nitrogen three times. 2,2′-Isopropylidenebis[(4R)-4-tert-butyl-2-oxazolidine] (2.39 g, 8.03 mmol) was added, followed by copper(I) trifluoromethanesulfonate benzene complex (4.49 g, 8.03 mmol). The green suspension was stirred at room temperature for about 2 hours and was then filtered. The filtrate was added to a 4-neck, 5-L, round bottom flask equipped with a mechanical stirrer, thermocouple, nitrogen bubbler, and addition funnel. Then, 4-bromostyrene (150 g, 0.803 mol) was added to this solution and the reaction was cooled to 0° C. via an ice/water bath. Ethyl diazoacetate (167 mL, 1.606 mol) was dissolved in 1675 mL of MTBE and the solution was evacuated/flushed with nitrogen three times. This solution was then added to an addition funnel and added dropwise to the reaction mixture. A slight exotherm was observed. The ethyl diazoacetate was allowed to add slowly over the weekend and the reaction slowly warmed to room temperature. The reaction was poured into a large extractor and diluted with 4L MTBE. The organics were washed with 2×1 L 3% aq. ammonium hydroxide and 2 L brine, dried over anhydrous magnesium sulfate, filtered, and concentrated. The residue was dissolved in heptane and a small amount of dichloromethane, injected onto an ISCO 1500 g column prepacked in heptane. The column was eluted with 100% heptane over 1. column volume, 0-20% ethyl acetate/heptane over 6.5 column volumes, and held at 20% ethyl acetate/heptane over 8 column volumes. The product containing fractions were collected and concentrated to give 191 g (yield 88%) of the title compound. 1H NMR (500 MHz, (CDCl₃): 7.42 (d, 2H), 7.01 (d, 2H), 4.21 (q, 2H), 2.49 (m, 1H), 1.88 (m, 1H), 1.62 (m, 2H), 1.25 (t, 3H).

The compounds in Table 5 were prepared from the appropriate starting materials using the procedure for Example 12.

TABLE 5

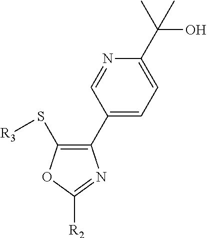

| | | | LCMS: |
|---|---|---|---|
| | | | found m/e |
| Example | R₂ | R₃ | (M + H) |
| 57 | 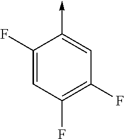 | 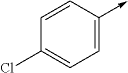 | 477.1 |
| 58 | 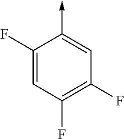 | 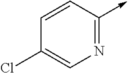 | 478.1 |
| 59 | 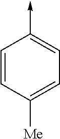 | 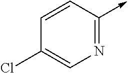 | 438.1 |
| 60 | 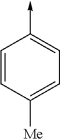 | 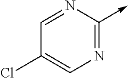 | 439.1 |
| 61 | 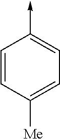 | 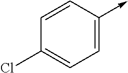 | 437.2 |
| 62 | 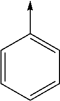 | 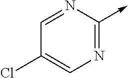 | 425.1 |
| 63 | 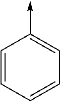 | 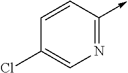 | 424.2 |
| 64 | 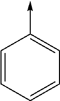 | 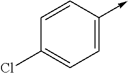 | 423.1 |
| 65 | 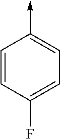 | 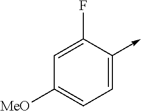 | 455.0 |
| 66 | 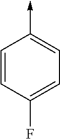 | 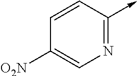 | 453.0 |
| 67 | 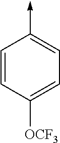 | 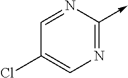 | 508.9 |
| 68 | 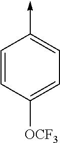 | 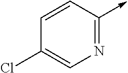 | 507.9 |
| 69 | 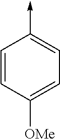 | 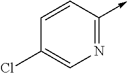 | 454.0 |

### Example 70

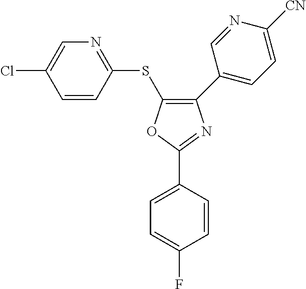 [SMILES: N#Cc1ccc(-c2nc(-c3ccc(F)cc3)oc2Sc2ccc(Cl)cn2)cn1]

### 5-{5-[(5-chloropyridin-2-yl)sulfanyl]-2-(4-fluorophenyl)-1,3-oxazol-4-yl}pyridine-2-carbonitrile

The title compound was prepared in an analogous manner to Example 12 starting with Intermediate 33. LC/MS: m/e 409.9 (M+H)⁺. ¹H NMR (500 MHz, Acetone-d6): δ 7.39 (m, 2H), 7.48 (d, J=8.5 Hz, 1H), 7.83 (m, 1H), 8.05 (d, J=8.5 Hz, 1H), 8.24 (m, 2H), 8.45 (d, J=2.5 Hz, 1H), 8.72 (m, 1H), 9.44 (d, J=1.5 Hz, 1H).

### Example 71

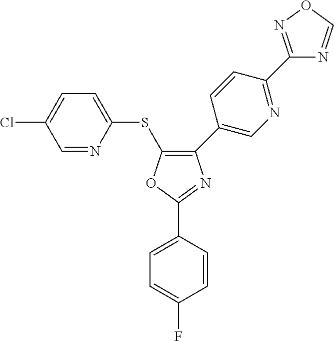 [SMILES: Fc1ccc(-c2nc(-c3ccc(-c4ncon4)nc3)c(Sc3ccc(Cl)cn3)o2)cc1]

### 5-Chloro-2-({2-(4-fluorophenyl)-4-[6-(1,2,4-oxadiazol-3-yl)pyridin-3-yl]-1,3-oxazol-5-yl}sulfanyl)pyridine

To Example 70 (100 mg, 0.25 mmol) in 10 mL EtOH was added 1.0 mL of 50 wt % aqueous NH₂OH and 15 mg of K₂CO₃. The reaction was heated to 120° C. for 5 min via microwave irradiation. The reaction mixture was concentrated to dryness and the residue was dissolved in 5 mL triethylorthoformate, 10 mL EtOH and 1 mL of TFA. The reaction was heated to 100° C. for 10 min via microwave irradiation. The volatiles were removed and the residue was purified on silica gel to afford the title compound (64 mg). LC/MS: m/e 452.0 (M+H)⁺. ¹H NMR (500 MHz, Acetone-d6): δ 7.37-7.41 (m, 3H), 7.82 (m, 1H), 8.27 (m, 2H), 8.47 (d, J=2.0 Hz, 1H), 8.69 (d, J=6.5 Hz, 1H), 9.47 (s, 1H).

### Example 72

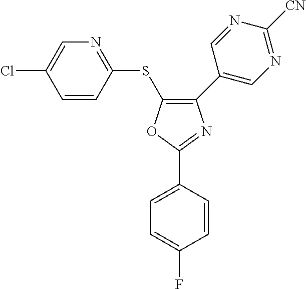 [SMILES: N#Cc1ncc(-c2nc(-c3ccc(F)cc3)oc2Sc2ccc(Cl)cn2)cn1]

### 5-{5-[(5-Chloropyridin-2-yl)sulfanyl]-2-(4-fluorophenyl)-1,3-oxazol-4-yl}pyrimidine-2-carbonitrile

The title compound was prepared in an analogous manner to Example 12 starting with Intermediate 31. LC/MS: m/e 410.0 (M+H)⁺. ¹H NMR (500 MHz, Acetone-d6): δ 7.41 (m, 2H), 7.53 (d, J=8.5 Hz, 1H), 7.84 (m, 1H), 8.26 (m, 2H), 8.45 (d, J=2.5 Hz, 1H), 9.61 (s, 2H).

### Example 73

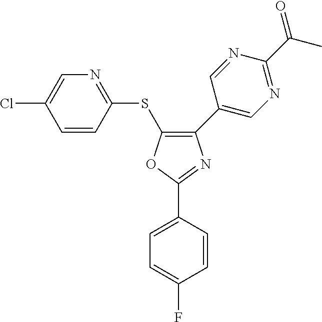 [SMILES: CC(=O)c1ncc(-c2nc(-c3ccc(F)cc3)oc2Sc2ccc(Cl)cn2)cn1]

### 1-(5-{5-[(5-Chloropyridin-2-yl)sulfanyl]-2-(4-fluorophenyl)-1,3-oxazol-4-yl}pyrimidin-2-yl)ethanone

A solution of 5-{5-[(5-chloropyridin-2-yl)sulfanyl]-2-(4-fluorophenyl)-1,3-oxazol-4-yl}pyrimidine-2-carbonitrile (Example 72) (87 mg, 0.21 mmol) in THF (5 mL) was treated with methylmagnesium bromide (0.7 mL, 2.1 mmol, 3.0 Min THF) at rt. Upon completion of the reaction as judged by TLC analysis, the solution was diluted with saturated aq NH₄Cl solution and extracted with EtOAc. The organic layer was removed, dried over MgSO₄, filtered and concentrated giving rise to an oil. The oil was purified on silica gel to afford the title compound (13 mg). LC/MS: m/e 427.0 (M+H)⁺. ¹H NMR (500 MHz, Acetone-d6): δ 2.70 (s, 3H), 7.41 (m, 2H), 7.51 (d, J=9.0 Hz, 1H), 7.83 (m, 1H), 8.27 (m, 2H), 8.45 (d, J=2.5 Hz, 1H), 9.57 (s, 21-1).

### Example 74

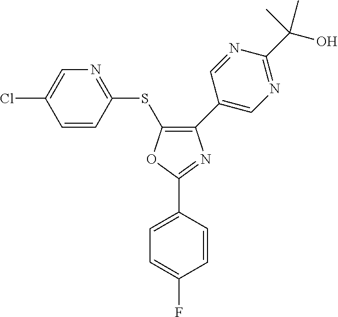 [SMILES: CC(C)(O)c1ncc(-c2nc(-c3ccc(F)cc3)oc2Sc2ccc(Cl)cn2)cn1]

### 2-(5-{5-[(5-Chloropyridin-2-yl)sulfanyl]-2-(4-fluorophenyl)-1,3-oxazol-4-yl}pyrimidin-2-yl)propan-2-ol

A solution of 1-(5-{5-[(5-chloropyridin-2-yl)sulfanyl]-2-(4-fluorophenyl)-1,3-oxazol-4-yl}pyrimidin-2-yl)ethanone (Example 73) (12 mg, 0.03 mmol) in THF (5 mL) was treated with methylmagnesium bromide (0.09 mL, 0.3 mmol, 3.0 M in THF) at rt. Upon completion of the reaction as judged by TLC analysis, the solution was diluted with saturated aq NH₄Cl solution and extracted with EtOAc. The organic layer was removed, dried over MgSO₄, filtered and concentrated giving rise to an oil. The oil was purified on silica gel to afford the title compound (6.3 mg). LC/MS: m/e 443.0 (M+H)⁺. ¹H NMR (500 MHz, Acetone-d6): δ 1.54 (s, 6H), 4.56 (s, 1H), 7.39 (m, 2H), 7.47 (d, J=8.5 Hz, 1H), 7.82 (m, 1H), 8.25 (m, 2H), 8.45 (d, J=2.5 Hz, 1H), 9.39 (s, 2H).

The compounds in Table 6 were prepared from the appropriate starting materials using the procedure for Example 12.

TABLE 6

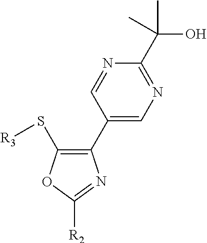

| | | | LCMS: found |
|---|---|---|---|
| Example | R₂ | R₃ | m/e (M + H) |
| 75 | 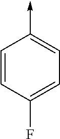 | 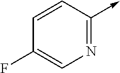 | 427.0 |
| 76 | 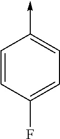 | 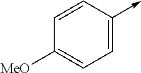 | 438.0 |
| 77 | 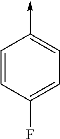 | 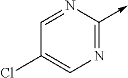 | 443.9 |

### Example 78

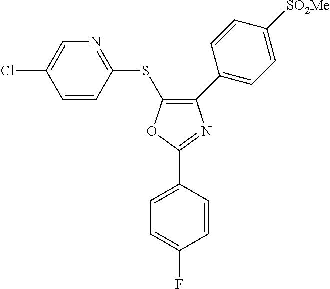 [SMILES: CS(=O)(=O)c1ccc(-c2nc(-c3ccc(F)cc3)oc2Sc2ccc(Cl)cn2)cc1]

### 5-Chloro-2-({2-(4-fluorophenyl)-4-[4-(methylsulfonyl)phenyl]-1,3-oxazol-5-yl}sulfanyl)pyridine

A stirred solution of Intermediate 29 (1.30 g, 3.30 mmol), 5-chloropyridine-2-thiol (573 mg, 3.90 mmol), and K₂CO₃ (1.36 g, 9.80 mmol) dissolved in 60 mL of NMP was heated to 60° C. for 1 h. After which point, the solution was diluted with dist. H₂O and EtOAc. The organic layer was removed followed by drying over MgSO₄, filtration, and concentration giving rise to an oil. The oil was purified on silica gel to afford the title compound (130 mg). LC/MS: m/e 460.7 (M+H)⁺. ¹H NMR (500 MHz, CDCl₃): δ 3.09 (s, 3H), 7.05 (d, J=8.5 Hz, 1H), 7.22 (m, 2H), 7.56 (m, 1H), 8.01 (d, J=8.5 Hz, 2H), 8.19 (m, 2H), 8.37 (d, J=8.5 Hz, 2H), 8.41 (d, J=2.5 Hz, 1H).

### Example 79

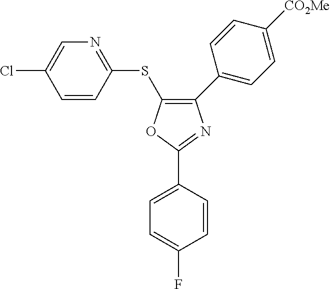 [SMILES: COC(=O)c1ccc(-c2nc(-c3ccc(F)cc3)oc2Sc2ccc(Cl)cn2)cc1]

### Methyl-4-{5-[(5-chloropyridin-2-yl)sulfanyl]-2-(4-fluorophenyl)-1,3-oxazol-4-yl}benzoate

A stirred solution of Intermediate 27 (500 mg, 1.30 mmol), 5-chloropyridine-2-thiol (290 mg, 2.00 mmol), and K₂CO₃ (551 mg, 4.00 mmol) dissolved in 20 mL of NMP was heated to 80° C. for 12 h. After which point, the solution was diluted with dist. H₂O and EtOAc. The organic layer was removed followed by drying over MgSO₄, filtration, and concentration giving rise to an oil. The oil was purified on silica gel to afford the title compound (330 mg). LC/MS: m/e 440.9 (M+H)⁺. ¹HNMR (500 MHz, CDCl₃): δ 3.95 (s, 3H), 7.02 (d, J=8.5 Hz, 1H), 7.22 (m, 2H), 7.56 (m, 1H), 8.11 (d, J=8.5 Hz, 2H), 8.13 (m, 2H), 8.25 (d, J=8.5 Hz, 2H), 8.43 (d, J=2.5 Hz, 1H).

### Example 80

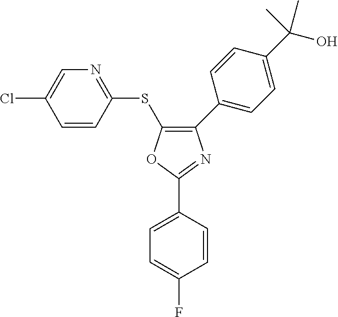 [SMILES: CC(C)(O)c1ccc(-c2nc(-c3ccc(F)cc3)oc2Sc2ccc(Cl)cn2)cc1]

### 2-(4-{5-[(5-Chloropyridin-2-yl)sulfanyl]-2-(4-fluorophenyl)-1,3-oxazol-4-yl}phenyl)propan-2-ol

A solution of methyl-4-{5-[(5-chloropyridin-2-yl)sulfanyl]-2-(4-fluorophenyl)-1,3-oxazol-4-yl}benzoate (Example 79) (127 mg, 0.29 mmol) in THF (10 mL) was treated with methylmagnesium bromide (0.50 mL, 1.4 mmol, 3.0 M in THF) at rt. Upon completion of the reaction as judged by TLC analysis, the solution was diluted with saturated aq NH₄Cl solution and extracted with EtOAc. The organic layer was removed, dried over MgSO₄, filtered and concentrated giving rise to an oil. The oil was purified on silica gel to afford the title compound (100 mg). LC/MS: m/e 441.0 (M+H)⁺.

### Example 81

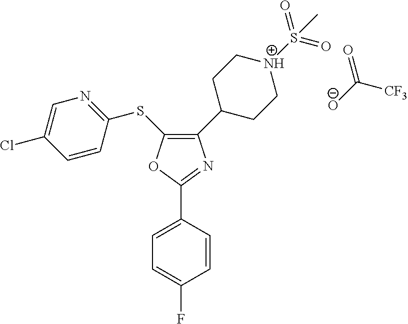 [SMILES: CS(=O)(=O)[NH+]1CCC(c2nc(-c3ccc(F)cc3)oc2Sc2ccc(Cl)cn2)CC1 ; O=C([O-])C(F)(F)F]

### 4-{5-[(5-Chloropyridin-2-yl)sulfanyl]-2-(4-fluorophenyl)-1,3-oxazol-4-yl}-1-(methylsulfonyl)piperidinium trifluoroacetate

A solution of Intermediate 35 (100 mg, 0.925 mmol) in CH₂Cl₂ (10 mL) was stirred at rt for 16 h. Upon completion of the reaction as judge by TLC, the solution was diluted with CH₂Cl₂ (20 mL) and sat aq. Na₂S₂O₃ (30 mL). The organic layer was removed, dried over MgSO₄, filtered, and concentrated to afford the corresponding bromide. The material was taken onto the next step directly. At this point, a solution of 5-chloropyridine-2-thiol (79 mg, 0.564 mmol) in DME (2 mL) was added K₂CO₃ (113 mg, 0.818 mmol) and stirred at rt for 15 min. A solution of the freshly prepared bromide (110 mg, 0.273 mmol), neocuproine (14.0 mg, 0.068 trawl) and CuI (13 mg, 0.068 mmol) in DME (2 mL) was added to the mixture and heated to 90° C. for 2 h. The solution was allowed to cool to rt, concentrated under vacuum and the residue was purified by reverse phase HPLC to afford 9 mg of the final compound as a TPA salt, LCMS: m/z 468.0 (M+H)⁺.

The compounds in Table 7 were prepared from the appropriate starting materials using the procedure for Example 12.

TABLE 7

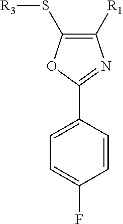

| | | | LCMS: |
|---|---|---|---|
| | | | found m/e |
| Example | R₁ | R₃ | (M + H) |
| 82 | 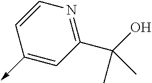 | 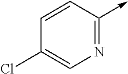 | 442.1 |
| 83 | 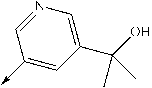 | 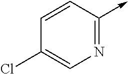 | 442.1 |

### Example 84

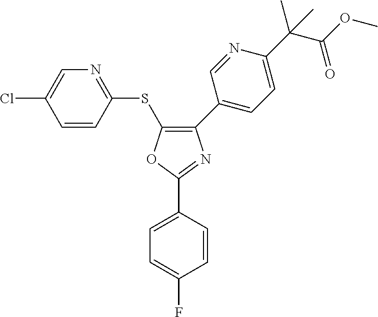 [SMILES: COC(=O)C(C)(C)c1ccc(-c2nc(-c3ccc(F)cc3)oc2Sc2ccc(Cl)cn2)cn1]

### Methyl 2-(5-{5-[(5-chloropyridin-2-yl)sulfanyl]-2-(4-fluorophenyl)-1,3-oxazol-4-yl}pyridin-2-yl)-2-methylpropanoate

The title compound was prepared in an analogous manner to Example 12 starting with Intermediate 37, LC/MS: m/e 484.1 (M+H)⁺.

### Example 85

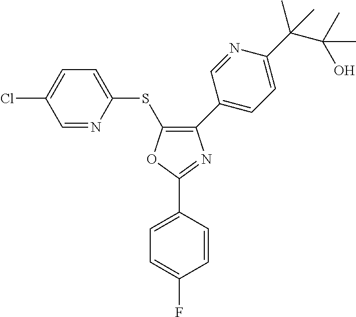 [SMILES: CC(C)(O)C(C)(C)c1ccc(-c2nc(-c3ccc(F)cc3)oc2Sc2ccc(Cl)cn2)cn1]

### 3-(5-{5-[(5-chloropyridin-2-yl)sulfanyl]-2-(4-fluorophenyl)-1,3-oxazol-4-yl}pyridin-2-yl)-2,3-dimethylbutan-2-ol

The title compound was prepared in an analogous manner to Example 80 starting with Example 84. LC/MS: m/e 484.2 (M+H)⁺. ¹H NMR (500 MHz, acetone-d6): δ 1.04 (s, 6H), 1.41 (s, 6H), 7.39 (m, 3H), (7.36 (d, J=8 Hz, 1H), 7.80 (dd, J=2.5, 8.5 Hz, 1H), 8.24 (m, 2H), 8.44 (m, 2H), 9.23 (d, J=1.5 Hz, 1H).

### Example 86

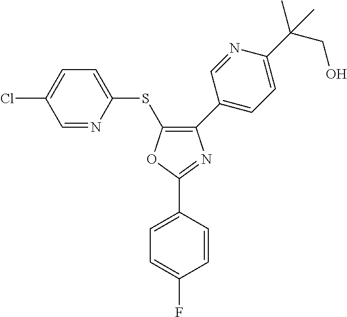 [SMILES: CC(C)(CO)c1ccc(-c2nc(-c3ccc(F)cc3)oc2Sc2ccc(Cl)cn2)cn1]

### 2-(5-{5-[(5-chloropyridin-2-yl)sulfanyl]-2-(4-fluorophenyl)-1,3-oxazol-4-yl}pyridin-2-yl)-2-methylpropan-1-ol

To a solution of Example 84 (120 mg, 0.2 mmol) in THF (10 mL) at −78° C. was added DIBAl—H (1.0M/toluene, 0.6 mL, 0.6 mmol). The resulting solution was stirred at −78° C. for 1 h. The reaction mixture was then poured into a vigorously stirred Rochelle salt solution/EtOAc (1:1). Upon clarification of the organic layer, the layers were separated, dried over MgSO₄, filtered, concentrated, and purified on silica gel to afford the title compound (95.7 mg). LC/MS: m/e 456.1 (M+H)⁺. ¹H NMR (500 MHz, acetone-d6): δ 1.35 (s, 6H), 3.73 (d, J=5.5 Hz, 2H), 4.08 (t, J=5.5 Hz, 1H) 7.40 (m, 3H), 7.58 (d, J=7.5 Hz, 1H), 7.82 (dd, J=3, 9 Hz, 1H), 8.25 (m, 2H), 8.39 (dd, J=2.5, 8.5 Hz, 1H), 8.47 (d, 2.5J=2.5 Hz, 1H), 9.21 (s, 1H).

### Example 87

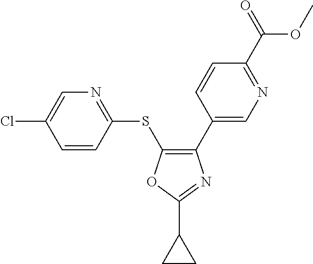 [SMILES: COC(=O)c1ccc(-c2nc(C3CC3)oc2Sc2ccc(Cl)cn2)cn1]

### Methyl 5-{5-[(5-chloropyridin-2-yl)sulfanyl]-2-cyclopropyl-1,3-oxazol-4-yl}pyridine-2-carboxylate

To a solution of Intermediate 45 (2.2 g, 6.8 mmol) in NMP (65 mL) at rt was added 5-chloropyridine-2-thiol (1.19 g, 8.17 mmol) and K₂CO₃ (2.82 g, 20.4 mmol). The resulting solution was heated at 60° C. overnight. Upon completion of the reaction as judged by LC/MS analysis, the reaction was dilute with water, extract with EtOAc, the combined organic layers was washed with brine, dried over MgSO₄, filtered, concentrated and purified on silica gel to afford the title compound (2.54 g). LC/MS: n2/e 387.9 (M+H)⁺.

### Example 88

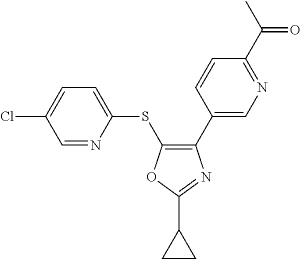 [SMILES: CC(=O)c1ccc(-c2nc(C3CC3)oc2Sc2ccc(Cl)cn2)cn1]

### 1-(5-{5-[(5-Chloropyridin-2-yl)sulfanyl]-2-cyclopropyl-1,3-oxazol-4-yl}pyridin-2-yl)ethanone

To a solution of Intermediate 87 (2.54 g, 6.55 mmol) in THF (260 mL) at rt was added MeMgBr (3.0M/Et₂O, 21.8 mL, 65.5 mmol), the resulting mixture was stirred at rt for 2 h. Upon completion of the reaction as judged by TLC analysis, the reaction was quenched by addition of sat.NH₄Cl solution, extracted with EtOAc, the organic layer was washed with brine, dried over MgSO₄, filtered, concentrated and purified on silica gel to afford the title compound (188 mg), LC/MS: m/e 371.8 (M+H)⁺.

### Example 89

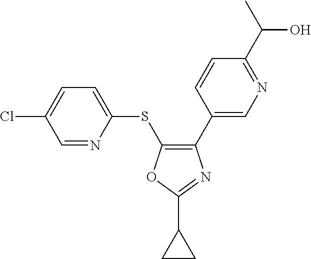 [SMILES: CC(O)c1ccc(-c2nc(C3CC3)oc2Sc2ccc(Cl)cn2)cn1]

### 1-(5-{5-[(5-Chloropyridin-2-yl)sulfanyl]-2-cyclopropyl-1,3-oxazol-4-yl}pyridin-2-yl)ethanol

To a solution of Example 88 (16 mg, 0.04 mmol) in MeOH (10 mL) at It was added NaBH₄ (1.6 mg, 0.04 mmol). The resulting solution was stirred at it for 1 h. Upon completion of the reaction as judged by TLC analysis, the reaction was concentrated to dryness and purified on silica gel to afford the titled compound (12 mg). LC/MS: m/e 373.9 (M+H)⁺.

### Example 90

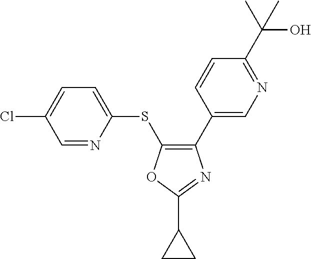 [SMILES: CC(C)(O)c1ccc(-c2nc(C3CC3)oc2Sc2ccc(Cl)cn2)cn1]

### 2-(5-{5-[(5-Chloropyridin-2-yl)sulfanyl]-2-cyclopropyl-1,3-oxazol-4-yl}pyridin-2-yl)propan-2-ol

To a solution of Example 87 (2.54 g, 6.5 mmol) in THF (260 mL) at rt was added MeMgBr (3.0M/Et₂O, 21.8 mL, 65.5 mmol). The resulting mixture was stirred at rt for 2 h. Upon completion of the reaction as judged by TLC analysis, the reaction was quenched by addition of sat.NH₄Cl solution and extracted with EtOAc. The organic layer was washed with brine, dried over MgSO₄, filtered, concentrated and purified on silica gel to afford the title compound (1.77 g). LC/MS: m/e 387.9 (M+H)⁺. ¹H NMR (500 MHz, CDCl₃): δ 1.22 (m, 4H), 1.56 (s, 6H), 2.19 (m, 1H), 4.85 (s, 1H), 6.96 (d, J=8.5 Hz, 1H), 7.42 (d, J=8 Hz, 1H), 7.55 (dd, J=2.5, 8.5 Hz, 1H), 8.32 (dd, J=2, 8.5 Hz, 1H), 8.43 (d, J=2.5 Hz, 1H), 9.14 (d, J=1.5 Hz, 1H).

The compounds in Table 8 were prepared from the appropriate starting materials using the procedure for Example 12.

TABLE 8

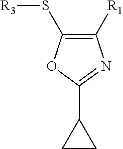

| | | | LCMS: |
|---|---|---|---|
| | | | found m/e |
| Example | R₁ | R₃ | (M + H) |
| 91 | 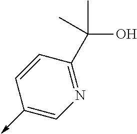 | 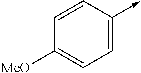 | 383.1 |
| 92 | 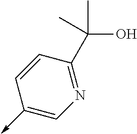 | 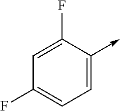 | 388.3 |
| 93 | 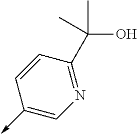 | 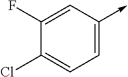 | 405.1 |
| 94 | 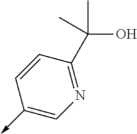 | 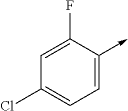 | 405.1 |
| 95 | 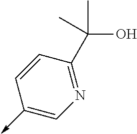 | 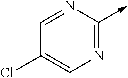 | 389.0 |

### Example 96

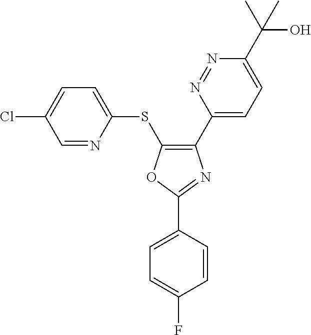 [SMILES: CC(C)(O)c1ccc(-c2nc(-c3ccc(F)cc3)oc2Sc2ccc(Cl)cn2)nn1]

### 2-(6-{5-[(5-Chloropyridin-2-yl)sulfanyl]-2-(4-fluorophenyl)-1,3-oxazol-4-yl}pyridazin-3-yl)propan-2-ol

The target compound was prepared in an analogous manner to Example 12 except that Intermediate 12 was replaced with Intermediate 47, LC/MS: m/e 443.2 (M+H)⁺. NMR (500 MHz, Acetone-d6): δ 1.64 (s, 6H), 4.70 (s, 1H), 7.38 (t, J=8.5 Hz, 2H), 7.43 (d, J=8.5 Hz, 1H), 7.76 (dd, J=2.5, 8.5 Hz, 1H), 8.09 (d, J=9 Hz, 1H), 8.22 (m, 2H), 8.27 (d, J=8.5 Hz, 1H), 8.43 (d, J=2.5 Hz, 1H).

### Example 97

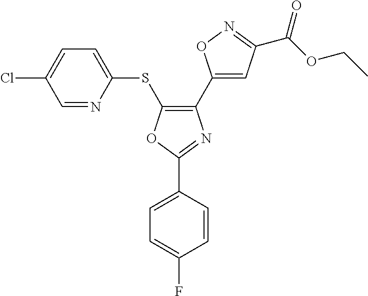 [SMILES: CCOC(=O)c1cc(-c2nc(-c3ccc(F)cc3)oc2Sc2ccc(Cl)cn2)on1]

### Ethyl 5-{5-[(5-chloropyridin-2-yl)sulfanyl]-2-(4-fluorophenyl)-1,3-oxazol-4-yl}isoxazole-3-carboxylate

The target compound was prepared in an analogous manner to Example 78 except that Intermediate 29 was replaced with Intermediate 52. LC/MS: m/e 445.9 (M+H)⁺,

### Example 98

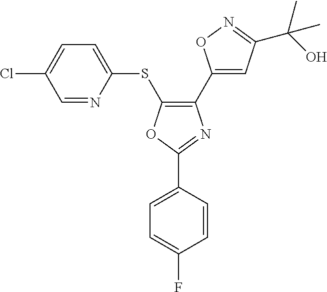 [SMILES: CC(C)(O)c1cc(-c2nc(-c3ccc(F)cc3)oc2Sc2ccc(Cl)cn2)on1]

### 2-(5-{5-[(5-Chloropyridin-2-yl)sulfanyl]-2-(4-fluorophenyl)-1,3-oxazol-4-yl}isoxazol-3-yl)propan-2-ol

The title compound was prepared in an analogous manner to Example 80 starting with Example 97, LC/MS: m/e 431.9 (M+H)⁺

### Example 99

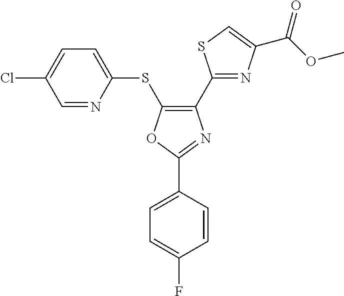 [SMILES: COC(=O)c1csc(-c2nc(-c3ccc(F)cc3)oc2Sc2ccc(Cl)cn2)n1]

### Methyl 2-{5-[(5-chloropyridin-2-yl)sulfanyl]-2-(4-fluorophenyl)-1,3-oxazol-4-yl}-1,3-thiazole-4-carboxylate

The target compound was prepared in an analogous manner to Example 78 except that Intermediate 29 was replaced with Intermediate 54, LC/MS: m/e 447.9 (M+H)⁺

### Example 100

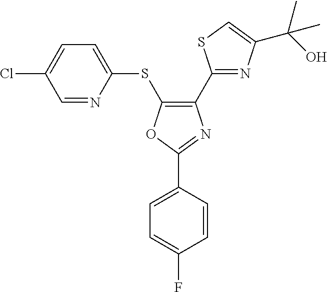 [SMILES: CC(C)(O)c1csc(-c2nc(-c3ccc(F)cc3)oc2Sc2ccc(Cl)cn2)n1]

### 2-(2-{5-[(5-Chloropyridin-2-yl)sulfanyl]-2-(4-fluorophenyl)-1,3-oxazol-4-yl}-1,3-thiazol-4-yl)propan-2-ol

The title compound was prepared in an analogous manner to Example 80 starting with Example 99, LC/MS: m/e 4479 (M+H)⁺.

### Example 101

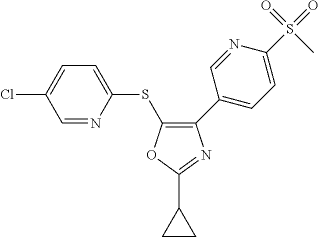 [SMILES: CS(=O)(=O)c1ccc(-c2nc(C3CC3)oc2Sc2ccc(Cl)cn2)cn1]

### 5-Chloro-2-({2-cyclopropyl-4-[6-(methylsulfonyl)pyridin-3-yl]-1,3-oxazo 1-5-yl}sulfanyl)pyridine

The target compound was prepared in an analogous manner to Example 78 except that Intermediate 29 was replaced with Intermediate 66, LC/MS: m/e 407.8 (M+H)⁺. ¹H NMR (500 MHz, CDCl₃): δ 1.22 (m, 4H), 2.20 (m, 1H), 3.25 (s, 3H), 7.04 (d, J=8.5 Hz, 1H), 7.59 (dd, J=2.5, 8.5 Hz, 1H), 8.12 (d, J=8 Hz, 1H), 8.46 (d, J=2.Hz, 1H), 8.59 (dd, J=2, 8.5 Hz, 1H), 9.38 (s, 1H).

### Example 102

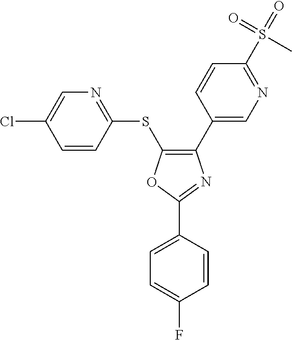 [SMILES: CS(=O)(=O)c1ccc(-c2nc(-c3ccc(F)cc3)oc2Sc2ccc(Cl)cn2)cn1]

### 5-Chloro-2-({2-(4-fluorophenyl)-4-[6-(methylsulfonyl)pyridin-3-yl]-1,3-oxazol-5-yl}sulfanyl)pyridine

The target compound was prepared in an analogous manner to Example 78 except that Intermediate 29 was replaced with Intermediate 56. LC/MS: m/e 461.8 (M+H)⁺ NMR (500 MHz, CDCl₃): δ 3.28 (s, 3H), 7.14 (d, J=2.5 Hz, 1H), 7.24 (t, J=8.5 Hz, 2H), 7.60 (dd, J=2.5, 8.5 Hz, 1H), 8.18 (m, 3H), 8.40 (d, J=2.5 Hz, 1H), 8.72 (dd, J=2, 8 Hz, 1H), 9.49 (d, J=2 Hz, 1H).

The compounds in Table 9 were prepared from the appropriate starting materials using the procedure for Example 78.

TABLE 9

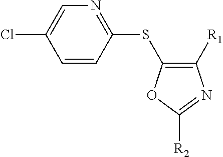

| | | | LCMS: |
|---|---|---|---|
| | | | found m/e |
| Example | R₁ | R₂ | (M + H) |
| 103 | 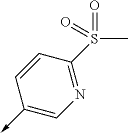 | 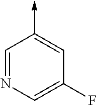 | 462.7 |
| 104 | 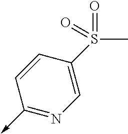 | 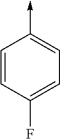 | 461.7 |
| 105 | 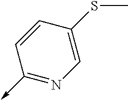 | 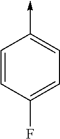 | 429.8 |
| 106 A and 106B | 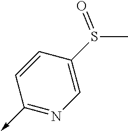 | 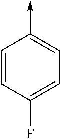 | 445.8 |

Note: Example 106 is racemic

### Example 107

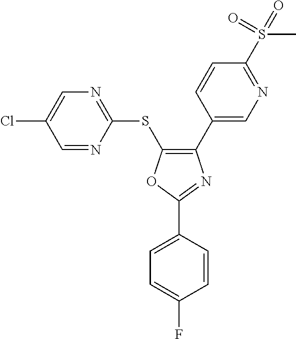 [SMILES: CS(=O)(=O)c1ccc(-c2nc(-c3ccc(F)cc3)oc2Sc2ncc(Cl)cn2)cn1]

### 5-Chloro-2-({2-(4-fluorophenyl)-4-[6-(methylsulfonyl)pyridin-3-yl]-1,3-oxazol-5-yl}sulfanyl)pyrimidine

The target compound was prepared in an analogous manner to Example 78 except that Intermediate 56 was coupled with 5-chloropyrimidine-2-thiol. LC/MS: n′ t/e 461.8 (M+H)⁺ NMR (500 MHz, CDCl₃): δ 3.28 (s, 3H), 7.14 (d, J=2.5 Hz, 1H), 7.24 (t, J=8.5 Hz, 2H), 7.60 (dd, J=2.5, 8.5 Hz, 1H), 8.18 (m, 3H), 8.40 (d, J=2.5 Hz, 1H), 8.72 (dd, J=2, 8 Hz, 1H), 9.49 (d, J=2 Hz, 1H).

### Example 108

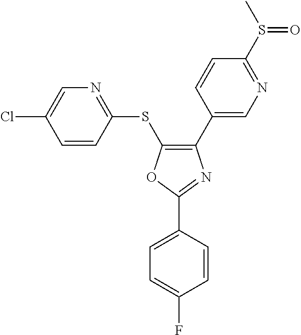 [SMILES: CS(=O)c1ccc(-c2nc(-c3ccc(F)cc3)oc2Sc2ccc(Cl)cn2)cn1]

### (R)-5-Chloro-2-({2-(4-fluorophenyl)-4-[6-(methylsulfinyl)pyridin-3-yl]-1,3-oxazol-5-yl}sulfanyl)pyridine and (S)-5-Chloro-2-({2-(4-fluorophenyl)-4-[6-methylsulfinyl)pyridin-3-yl]-1,3-oxazol-5-yl}sulfanyl)pyridine

The target compound was prepared in an analogous manner to Example 78 except that Intermediate 29 was replaced with Intermediate 60, LC/MS: m/e 445.8 (M+H)⁺

### Example 109

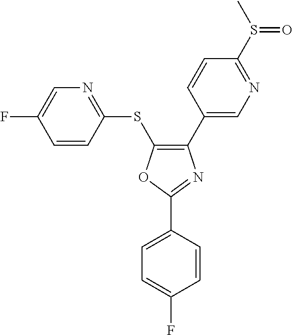 [SMILES: CS(=O)c1ccc(-c2nc(-c3ccc(F)cc3)oc2Sc2ccc(F)cn2)cn1]

### (R)-5-Fluoro-2-({2-(4-fluorophenyl)-4-[6-(methylsulfinyl)pyridin-3-yl]-1,3-oxazol-5-yl}sulfanyl)pyridine and (S)-5-Fluoro-2-({2-(4-fluorophenyl)-4-[6-methylsulfinyl)pyridin-3-yl]-1,3-oxazol-5-yl}sulfanyl)pyridine

The target compound was prepared in an analogous manner to Example 108 starting with Intermediate 60 and replacing 5-chloropyridine-2-thiol with 5-fluoropyridine-2-thiol. LC/MS: m/e 445.8 (M+H)+

### Example 110

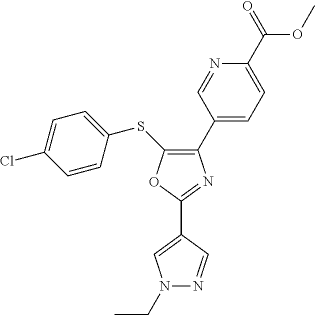 [SMILES: CCn1cc(-c2nc(-c3ccc(C(=O)OC)nc3)c(Sc3ccc(Cl)cc3)o2)cn1]

### Methyl 5-(5-[(4-chlorophenyl)sulfanyl]-2-(1-ethyl-1H-pyrazol-4-yl)-1,3-oxazol-4-yl)pyridine-2-carboxylate

The title compound was prepared in an analogous manner to Example 12 starting with Intermediate 61, LC/MS: m/e 441.2 (M+H)⁺.

### Example 111

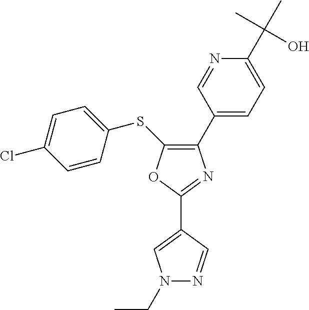 [SMILES: CCn1cc(-c2nc(-c3ccc(C(C)(C)O)nc3)c(Sc3ccc(Cl)cc3)o2)cn1]

### 2-(5-{5-[4-chlorophenyl)sulfanyl]-2-(1-ethyl-1H-pyrazol-4-yl)-1,3-oxazol-4-yl)pyridin-2-yl}propan-2-ol

The title compound was prepared in an analogous manner to Example 80 starting with Example 110, LC/MS: m/e 441.3 (M+H)⁺.

### Example 112

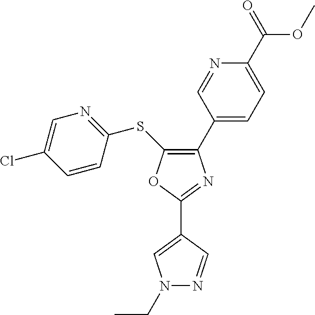 [SMILES: CCn1cc(-c2nc(-c3ccc(C(=O)OC)nc3)c(Sc3ccc(Cl)cn3)o2)cn1]

### Methyl 5-{5-[(5-chloropyridin-2-yl)sulfanyl]-2-(1-ethyl-1H-pyrazol-4-yl)-1,3-oxazo-4-yl)pyridine-2-carboxylate

The title compound was prepared in an analogous manner to Example 12 starting with Intermediate 64 and 4-Chlorobenzenethiol was replaced with 5-chloropyridine-2-thiol, LC/MS: m/e 441.9 (M+H)⁺.

### Example 113

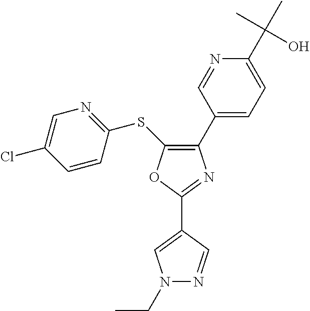 [SMILES: CCn1cc(-c2nc(-c3ccc(C(C)(C)O)nc3)c(Sc3ccc(Cl)cn3)o2)cn1]

### 2-(5-{5-[(5-chloropyridin-2-yl)sulfanyl]-2-(1-ethyl-1H-pyrazol-4-yl)-1,3-oxazol-4-yl}pyridin-2-yl}propan-2-ol

The title compound was prepared in an analogous manner to Example 80 starting with Example 112, LC/MS: m/e 442.1 (M+H)⁺.

### Example 114

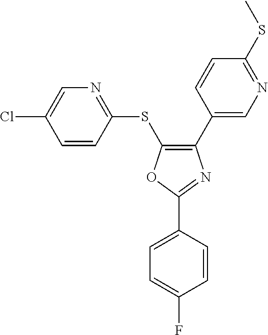 [SMILES: CSc1ccc(-c2nc(-c3ccc(F)cc3)oc2Sc2ccc(Cl)cn2)cn1]

### 5-Chloro-2-({2-(4-fluorophenyl)-4-[6-(methylsulfanyl)pyridin-3-yl]-1,3-oxazol-5-yl}sulfanyl)pyridine

The target compound was prepared in an analogues manner to Example 78 except that Intermediate 29 was replaced with Intermediate 58, LC/MS: m/e 429.8 (M+H)⁺. NMR (500 MHz, CDCl₃): δ 2.62 (s, 3H), 7.02 (d, J=8.5 Hz, 1H), 7.55 (t, J=8.5 Hz, 2H), 7.26 (d, J=8.5 Hz, 1H), 7.55 (dd, J=2.5, 8.5 Hz, 1H), 8.17 (m, 2H), 8.24 (dd, J=2.5, 8.5 Hz, 1H), 8.42 (d, J=2.5 Hz, 1H), 9.20 (s, 1H).

### Example 115

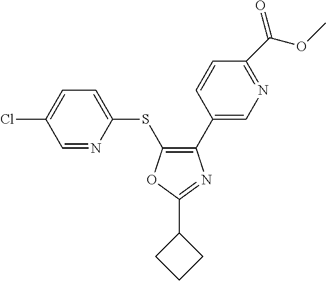 [SMILES: COC(=O)c1ccc(-c2nc(C3CCC3)oc2Sc2ccc(Cl)cn2)cn1]

### Methyl 5-{5-[(5-chloropyridin-2-yl)sulfanyl]-2-cyclobutyl-1,3-oxazol-4-yl}pyridine-2-carboxylate

The target compound was prepared in an analogous manner to Example 87 except that Intermediate 45 replaced Intermediate 69, LC/MS: m/e 401.9 (M+H)⁺

### Example 116A and Example 116B

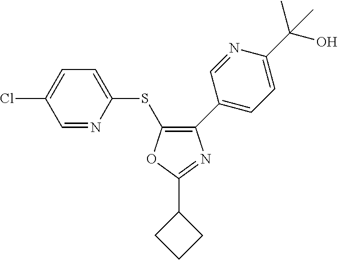 [SMILES: CC(C)(O)c1ccc(-c2nc(C3CCC3)oc2Sc2ccc(Cl)cn2)cn1]

### 2-(5-{5-[5-Chloropyridin-2-yl)sulfanyl]-2-cyclobutyl-1,3-oxazol-4-yl}pyridin-2-yl)propan-2-ol

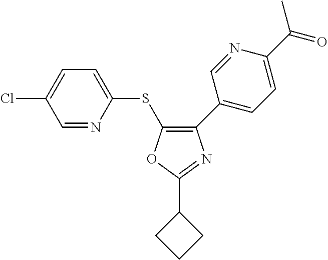 [SMILES: CC(=O)c1ccc(-c2nc(C3CCC3)oc2Sc2ccc(Cl)cn2)cn1]

### 1-(5-{5-[5-Chloropyridin-2-yl)sulfanyl]-2-cyclobutyl-1,3-oxazol-4-yl}pyridin-2-yl)ethanone

To a solution of Example 115 (264 mg, 0.6 mmol) in THF (20 ml) at rt was added MeMgBr(3.0M/Et₂O, 2.19 mL, 6.6 mmol) and the resulting mixture was stirred at rt for 2 h. Upon completion of the reaction as judged by TLC analysis, the reaction was quenched by addition of sat.NH₄Cl solution, extracted with EtOAc, the organic layer was washed with brine, dried over MgSO₄, filtered, concentrated and purified on silica gel to afford the title compound (201 mg) along with methyl ketone as a byproduct.

For 116A: LC/MS: m/e 401.9 (M+H)⁺. ¹H NMR (500 MHz, CDCl₃): δ 1.57 (s, 6H), 2.12 (m, 2H), 2.51 (m, 4H), 3.76 (m, 1H), 4.88 (s, 1H), 6.97 (d, J=8.5 Hz, 1H), 7.43 (d, J=8.5 Hz, 1H), 7.56 (dd, J=3.0, 8.5 Hz, 1H), 8.34 (dd, J=2.5, 8.5 Hz, 1H), 8.42 (d, J=2.5 Hz, 1H), 9.16 (d, J=1.5 Hz, 1H).

For 116B: m/e 385.9 (M+H)⁺. NMR (500 MHz, CDCl₃): δ 2.10 (m, 2H), 2.51 (m, 4H), 2.74 (s, 3H), 3.77 (m, 1H), 4.90 (s, 1H), 7.02 (d, J=8.5 Hz, 1H), 7.56 (d, J=7.0 Hz, 1H), 8.10 (d, J=8.5 Hz, 1H), 8.41 (s, 1H), 8.50 (d, J=8.0 Hz, 1H), 9.32 (s, 1H).

### Example 117

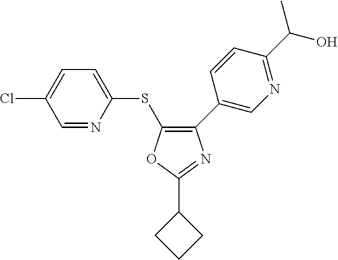 [SMILES: CC(O)c1ccc(-c2nc(C3CCC3)oc2Sc2ccc(Cl)cn2)cn1]

### (R)-1-(5-{5-[(5-Chloropyridin-2-yl)sulfanyl]-2-cyclobutyl-1,3-oxazol-4-yl}pyridin-2-yl)ethanol and (S)-1-(5-{5-[(5-Chloropyridin-2-yl)sulfanyl]-2-cyclobutyl-1,3-oxazol-4-yl}pyridin-2-yl)ethanol

The target compound was prepared in an analogous manner to Example 89 except that Example 88 was replaced with Example 116B, LC/MS: m/e 387.9 (M+H)⁺. ¹H NMR (500 MHz, CDCl₃): δ 1.52 (d, J=6.5 Hz, 3H), 2.08 (m, 2H), 2.50 (m, 4H), 3.75 (m, 1H), 4.13 (br, 1H), 4.93 (m, 1H), 6.95 (d, J=9 Hz, 1H), 7.33 (d, J=8 Hz, 1H), 7.54 (dd, J=2.5, 8.5 Hz, 1H), 8.33 (dd, J=2.0, 8.0 Hz, 1 Hz), 8.40 (d, J=2 Hz, 1H), 8.18 (d, J=1.5 Hz, 1H).

### Example 118

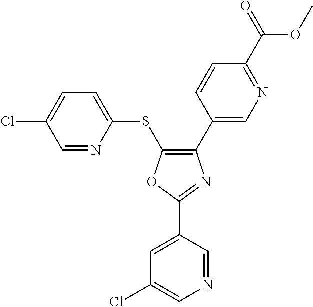 [SMILES: COC(=O)c1ccc(-c2nc(-c3cncc(Cl)c3)oc2Sc2ccc(Cl)cn2)cn1]

### Methyl 5-{2-(5-chloropyridin-3-yl)-5-[(5-chloropyridin-2-yl)sulfanyl]-1,3-oxazol-4-yl}pyridine-2-carboxylate

The target compound was prepared in an analogous manner to Example 78 except that Intermediate 29 was replaced with Intermediate 72, LC/MS: m/e 458.8 (M+H)⁺.

### Example 119

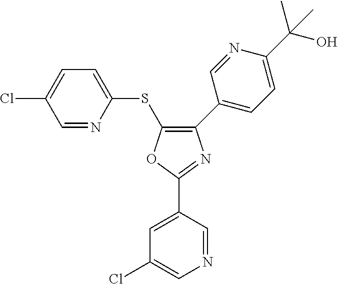 [SMILES: CC(C)(O)c1ccc(-c2nc(-c3cncc(Cl)c3)oc2Sc2ccc(Cl)cn2)cn1]

### 2-(5-{2-(5-Chloropyridin-3-yl)-5-[(5-chloropyridin-2-yl)sulfanyl]-1,3-oxazol-4-yl}pyridin-2-yl)propan-2-ol

The title compound was prepared in an analogous manner to Example 80 starting with Example 118. LC/MS: m/e 458.8 (M+H)⁺. ¹H NMR (500 MHz, CDCl₃): δ 1.60 (s, 6H), 4.81 (s, 1H), 7.13 (d, J=8.5 Hz, 1H), 7.49 (d, J=8.0 Hz, 1H), 7.60 (dd, J=2.5, 8.5 Hz, 1H), 8.44 (m, 3H), 8.74 (d, J=2.5 Hz, 1H), 9.27 (dd, J=2.0, 6.5 Hz, 2H).

### Example 120

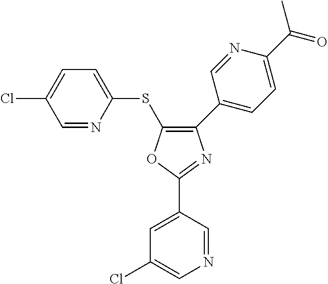 [SMILES: CC(=O)c1ccc(-c2nc(-c3cncc(Cl)c3)oc2Sc2ccc(Cl)cn2)cn1]

### 1-(5-{2-(5-Chloropyridin-3-yl)-5-[(5-chloropyridin-2-yl)sulfanyl]-1,3-oxazol-4-yl}pyridin-2-yl)ethanone

The target compound was prepared in an analogous manner to Example 116 B starting with Example 118. LC/MS: m/e 442.8 (M+H)⁺. ¹H NMR (500 MHz, CDCl₃): δ 2.77 (s, 3H), 7.17 (d, J=8.0 Hz, 1H), 7.61 (d, J=7.5 Hz, 1H), 8.14 (d, J=7.5 Hz, 1H), 8.39 (s, 1H), 8.44 (s, 1H), 8.58 (d, J=7.5 Hz, 1H), 8.75 (s, 1H), 9.28 (s, 1H), 9.45 (s, 1H).

### Example 121

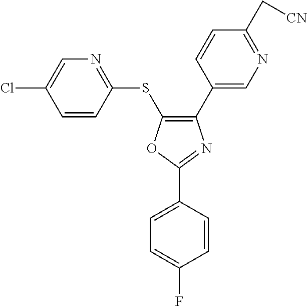 [SMILES: N#CCc1ccc(-c2nc(-c3ccc(F)cc3)oc2Sc2ccc(Cl)cn2)cn1]

### (5-{5-[(5-Chloropyridin-2-yl)sulfanyl]-2-(4-fluorophenyl)-1,3-oxazol-4-yl}pyridin-2-yl)acetonitrile

The target compound was prepared in an analogous manner to Example 78 except that Intermediate 29 was replaced with Intermediate 74. LC/MS: m/e 422.8 (M+H)⁺. ¹H NMR (500 MHz, CDCl₃): δ 4.00 (s, 3H), 7.08 (d, J=9.0 Hz, 1H), 7.23 (t, j=8.5 Hz, 2H), 7.53 (d, J=8.5 Hz, 1H), 7.57 (dd, J=2.5, 8.0 Hz, 1H), 8.19 (m, 2H), 8.41 (d, J=2.5 Hz, 1H), 8.49 (dd, J=2.0, 8.0 Hz, 1H), 9.33 (d, J=2.5 Hz 1H).

### Example 122

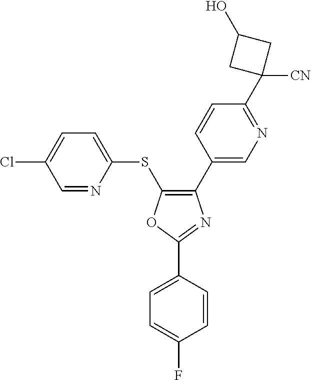 [SMILES: N#CC1(c2ccc(-c3nc(-c4ccc(F)cc4)oc3Sc3ccc(Cl)cn3)cn2)CC(O)C1]

### 1-(5-{5-[(5-Chloropyridin-2-yl)sulfanyl]-2-(4-fluorophenyl)-1,3-oxazol-4-yl}pyridin-2-yl)-3-hydroxycyclobutanecarbonitrile

The target compound was prepared in an analogous manner to Example 78 except that

Intermediate 29 was replaced with Intermediate 76, LC/MS: m/e 478.9 (M+H)⁺. ¹H NMR (500 MHz, Acetone-d6): δ 1.68 (m, 1H), 2.01 (m, 1H), 2.33 (m, 1H), 3.73 (m, 1H), 4.03 (m, 1H), 7.40 (m, 3H), 7.80 (m, 2H), 8.24 (m, 2H), 8.46 (d, J=2.5 Hz, 1H), 8.52 (dd, J=2.5, 8.5 Hz, 1H), 9.18 (s, 1H).

### Example 123

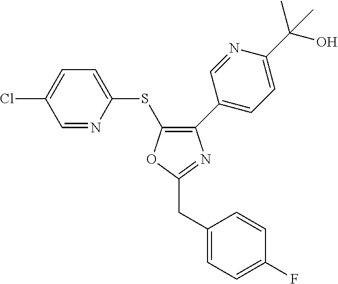 [SMILES: CC(C)(O)c1ccc(-c2nc(Cc3ccc(F)cc3)oc2Sc2ccc(Cl)cn2)cn1]

### 2-(5-{5-[(5-Chloropyridin-2-yl)sulfanyl]-2-(4-fluorobenzyl)-1,3-oxazol-4-yl}pyridin-2-yl)propan-2-ol

The target compound was prepared in an analogous manner to Example 78 except that Intermediate 29 was replaced with Intermediate 78, LC/MS: m/e 456.0 (M+H)⁺. ¹H NMR (500 MHz, Acetone-d6): δ 1.51 (m, 1H), 4.31 (s, 2H), 4.59 (s, 1H), 7.15 (t, J=8.5 Hz, 2H), 7.23 (d, J=8.5 Hz, 1H), 7.47 (m, 2H), 7.50 (d, J=8.5 Hz, 1H), 7.79 (dd, J=2.5, 8.5 Hz, 1H), 8.33 (dd, J=2.5, 8.5 Hz, 1H), 8.44 (d, J=2.5 Hz, 1H), 9.10 (d, J=2.0 Hz, 1H).

### Example 124

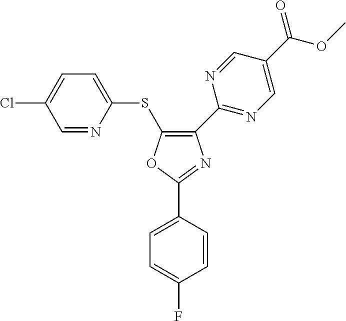 [SMILES: COC(=O)c1cnc(-c2nc(-c3ccc(F)cc3)oc2Sc2ccc(Cl)cn2)nc1]

### Methyl 2-{5-[(5-chloropyridin-2-yl)sulfanyl]-2-(4-fluorophenyl)-1,3-oxazol-4-yl}pyrimidine-5-carboxylate

The target compound was prepared in an analogous manner to Example 78 except that Intermediate 29 was replaced with Intermediate 80, LC/MS: m/e 442.9 (M+H)⁺. ¹H NMR (500 MHz, CDCl₃): δ 4.02 (s, 3H), 7.20 (t, J=8.5 Hz, 2H), 7.27 (m, 1H), 7.60 (dd, J=2.5, 8.5 Hz, 1H), 8.19 (m, 2H), 8.44 (d, J=2.5 Hz, 1H), 9.39 (s, 2H).

The Examples in Table 10 were prepared following the procedures described in Example 50, Step F.

TABLE 10

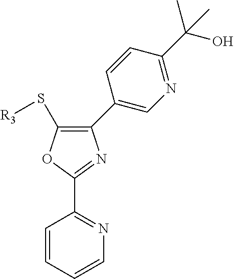

| | | LCMS: |
|---|---|---|
| | | found m/e |
| Example | R₃ | (M + H) |
| 125 | 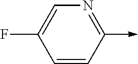 | 409.2 |
| 126 | 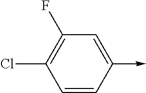 | 442.1 |
| 127 | 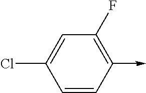 | 442.1 |
| 128 | 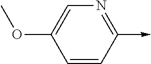 | 421.2 |
| 129 | 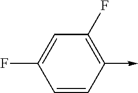 | 426.1 |
| 130 | 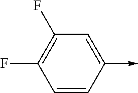 | 426.1 |
| 131 | 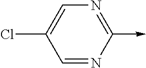 | 426.1 |
| 132 | 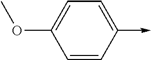 | 420.2 |

### Example 133

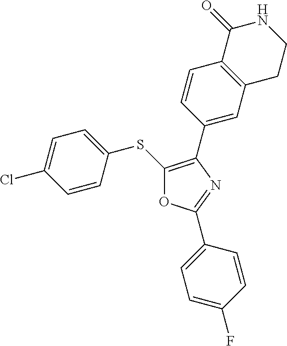 [SMILES: O=C1NCCc2cc(-c3nc(-c4ccc(F)cc4)oc3Sc3ccc(Cl)cc3)ccc21]

### 6-{5-[(4-Chlorophenyl)sulfanyl]-2-(4-fluorophenyl)-1,3-oxazol-4-yl}-3,4-dihydroisoquinolin-1(2H)-one

The title compound was prepared in an analogous manner to Example 12 starting with Intermediate 82, LC/MS: m/e 451.2 (M+H)⁺.

### Example 134

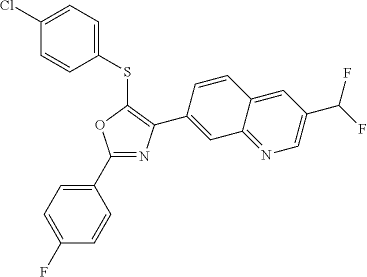 [SMILES: Fc1ccc(-c2nc(-c3ccc4cc(C(F)F)cnc4c3)c(Sc3ccc(Cl)cc3)o2)cc1]

### 7-{5-[(4-Chlorophenyl)sulfanyl]-2-(4-fluorophenyl)-1,3-oxazol-4-yl}-3-(difluoromethyl)quinoline

Intermediate 86 was dissolved 4-chlorothiophenol (23 mg, 0.157 mmol) in NMP (1 mL) and added a 60% oil dispersion of NaH (6.3 mg, 0.157 mmol). Vigorous gas evolution and reaction mixture became dark purple in color. Stirred at rt for 20 min. then combined a solution of intermediate (36 mg, 0.071 mmol) in NMP (1 mL), the above prepared thiolate solution and CuI (13.6 mg, 0.071 mmol) in a sealed vial, degassed with N₂, sealed with a Teflon stopper and heated to 120° C. Heated for 7 h then cooled to rt and stirred overnight. Diluted with sat'd. NaHCO₃ (9 mL), and cone NH₃ (1 mL) and extracted with EtOAc (3×). Washed extracts with brine (1×), dried over MgSO₄, filtered, evaporated, and dried under high vac. at rt. The amber oil was purified by prep TLC (SiO₂, 20×20 cm, 1000 microns, 3 plates; hexane-EtOAc, 3:1) to afford the title compound (26 mg), LC/MS: m/e 482.9 (M+H)⁺. ¹H NMR (500 MHz, CDCl₃) δ 6.94 (t, J=55.85 Hz, 1H), 7.24 (t, J=8.55 Hz, 2H), 7.3 (m, 4H), 8.01 (d, J=8.5 Hz, 1H), 8.22 (m, 2H), 8.36 (s, 1H), 8.52 (d, J=8.5 Hz, 1H), 9.06 (s, 1H), 9.1 (s, 1H).

### Example 135

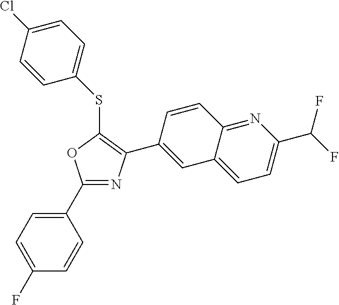 [SMILES: Fc1ccc(-c2nc(-c3ccc4nc(C(F)F)ccc4c3)c(Sc3ccc(Cl)cc3)o2)cc1]

### 6-{5-[(4-Chlorophenyl)sulfanyl]-2-(4-fluorophenyl)-1,3-oxazol-4-yl}-2-(difluoromethyl)quinoline

The title compound was prepared in an analogous manner to Example 12 starting with Intermediate 90, LC/MS: m/e 483.1 (M+H)⁺. ¹H NMR (500 MHz, CDCl₃) δ 6.84 (t, J=55.35, 1H), 7.25 (t, J=8.6 Hz, 2H), 7.3 (m, 4H), 7.8 (d, J=8.5 Hz, 1H), 8.22 (m, 3H), 8.42 (d, J=8.7 Hz, 1H), 8.69 (dd, J=1.8, 8.9 Hz, 1H), 8.72 (s, 1H).

### Example 136

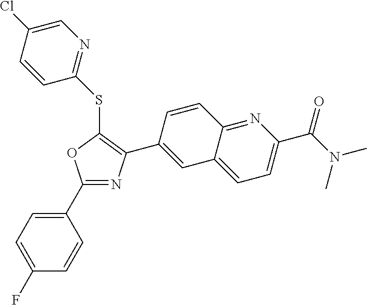 [SMILES: CN(C)C(=O)c1ccc2cc(-c3nc(-c4ccc(F)cc4)oc3Sc3ccc(Cl)cn3)ccc2n1]

### 6-{5-[(5-Chloropyridin-2-yl)sulfanyl]-2-(4-fluorophenyl)-1,3-oxazo-4-yl}-N,N-dimethylquinoline-2-carboxamide

The target compound was prepared in an analogous manner to Example 12 starting with Intermediate 93. LC/MS: m/e 505.1 (M+H)⁺. ¹H NMR (500 MHz, CDCl₃) δ 3.21 (s, 3H), 3.24 (s, 3H), 7.085 (d, J=8.7 Hz, 1H), 7.25 (t, J=8.2 Hz, 2H), 7.56 (d, J=9.1 Hz, 1H), 7.77 (d, J=8.3 Hz, 1H), 8.19 (d, J=8.4 Hz, 1H), 8.24 (m, 2H), 8.35 (d, 8.8 Hz, 1H), 8.45 (s, 1H), 8.6 (d, J=9.2 Hz, 1H), 8.69 (s, 1H).

### Example 137

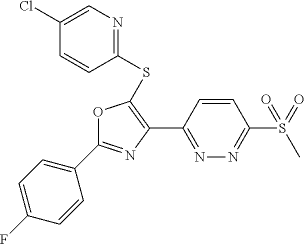 [SMILES: CS(=O)(=O)c1ccc(-c2nc(-c3ccc(F)cc3)oc2Sc2ccc(Cl)cn2)nn1]

### 3-{5-[(5-Chloropyridin-2-yl)sulfanyl]-2-(4-fluorophenyl)-1,3-oxazol-4-yl}-6-(methylsulfonyl)pyridazine

The target compound was prepared in an analogous manner to the Example 12 starting with Intermediate 97, LC/MS: m/e 462.8 (M+H)⁺. ¹H NMR (500 MHz, CDCl₃) δ 3.50 (s, 3H), 7.23 (m, 2H), 7.33 (d, J=8.7 Hz, 1H), 7.63 (dd, J=2.1, 8.5 Hz, 1H), 8.15. (m, 2H), 8.3 (d, J=8.8 Hz, 1H), 8.42 (s, 1H), 8.58 (t, J=8.7 Hz, 1H).

### Example 138

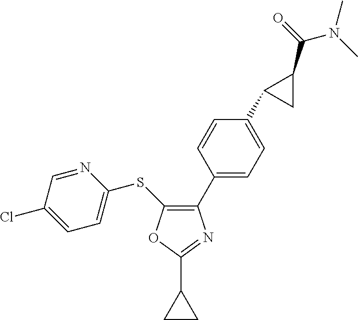 [SMILES: CN(C)C(=O)[C@H]1C[C@@H]1c1ccc(-c2nc(C3CC3)oc2Sc2ccc(Cl)cn2)cc1]

### (1S,2S)-2-(4-{5-[(5-chloropyridin-2-yl)thio]-2cyclopropyl-1,3-oxazol-4-yl}phenyl)-N,N-dimethylcyclopropanecarboxamide

Step A.

A solution of Intermediate 24 (478 mg, 1.858 mmol), PdCl₂ (dppf)-CH₂Cl₂ Adduct (68 mg, 0.093 mmol), dppf (51 mg, 0.093 mmol), KOAc (oven dried) (547 mg, 5.57 mmol), bis(pinacolato)diboron (613 mg, 2.415 mmol) in dioxane (4.3 mL) was placed under an atmosphere of nitrogen and heated at 150° C. for 20 min via microwave irradiation. To this mixture was added Intermediate 98 (500 mg, 1.858 mmol), bis(triphenylphosphine)palladium (II) chloride (130 mg, 0.186 mmol), sodium carbonate (1 mL of 1 M aqueous solution). The mixture was heated at 150° C. for 45 min via microwave irradiation. Water was added and the mixture was extracted with ethyl acetate. The organics were dried (MgSO₄) and concentrated. The residue was subject to silica column (0-30% EtOAc in hexanes) to afford ethyl (1S,2S)-2-[4-(2-cyclopropyl-1,3-oxazol-4-yl)phenyl]cyclopropanecarboxylate (239 mg, 43%). LC/MS: m/z 298.1 (M+H)+.

Step B.

A solution of the product from the previous step (400 mg, 1.345 mmol) and NBS (311 mg, 1.749 mmol) in CH₂Cl₂ (4.5 mL) was stirred at rt for 3 h. Upon completion of the reaction, the solution was diluted with sat aq NaS₂O₃ solution. The organic layer was removed, dried over MgSO₄, filtered and concentrated giving rise to an oil. The oil was purified on silica gel to afford the ethyl (1S,2S)-2-[4-(5-bromo-2-cyclopropyl-1,3-oxazol-4-yl)phenyl]cyclopropanecarboxylate (335 mg, 66%), LC/MS: m/z 376.2 (M+H)⁺.

Step C.

A solution of 5-chloropyridine-2-thiol (201 mg, 1.382 mmol) dissolved in 2 mL of NMP was treated with NaH (55 mg, 1.382 mmol). The resulting solution was stirred for 30 min at rt before the product from the previous step (260 mg, 0.691 mmol) and CuI (132 mg, 0.691 mmol) were added. The resulting dark solution was heated to 120° C. for 16 h. After which point, the solution was poured into a rapidly stirred solution of 9:1 NH₄Cl:NH₄OH and EtOAc. Upon clarification of the organic layer, removal of the organic layer was followed by drying over MgSO₄, filtration and concentration giving rise to an oil. The oil was purified on silica gel to afford ethyl (1S,2S)-2-(4-{5-[(5-chloropyridin-2-yl)thio]-2-cyclopropyl-1,3-oxazol-4-yl}phenyl)cyclopropanecarboxylate. LC/MS: m/z 441.1 (M+H)⁺.

Step D.

The product from the previous step (140 mg, 0.318 mmol) was dissolved in 1 mL of acetonitrile, to which was added 1 mL of water, followed by excess KOH pellets. The reaction was stirred at 80° C. for 3 h. After it was cooled to rt, the pH of the reaction mixture was adjusted to 6 with concentrated HCl. EtOAc was added, and the mixture was washed with water and brine, dried, and concentrated to dryness to afford (1S,2S)-2-(4-{5-[(5-chloropyridin-2-yl)thio]-2-cyclopropyl-1,3-oxazol-4-yl}phenyl)cyclopropanecarboxylic acid which was used in the next step with out further purification. LC/MS: m/z 413.1 (M+H)+.

Step E.

The product from the previous step (30 mg, 0.073 mmol), HOBT (28 mg, 0.182 mmol), and EDC (35 mg, 0.182 mmol) were dissolved in 1 mL of DMF, to which were added Hunig's base (0.075 mL, 0.436 mmol) and dimethyl amine (2 M THF solution, 0.363 mL, 0.727 mmol). The reaction was heated at 75° C. for 45 min. Upon cooling to rt, the reaction was diluted with EtOAc and the reaction mixture was washed with water and brine, dried, and concentrated to dryness. The title compound was crystallized by dissolving in hot methanol then slowly cooling to −20° C. LC/MS: m/z 440.1 (M+H)+, ¹H NMR (500 MHz, CD₃OD): δ 8.39 (s, 1H), 7.84 (d, 2H), 7.71 (d, 1H), 7.20 (d, 2H), 7.06 (d, 1H), 3.16 (s, 3H), 2.97 (s, 3H), 4.22 (m, 1H), 2.4-2.2 (br, 2H), 1.6-1.1 (br, 2H).

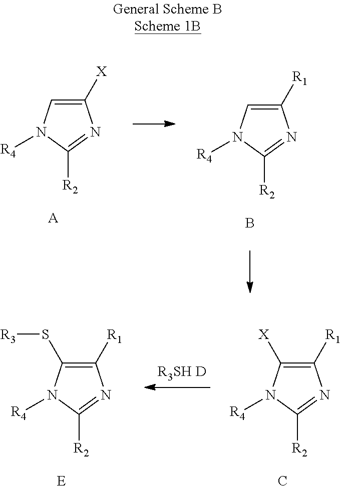

In Scheme 1 B, an appropriately substituted, commercially available imidazole A where X=Br or I is reacted with a coupling partner containing R_{I} under palladium mediated cross coupling conditions to provide B. B was converted to C through standard halogenation reactions using NIS or NCS. Finally, sulfide formation between C and thiol D catalyzed by copper or palladium afforded the final product E.

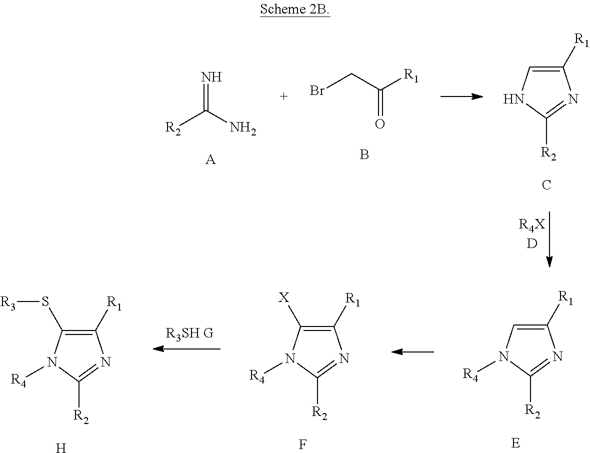

Scheme 2B illustrates the synthesis of examples where the appropriately substituted imidazole is not commercially available. In this case, amidine A and α-bromoketone B are refluxed in THF/water in the presence of NaHCO₃ to afford imidazole C, which is alkylated with R₄X to give E. Once the substituted imidazole E is reached, the remaining steps are the same as those described in Scheme 1.

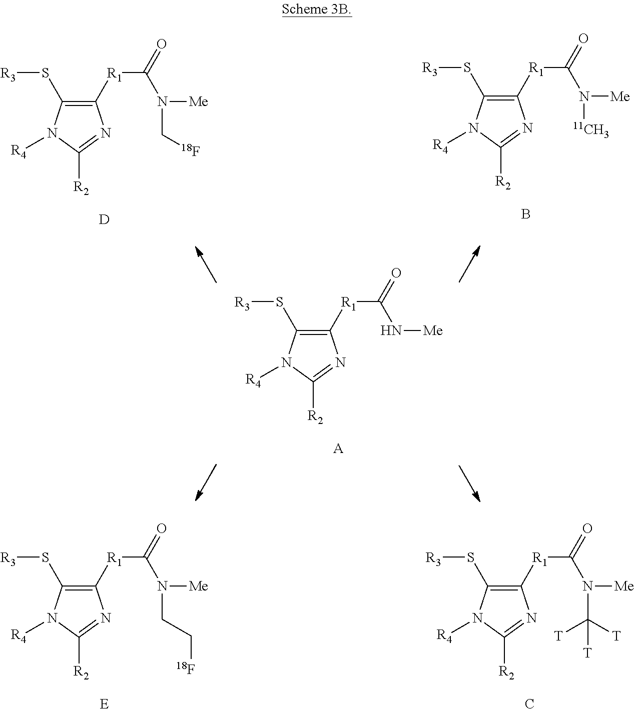

In Scheme 3B, the secondary amide in A is alkylated in the presence of base such as NaH with an appropriate radionuclide-containing reagent, such as [¹¹C]-methyl iodide, [³H]-methyl iodide, [¹⁸F]-fluoromethylbromide, or [¹⁸F]-fluoroethylbromide, to afford tertiary amide B, C, D or E, respectively.

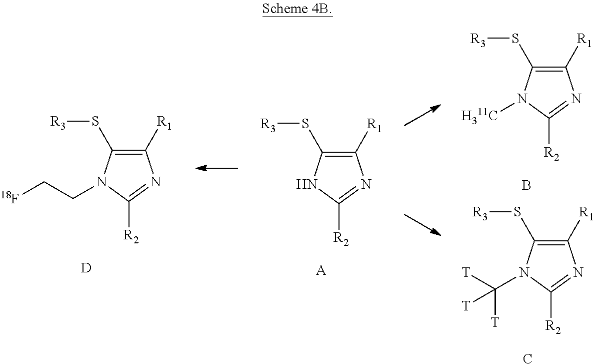

Similarly in Scheme 4B, the imidazole A is alkylated in the presence of base such Cs₂CO₃ or K₂CO₃ with an appropriate radionuclide-containing reagent, such as [¹¹C]-methyl iodide, [³H]-methyl iodide, or [¹⁸F]-fluoroethylbromide, to afford N-substituted imidazoles B, C, or D, respectively.

### Intermediate 1B

### 5-Chloropyridine-2-thiol

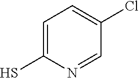

2,5-Dichloropyridine (5.0 g) and thiourea (2.57 g) were suspended in 50.0 mL of EtOH and the mixture was heated at 95° C. After 22 h, the reaction solution was cooled, was slowly added a solution of 2.84 g of KOH in 5.0 mL of water. The solution was heated at 95° C. for 2 h, cooled, poured into 100 mL of 0.5 N NaOH, made acidic with acetic acid. The product was extracted with dichloromethane, washed with water, dried over MgSO₄, and filtered. The organic layer was concentrated to give 2.3 g of the title compound. 1H NMR (500 MHz, (CD₃OD): 7.78 (s, 1H), 7.44 (d, 1H), 7.39 (d, 1H), 4.39 (s, 1H). LCMS: m/z 146.0 (M+H)⁺.

### Intermediate 2B

### Ethyl (1S,2S)-2-(4-bromophenyl)cyclopropanecarboxylate

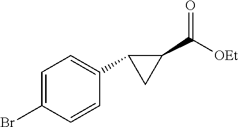 [SMILES: CCOC(=O)[C@H]1C[C@@H]1c1ccc(Br)cc1]

To a 1-neck, 1-L round bottom flask equipped with a magnetic stirrer was added 265 mL methyl tert-butyl ether. The flask was evacuated and flushed with nitrogen three times. 2,2′-Isopropylidenebis[(4R)-4-tert-butyl-2-oxazolidine] (2.39 g, 8.03 mmol) was added, followed by copper(I) trifluoromethanesulfonate benzene complex (4.49 g, 8.03 mmol). The green suspension was stirred at room temperature for about 2 hours and was then filtered. The filtrate was added to a 4-neck, 5-L, round bottom flask equipped with a mechanical stirrer, thermocouple, nitrogen bubbler, and addition funnel. Then, 4-bromostyrene (150 g, 0.803 mol) was added to this solution and the reaction was cooled to 0° C. via an ice/water bath. Ethyl diazoacetate (167 mL, 1.606 mol) was dissolved in 1675 mL of MTBE and the solution was evacuated/flushed with nitrogen three times. This solution was then added to an addition funnel and added dropwise to the reaction mixture. A slight exotherm was observed. The ethyl diazoacetate was allowed to add slowly over the weekend and the reaction slowly warmed to room temperature. The reaction was poured into a large extractor and diluted with 4L MTBE. The organics were washed with 2×1 L 3% aq. ammonium hydroxide and 2L brine, dried over anhydrous magnesium sulfate, filtered, and concentrated. The residue was dissolved in heptane and a small amount of dichloromethane, injected onto an ISCO 1500 g column prepacked in heptane. The column was eluted with 100% heptane over 1 column volume, 0-20% ethyl acetate/heptane over 6.5 column volumes, and held at 20% ethyl acetate/heptane over 8 column volumes. The product containing fractions were collected and concentrated to give 191 g (yield 88%) of the title compound. 1H NMR (500 MHz, (CDCl₃): 7.42 (d, 2H), 7.01 (d, 2H), 4.21 (q, 2H), 2.49 (m, 1H), 1.88 (m, 1H), 1.62 (m, 2H), 1.25 (t, 3H).

### Intermediate 3B

### Ethyl (1S,2S)-2-[4-(1H-imidazol-4-yl)phenyl]cyclopropanecarboxylate

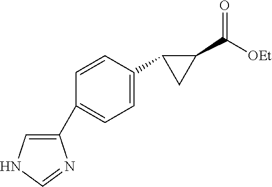 [SMILES: CCOC(=O)[C@H]1C[C@@H]1c1ccc(-c2c[nH]cn2)cc1]

Step 1:

3 M EtMgBr in diethyl ether (4.58 mL, 13.75 mmol) was added slowly to a solution of 4-iodo-1-trityl-1H-imidazole (5 g, 11.46 mmol) 100 mL of THF and stirred at rt. After 30 min, ZnCl₂ (3.12 g, 23 mmol) was added and the mixture was stirred at rt for 1 h. The Intermediate 2 (3.08 g, 11.46 mmol) was added, followed by Pd(PPh₃)₄ (662 mg, 0.573 mmol), and the reaction mixture was heated at reflux for 4 hours. At this point, the LCMS indicated 100% conversion to product (rt=1.19 min). The reaction was cooled to rt, quenched with aqueous NH₄Cl (30 ml). The inorganic salts crashed out, which was removed by filtration. The aqueous layer was separated, and the organic was washed with water (30 mL) and brine (30 mL). The organic layer was dried (MgSO₄), filtered, and evaporated in vacuo. The residue was purified by flash chromatography (10-80% EA in hexanes) to give 4.1 g (yield 71.8%) of ethyl (1S,2S) -2-[4-(1-trityl-1H-imidazol-4-yl)phenyl]cyclopropanecarboxylate. LCMS: m/z 499 (M+H)+.

Step 2:

Ethyl (1S,2S)-2-[4-(1-trityl-1H-imidazol-4-yl)phenyl]cyclopropanecarboxylate (4.1 g, from Step 1) was suspended in 30 mL of methanol and 30 mL of 1N HCl. The reaction mixture was heated at reflux for 2 hours. The solvent was evaporated in vacuo and the residue was triturated with ether (100 mL). The liquid organic layer was discarded. The solid was the desired product HCl salt. To the solid were added 100 mL EtOAc and 13 mL of 1N NaOH to release the free base. The aqueous/organic mixture was shaken in a separation funnel. The aqueous layer was discarded, and the organic layer was washed with brine, dried with MgSO₄, filtered, and concentrated in vacuo to give the title compound (1.4 g, 66.4%). 1H NMR (500 MHz, (CD₃OD): 7.98 (s, 1H), 7.58 (d, 2H), 7.39 (s, 1H), 7.17 (d, 2H), 4.18 (q, 2H), 2.43 (m, 1H), 1.86 (m, 1H), 1.57 (m, 1H), 1.37 (m, 1H), 1.24 (t, 3H). LCMS: m/z 257 (M+H)⁺.

### Intermediate 4B

### 2-(4-{5-[(5-Chloropyridin-2-yl)thio]-1-methyl-1H-imidazol-4-yl}phenyl)cyclopropanecarboxylic acid

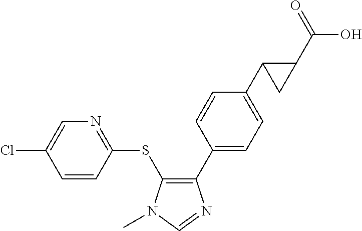 [SMILES: Cn1cnc(-c2ccc(C3CC3C(=O)O)cc2)c1Sc1ccc(Cl)cn1]

Step 1:

A solution of 2-bromo-1-(4-bromophenyl)ethanone (8 g, 28.8 mmol) in 30 mL of formamide was stirred at 140° C. for 24 hrs. The reaction was cooled to rt and diluted with EtOAc. The reaction mixture was washed with aqueous NaHCO₃, water (3 times), and brine, dried over MgSO₄, and concentrated to give 3.1 g of crude 4-(4-bromophenyl)-1H-imidazole that was used in the next step without further purification.

Step 2:

To a solution of Step 1 product (3.1 g, 13.90 mmol) in 50 mL of THF was added iodomethane (1.74 mL, 27.8 mmol) and cesium carbonate (5.43 g, 16.68 mmol). The reaction was stirred at rt overnight. EtOAc (150 mL) was added to the reaction, and the mixture was washed with water (2 times) and brine, dried over MgSO₄, and concentrated to dryness. The residue was purified by silica column (10-80% EtOAc in hexanes) to give 2.8 g (yield 85%) 4-(4-bromophenyl)-1-methyl-1H-imidazole. LCMS: [M+1]⁺=237.

Step 3:

To a solution of 4-(4-bromophenyl)-1-methyl-1H-imidazole (Step 2 product, 2.8 g, 9.45 mmol) in dichloromethane (30 mL) was added N-iodosuccinimide (1.913 g, 8.50 mmol) and six drops of trifluoroacetic acid. The reaction mixture was stirred at rt for 16 h. The mixture was neutralized with aqueous sodium bicarbonate and the organics were extracted with dichloromethane. The organics were then washed with aqueous sodium thiosulfate, followed by three washes with water then dried (MgSO₄). The solvent was concentrated to afford 4-(4-bromophenyl)-5-iodo-1-methyl-1H-imidazole, which was used with out further purification. LCMS: [M+1]⁺=363.

Step 4:

To a dry suspension of the product from the previous step (3.4 g, 9.45 mmol), potassium carbonate (2.61 g, 18.90 mmol), copper (1) iodide (0.18 g, 0.945 mmol), and Intermediate 1 (2.064 g, 14.17 mmol) in 31.5 mL isopropanol under an atmosphere of nitrogen was added ethylene glycol (1.054 mL, 18.90 mmol). The reaction mixture was stirred at 80° C. for 16 h. Water was added and the mixture was extracted with ethyl acetate. The organics were dried (MgSO₄), concentrated, and purified on 100 g of silica gel eluting a gradient of 20-100% ethyl acetate in hexanes to give rise to 2-{[4-(4-bromophenyl)-1-methyl-1 h-imidazol-5-yl]thio}-5-chloropyridine (1.9 g, 5.0 mmol), LCMS: [M+1]⁺=380.

Step 5:

A solution of Pd₂(dba)₃ (0.481 g, 0.525 mmol), tri-tert-butylphosphonium tetrafluoroborate (0.305 g, 1.051 mmol) in DMF (15 mL) was stirred at rt for 10 min. Then the product from the previous step (1 g, 2.63 mmol) was added and the resulting mixture was stirred at rt for another 10 min before adding N-cyclohexyl-N-methylcyclohexanamine (1.350 mL, 6.30 mmol), methyl acrylate (2.3 mL, 25.4 mmol), and DMF (50 mL). After stirring at rt for 15 min, the reaction was heated to 120° C. for 1 h. After cooling to rt water was added and the mixture was extracted with ethyl acetate. The organics were dried (MgSO₄), concentrated, and purified on 40 g of silica gel eluting a gradient of 50-100% ethyl acetate in hexanes to give rise to methyl 3-(4-{5-[(5-chloropyridin-2-yl)thio]-1-methyl-1H-imidazol-4-yl}phenyl)acrylate (0.9 g, 2.3 mmol), LCMS: [M+1]⁺=386.

Step 6:

A solution of sodium hydride (60% in mineral oil), (0.233 g, 5.83 mmol) and trimethylsulfoxonium iodide (1.540 g, 7.00 mmol) in DMSO (40 mL) was stirred at rt for 1 hr. the product from the previous step (0.9 g, 2.3 mmol) was added and the resulting mixture was stirred at rt for 30 min before heating to 50° C. for 30 min. Water was added and the mixture was extracted with ethyl acetate. The organics were dried (MgSO₄) and concentrated to afford methyl 2-(4-{5-[(5-chloropyridin-2-yl)thio]-1-methyl-1H-imidazol-4-yl}phenyl)cyclopropanecarboxylate (0.5 g, 1.250 mmol) LCMS: [M+1]⁺=400.

Step 7:

To a solution of the product from the previous step (0.5 g, 1.250 mmol) in ethanol (22 mL) and water (8 mL) was added excess potassium hydroxide. The resulting mixture was heated to reflux for 1 h, cooled, neutralized with aqueous ammonium chloride, and extracted several times with ethyl acetate affording the title compound as a crude residue which could be used in the next Step with out further purification. Alternatively, the residue can be purifed by reverse phase HPLC. he fractions containing the product were collected, diluted with ethyl acetate, and washed with aqueous sodium bicarbonate, water, and brine. The organic layer was dried (MgSO₄), filtered, and concentrated to afford the title compound. ¹H NMR (500 MHz), [(CD₃)₂CO]: 8.43 (s, 1H), 8.00 (s, 2H), 7.96 (d, 2H), 7.73 (d, 1H), 7.18 (d, 2H), 6.95 (d, 1H), 3.71 (s, 3H), 2.44 (m, 1H), 1.89 (m, 1H), 1.50 (m, 1H), 0.96 (m, 1H). LCMS: [M+1]⁺=385.

### Intermediate 5B

### Ethyl (1S,2S)-2-[4-(5-iodo-1-methyl-1H-imidazol-4-yl)phenyl]cyclopropanecarboxylate

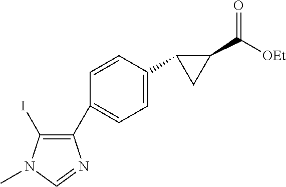 [SMILES: CCOC(=O)[C@H]1C[C@@H]1c1ccc(-c2ncn(C)c2I)cc1]

Step 1:

3 M EtMgBr in diethyl ether (6.27 mL, 18.81 mmol) was added slowly to a solution of 4-iodo-1-methyl-1H-imidazole (3.26 g, 15.67 mmol) 100 mL of THF and stirred at rt. After 30 min, ZnCl₂ (4.27 g, 31.3 mmol) was added and the mixture was stirred at rt for 1 h. The Intermediate 2 (4.22 g, 15.67 mmol) was added, followed by Pd(PPh₃)₄ (906 mg, 0.784 mmol), and the reaction mixture was heated at reflux for 4 hours. At this point, the LCMS indicated 100% conversion to product (rt=0.95 min). The reaction was cooled to rt, quenched with aqueous NH₄Cl (30 ml). The inorganic salts crashed out, which was removed by filtration. The aqueous layer was separated, and the organic was washed with water (30 mL) and brine (30 mL). The organic layer was dried (MgSO₄), filtered, and evaporated in vacuo. The residue was re-dissolved in DCM (200 mL) and the organic layer was washed with water (2×) and brine (to get rid of some Br-containing inorganic species). The DCM layer was dried (MgSO₄), filtered, and evaporated in vacuo. The residue was purified by flash chromatography (60-90% EtOAc in hexanes) to afford 2.9 g (yield 68%) of ethyl (1S,2S)-2-[4-(1-methyl-1H-imidazol-4-yl)phenyl]cyclopropanecarboxylate. LCMS: m/z 271 (M+H)+.

Step 2:

To a solution of Ethyl (1S,2S)-2-[4-(1-methyl-1H-imidazol-4-yl)phenyl]cyclopropanecarboxylate (product of Step 1, 2.8 g, 10.36 mmol) in dichloromethane (104 mL) was added N-iodosuccinimide (2.1 g, 9.33 mmol). The reaction mixture was stirred at rt for 16 h. The mixture was diluted with dichloromethane and washed with aqueous sodium thiosulfate, followed by three washes with water then dried (MgSO₄). The solvent was concentrated to afford the title compound as an orange oil which could be used in the next Step without further purification, LCMS: [M+1]⁺=396.

### Intermediate 6B

### (1S,2S)-2-(4-{5-[(5-Chloropyridin-2-yl)thio]-1-methyl-1H-imidazol-4-yl}phenyl)cyclopropanecarboxylic acid

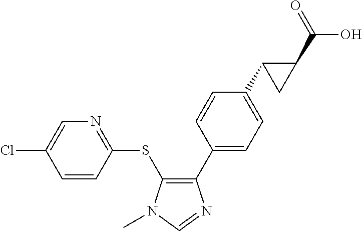 [SMILES: Cn1cnc(-c2ccc([C@H]3C[C@@H]3C(=O)O)cc2)c1Sc1ccc(Cl)cn1]

The title compound was prepared starting with Intermediate 5 and following the same procedure as described for Intermediate 4 (Steps 4 and 7). ¹H NMR (500 MHz), [(CD₃)₂CO]: 8.43 (s, 1H), 8.00 (s, 2H), 7.96 (d, 2H), 7.73 (d, 1H), 7.18 (d, 2H), 6.95 (d, 1H), 3.71 (s, 3H), 2.44 (m, 1H), 1.89 (m, 1H), 1.50 (m, 1H), 0.96 (m, 1H). LCMS: [M+1]⁺=386.

### Intermediate 7B

### (1S,2S)-2-(4-{5-[(4-Chlorophenyl)thio]-1-methyl-1H-imidazol-4-yl}phenyl)cyclopropanecarboxylic acid

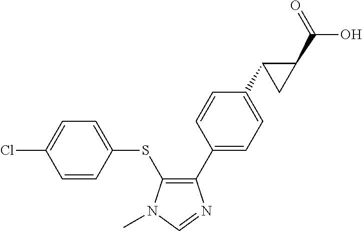 [SMILES: Cn1cnc(-c2ccc([C@H]3C[C@@H]3C(=O)O)cc2)c1Sc1ccc(Cl)cc1]

The title compound was prepared starting with 4-chlorothiophenol and Intermediate 5B following the same procedure as described for Intermediate 4 (Steps 4 and 7), LCMS: [M+1]⁺=385.

### Intermediate 8B

### (1R,2R)-2-[4-(5-Iodo-1,2-dimethyl-1H-imidazol-4-yl)-phenyl]cyclopropanecarboxylate

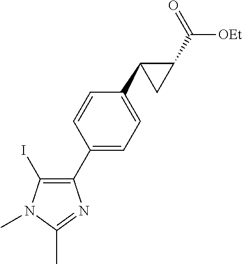 [SMILES: CCOC(=O)[C@@H]1C[C@H]1c1ccc(-c2nc(C)n(C)c2I)cc1]

Step 1:

A solution of 2 g (7.43 mmol) of ethyl (1R,2R)-2-(4-bromophenyl)cyclopropanecarboxylate (the enantiomer of Intermediate 2 that was made in the same way but using 2,2′-Isopropylidenebis[(4S)-4-tert-butyl-2-oxazolidine]), PdCl₂ (dppf)-CH₂Cl₂ Adduct (0.303 g, 0.372 mmol), dppf (0.206 g, 0.372 mmol), potassium acetate (oven dried) (2.188 g, 22.29 mmol), bis(pinacolato)diboron (2.453 g, 9.66 mmol) in dioxane (17 mL) was placed under an atmosphere of nitrogen and heated at 150° C. for 20 min via microwave irradiation. Water was added and the mixture was extracted with ethyl acetate. The organics were dried (MgSO₄), concentrated, and purified on 50 g of silica gel eluting a gradient of 0-20% ethyl acetate in hexanes to give rise to ethyl (1R,2R)-2-[4-(4,4,5,5-tetramethyl-1,3,2-dioxaborolan-2-yl)phenyl]cyclopropanecarboxylate (2.4 g, 7.59 mmol). ¹H NMR (500 MHz), [(CD₃)₂CO]: 7.67 (d, 2H), 7.20 (d, 2H), 4.15 (m, 1H), 2.06 (m, 1H), 1.33 (s, 12H), 1.24 (m, 2H).

Step 2:

To a solution of ethyl the product from the previous step (0.5 g, 1.581 mmol), 4-bromo-1,2-dimethyl-1H-imidazole (0.692 g, 3.95 mmol), and tetrakis (0.365 g, 0.316 mmol), was added sodium carbonate (3.2 mL of 2M aqueous solution). The mixture was heated at 150° C. for 45 min via microwave irradiation. Water was added and the mixture was extracted with ethyl acetate. The organics were dried (MgSO₄) and concentrated to afford ethyl (1R,2R)-2-[4-(1,2-dimethyl-1H-imidazol-4-yl)phenyl]cyclopropanecarboxylate which was used in the next Step without further purification. LCMS: [M+1]⁺=284.

Step 3:

To a solution of ethyl the product from the previous step (0.45 g, L583 mmol) in dichloromethane (5 mL) was added N-iodosuccinimide (0.427 g, 1.90 mmol) and three drops of trifluoroacetic acid. The reaction mixture was stirred at rt for 1 h. The mixture was neutralized with aqueous sodium bicarbonate and the organics were extracted with dichloromethane. The organics were then washed with aqueous sodium thiosulfate, followed by three washes with water then dried (MgSO₄). The solvent was concentrated to afford the title compound, which was used with out further purification LCMS: [M+1]⁺=410.

### Intermediate 9B

### 4-(4-Bromophenyl)-2-cyclopropyl-1-methyl-1H-imidazole

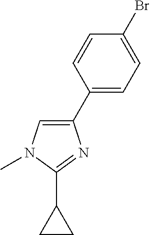 [SMILES: Cn1cc(-c2ccc(Br)cc2)nc1C1CC1]

Step 1:

To a 3-neck flask containing cyclopropylamidine HCl salt (5.99 g, 50 mmol), NaHCO₃ (10 g, 119 mmol), THF (40 mL), and water (10 mL) was added the solution of 2-bromo-1-(4-bromophenyl)ethanone (15.2 g, 55 mmol) in 30 mL of THF using addition funnel under reflux. After the addition was completed, the reaction mixture was heated at reflux overnight. THF was striped off and EtOAc was added. The mixture was washed with water and brine. The organic layer was dried and concentrated to give an oil. The crude product was purified by silica column eluting with 1:1:1 mixture of EtOAc/DCM/hexanes to afford 2.43 g (yield 18%) of 4-(4-bromophenyl)-2-cyclopropyl-1H-imidazole. LCMS: [M+1]⁺=263.

Step 2:

To a solution of 4-(4-bromophenyl)-2-cyclopropyl-1H-imidazole (2.43 g, 9.23 mmol) and cesium carbonate (6.02 g, 18.47 mmol) in THF (30 mL) was added iodomethane (1.27 mL, 20.31 mmol). The reaction was stirred at rt for 19 hours. Water was added and the mixture was extracted with ethyl acetate. The organics were dried (MgSO₄) and concentrated to afford the title compound which was used without further purification, LCMS: [M+1]⁺=277.

### Intermediate 10B

### Ethyl (1S,2S)-2-{4-[1-(2-fluoroethyl)-5-iodo-1H-imidazol-4-yl]phenyl}cyclopropanecarboxylate

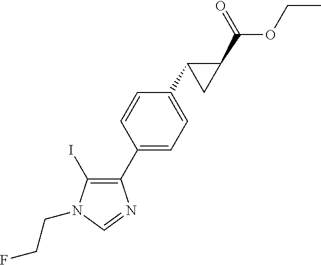 [SMILES: CCOC(=O)[C@H]1C[C@@H]1c1ccc(-c2ncn(CCF)c2I)cc1]

Step 1:

To a solution of Intermediate 3 (0.5 g, 1.95 mmol) in 4 mL of DMF was added 1-fluoro-2-iodoethane (0.34 g, 1.95 mmol) and cesium carbonate (0.7 g, 2.15 mmol). The reaction was stirred at 90° C. for 3 hours. EtOAc (50 mL) was added to the reaction, and the mixture was washed with water (2 times) and brine, dried over MgSO₄, and concentrated to dryness. The residue was purified by silica column (10-80% EtOAc in hexanes) to give 0.45 g (yield 76%) of ethyl (1S,2S)-2-{4-[1-(2-fluoroethyl)-1H-imidazol-4-yl]phenyl}cyclopropanecarboxylate. LCMS: [M+1]⁺=303.

Step 2:

To a solution of ethyl (1S,2S)-2-{4-[1-(2-fluoroethyl)-1H-imidazol-4-yl]phenyl}cyclopropanecarboxylate (450 mg, 1.488 mmol) in dichloromethane (5 mL) was added N-iodosuccinimide (352 mg, 1.563 mmol) and three drops trifluoroacetic acid. The reaction was stirred at rt for 3 h. The mixture was neutralized with aqueous sodium bicarbonate and the organics were extracted with dichloromethane. The organics were then washed with aqueous sodium thiosulfate, followed by three washes with water. The organics were dried (MgSO₄), concentrated, and punted on 20 g of silica gel eluting a gradient of 35-100% ethyl acetate in hexanes to give rise to the title compound as a brown oil (110 mg, 0.257 mmol), LCMS: [M+1]⁺=429.

### Intermediate 11B

(1S,2S)-2-(4-{5-[(5-Chloropyridin-2-yl)thio]-1-methyl-1H-imidazol-4-yl}phenyl)cyclopropanecarbohydrazide

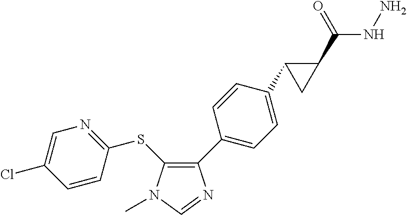 [SMILES: Cn1cnc(-c2ccc([C@H]3C[C@@H]3C(=O)NN)cc2)c1Sc1ccc(Cl)cn1]

Step 1:

Starting with Intermediate 5 and following the same procedure as described for Intermediate 4 (Step 4), ethyl (1S,2S)-2-(4-{5-[(5-chloropyridin-2-yl)thio]-1-methyl-1H-imidazol-4-yl}phenyl)cyclopropanecarboxylateIntermediate was prepared, LCMS: [M+1]⁺=414.

Step 2:

The product from the previous Step (0.5 g, 1.208 mmol) was suspended in ethanol (3 mL) and hydrazine hydrate (2 mL), and heated at reflux for 6 h. Volatiles were evaporated in vacuo to. afford the title compound. LCMS: [M+1]⁺=400.

### Example 1B

### 2-(4-{5[(5-Chloropyridin-2yl-)thio]-1-methyl-1H-imidazol-4-yl}phenyl)-N,N-dimethylcyclopropanecarboxamide

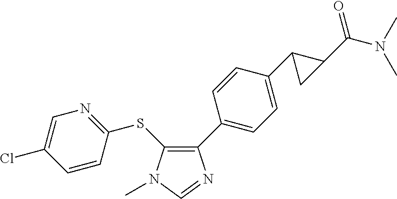 [SMILES: CN(C)C(=O)C1CC1c1ccc(-c2ncn(C)c2Sc2ccc(Cl)cn2)cc1]

To a solution of Intermediate 4 (50 mg, 0.130 mmol), 1-hydroxybenzotriazole hydrate (24 mg, 0.155 mmol), N,N′-diisopropylcarbodiimide (20 mg, 0.155 mmol), and dimethylamine hydrochloride (63 mg, 0.777 mmol) in DMF (1 mL) was added Hunig's base (0.226 mL, 1.296 mmol). The resulting mixture was heated to 80° C. for 30 min and the mixture was subjected to reverse phase HPLC. The fractions containing the product were collected and concentrated. If the trifluoroacetic acid salt was desired, the solvent could be removed via lyophilizes. If the free base was desired, the residue was diluted with ethyl acetate, washed with aqueous sodium bicarbonate, water, and brine. The organic layer was dried (MgSO₄), filtered, and concentrated to afford the title compound. 1H NMR (500 MHz), [(CD₃)₂CO]: 8.43 (s, 1H), 7.99 (s, 1H), 7.92 (d, 2H), 7.71 (d, 1H), 7.15 (d, 2H), 6.93 (d, 1H), 3.71 (s, 6H), 3.15 (s, 3H), 2.32 (m, 1H), 2.21 (m, 1H), 1.46 (m, 1H), 1.21 (m, 1H). LCMS: [M+1]⁺=413. Hman FAAH lysate assay: IC₅₀=1.4 nM.

The Examples in Table 1 were prepared following the procedures described in Example 1 using the appropriate amine and Intermediate 4 as the starting materials.

TABLE 1

| | | | | hFAAH |
|---|---|---|---|---|
| | | LCMS | | lysate |
| | | rt | | IC₅₀ |
| Example | Compound structure | (min) | M + 1 | (nM) |
| 2B | 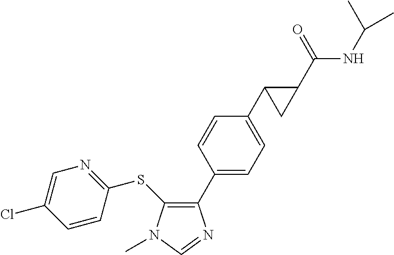 [SMILES: CC(C)NC(=O)C1CC1c1ccc(-c2ncn(C)c2Sc2ccc(Cl)cn2)cc1] | 1.04 | 427 | 6.3 |
| 3B | 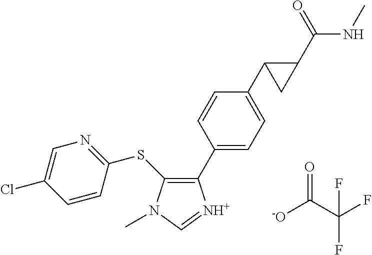 | 0.99 | 399 | 1.4 |
| 4B | 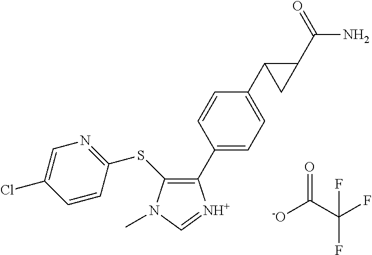 | 2.18* | 385 | 2.6 |

*LCMS 5 min method.

### Example 5B

### (1S,2S)-2-(4-{5-[(5-Chloropyridin-2-yl)thio]-1-methyl-1H-imidazol-4-yl}phenyl)-N,N-dimethylcyclopropanecarboxamide

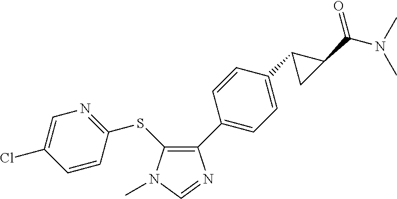 [SMILES: CN(C)C(=O)[C@H]1C[C@@H]1c1ccc(-c2ncn(C)c2Sc2ccc(Cl)cn2)cc1]

To a solution of Intermediate 6 (100 mg, 0.259 mmol), 1-hydroxybenzotriazole hydrate (99 mg, 0.648 mmol), N-[3-(dimethylamino)propyl]-N′-ethylcarbodiimide hydrochloride (124 mg, 0.648 mmol), and dimethylamine (2M in THF) (3 mL, 1.500 mmol) in dioxane (1 Ml) was added Hunig's base (0.272 Ml, 1.555 mmol). Te resulting mixture was heated to 80° C. for 30 min and the mixture was subjected to reverse phase HPLC. The fractions containing the product were collected and concentrated. If the trifluoroacetic acid salt was desired, the solvent could be removed via lyophilizer. If the free base was desired, the residue was diluted with ethyl acetate, washed with aqueous sodium bicarbonate, water, and brine. The organic layer was dried (MgSO₄), filtered, and concentrated to afford the title compound. 1H NMR (500 MHz), [(CD₃)₂CO]: 8.43 (s, 1H), 7.99 (s, 1H), 7.92 (d, 2H), 7.71 (d, 1H), 7.15 (d, 2H), 6.93 (d, 1H), 3.71 (s, 6H), 3.15 (s, 3H), 2.32 (m, 1H), 2.21 (m, 1H), 1.46 (m, 1H), 1.21 (m, 1H). LCMS: [M+1]⁺=413. Human FAAH lysate assay: IC₅₀=1.0 nM.

The Examples in Table 2B were prepared following the procedures described in Example 5 using the appropriate amine and Intermediate 6 as the starting materials.

TABLE 2

| | | | | hFAAH |
|---|---|---|---|---|
| | | LCMS | | lysate |
| | | rt | | IC₅₀ |
| Example | Compound structure | (min) | M + 1 | (nM) |
| 6B | 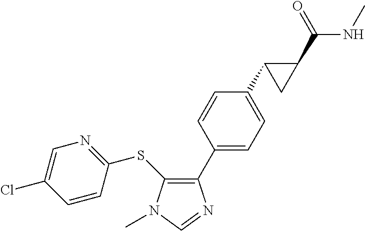 [SMILES: CNC(=O)[C@H]1C[C@@H]1c1ccc(-c2ncn(C)c2Sc2ccc(Cl)cn2)cc1] | 1.05 | 399 | 1.1 |
| 7B | 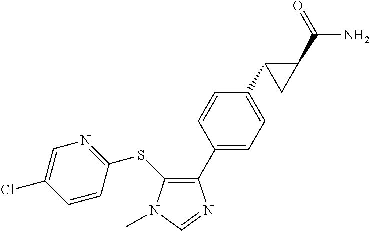 [SMILES: Cn1cnc(-c2ccc([C@H]3C[C@@H]3C(N)=O)cc2)c1Sc1ccc(Cl)cn1] | 1.15 | 385 | 3.3 |
| 8B | 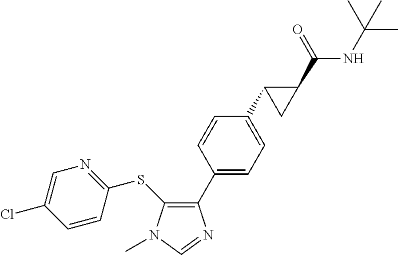 [SMILES: Cn1cnc(-c2ccc([C@H]3C[C@@H]3C(=O)NC(C)(C)C)cc2)c1Sc1ccc(Cl)cn1] | 1.09 | 441 | 195.7 |

### Example 9B

(1S,2S)-2-(4-{5-[(4-Chlorophenyl)thio]-1-methyl-1H-imidazol-4-yl}phenyl)cyclopropanecarboxamide

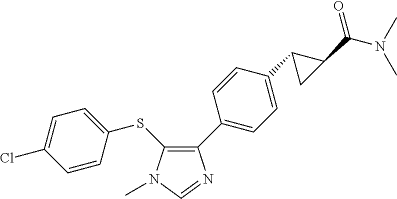 [SMILES: CN(C)C(=O)[C@H]1C[C@@H]1c1ccc(-c2ncn(C)c2Sc2ccc(Cl)cc2)cc1]

The title compound was prepared starting with Intermediate 7B following the same procedure as described for Example 5. ¹H NMR (500 MHz), [(CD₃)₂CO]: 7.97 (br, 3H), 7.32 (d, 2H), 7.16 (d, 2H), 7.05 (d, 2H), 3.66 (s, 3H), 3.14 (s, 3H), 3.02 (s, 3H), 2.34 (m, 1H), 2.21 (m, 1H), 1.47 (m 1H), 1.21 (m, 1H). LCMS: [M+H]⁺=412 Human FAAH lysate assay: IC₅₀ 0.3 nM.

### Example 1013

### (1S,2S)-2-(4-{5-[(5-Chloropyridin-2-yl)thio]-1-methyl-1H-imidazol-4-yl}phenyl)-N-(2-fluoroethyl)cyclopropanecarboxamide

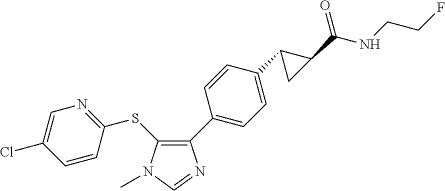 [SMILES: Cn1cnc(-c2ccc([C@H]3C[C@@H]3C(=O)NCCF)cc2)c1Sc1ccc(Cl)cn1]

Starting with 2-fluoroethanamine hydrochloride and Intermediate 6B following the same synthetic procedure as described for Example 5 followed by purification via recrystallization from methanol the title compound was prepared. ¹H NMR (500 MHz), [CDCl₃]: 8.30 (s, 1H), 7.82 (br, 3H), 7.60 (d, 1H), 7.19 (d, 2H), 6.80 (d, 1H), 4.60 (m, 1H), 4.50 (m, 1H), 3.90 (s, 3H), 3.60 (br, 2H), 2.45 (m, 1H), 1.85 (m, 1H), 1.65 (m, 1H), 1.20 (m, 1H). LCMS: [M+1]⁺=430. Human FAAH lysate assay: IC₅₀=1.5 nM.

### Example 11B

### (1S,2S)-2-(4-{5-[(5-Chloropyridin-2-yl)thio]-1-methyl-1H-imidazol-4-yl}phenyl)-N-(2-fluoroethyl)-N-methylcyclopropanecarboxamide

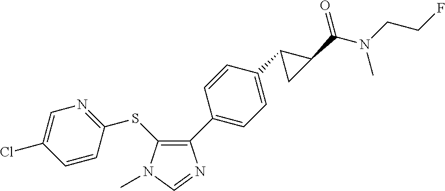 [SMILES: CN(CCF)C(=O)[C@H]1C[C@@H]1c1ccc(-c2ncn(C)c2Sc2ccc(Cl)cn2)cc1]

To a solution of Example 10 (10 mg, 0.023 mmol) in DMF (1 Ml) was added sodium hydride (60% in mineral oil), (6 mg, 0.139 mmol) and iodomethane (0.009 Ml, 0.139 mmol). The reaction mixture was stirred at rt for 30 min. Water was added and the mixture was extracted with ethyl acetate. The organics were dried (MgSO₄), concentrated, and purifed on 4 g of silica gel eluting a gradient of 0-5% triethylamine in ethyl acetate to give rise to the title compound. 1H NMR (500 MHz), [CD₃OD]: 8.34 (s, 1H), 8.04 (s, 1H), 7.73 (m, 2H), 7.63 (d, 1H), 7.14 (m, 2H), 6.92 (d, 1H), 4.58 (m, 1H), 4.48 (m, 1H), 3.66 (s, 3H), 3.19 (s, 3H), 3.00 (br, 2H), 2.37 (m, 1H), 2.18 (m, 1H), 1.53 (m, 1H), 1.31 (m, 1H). LCMS: [M+1]⁺=445. Human FAAH lysate assay: IC₅₀=3.0 nM.

### Example 12B

### (1R,2R)-2-(4-{5-[(5-Chloropyridin-2-yl)thio]-1,2-dimethyl-1H-imidazol-4-yl}phenyl)-N,N-dimethylcyclopropanecarboxamide

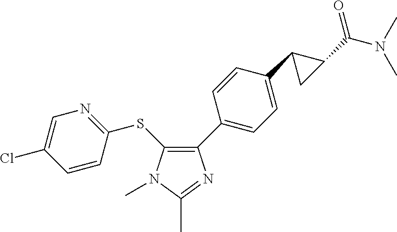 [SMILES: Cc1nc(-c2ccc([C@@H]3C[C@H]3C(=O)N(C)C)cc2)c(Sc2ccc(Cl)cn2)n1C]

Step 1:

Starting with Intermediate 8 following the same procedure as described for Intermediate 4 (Steps 4 and 7), (1R,2R)-2-(4-{5-[(5-chloropyridin-2-yl)thio]-1,2-dimethyl-1H-imidazol-4-yl}phenyl)cyclopropanecarboxylic acid was prepared.

Step 2:

The title compound was prepared starting with the product from the previous step following the same procedure as described for Example 5. ¹H NMR (500 MHz), [(CD₃)₂CO]: 8.45 (s, 1H) 7.84 (d, 2H), 7.82 (d, 1H), 731 (d, 1H), 725 (d, 2H), 3.79 (s, 3H), 3.15 (s, 3H), 2.91 (s, 3H), 2.78 (s, 3H), 2.36 (m, 1H), 228 (m, 1H), L48 (m, 1H), 1.25 (m, 1H). LCMS: [M+1]⁺=427. Human FAAH lysate assay: IC₅₀=13.6 nM.

### Example 13B

### 5-[(5-Chloropyridin-2-yl)thio]-2-cyclopropyl-4-(4-{2-[(dimethylamino)carbonyl]cyclopropyl}phenyl)-1-methyl-1H-imidazol-3-ium trifluoroacetate

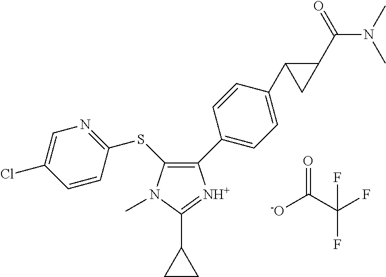

The title compound was prepared starting with Intermediate 9 following the same procedure as described for Example 1. 1H NMR (500 MHz), [(CD₃)₂CO]: 8.45 (s, 1H), 7.78 (br, 3H), 7.20 (br, 3H), 3.87 (s, 3H), 2.33 (m, 1H), 2.30 (m, 1H), 2.24 (m, 1H) 1.47 (m, 1H), 1.32 (m, 2H), 1.23-1.18 (br, 3H). LCMS: [M+1]⁺=453. Human FAAH lysate assay: IC₅₀=48.3 nM.

### Example 14B

### 5-[(5-Chloropyridin-2-yl)thio]-4-(4-{(1S,2S)-2-[(dimethylamino)carbonyl]cyclopropyl}phenyl)-1-(2-fluoroethyl)-1H-imidazol-3-ium trifluoroacetate

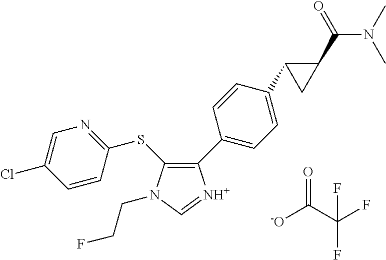

Step 1:

Starting with Intermediate 10B following the same procedure as described for Intermediate 4 (Steps 4 and 7), (1S,2S)-2-{4-[5-[(5-chloropyridin-2-yl)thio]-1-(2-fluoroethyl)-1H-imidazol-4-yl]phenyl}cyclopropanecarboxylic acid was prepared.

Step 2:

The title compound was prepared starting with the product from the previous step following the same procedure as described for Example 5. ¹H NMR (500 MHz), [CD₃COD]: 8.37 (s, 1H), 7.76 (d, 1H), 7.67 (br, 2H), 7.27 (br, 4H), 4.77 (m, 1H), 4.68 (m, 1H), 4.60 (m, 1H), 4.55 (m, 1H), 3.16 (s, 3H), 2.96 (s, 3H), 2.40 (m, 1H), 2.24 (m, 1H), 1.54 (m, 1H), 1.34 (m, 1H). LCMS: [M+1]⁺=445. Human FAAH lysate assay: IC₅₀=3.2 nM.

The Examples in Table 3B were prepared following the procedures described in Example 5 using the appropriate amine and (1S,2S)-2-{4-[5-[(5-chloropyridin-2-yl)thio]-1-(2-fluoroethyl)-1H-imidazol-4-yl]phenyl}cyclopropanecarboxylic acid (Example 14, Step 1) as the starting materials.

TABLE 3B

| | | | | hFAAH |
|---|---|---|---|---|
| | | LCMS | | lysate |
| | | rt | | IC₅₀ |
| Example | Compound structure | (min) | M + 1 | (nM) |
| 15B | 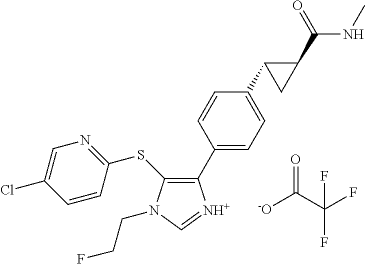 | 2.44* | 431 | 5.8 |
| 16B | 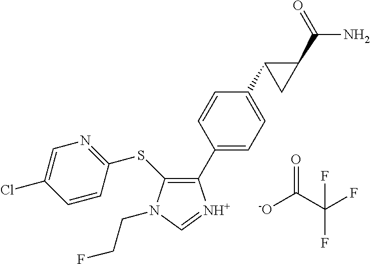 | 2.33* | 417 | 12.7 |

*LCMS 5 min method.

### Example 17B

### 5-[(1S,2S)-2-(4-{5-[(5-Chloropyridin-2-yl)thio]-1-methyl-1H-imidazol-4-yl}phenyl)cyclopropyl]-1,3,4-oxadiazol-2(3H)-one

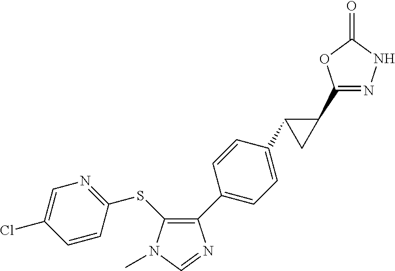 [SMILES: Cn1cnc(-c2ccc([C@H]3C[C@@H]3c3n[nH]c(=O)o3)cc2)c1Sc1ccc(Cl)cn1]

Intermediate 11B (275 mg, 0.688 mmol) was dissolved in THF (0.5 mL), to which was added phosgene (PhMe solution, 1.375 mmol) at −78° C. After it was stirred at −78° C. for 30-60 min, the reaction was quenched with aq NaHCO₃ and the product was extracted with EtOAc. The organic layer was washed with water, brine, dried over MgSO₄, filtered, and concentrated. The residue was purified by reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. 1H NMR (500 MHz), [(CD₃)₂SO]: 8.47 (s, 1H), 8.09 (s, 1H), 7.79 (br, 3H), 7.18 (d, 2H), 6.93 (d, 1H), 2.44 (m, 1H), 2.17 (br, 4H), 1.52 (m, 1H), 1.45 (m, 1H). LCMS: [M+1]⁺=426. Human FAAH lysate assay: IC₅₀=4.5 nM.

### Example 18B

### 5-Chloro-2-[(4-{4-[(1S,2S)-2-(5-methoxy-1,3,4-oxadiazol-2-yl)cyclopropyl]phenyl}-1-methyl-1H-imidazol-5-yl)thio]pyridine

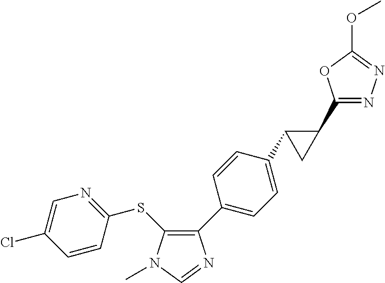 [SMILES: COc1nnc([C@H]2C[C@@H]2c2ccc(-c3ncn(C)c3Sc3ccc(Cl)cn3)cc2)o1]

To a solution of Intermediate 11B (45 mg, 0.113 mmol) in tetramethoxymethane (2 mL) was added two drops of trifluoroacetic acid. The mixture was heated to reflux for 30 min. The volatiles were evaporated and the residue purified by reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. 1H NMR (500 MHz), [(CD₃)₂SO]: 8.88 (s, 1H), 8.47 (s, 1H), 7.83 (d, 2H), 7.29 (br, 2H), 7.15 (br, 2H), 3.15 (s, 3H), 2.57-2.48 (br, 1H), 2.42 (s, 3H), 1.63 (br, 2H), 1.27 (m, 1H). LCMS: [M+1]⁺=440. Human FAAH lysate assay: IC₅₀ 15.6 nM.

### Example 19B

### 5-Chloro-2-[(1-methyl-4-{4-[(1S,2S)-2-(5-methyl-1,3,4-oxadiazol-2-yl)cyclopropyl]phenyl}-1H-imidazol-5-yl)thio]pyridine

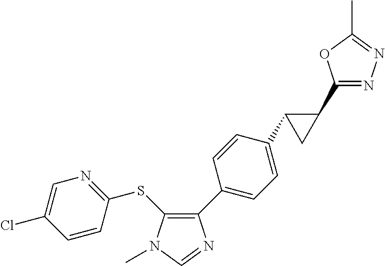 [SMILES: Cc1nnc([C@H]2C[C@@H]2c2ccc(-c3ncn(C)c3Sc3ccc(Cl)cn3)cc2)o1]

Starting with Intermediate 11B and trimethyl orthoacetate, the title compound was prepared following the procedure described in Example 18. 1H NMR (500 MHz), [(CD₃)₂CO]: 8.43 (s, 1H), 8.01 (s 1H), 7.97 (d, 2H), 7.71 (s, 1H), 7.23 (d, 2H), 6.95 (d, 1H), 3.69 (s, 3H), 3.61 (m, 1H), 2.45 (s, 3H), 1.69 (m, 1H), 1.64 (m, 1H), 0.89 (m, 1H). LCMS: [M+1]⁺=424. Human FAAH lysate assay: IC₅₀=46 nM.

### Example 20B

### (1S,2S)-2-(4-{5-[(5-Chloropyridin-2-yl)thio]-1H-imidazol-4-yl}phenyl)-N,N-dimethylcyclopropanecarboxamide

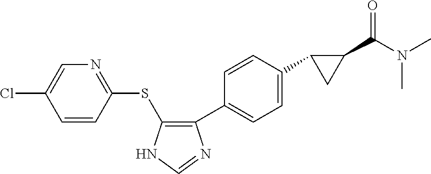 [SMILES: CN(C)C(=O)[C@H]1C[C@@H]1c1ccc(-c2nc[nH]c2Sc2ccc(Cl)cn2)cc1]

Step 1:

Intermediate 3B (860 mg, 3.36 mmol) was dissolved in dichloromethane (15 mL), to which was added NIS (679 mg, 3.02 mmol). The reaction was stirred at rt for 30 min, then it was diluted with dichloromethane (60 mL) and quenched with aqueous NaHCO₃ (30 mL). After the layers were separated, the organic layer was washed with aqueous Na₂S₂O₃, water (×2), and brine, dried over MgSO₄, filtered, and concentrated in vacuo. The crude product was used in the next step without further purification. LCMS: min [M+1]=383.

Step 2:

A microwave tube was charged with CuI (2 mg, 0.01 mmol), 1,10-phenanthroline (2 mg, 0.11 mmol), K₂CO₃ (14 mg, 0.11 mmol), the above Step 1 product (20 mg, 0.05 mmol), Intermediate 1 (9 mg, 0.06 mmol), evacuated, and backfilled with N₂ (three cycles). The tube was sealed and DMSO (1 mL) was added under N₂. The sealed tube was put into the oil bath that was preheated to 100° C., and the reaction mixture was stirred at this temperature for 4 h. After it was cooled to rt, the reaction mixture was partitioned between 10 mL of aqueous NaCl and 20 mL of EtOAc. The organic layer was separated, and the aqueous layer was extracted with 10 mL of EtOAc. The combined organic layers were washed with water, brine, dried, and concentrated. The residue was purified by silica column eluting with 70-100% EtOAc in hexanes to afford 5 mg (26% yield) of ethyl (1S,2S)-2-(4-{5-[(5-chloropyridin-2-yl)thio]-1H-imidazol-4-yl}phenyl)cyclopropanecarboxylate. LCMS: [M+1]=400.

Step 3:

Ethyl (1S,2S)-2-(4-{5-[(5-chloropyridin-2-yl)thio]-1H-imidazol-4-yl}phenyl)cyclopropanecarboxylate (86 mg, 0.215 mmol) was dissolved in 6 mL of acetonitrile, to which was added 2 mL of water, followed by excess KOH pellets. The reaction was stirred at 80° C. for 30 min. After it was cooled to rt, the pH of the reaction mixture was adjusted to 6 with concentrated HCl. EtOAc (50 mL) was added, and the mixture was washed with water and brine, dried, and concentrated to dryness to afford the corresponding acid that was used in the next step with out further purification. LCMS: m/z 372.0 (M+H)⁺.

Step 4:

(1S,2S)-2-(4-{5-[(5-chloropyridin-2-yl)thio]-1H-imidazol-4-yl}phenyl)cyclopropanecarboxylic acid (Step 3 product, 73 mg, 0.196 mmol), HOBT (60 mg, 0.393 mmol), and EDC (113 mg, 0.589 mmol) were dissolved in 3 mL of DMF, to which were added Hunig's base (0.206 mL, 1.178 mmol) and dimethyl amine (2 M THE solution, 0.294 mL, 0.589 mmol). The reaction was heated at 80° C. for 30-60 min. Upon cooling to rt, the reaction was diluted with EtOAc (80 mL), and the reaction mixture was washed with water (2-3 times) and brine, dried, and concentrated to dryness. The residue was purified by silica column (80-100% EtOAc in hexanes) to give 62 mg (79%) of the title compound. 1H NMR (500 MHz, (CD₃OD): 8.38 (s, 1H), 7.97 (s, 1H), 7.65 (d, 2H), 7.59 (d, 1H), 7.18 (d, 2H), 6.82 (d, 1H), 3.16 (s, 3H), 2.97 (s, 3H), 2.38 (m, 1H), 2.19 (m, 1H), 1.54 (m, 1H), 1.27 (m, 1H). LCMS: m/z 399 (M+H)⁺. Human FAAH lysate assay: IC₅₀=649.6 nM.

### Example 21B

### Methyl (1S,2S)-2-(4-{5-[(5-chloropyridin-2-yl)thio]-1-methyl-1H-imidazol-4-yl}phenyl)cyclopropanecarboxylate

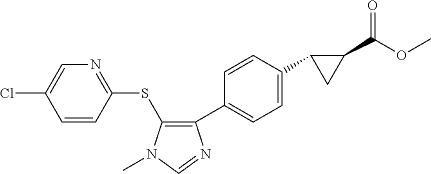 [SMILES: COC(=O)[C@H]1C[C@@H]1c1ccc(-c2ncn(C)c2Sc2ccc(Cl)cn2)cc1]

Intermediate 6B (30 mg, 0.078 mmol) was dissolved in 1 mL of dichloromethane and 1 mL of MeOH, to which was added dropwise trimethylsilyl diazomethane (2 M ether solution) at 0° C. until the organge-yellow color persisted. The reaction mixture was concentrated and the residue was purified by silica column (20-80% EtOAc in hexanes) to afford the title compound. LCMS: m/z 400 (M+H)⁺. Human FAAH lysate assay: IC₅₀=0.2 nM.

### Example 22B

### 2-[(1S,2S)-2-(4-{5-[(5-Chloropyridin-2-yl)thio]-1-methyl-1H-imidazol-4-yl}phenyl)cyclopropyl]propan-2-ol

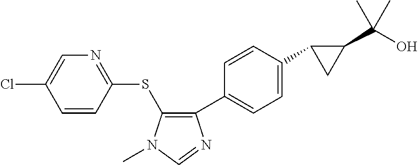 [SMILES: Cn1cnc(-c2ccc([C@H]3C[C@@H]3C(C)(C)O)cc2)c1Sc1ccc(Cl)cn1]

The product of Example 21B (15 mg, 0.038 mmol) was dissolved in 1 mL of THF, to which was added MeMgBr (3 M ether solution, 0.075 mL, 0.225 mmol) at 0° C. After stirring at 0° C. for 10 min, the reaction was quenched with aqueous NH₄Cl. EtOAc (20 mL) was added, and the mixture was washed with water and brine, dried, and concentrated to dryness. The residue was purified by silica column (70-100% EtOAc in hexanes) to afford the title compound. ¹H NMR (500 MHz, (CDCl₃): 8.41 (s, 1H), 8.39 (s, 1H), 7.86 (d, 2H), 7.51 (d, 1H), 7.13 (d, 2H), 6.86 (d, 1H), 3.78 (s, 3H), 1.95 (m, 1H), 1.31 (s, 6H), 1.29 (m, 1H), 1.05 (m, 1H), 0.86 (m, 1H). LCMS: In/z 400 (M+H)⁺. Human FAAH lysate assay: IC₅₀=8.7 nM.

### Example 23B

### [(1S,2S)-2-(4-{5-[(5-Chloropyridin-2-yl)thio]-1-methyl-1H-imidazol-4-yl}phenyl)cyclopropyl]methanol

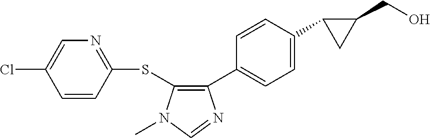 [SMILES: Cn1cnc(-c2ccc([C@H]3C[C@@H]3CO)cc2)c1Sc1ccc(Cl)cn1]

Step 1:

A flask was charged with Intermediate 5B (4.02 g, 10.15 mmol), Intermediate 1 (1.77 g, 12.17 mmol), CuI (97 mg, 0.51 mmol), and K₂CO₃ (2.8 g, 20.29 mmol), evacuated, and backfilled with N₂ (three cycles). Under N₂, DME (50 mL) was added and the reaction was heated at 80° C. overnight. After it was cooled to rt, the reaction mixture diluted with 150 mL of EtOAc. The reaction mixture was washed with water, brine, dried, and concentrated. The residue was purified by silica column eluting with 40-80% EtOAc in hexanes to afford 3.75 g (89% yield) of ethyl (1S,2S)-2-(4-{5-[(5-chloropyridin-2-yl)thio]-1-methyl-1H-imidazol-4-yl}phenyl)cyclopropanecarboxylate. LCMS: m/z 414 (M+H)+.

Step 2:

The product of Step 1 (150 mg, 0.362 mmol) was dissolved in THF (2 mL), to which was added 100 mg of LAH (2.63 mmol) at 0° C. The reaction was stirred at 0° C. for 10 min and was quenched by Fischer work up: careful successive dropwise addition of 0.1 mL of water, 0.1 mL of 15% NaOH solution, and 0.3 mL of water provided a granular inorganic precipitate that was easy to rinse and filter. The granular precipitate was filtered and washed with EtOAc. The combined organic solution was concentrated to give the crude product that was purified by column (95-100% EtOAc in hexanes) to afford the title compound. ¹H NMR (500 MHz, (CDCl₃): 8.39 (s, 1H), 8.05 (s, 1H), 7.86 (d, 2H), 7.48 (d, 1H), 7.06 (d, 2H), 6.81 (d, 1H), 3.67 (s, 3H), 3.61 (d, 2H), 3.02 (broad s, 1H), 1.83 (m, 1H), 1.45 (m, 1H), 0.97 (m, 2H). LCMS: m/z 372 (M+H)+. Human FAAH lysate assay: IC₅₀=88.4 nM.

### Example 24B

### 5-Chloro-2-[(4-{4-[(1S,2S)-2-(methoxymethyl)cyclopropyl]phenyl}-1-methyl-1H-imidazol-5-yl)thio]pyridine

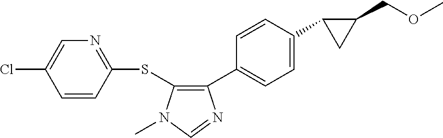 [SMILES: COC[C@H]1C[C@@H]1c1ccc(-c2ncn(C)c2Sc2ccc(Cl)cn2)cc1]

[(1S,2S)-2-(4-{5-[(5-Chloropyridin-2-yl)thio]-1-methyl-1H-imidazol-4-yl}phenyl)cyclopropyl]methanol (product of Example 23, 20 mg, 0.054 mmol) was dissolved in DMF (1 mL), to which was added NaH (0.25 mmol) and MeI (0.25 mmol) at 0° C. The reaction was warmed to rt and stirred for 30 min. The reaction was quenched with 1 mL of aqueous NH₄Cl and diluted with 5 mL of EtOAc. The mixture was washed with water (two times) and brine. The organic layer was dried and concentrated to give the crude product that was purified by column (60-100% EtOAc in hexanes) to afford the title compound. ¹H NMR (500 MHz, (CDCl₃): 9.82 (s, 1H), 8.37 (s, 1H), 7.88 (d, 2H), 7.58 (d, 1H), 7.15 (d, 2H), 7.07 (d, 1H), 3.97 (s, 3H), 3.42 (d, 2H), 3.39 (s, 3H), 1.82 (m, 1H), 1.44 (m, 1H), 0.99 (m, 2H). LCMS: m/z 386 (M+H)+. Human FAAH lysate assay: IC₅₀=35.5 nM.

### Example 25B

### 5-Chloro-2-{[4-(4-{(1S,2S)-2-[(2-fluoroethoxy)methyl]cyclopropyl}phenyl)-1-methyl-1H-imidazol-5-yl]thio}pyridine

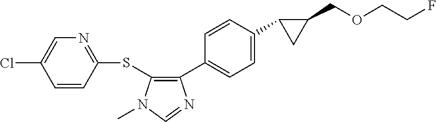 [SMILES: Cn1cnc(-c2ccc([C@H]3C[C@@H]3COCCF)cc2)c1Sc1ccc(Cl)cn1]

[(1S,2S)-2-(4-{5-[(5-Chloropyridin-2-yl)thio]-1-methyl-1H-imidazol-4-yl}phenyl)cyclopropyl]methanol (product of Example 23, 20 mg, 0.054 mmol) was dissolved in DMF (1 mL), to which was added NaH (0.25 mmol) and 1-fluoro-2-iodoethane (0.25 mmol) at 0° C. The reaction was warmed to rt and then heated at 55° C. for 2 hours. After it was cooled t rt, the reaction was quenched with 1 mL of aqueous NH₄Cl and diluted with 5 mL of EtOAc. The mixture was washed with water (two times) and brine. The organic layer was dried and concentrated to give the crude product that was purified by column (40-100% EtOAc in hexanes) to afford the title compound. ¹H NMR (500 MHz, (CDCl₃): 9.83 (s, 1H), 8.38 (s, 1H), 7.88 (d, 2H), 7.63 (d, 1H), 7.19 (d, 1H), 7.18 (d, 2H), 4.63 (m, 1H), 4.58 (m, 1H), 4.02 (s, 3H), 3.76 (m, 1H), 4.71 (m, 1H), 3.57 (d, 2H), 1.85 (m, 1H), 1.46 (m, 1H), 1.03 (m, 2H). LCMS: m/z 418 (M+H)+. Human FAAH lysate assay: IC₅₀=69.3 nM.

### Example 26B

### [³H](1S,2S)-2-(4-{5-[(5-chloropyridin-2-yl)thio]-1-methyl-1H-imidazol-4-yl}phenyl)-N,N-dimethylcyclopropanecarboxamide

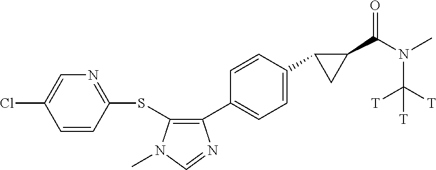

(1S,2S)-2-(4-{5-[(5-chloropyridin-2-yl)thio]-1-methyl-1H-imidazol-4-yl}phenyl)-N-methylcyclopropanecarboxamide (product of Example 6, 1 mg, 0.0025 mmol) was dissolved in DMF (200 uL, anhydrous) and cooled in ice/water bath under nitrogen. NaH (1M, 100 ug) was mixed with 50 uL of THF and added to the solution and the cooling bath was changed to dry ice/methanol. The reaction mixture was stirred vigorously for 20 minutes and then [³H]MeI in toluene (100 mCi, 80 Ci/mmol, 50 uL) was added with syringe. The syringe was rinsed by 2×50 uL toluene and all the rinse solutions were added to the reaction mixture. The reaction solution was kept stirring in dry ice/methanol bath for 1 hour and then in room temperature for overnight. HPLC showed the methylated product. The reaction solution was dried thoroughly over rotary evaporator and the residue was dissolved in 80% ACN/water (1% TFA). HPLC and LC-MS showed 30% product with other by-products and starting material. The mixture was purified by semi-Prep HPLC: Phenomenex Luna Phenyl-Hexyl, 4 mL/min, 254 nm, 70% Aq (0.1% TFA): 30% ACN, isocratic to give desired product 3H-L-002311600 in Tr=˜12.9 min. After sep-pak extraction, the tracer was stored in degassed Ethanol as 0289561-0003. Collect 3.66 mCi/11.5 mL EtOH. SA=66.76 Ci/mmol. HPLC analysis: a. Phenomenex Polar-RP 80A, 1.0 mL/min, 254 nm, 25-45% ACN/water (0.1% TFA) in 20 min, Tr-16.9 min. b. Phenomenex Luna Phenyl-Hexyl, 1.0 mL/min, 254 nm, 30% ACN/water (0.1% TFA) in 20 min, Tr=11.5 min.

### Example 27B

### [³H](1S,2S)-2-(4-{5-[(5-chloropyridin-2-yl)thio]-1-methyl-1H-imidazol-4-yl}phenyl)-N,N-dimethylcyclopropanecarboxamide

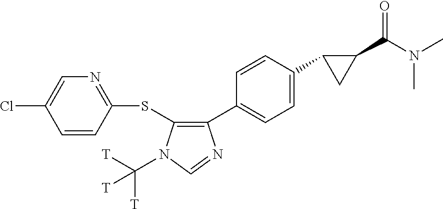

To a 2 mL HPLC vial with stir bar was added the (1S,2S)-2-(4-{5-[(5-Chloropyridin-2-yl)thio]-1H-imidazol-4-yl}phenyl)-N,N-dimethylcyclopropanecarboxamide (product of Example 20B, 1 mg, 0.0025 mmol), Cs₂CO₃ (2.45 mg, 0.0075 mmol), and DMF (0.2 mL), followed by the addition of an ampule of CT₃I (ampule wars washed with 0.1 mL of DMF and that was added to the reaction as well). The mixture was stirred for 2 hours. The crude material was diluted with EtOH and ACN and filtered. The filtrate was concentrated in vacuo to remove volatiles. The residue was purified by RP HPLC (Synergi Polar RP 80A, 4u, 10×250 mm, 5 ml/min, 45% ACN/H2O, PDA detector, 2×0.2 ml injections). The first injection, after solvent switch via C18 sep-pak filtration, yielded 33.88 mCi @ 75.83 Ci/mmol and was delivered in 10 mL abs EtOH. Purity was checked by same column (4.6×250 mm, 1 ml/min, 254 and 220 nm). The second injection was also retained (˜30 mCi in 10 ml EtOH).

### Example 28B

### [¹¹C] (1S,2S)-2-(4-{5-[(5-chloropyridin-2-yl)thio]-1-methyl-1H-imidazol-4-yl}phenyl)-N,N-dimethylcyclopropanecarboxamide

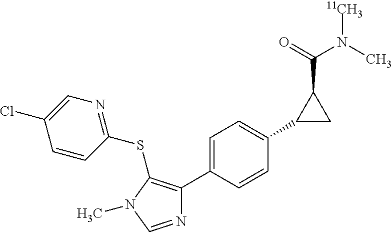

Step 1:

Synthesis of [¹¹C]iodomethane. [¹¹C]CO₂ was produced using a Siemens RDS-111 cyclotron and the [¹¹C]CO₂ was converted to [¹¹C]MeI using a GE Medical Systems TRACERlab FCX system.

Step 2:

[¹¹C]MeI (from Step 1) was trapped in a RT mixture of (1S,2S)-2-(4-{5-[(5-chloropyridin-2-yl)thio]-1-methyl-1H-imidazol-4-yl}phenyl)-N-methylcyclopropane-carboxamide (product of Example 6, 0.25 mg) in DMF (0.25 mL) containing 16 ul of NaH (0.5 g/20 mL DMF). The reaction mixture was transferred to a 2 mL v-vial at 65° C., heated for 5 minutes, diluted with H₂O (0.8 mL) and injected onto the HPLC (Gemini C18, 10×150 mm, Phenomenex). The desired peak was eluted with a solvent system containing 25% A and 75% B (A=MeCN, B=95:5:0.1 H₂O:MeCN:TFA, 5 ml/min, retention time ˜6.5 minutes) and collected in a heated round bottom flask on a rotary evaporator. The solution was concentrated and vacuum transferred to a septum capped 5 mL v-vial. The round bottom flask was rinsed with ethanol (0.1 mL) and saline (1-2 mL) and vacuum transferred to the same v-vial to give 13.6 mCi of [¹¹C] (1S,2S)-2-(4-{5-[(5-chloropyridin-2-yl)thio]-1-methyl-1H-imidazol-4-yl}phenyl)-N,N-dimethyl-cyclopropanecarboxamide.

### Example 29B

### (1S,2S)-2-(4-{5-[(5-chloropyridin-2-yl)thio]-1-[¹¹C]methyl-1H-imidazol-4-yl}phenyl)-N,N-dimethylcyclopropanecarboxamide

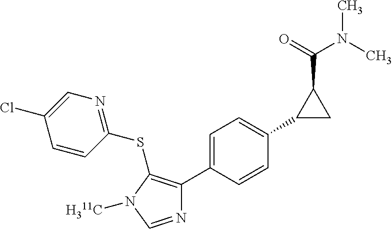

[¹¹C]MeI (synthesized by the same procedure disclosed in Example 28B) was trapped in a RT mixture of (1S,2S)-2-(4-{5-[(5-chloropyridin-2-yl)thio]-1H-imidazol-4-yl}phenyl)-N,N-dimethylcyclopropanecarboxamide (product of Example 20, 0.20 mg) in DMF (0.20 mL) containing Cs₂CO3. The reaction mixture was transferred to a 2 mL v-vial at 65° C., heated for 5 minutes, diluted with H₂O (0.8 mL) and injected onto the HPLC (Gemini C18, 10×150 mm, Phenomenex). The desired peak was eluted with a solvent system containing 22% A and 78% B (A=MeCN, B=95:5:0.1 H₂O:MeCN:TFA, 5 ml/min, retention time ˜11.5 minutes) and collected in a heated round bottom flask on a rotary evaporator. The solution was concentrated and vacuum transferred to a septum capped 5 mL v-vial. The round bottom flask was rinsed with ethanol (0.1 mL) and saline (1-2 mL) and vacuum transferred to the same v-vial to give 35.4 mCi of (1S,2S)-2-(4-{5-[(5-chloropyridin-2-yl)thio]-1-[¹¹C]methyl-1H-imidazol-4-yl}phenyl)-N,N-dimethylcyclopropanecarboxamide.

### Intermediate 12B

### (1S,2S)-2-(4-{5-[(5-chloropyridin-2-yl)thio]-1-methyl-1H-imidazol-4-yl}phenyl)cyclopropanecarbonitrile

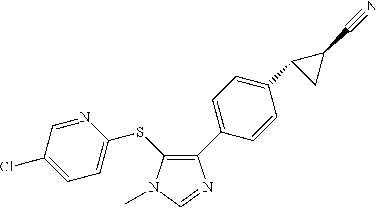 [SMILES: Cn1cnc(-c2ccc([C@H]3C[C@@H]3C#N)cc2)c1Sc1ccc(Cl)cn1]

To a solution of Example 4B (300 mg, 0.779 mmol) in trimethyl phosphate (1 mL, 8.64 mmol) at 0° C. was added trichloromethyl chloroformate (0.145 mL, 1.169 mmol) dropwise. The mixture was then heated to 60° C. to complete the reaction and drive off phosgene. The mixture was neutralized with aqueous sodium bicarbonate and the organics were extracted with EtOAc. The organics were then washed with water and brine, then dried (MgSO₄). The solvent was concentrated to afford the title compound, which was used with out further purification, LCMS: [M+1]⁺=367.

### Intermediate 13B

### (1S,2S)-2-(4-{5-[(5-chloropyridin-2-yl)thio]-1-methyl-1H-imidazol-4-yl}phenyl)-N-hydroxycyclopropanecarboximidamide

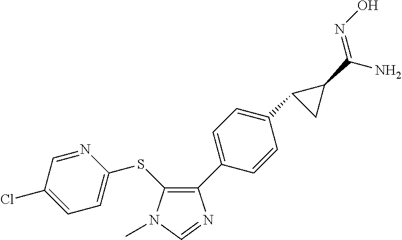 [SMILES: Cn1cnc(-c2ccc([C@H]3C[C@@H]3/C(N)=N/O)cc2)c1Sc1ccc(Cl)cn1]

To a solution of Intermediate 11B (30 mg, 0.082 mmol) in 2 mL EtOH was added 0.25 mL of 50% aqueous NH₂OH and catalytic amount of K₂CO₃. The reaction was heated at 120° C. for 1 h via microwave irradiation. The reaction mixture was concentrated to dryness to afford the title compound which was used with out further purification, LCMS: [M+1]⁺=400.

### Intermediate 14B

### 5-{(1S,2S)-2-[4-(5-iodo-1-methyl-1H-imidazol-4-yl)phenyl]cyclopropyl}-1,3,4-oxadiazol-2(3H)-one

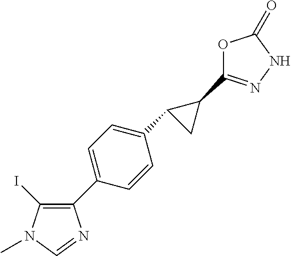 [SMILES: Cn1cnc(-c2ccc([C@H]3C[C@@H]3c3n[nH]c(=O)o3)cc2)c1I]

Step 1:

Starting with Intermediate 513 and following the same procedure as described for Intermediate 11 (Step 2), (1S,2S)-2-[4-(5-iodo-1-methyl-1H-imidazol-4-yl)phenyl]cyclopropanecarbohydrazide was prepared, LCMS: [M+1]⁺=383

Step 2:

The title compound was prepared starting with the product from the previous step and following the procedure described in Example 17B, LCMS: [M+1]⁺=409.

### Example 3013

### 5-[(1S,2S)-2-(4-{5-[(5-chloropyridin-2-yl)thio]-1-methyl-1H-imidazol-4-yl}phenyl)cyclopropyl]-3-methyl-1,3,4-oxadiazol-2(3H)-one

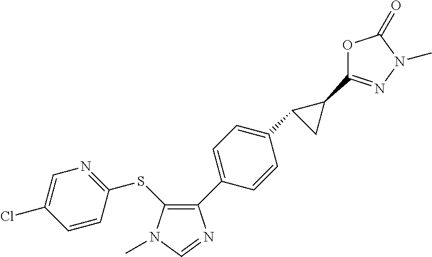 [SMILES: Cn1cnc(-c2ccc([C@H]3C[C@@H]3c3nn(C)c(=O)o3)cc2)c1Sc1ccc(Cl)cn1]

To a solution of Example 17B (5 mg, 0.012 mmol) and excess cesium carbonate in 0.5 mL DMF was added 2 drops of iodomethane. The reaction was stirred at rt for 16 h before filtering and subjecting to purification via reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. ¹H NMR (500 MHz), [(CD₃)₂CO]: 8.43 (s, 1H), 8.13 (s, 1H), 7.96 (d, 2H), 7.75 (d, 1H), 7.24 (d, 2H), 7.02 (d, 1H), 3.75 (s, 3H), 3.30 (s, 3H) 2.56 (m, 1H), 2.20 (m, 1H), 1.60 (br, 2H). LCMS: [M+1]⁺=440. Human FAAH lysate assay: IC₅₀=170.6 nM.

### Example 31B

### 5-[(1S,2S)-2-(4-{5-[(5-chloropyridin-2-yl)thio]-1-methyl-1H-imidazol-4-yl}phenyl)cyclopropyl]-1,3,4-oxadiazole-2(3H)-thione

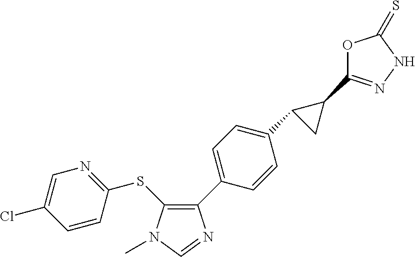 [SMILES: Cn1cnc(-c2ccc([C@H]3C[C@@H]3c3n[nH]c(=S)o3)cc2)c1Sc1ccc(Cl)cn1]

To a solution of Intermediate 11B (50 mg, 0.125 mmol) and 1,1′-carbonothioylbis(1H-imidazole) (50 mg, 0281 mmol) in DCM (1 mL) was added TEA (0.05 mL, 0.359 mmol). The reaction was stirred at rt for 1 hr before evaporating the solvent and subjecting the residue to purification via reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. ¹H NMR (500 MHz), [(CD₃)₂CO]: 8.42 (s, 1H), 8.00-7.93 (br, 2H), 7.71 (d, 1H), 7.21 (d, 2H), 6.95 (d, 2H) 3.70 (s, 3H), 2.40 (m, 1H), 1.98 (m, 1H), 1.55 (m, 1H), 1.30 (m, 2H). LCMS: [M+1]⁺=442. Human FAAH lysate assay: IC₅₀=677.3. nM.

### Example 32B

### 5-[(1S,2S)-2-(4-{5-[(5-chloropyridin-2-yl)thio]-1-methyl-1H-imidazol-4-yl}phenyl)cyclopropyl]-2,4-dihydro-3H-1,2,4-triazole-3-thione

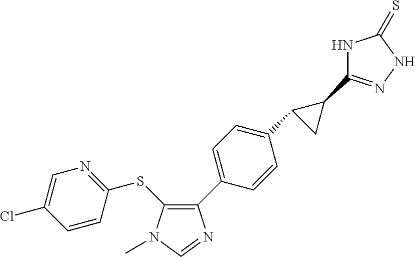 [SMILES: Cn1cnc(-c2ccc([C@H]3C[C@@H]3c3n[nH]c(=S)[nH]3)cc2)c1Sc1ccc(Cl)cn1]

A solution of Intermediate 11B (100 mg, 0.250 mmol) and excess potassium isothiocynate in acetic acid (1 mL) and water (1 mL) was heated to 60° C. for 3 h. The pH was adjusted to 10 with an aqueous solution of NaOH (5N) and the solution was refluxed for 2 h before filtering and subjecting to purification via reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. ¹H NMR (500 MHz), [CDCl₃]: 8.42 (s, 1H), 8.38 (s, 1H), 7.73 (d, 2H), 7.48 (d, 1H), 6.91 (d, 2H), 6.82 (d, 1H), 3.68 (s, 3H), 2.46 (m, 1H), 1.95 (m, 1H), 1.52 (m, 1H), 1.28 (m, 2H). LCMS: [M+1]⁺=441. Human FAAH lysate assay: IC₅₀=304 nM.

### Example 33B

### 5-[(1S,2S)-2-(4-{5-[(5-chloropyridin-2-yl)thio]-1-methyl-1H-imidazol-4-yl}phenyl)cyclopropyl]-2,4-dihydro-3H-1,2,4-triazol-3-one

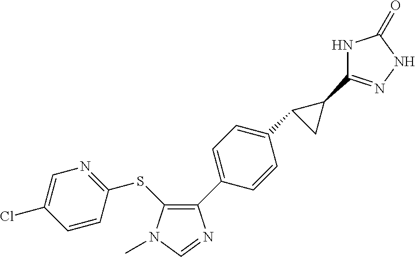 [SMILES: Cn1cnc(-c2ccc([C@H]3C[C@@H]3c3n[nH]c(=O)[nH]3)cc2)c1Sc1ccc(Cl)cn1]

A solution of Intermediate 11B (100 mg, 0.250 mmol) and excess potassium isocyante in acetic acid (1 mL) and water (1 mL) was stirred at rt for 3 h. The pH was adjusted to 10 with an aqueous solution of NaOH (5N) and the solution was refluxed for 2 h before filtering and subjecting to purification via reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. 1H NMR (500 MHz), [CDCl₃]: 8.38 (br, 2H), 7.87 (d, 2H), 7.50 (d, 1H), 7.09 (d, 2H), 6.83 (d, 1H), 6.90 (s, 3H), 1.84 (br, 2H), 1.29 (m, 2H). LCMS: [M+1]⁺=425. Human FAAH lysate assay: IC₅₀=545.3 nM.

### Example 34B

### 5-[(1S,2S)-2-(4-{5-[(5-chloropyridin-2yl)thio]-1-methyl-1H-imidazol-4-yl}phenyl)cyclopropyl]-4-methyl-2,4-dihydro-3H-1,2,4-triazol-3-one

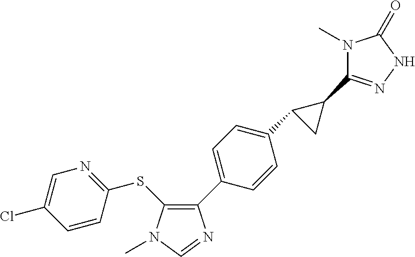 [SMILES: Cn1cnc(-c2ccc([C@H]3C[C@@H]3c3n[nH]c(=O)n3C)cc2)c1Sc1ccc(Cl)cn1]

The title compound was prepared according to the procedure described for Example 33B using methyl isocyanate. ¹H NMR (500 MHz), [CDCl₃]: 10.30 (s, 1H), 8.40 (s, 1H), 7.90 (d, 2H), 7.25 (s, 1H), 7.45 (d, 1H), 7.15 (d, 2H), 6.80 (d, 1H), 3.65 (s, 3H), 3.25 (s, 3H), 2.40 (m, 1H), 1.90 (m, 1H), 1.70 (m, 1H), 1.45 (m, 1H). LCMS: [M+1]⁺=439. Human FAAH lysate assay: IC₅₀=893.5 nM.

### Example 35B

### 5-chloro-2-[(1-methyl-4-{4-[(1S,2S)-2-(1,2,4-oxadiazol-3-yl)cyclopropyl]phenyl}-1H-imidazol-5-yl)thio]pyridine

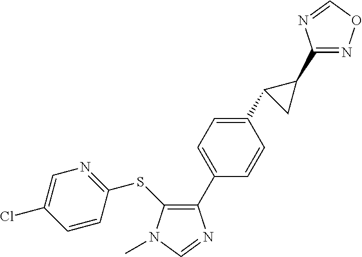 [SMILES: Cn1cnc(-c2ccc([C@H]3C[C@@H]3c3ncon3)cc2)c1Sc1ccc(Cl)cn1]

Intermediate 13B (30 mg, 0.075 mmol) was dissolved in 2 mL triethylorthoformate. A catalytic amount of TFA was added and the reaction was heated at 130° C. for 3 h. The volatiles were removed and the residue was purified by reverse phase HPLC. The fractions containing the product were collected, diluted with ethyl acetate, and washed with aqueous sodium bicarbonate, water, and brine. The organic layer was dried (MgSO₄), filtered, and concentrated to afford the title compound. 1H NMR (500 MHz), [CDCl₃]: 8.60 (s, 1H), 8.39 (s, 1H), 8.01 (s, 1H), 7.96 (d, 2H), 7.50 (d, 1H), 7.17 (d, 2H), 6.80 (d, 1H), 3.69 (s, 3H), 2.59 (m, 1H), 2.47 (m, 1H), 1.73 (m, 1H), 1.56 (m, 1H). LCMS: [M+1]⁺=410. Human FAAH lysate assay: IC₅₀=58.38 nM

### Example 36B

### 5-[(5-chloropyridin-2-yl)thio]-1-methyl-4-{4-[(1S,2S)-2-(5-oxo-4,5-dihydro-1,2,4-oxadiazol-3-yl)cyclopropyl]phenyl}-1H-imidazol-3-ium trifluoroacetate

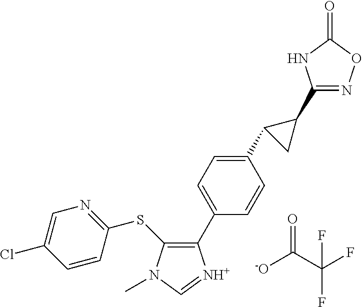

To a solution of Intermediate 13B (107 mg, 0.268 mmol) in pyridine (1 mL) was added ethyl chloroformate (0.025 mL, 0.268 mmol). The reaction was heated to 100° C. for 2 h. The volatiles were evaporated and the residue was purified via reverse phase HPLC. The fractions containing the product were evaporated to afford the title compound. ¹H NMR (500 MHz), [CD₃OD]: 8.38 (s, 1H), 7.77 (d, 1H), 7.69 (br, 3H), 7.29 (br, 3H), 3.83 (s, 3H), 2.54 (m, 1H), 2.13 (m, 1H), 1.65 (m, 1H), 1.60 (m, 1H). LCMS: [M+1]⁺=426. Human FAAH lysate assay: IC₅₀=95.45 nM.

### Example 37B

### 5-[(5-chloropyridin-2-yl)thio]-1-methyl-4-{4-[(1S,2S)-2-(2H-tetrazol-5-yl)cyclopropyl]phenyl}-1H-imidazol-3-ium trifluoroacetate

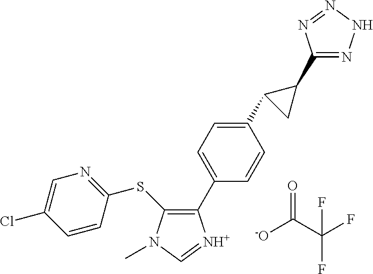

To a dry solution of Intermediate 12 (100 mg, 0.273 mmol) and trimethyltin azide (0.231 mL, 1.363 mmol) in xylene (1 mL) was heated under an atmosphere of nitrogen to 140° C. for 2 h. The volatiles were evaporated and the residue was purified via reverse phase HPLC. The fractions containing the product were evaporated to afford the title compound. 1H NMR (500 MHz), [CD₃OD]: 9.04 (hr, 1H), 8.38 (s, 1H), 7.78 (d, 1H), 7.67 (br, 3H), 7.28 (br, 3H), 3.86 (s, 3H), 2.64 (m, 1H), 2.53 (m, 1H), 1.80-4.75 (br, 2H). LCMS: [M+1]⁺=410. Human FAAH lysate assay: IC₅₀—167.3 nM.

### Example 38B

### 5-chloro-2-[(1-methyl-4-{4-[(1 S,2S)-2-(2-methyl-2H-tetrazol-5-yl)cyclopropyl]phenyl}-1H-imidazol-5-yl)thio]pyridine

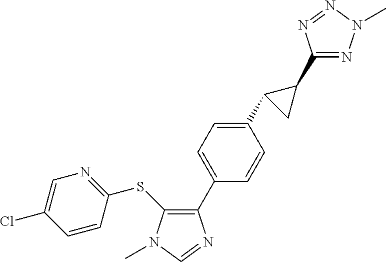 [SMILES: Cn1nnc([C@H]2C[C@@H]2c2ccc(-c3ncn(C)c3Sc3ccc(Cl)cn3)cc2)n1]

To a solution of Example 37B (10 mg, 0.024 mmol) and excess potassium carbonate in DMF (0.5 mL) was added 3 drops of iodomethane. The reaction was stirred at it for 1 h, before filtering and subjecting to purification via reverse phase HPLC. The fractions containing the major product were collected, diluted with ethyl acetate, and washed with aqueous sodium bicarbonate, water, and brine. The organic layer was dried (MgSO₄), filtered, and concentrated to afford the title compound. ¹H NMR (500 MHz), [CD₃O]: 8.38 (s, 1H), 7.78 (d, 2H), 7.76 (br, 3H), 7.30 (br, 3H), 3.83 (s, 3H), 3.66 (s, 3H), 2.66 (m, 1H), 2.45 (m, 1H), 1.85-1.72 (br, 2H). LCMS: [M+1]⁺=424. Human FAAH lysate assay: IC₅₀=37 nM

The examples in Table 4B were prepared following the procedure described for Intermediate 4 (Step 4) using Intermediate 14B and the appropriate thiol as starting materials.

TABLE 4

| | | | | hFAAH |
|---|---|---|---|---|
| | | LCMS | | lysate |
| | | rt | | IC₅₀ |
| Example | Compound structure | (min) | M + 1 | (nM) |
| 39B | 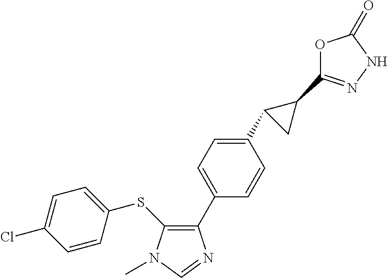 [SMILES: Cn1cnc(-c2ccc([C@H]3C[C@@H]3c3n[nH]c(=O)o3)cc2)c1Sc1ccc(Cl)cc1] | 1.10 | 425 | 24 |
| 40B | 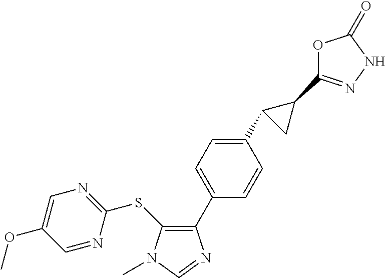 [SMILES: COc1cnc(Sc2c(-c3ccc([C@H]4C[C@@H]4c4n[nH]c(=O)o4)cc3)ncn2C)nc1] | 1.00 | 423 | 33 |
| 41B | 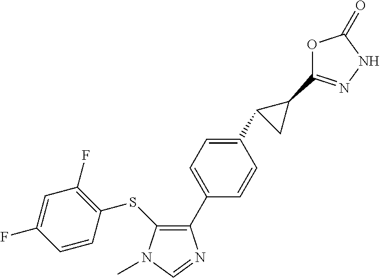 [SMILES: Cn1cnc(-c2ccc([C@H]3C[C@@H]3c3n[nH]c(=O)o3)cc2)c1Sc1ccc(F)cc1F] | 1.08 | 427 | 1.3 |
| 42B | 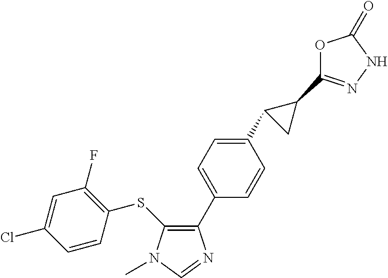 [SMILES: Cn1cnc(-c2ccc([C@H]3C[C@@H]3c3n[nH]c(=O)o3)cc2)c1Sc1ccc(Cl)cc1F] | 1.11 | 443 | 1.8 |
| 43B | 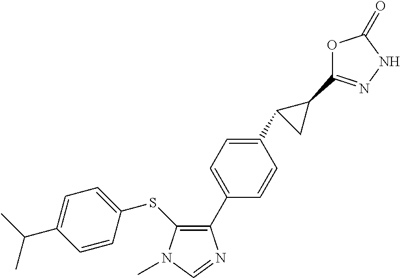 [SMILES: CC(C)c1ccc(Sc2c(-c3ccc([C@H]4C[C@@H]4c4n[nH]c(=O)o4)cc3)ncn2C)cc1] | 1.13 | 433 | 20 |

### MicroPET Camera Imaging

One rat is anesthetized (ketamine/ace-promazine), positioned in the camera, its tail vein cannulated for ease of injection. A 50pe catheter is placed in the femoral vein for collecting blood samples. Another rat is orally administrated with an unlabeled fatty acid amide hydrolase (FAAH) inhibitor 2 hr prior to injection of radiotracer to demonstrate non-specific binding and dose occupancy. 1 mCi/rat of an ¹¹C labeled FAAH inhibitor is injected via its tail vein, and the catheters flushed with several mLs of normal saline. One rat is scanned at a time. Acquisition of images is started as the radiotracer was injected. Images are acquired for 90 minutes and the rat is subsequently euthanized with sodium pentobarbital. Regions of interest (ROIs) are drawn on the summed image which includes the brain, then used to analyze the count rates in subsequent images. Count-rates are converted to %-dose/ROI by dividing the count-rate in the ROI by that of the whole rat, which is then multiplied by 100.

At the time of injection, blood is collected from the femoral catheter and two drops of blood is collected into each tube for the first two minutes, then 300 microliter samples of blood are taken for metabolite correction and determination of radioactivity in plasma and whole blood at 5, 15, 30, 45, 60, and 90 minutes. 300 microliter plasma samples are taken for plasma drug concentration determinations from the rat preinjected with the unlabeled fatty acid amide hydrolase inhibitor right before the injection of PET tracer and after 90 minutes scanning.

### PET Imaging in Rhesus Monkey:

A fasted Rhesus monkey (7-11 kg) is anesthetized with ketamine I.M. (15 mpk) and the monkey is placed in the PET camera bed. An I.V. catheter is inserted into the right saphenous vein. For arterial sampling, the right femoral area is aseptically prepared and an arterial catheter is placed and fixed with sutures. Subsequent anesthesia is maintained with Isoflurane. The animal is intubated and placed on Isoflurane (2-2.5%) with ventilated medical grade compressed air at approximately 23 respirations per minute for the duration of the study. The I:E ratio, volume and rate of respiration is adjusted to maintain CO2 levels ˜40 mmHg and SpO2 levels 95 to 100%. A temperature probe, pulse oximeter, and end tidal CO₂ monitor are connected. Body temperature is maintained by placing the animal on a K-module heating pad and placing another pad on top and the animal is positioned inside the camera gantry supine, head first. General fluid therapy is maintained with 1 ml/min Lactated Ringer's IV throughout study. An aliquot of ¹¹C labeled FAAH inhibitor is injected IV with emission imaging beginning at the time of injection and continuing for 90 minutes.

Whole blood samples are collected via arterial catheter into Heparin tubes for determination of radioactivity in whole blood and plasma. Samples are centrifuged and 20 ul whole blood and plasma are counted 10, 20, 30, 45, 60, 90, and 120 seconds post PET ligand injection. Samples of blood (0.5 ml) are taken for metabolite correction and determination of radioactivity in plasma and whole blood at 3, 5, 15, 30, 60, and 90 minutes.

In a separate experiment, a fasted rhesus monkey is orally dosed with an unlabeled FAAH inhibitor (vehicle: Imwitor/Tween) 21 hr prior to injection of radiotracer. A plasma sample (1 ml) is taken for plasma drug concentration determinations at 20.5, 21, 22, 22.5 hr. At 21 hr, an aliquot of ¹¹C labeled FAAH inhibitor is injected IV and emission imaging begins at the time of injection and continues for 90 minutes following the same protocol as above. Occupancy is determined by comparing tracer binding in various regions of the brain after dosing with the FAAH inhibitor, to tracer binding in the same regions of the brain in the absence of FAAH inhibitor.

### Combination Study

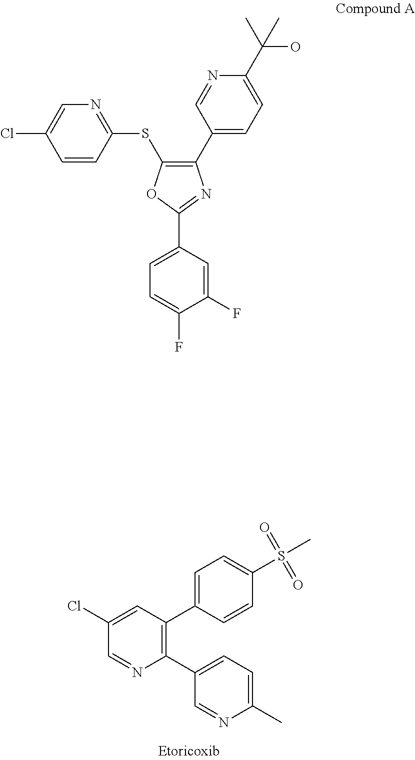 [SMILES: CC(C)(O)c1ccc(-c2nc(-c3ccc(F)c(F)c3)oc2Sc2ccc(Cl)cn2)cn1 ; Cc1ccc(-c2ncc(Cl)cc2-c2ccc(S(C)(=O)=O)cc2)cn1]

Standard isobolographic analysis methods (Tallarida et al. 1989) were used to assess whether the FAAH inhibitor Compound A demonstrates additive or synergistic interaction when co-dosed with etoricoxib in the Complete Freunds Adjuvant model of inflammatory pain in the rat (Eid et al. 2008). Etoricoxib and Compound A were tested in PO dose ratios of 1:1, 0.3:1, and 3:1.

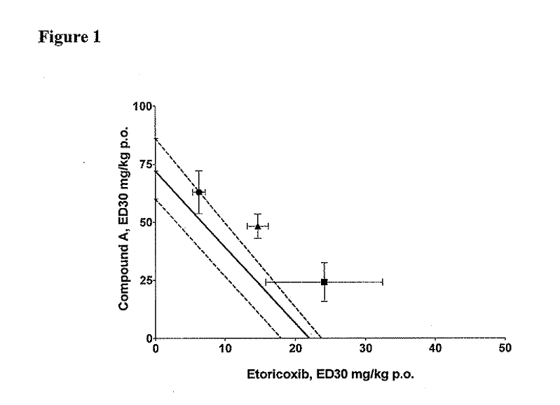

Overall, the data demonstrate that there is not a significant difference between the observations recorded and the predicted line of additivity (FIG. 1). Therefore these data indicate that etoricoxib and the FAAH inhibitor compound A are additive in the their analgesic effect against inflammatory pain.
Tallarida R. J., et al. Statistical analysis of drug-drug and site-site interactions with isobolograms. Life Sci., 45(11): 947-961 (1989).
Eid, S., et al. HC-030031, a TRPA1 selective antagonist, attenuates inflammatory- and neuropathy-induced mechanical hypersensitivity. Molecular Pain, 4: 48 (2008).

What is claimed is:

1. A pharmaceutical compositions comprising:
a FAAH inhibiting compound of formula I: 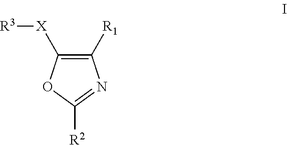
or a pharmaceutically acceptable salt thereof wherein:
X is S or SO;
n is 0, 1 or 2;
R¹ is selected from the group consisting of:
(1) aryl, and
(2) HET¹,
wherein R¹ is optionally mono or di-substituted with substituents R⁴ and R⁵; and wherein R⁴ and R⁵ are independently selected from the group consisting of:
(a) halo,
(b) —CN,
(c) mono, di or tri-halo C₁₋₄ alkyl,
(d) mono, di or tri-halo OC₁₋₄ alkyl,
(d) —OC₁₋₄ alkyl, optionally substituted with hydroxyl, halo or amino,
(e) —C₁₋₄alkyl optionally substituted with one or two substituents selected from hydroxyl, CN, —CHF₂ and —CF₃,
(f) —C₁₋₂alkyl-C₃₋₆cycloalkyl optionally substituted with hydroxy, halo or CN,
(g) —S(O)ₙC₁₋₄alkyl,
(h) —S(O)ₙNR⁶R⁷,
(i) —C(O)—NH—NR⁸R⁹,
(j) —C(O)—OH,
(k) —C(O)—OC₁₋₄alkyl, optionally substituted with halo or hydroxy,
(l) —C(O)—NR¹⁰R¹¹,
(m) —C(O)—C₁₋₄alkyl optionally mono, di or tri substituted with halo,
(o) —C(NR¹²)—NR¹³R¹⁴,
(p) HET⁴,
(q) aryl,
(r) —C(O)—NH—NH—C(O)H,
(s) —CH₂—C(O)—O—C₁₋₄alkyl, whereas the CH₂ may be optionally substituted with C₁₋₄alkyl or OH
(t) —CH₂—C(O)NR¹⁵R¹⁶, whereas the CH₂ may be optionally substituted with C₁₋₄alkyl or OH, and
(u) —NR¹⁷R¹⁸,
wherein choices (p) and (q) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃alkyl;
(9) —C(O)—NR¹⁹R²⁰,
(10) —NH₂,
(11) Oxo,
(12) ═S,
with the proviso that the substituent on choice (q) is other than oxo or ═S,
wherein R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰, are each independently selected from H and C₁₋₄alkyl,
or
R⁶ and R⁷ or R⁸ and R⁹ or R¹⁰ and R¹¹ or R¹³ and R¹⁴ or R¹⁵ and R¹⁶ or R¹⁷ and R¹⁸ or R¹⁹ and R²⁰ are joined together to form a ring with the nitrogen to which they are attached there is fainted a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl and —S(O)nC₁₋₄alkyl;
R² is selected from the group consisting of
(1) aryl,
(2) HET³,
(3) —CH₂-aryl,
(4) —CH₂—HET³,
(5) —C₁₋₆alkyl, and
(6) —C₃₋₆cycloalkyl,
wherein R² is optionally mono or di-substituted with substituents independently selected from the group consisting of
(a) halo,
(b) —CN,
(c) —OH,
(d) —C₁₋₄alkyl optionally substituted with hydroxy, halo or cyano,
(e) —CF₃,
(f) —OC₁₋₄alkyl optionally substituted with hydroxyl or halo,
(g) —C(O)O—C₁₋₃alkyl and
(h) —S-aryl, optionally substituted with halo, C₁₋₄alkyl or —OC₁₋₄alkyl;
R³ is selected from the group consisting of
(1) aryl,
(2) HET⁵, and
(3) C₃₋₆cycloalkyl,
wherein R³ is optionally mono or di-substituted with substituents independently selected from the group consisting of
(a) hydroxy,
(b) halo,
(c) —C₃₋₆cycloalkyl,
(d) —OC3-5cycloalkyl,
(e) —C₁₋₄alkyl,
(f) —OC₁₋₄ alkyl,
(g) —C(O)CH₃
(h) mono, di or tri-halo C₁₋₄ alkyl,
(i) mono, di or tri-halo —OC₁₋₄ alkyl, and
(j) —S(O)ₙ—C₁₋₄ alkyl;
wherein aryl is as a mono- or bi-cyclic aromatic ring system; and HET¹, HET³, HET⁴ and HET⁵ are each independently a 5 to 10-membered aromatic, partially aromatic or non-aromatic mono- or bicyclic ring, or N-oxide thereof, said containing 1 to 4 heteroatoms selected from O, S and N, and optionally substituted with 1 to 2 oxo groups
or a FAHH inhibiting compound of formula II 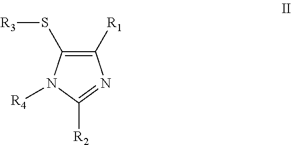
or a pharmaceutically acceptable salt thereof wherein:
n=0, 1 or 2
R₁ is selected from the group consisting of
(1) phenyl, and
(2) HET₁,
wherein choice (1) and (2), is substituted with 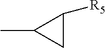
wherein R₅ is selected from the group consisting of
(a) halo,
(b) —CN,
(c) halo C₁₋₄ alkyl,
(d) —OC₁₋₄ alkyl, optionally substituted with hydroxy, halo or amino,
(e) —C₁₋₄alkyl optionally substituted with one or two substituents selected from hydroxyl, CN, —CHF₂ and —CF₃,
(f) —C₁₋₂alkyl-C₃₋₆cycloalkyl optionally substituted with hydroxy, halo or CN,
(g) —S(O)ₙC₁₋₄alkyl,
(h) —S(O)ₙNR₆R₇,
(i) —C(O)—OH,
(j) —C(O)—OC₁₋₄alkyl, optionally substituted with halo or hydroxy,
(k) —C(O)—NR₁₀R₁₁,
(l) —C(O)—C₁₋₄alkyl optionally mono, di or tri substituted with halo,
(m) HET2,
(n) aryl,
(o) —CH₂—C(O)—O—C₁₋₄alkyl, whereas the CH₂ may be optionally substituted with C₁₋₄ alkyl or OH
(t) —CH₂—C(O)NR¹⁵R¹⁶, whereas the CH₂ may be optionally substituted with C₁₋₄ alkyl or OH, and
(u) —NR₁₇R₁₈,
wherein choices (m) and (m) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)—NR₁₉R₂₀,
(9) —NH₂,
(10) Oxo,
(11) ═S,
wherein R₆, R₇, R₁₀, R₁₁, R₁₅, R₁₆, R₁₇, R₁₈, R₁₉ and R₂₀ are each independently selected from H and C₁₋₄alkyl, wherein C₁₋₄alkyl is optionally mono-, di-, or tri-substituted with halo,
or
R₆ and R₇ or R₁₀ and R₁₁ or R₁₅ and R₁₆ or R₁₇ and R₁₈ or R₁₉ and R₂₀ are joined together so that together with the atoms to which they are attached there is formed a 5-membered heterocyclic ring of 4 to 7 atoms, said ring containing 1, 2, 3 or 4 heteroatoms selected from N, O and S, said ring being optionally mono or di-substituted with substituents independently selected from halo, hydroxyl, oxo, C₁₋₄alkyl, hydroxyC₁₋₄alkyl, haloC₁₋₄alkyl, —C(O)—C₁₋₄alkyl and —S(O)nC₁₋₄alkyl;
R₂ is selected from the group consisting of
(1) hydrogen,
(2) aryl,
(3) HET₃,
(4) —CH₂-aryl,
(5) —CH₂—HET₃,
(6) —C₁₋₆alkyl, and
(7) —C₃₋₆cycloalkyl,
wherein choice (2), (3), (4), (5), (6) and (7) is optionally mono or di-substituted with substituents independently selected from the group consisting of
(a) halo,
(b) —CN,
(c) —OH,
(d) —C₁₋₄alkyl optionally substituted with hydroxy, halo or cyano,
(e) —CF₃,
(f) —OC₁₋₄allyl optionally substituted with hydroxyl or halo,
(g) —C(O)O—C₁₋₃alkyl;
R₃ is selected from the group consisting of:
(1) aryl,
(2) HET₄, and
(3) C₃₋₆cycloalkyl,
wherein choice (1), (2) and (3) are each optionally mono or di-substituted with substituents independently selected from the group consisting of
(a) hydroxy,
(b) halo,
(c) —C₃₋₆cycloalkyl,
(d) —OC3-5cycloalkyl,
(e) —C₁₋₄ alkyl,
(f) —OC₁₋₄ alkyl,
(g) —C(O)CH₃
(h) mono, di or tri-halo C₁₋₄ alkyl,
(i) mono, di or tri-halo —OC₁₋₄ alkyl, and
(j) —S(O)ₙ—C₁₋₄ alkyl; and
R₄ is selected from the group consisting of:
(1) —C₁₋₄alkyl,
(2) -haloC₁₋₄alkyl,
(3) H; and
HET₁, HET₂, HET₃ and HET₄ are each independently a 5- to 10-membered aromatic, partially aromatic or non-aromatic mono- or bicyclic ring, containing 1-4 heteroatoms selected from O, S and N, and optionally substituted with 1-2 oxo groups.
Within this aspect there is a genus wherein
R₁ is selected from the group consisting of
(1) phenyl,
(2) pyridinyl,
(3) pyridazinyl,
(4) pyrimidinyl,
(5) pyrazinyl,
(6) thiazolyl,
(7) thienyl,
(8) pyrrolyl, and
(9) oxazolyl,
wherein choice of (1) to (9) is substituted with 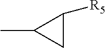
and wherein R₅, is selected from the group consisting of
(b) —CN,
(c) halo C₁₋₄ alkyl,
(d) —O—C₁₋₄alkyl, optionally substituted with hydroxyl, halo or amino
(e) —C₁₋₄alkyl optionally substituted with hydroxyl or CN,
(f) —C₁₋₂alkyl-C₃₋₆cycloalkyl optionally substituted with hydroxy,
(h) —S(O)ₙC₁₋₄alkyl wherein n is 1 or 2,
(i) —S(O)₂NR₆R₇,
(j) —C(O)—NR₁₀R₁₁,
(k) HET2,
(l) aryl, and
wherein choices (k) and (l) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH,
(8) —C(O)O—C₁₋₃alkyl, and
(9) —C(O)—NR₁₉R₂₀,
wherein R₆, R₇, R₁₀, R₁₁, R₁₉ and R₂₀, are each independently selected from H and C₁₋₄alkyl, wherein the C₁₋₄alkyl is optionally momo-, di-, or tri-substituted with halo;
and a second active agent which agent is useful for treating:
acute pain, chronic pain, neurogenic pain, migraine; pain caused by inflammation, and neuropathic pain, anxiety, an eating disorder, obesity, elevated intraocular pressure, glaucoma, a cardiovascular disorder, depression, an inflammatory disorder, asthma, Crohn's disease, and inflammatory bowel disease, food allergy, asthma, skin inflammation, emesis, allodynia. hyperalgesia, headache, visceral pain, dental pain, pain associated with burns, menstrual pain, dysmenhorrea, primary dysmenorrhea, rheumatoid arthritis, juvenile rheumatoid arthritis, osteoarthritis, post operative pain, gynecologic surgery, abdominal surgery, incisions, oral surgery and back pain, epilepsy and epileptiform-iduced damage, exposure to excitotoxic neurotoxins, excitotoxicity, ischaemic brain damage, cerebral ischaema, traumatic injury, depression, anxiety, sleep disorders, Alzheimer's disease, Parkinson s disease, Huntington's disease, amyotropic lateral sclerosis, multiple sclerosis, tourette-s syndrome, schizophrenia, glaucoma, pain, addiction, inflammation, allergic responses, eating disorders, low blood pressure, hypertension, respiratory problems, cancer tumour growth, chemotherapy complications, asphyxia, attention deficit disorder, and gastrointestinal diseases, including nausea and vomiting, gastric ulcers, secretory diarrhea, paralytic ileus, inflammatory bowel disease, colon cancer, gastro-oesophageal reflux conditions, pruritus, fatty liver disease, and non-alcoholic steatohepatitis, and irritable bowel syndrome.

2. A pharmaceutical composition according to claim 1 therein the second active agent is useful for treating acute pain, chronic pain, neurogenic pain, pain associated with migraine, migraine prophylaxis, pain caused by inflammation, neuropathic pain, post-herpetic neuralgia, pain due to chemotherapy-induced peripheral, neuropathy, pain due to HIV induced peripheral neuropathy, pain due to nucleoside reverse transcriptase inhibitor induced peripheral neuropathy, painful diabetic neuropathy, pain due to fibromyalgia, allodynia, hyperalgesia, visceral pain, dental pain, pain associated with burns, menstrual pain, dysmenhorrea, primary dysmenorrhea, rheumatoid arthritis, juvenile rheumatoid arthritis, osteoarthritic, post operative pain, gynecologic surgery, abdominal surgery, incisions, oral surgery and back pain, headache, migraine pain, depression, anxiety, sleep disorders, Alzheimer's disease, Parkinson's disease, an eating disorder and obesity.

3. A pharmaceutical composition according to claim 2 therein the second active agent is useful for treating osteoarthritis, rheumatoid arthritis, inflammatory pain, neuropathic and nociceptive pain, diabetic neuropathy, postherpetic neuralgia, skeletomuscular pain, and fibromyalgia, as well as acute pain, migraine, sleep disorder, Alzheimer disease, and Parkinson's disease.

4. A pharmaceutical composition according to claim 3 therein the second active agent is useful for treating inflammatory pain, neuropathic and nociceptive pain.

5. A pharmaceutical composition according to claim 4 wherein the second active agent is etoricoxib.

6. A pharmaceutical composition wherein the FAAH inhibitor of claim 1 is of formula I wherein
R¹ is selected from the group consisting of
(1) phenyl,
(2) pyridyl,
(3) pyridazinyl,
(4) pyrimidyl,
(5) pyrazinyl,
(6) thiazolyl,
(7) thienyl,
(8) pyrrolyl,
(9) oxazolyl, and
(10) oxadiazolyl;
wherein R¹ is optionally mono or di-substituted with substituents R⁴ and R⁵, wherein R⁴ and R⁵ are independently selected from the group consisting of:
(a) halo,
(b) —CN,
(c) mono, di or tri-halo C₁₋₄ alkyl,
(d) —O—C₁₋₄alkyl, optionally substituted with hydroxyl, halo or amino
(e) —C₁₋₄alkyl optionally substituted with hydroxyl or CN,
(f) —C₁₋₂alkyl-C₃₋₆cycloalkyl optionally substituted with hydroxy,
(h) —S(O)ₙC₁₋₄alkyl wherein n is 0, 1 or 2,
(i) —S(O)ₙNR⁶R⁷,
(j) —C(O)—NR¹⁰R¹¹,
(k) HET⁴,
(l) aryl, and
wherein choices (k) and (l) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH,
(8) —C(O)O—C₁₋₃alkyl, and
(9) —C(O)—NR¹⁹R²⁰,
wherein R⁶, R⁷, R¹⁰, R¹¹, R¹⁹ and R²⁰ are each independently selected from H and C₁₋₄alkyl.

7. A pharmaceutical composition wherein the FAAH inhibitor of claim 6 is of formula I wherein
R¹ is selected from the group consisting of:
(1) phenyl,
(2) pyridyl,
(3) pyrimidyl,
(4) pyrazinyl,
(5) pyridazinyl,
(6) 1,2,4-oxadiazolyl, and
(7) 1,3,4-oxadiazolyl,
optionally mono or di-substituted with substituents R⁴ and R⁵, which are independently selected from the group consisting of
(a) —C₁₋₄alkyl optionally substituted with hydroxy,
(b) —S(O)ₙC₁₋₄alkyl,
(c) —C(O)—NR¹⁰R¹¹,
(d) HET⁴, and
(e) halo,
wherein HET⁴ is optionally mono or di-substituted with substituents selected from:
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃allyl, and
(9) —C(O)—NR¹⁹R²⁰,
wherein R¹⁰, R¹¹, R¹⁹ and R²⁰ are each independently selected from H and C₁₋₄alkyl.

8. A pharmaceutical composition wherein the FAAH inhibitor of claim 1 is of formula I wherein
R² is selected from the group consisting of
(1) aryl,
(2) HET³,
(3) —CH₂aryl, and
(4) —CH₂HET³,
wherein R² is optionally mono or di-substituted with substituents independently selected from the group consisting of:
(a) halo,
(b) —CN,
(c) —OH,
(d) -Hydroxy
(e) —C₁₋₄alkyl,
(f) —C₁₋₄haloalkyl, and
(g) —OC₁₋₄alkyl, optionally substituted with halo or hydroxyl.

9. A pharmaceutical composition wherein the FAAH inhibitor of claim 8 is of formula I wherein
R² is selected from the group consisting of:
(1) aryl, and
(2) HET³,
wherein R² is optionally mono or di-substituted with substituents independently selected from the group consisting of
(a) halo,
(b) —CN,
(c) —OH,
(d) -hydroxy C₁₋₄alkyl,
(e) —CH₃,
(f) —CF₃, and
(g) —OCH₃.

10. A pharmaceutical composition wherein the FAAH inhibitor of claim 9 is of formula I wherein
R² is selected from the group consisting of:
(1) phenyl,
(2) pyridyl,
(3) pyridazinyl,
(4) pyrimidyl,
(5) pyrazinyl,
(6) thiazolyl,
(7) oxazolyl,
(8) pyrazolyl,
(9) 1,2,4-oxadiazolyl, and
(10) 1,3,4-oxadiazolyl,
wherein R² is optionally mono or di-substituted with halo, OC₁₋₄alkyl optially substituted with halogen, —C₁₋₄haloallyl, hydroxyl and CN.

11. A pharmaceutical composition wherein the FAAH inhibitor of claim 1 is of formula I wherein
R³ is selected from the group consisting of:
(1) aryl, and
(2) HET⁵,
wherein choice (1) and (2) are each optionally mono or di-substituted with substituents independently selected from the group consisting of:
(a) halo,
(b) —C₃₋₆cycloalkyl,
(c) —OC₁₋₄ alkyl,
(d) mono, di or tri-halo C₁₋₄ alkyl, and
(e) mono, di or tri-halo —OC₁₋₄alkyl.

12. A pharmaceutical composition wherein the FAAH inhibitor of claim 11 is of formula I wherein
R³ is selected from the group consisting of:
(1) phenyl,
(2) pyrimidyl,
(3) pyridyl,
wherein R³ is optionally mono or di-substituted with halo, haloC₁₋₄allyl, or —OC₁₋₄alkyl optionally substituted with halo.

13. A pharmaceutical composition wherein the FAAH inhibitor of claim 1 is of formula Ia 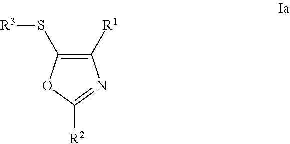
wherein
R¹ is selected from the group consisting of:
(1) phenyl,
(2) pyridyl,
(3) pyridazinyl,
(4) pyrimidyl,
(5) pyrazinyl,
(6) thiazolyl,
(7) thienyl,
(8) pyrrolyl,
(9) oxazolyl, and
(10) oxadiazolyl;
wherein R¹ is optionally mono or di-substituted with substituents R⁴ and R⁵, which are independently selected from the group consisting of
(a) halo,
(b) —CN,
(c) mono, di or tri-halo C₁₋₄ alkyl,
(d) —O—C₁₋₄alkyl, optionally substituted with hydroxyl, halo or amino
(e) —C₁₋₄alkyl optionally substituted with hydroxyl or CN,
(f) —C₁₋₂alkyl-C₃₋₆cycloalkyl optionally substituted with hydroxy,
(h) —S(O)ₙC₁₋₄alkyl wherein n is 0, 1 or 2,
(i) —S(O)ₙNR⁶R⁷,
(j) —C(O)—NR¹⁰R¹¹,
(k) HET⁴,
(l) aryl, and
wherein choices (k) and (l) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH,
(8) —C(O)O—C₁₋₃alkyl, and
(9) —C(O)—NR¹⁹R²⁰,
wherein R⁶, R⁷, R¹⁰, R¹¹, R¹⁹ and R²⁰, are each independently selected from H and C₁₋₄alkyl; R² is selected from the group consisting of:
(1) aryl,
(2) HET³,
(3) —C₁₋₆alkyl, and
(4) —C₃₋₆cycloalkyl,
wherein choice R² is optionally mono or di-substituted with substituents independently selected from the group consisting of
(a) halo,
(b) —CN,
(c) —OH,
(d) -hydroxy C₁₋₄alkyl,
(e) —C₁₋₄alkyl,
(f) —C₁₋₄haloalkyl, and
(g) —OC₁₋₄alkyl, optionally substituted with halo or hydroxyl; and
R³ is selected from the group consisting of:
(1) aryl, and
(2) HET⁵,
wherein choice (1) and (2) are each optionally mono or di-substituted with substituents independently selected from the group consisting of
(a) halo,
(b) —C₃₋₆cycloalkyl,
(c) —C₁₋₄alkyl,
(d) —OC₁₋₄alkyl,
(e) mono, di or tri-halo C₁₋₄ alkyl, and
(f) mono, di or tri-halo —OC₁₋₄ alkyl.

14. A pharmaceutical composition wherein the FAAH inhibitor of claim 13 is of formula Ia wherein
R¹ is selected from the group consisting of
(1) phenyl,
(2) pyridinyl,
(3) pyrimidinyl,
(4) pyrazinyl,
(5) pyridazinyl,
(6) 1,2,4-oxadiazolyl, and
(7) 1,3,4-oxadiazolyl,
optionally mono or di-substituted with substituents R⁴ and R⁵, which are independently selected from the group consisting of
(a) —C₁₋₄alkyl optionally substituted with hydroxy,
(b) —S(O)ₙC₁₋₄alkyl,
(c) —C(O)—NR¹⁰R¹¹,
(d) HET⁴, and
(e) halo,
wherein HET⁴ is optionally mono or di-substituted with substituents selected from:
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃alkyl, and
(9) —C(O)—NR¹⁹R²⁰,
wherein R¹⁰, R¹¹, R¹⁹ and R²⁰ are each independently selected from H and C₁₋₄alkyl.
R² is selected from the group consisting of:
(1) phenyl,
(2) pyridyl,
(3) pyridazinyl,
(4) pyrimidyl,
(5) pyrazinyl,
(6) thiazolyl,
(7) oxazolyl,
(8) pyrazolyl,
(9) 1,2,4-oxadiazolyl, and
(10) 1,3,4-oxadiazolyl,
wherein R² is optionally mono or di-substituted with halo, OC₁₋₄allyl optially substituted with halogen, —C₁₋₄haloalkyl, hydroxyl and CN; and
R³ is selected from the group consisting of
(1) phenyl,
(2) pyrimidyl,
(3) pyridyl,
wherein R³ is optionally mono or di-substituted with halo, haloC₁₋₄alkyl, or —OC₁₋₄alkyl optionally substituted with halo.

15. A pharmaceutical composition wherein the FAAH inhibitor of claim 14 is of formula Ia 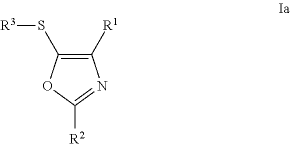
wherein:
R¹ is selected from the group consisting of:
(1) phenyl,
(2) pyridyl,
(3) pyridazinyl,
(4) pyrimidyl,
(5) pyrazinyl,
wherein R¹ is optionally mono or di-substituted with substituents R⁴ and R⁵, which are independently selected from the group consisting of
(a) halo,
(b) —CN,
(c) mono, di or tri-halo C₁₋₄ alkyl,
(d) —O—C₁₋₄alkyl, optionally substituted with hydroxyl, halo or amino
(e) —C(CH₃)₂—OH;
R² is selected from the group consisting of:
(1) phenyl,
(2) pyridyl,
(3) pyridazinyl,
(4) pyrimidyl,
(5) pyrazinyl,
(6) pyrazolyl,
wherein R² is optionally mono or di-substituted with halo, OC₁₋₄allyl optially substituted with halogen, —C₁₋₄haloalkyl, hydroxyl and CN; and
R³ is selected from the group consisting of:
(1) phenyl,
(2) pyrimidyl,
(3) pyridyl,
wherein R³ is optionally mono or di-substituted with halo, haloC₁₋₄alkyl, or —OC₁₋₄alkyl optionally substituted with halo.

16. A pharmaceutical composition wherein the FAAH inhibitor of claim 15 is of formula Ia wherein
R¹ is selected from the group consisting of:
(1) phenyl,
(2) pyridyl,
(3) pyrazinyl,
wherein R¹ is optionally mono or di-substituted with substituents R⁴ and R⁵, which are independently selected from the group consisting of:
(a) halo,
(b) —CN,
(c) mono, di or tri-halo C₁₋₄alkyl,
(d) —O—C₁₋₄alkyl, optionally substituted with hydroxyl, halo or amino
(e) —C(CH₃)₂—OH;
R² is selected from the group consisting of:
(1) phenyl,
(2) pyridyl,
wherein R² is optionally mono or di-substituted with halo, OC₁₋₄alkyl optially substituted with halogen, —C₁₋₄haloalkyl, hydroxyl and CN; and
R³ is selected from the group consisting of:
(1) phenyl,
(2) pyrimidyl,
(3) pyridyl,
wherein R³ is optionally mono or di-substituted with halo, haloC₁₋₄alkyl, or —OC₁₋₄alkyl optionally substituted with halo.

17. A pharmaceutical composition wherein the FAAH inhibitor of claim 1 is of formula II
wherein
R₁ is selected from the group consisting of:
(1) phenyl,
(2) pyridinyl,
(3) pyrimidinyl,
(4) pyrazinyl, and
(5) pyridazinyl,
wherein choice Of (1) to (5) is substituted with 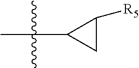
and R₅ is selected from the group consisting of
(a) —C₁₋₄alkyl optionally substituted with hydroxy,
(b) —S(O)₂C₁₋₄alkyl,
(c) —C(O)—NR₁₀R₁₁,
(d) HET₂, and
(e) halo,
wherein choice (d) is optionally mono or di-substituted with substituents selected from:
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃alkyl, and
(9) —C(O)—NR₁₉R₂₀,
wherein R₁₀, R₁₁, R₁₉ and R₂₀ are each independently selected from H and C₁₋₄alkyl, wherein C₁₋₄alkyl is optionally mono-, di-, or tri-substituted with halo.

18. A pharmaceutical composition wherein the FAAH inhibitor of claim 1 is of formula II wherein
R₂ is selected from the group consisting of:
(1) hydrogen,
(2) aryl,
(3) HET₃,
(4) —C₁₋₆alkyl, and
(5) —C₃₋₆cycloalkyl,
wherein choice (2), (3), (4) and (5) is optionally mono or di-substituted with substituents independently selected from the group consisting of
(a) halo,
(b) —CN,
(c) —OH,
(d) -hydroxy C₁₋₄alkyl,
(e) —C₁₋₄alkyl,
(f) —C₁₋₄haloalkyl, and
(g) —OC₁₋₄alkyl, optionally substituted with halo or hydroxyl.

19. A pharmaceutical composition wherein the FAAH inhibitor of claim 18 is of formula II wherein
R₂ is selected from the group consisting of:
(1) hydrogen,
(2) —C₁₋₆alkyl, and
(3) —C₃₋₆cycloalkyl,
wherein choice (2) and (3) are each optionally mono or di-substituted with substituents independently selected from the group consisting of
(a) halo,
(b) —CN,
(c) —OH,
(d) -hydroxy C₁₋₄alkyl,
(e) —CH₃,
(f) —CF₃, and
(g) —OCH₃.

20. A pharmaceutical composition wherein the FAAH inhibitor of claim 1 is of formula II wherein
R₃ is selected from the group consisting of:
(1) phenyl, and
(2) HET₄,
wherein choice (1) and (2) are each optionally mono or di-substituted with substituents independently selected from the group consisting of
(a) halo,
(b) —C₃₋₆cycloalkyl,
(c) —C₁₋₄ alkyl,
(d) —OC₁₋₄ alkyl,
(e) mono, di or tri-halo C₁₋₄ alkyl, and
(f) mono, di or tri-halo —OC₁₋₄ alkyl.

21. A pharmaceutical composition wherein the FAAH inhibitor of claim 20 is of formula II wherein
R₃ is selected from the group consisting of:
(1) phenyl,
(2) pyrimidinyl,
(3) pyridinyl,
(4) pyridazinyl,
(5) pyrazinyl,
wherein choices (1), (2), (3), (4) and (5) are each optionally mono or di-substituted with halo, haloC₁₋₄alkyl, or —OC₁₋₄alkyl optionally substituted with halo.

22. A pharmaceutical composition wherein the FAAH inhibitor of claim 1 is of formula IIa or IIb 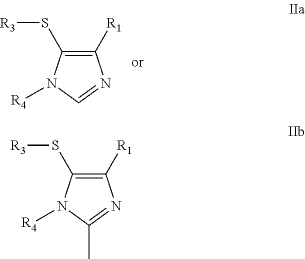
or a pharmaceutically acceptable salt thereof wherein:
R₁ is selected from the group consisting of:
(1) phenyl,
(2) pyridinyl,
(3) pyridazinyl,
(4) pyrimidinyl,
(5) pyrazinyl,
(6) thiazolyl,
(7) thienyl,
(8) pyrrolyl, and
(9) oxazolyl,
wherein choice of (1) to (9) is substituted with 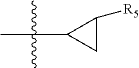
and R₅ is selected from the group consisting of
(a) —CN,
(b) halo C₁₋₄ alkyl,
(c) —O—C₁₋₄alkyl, optionally substituted with hydroxyl, halo or amino
(d) —C₁₋₄alkyl optionally substituted with hydroxyl or CN,
(e) —C₁₋₂alkyl-C₃₋₆cycloalkyl optionally substituted with hydroxy,
(g) —S(O)ₙC₁₋₄alkyl wherein n is 1 or 2,
(h) —S(O)₂NR₆R₇,
(i) —C(O)—NR₁₀R¹¹,
(j) HET2,
(k) aryl, and
wherein choices (j) and (k) are each optionally mono or di-substituted with substituents selected from
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH,
(8) —C(O)O—C₁₋₃alkyl, and
(9) —C(O)—NR₁₉R₂₀,
wherein R₆, R₇, R₁₀, R₁₁, R₁₉ and R₂₀, are each independently selected from H and C₁₋₄alkyl, wherein C₁₋₄alkyl is optionally tritiated or mono-, di-, or tri-substituted with halo, or
R₂ is selected from the group consisting of:
(1) hydrogen,
(2) aryl,
(3) HET₃,
(4) —C₁₋₆allyl, and
(5) —C₃₋₆cycloalkyl,
wherein choice (2), (3), (4) and (5) is optionally mono or di-substituted with substituents independently selected from the group consisting of
(a) halo,
(b) —CN,
(c) —OH,
(d) -hydroxy C₁₋₄alkyl,
(e) —C₁₋₄alkyl,
(f) —C₁₋₄haloalkyl, and
(g) —OC₁₋₄alkyl, optionally substituted with halo or hydroxyl; and
R₃ is selected from the group consisting of:
(1) phenyl, and
(2) HET₄,
wherein choice (1) and (2) are each optionally mono or di-substituted with substituents independently selected from the group consisting of
(a) halo,
(b) —C₃₋₆cycloalkyl,
(c) —C₁₋₄ alkyl,
(d) —OC₁₋₄alkyl,
(e) mono, di or tri-halo C₁₋₄ alkyl, and
(f) mono, di or tri-halo —OC₁₋₄ alkyl;
R₄ is selected from the group consisting of:
(1) —C₁₋₄alkyl, optionally tritiated, and
(3) H;

23. A pharmaceutical composition wherein the FAAH inhibitor of claim 22 is of formula IIa or IIb wherein
R₁ is selected from the group consisting of:
(1) phenyl,
(2) pyridinyl,
(3) pyrimidinyl,
(4) pyrazinyl, and
(5) pyridazinyl,
wherein choice (1) to (5) is substituted with 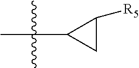
and R₅ is selected from the group consisting of
(a) —C₁₋₄alkyl optionally substituted with hydroxy,
(b) —S(O)₂C₁₋₄alkyl,
(c) —C(O)—NR₁₀R₁₁, and
(d) HET₂,
wherein choice (d) is optionally mono or di-substituted with substituents selected from:
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃alkyl, and
(9) —C(O)—NR₁₉R₂₀,
wherein R₁₀, R₁₁, R₁₉ and R₂₀ are each independently selected from H and C₁₋₄alkyl, wherein C₁₋₄alkyl is optionally tritiated or mono-, di-, or tri-substituted with halo, or
R₂ is selected from the group consisting of:
(1) hydrogen,
(2) —C₁₋₆alkyl, and
(3) —C₃₋₆cycloalkyl,
wherein choice (2) and (3) are each optionally mono or di-substituted with substituents independently selected from the group consisting of
(a) halo,
(b) —CN,
(c) —OH,
(d) -hydroxy C₁₋₄alkyl,
(e) —CH₃,
(f) —CF₃, and
(g) —OCH₃;
R₃ is selected from the group consisting of:
(1) phenyl,
(2) pyrimidinyl,
(3) pyridinyl,
(4) pyrazinyl, and
(5) pyridazinyl,
wherein choices (1), (2), (3), (4) and (5) are each optionally mono or di-substituted with halo, haloC₁₋₄alkyl, or —OC₁₋₄alkyl optionally substituted with halo.

24. A pharmaceutical composition wherein the FAAH inhibitor of claim 23 is of formula IIa or IIb wherein
R₁ is selected from the group consisting of
(1) phenyl, and
(2) pyridinyl,
wherein choice (1) and (2) is substituted with 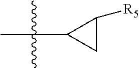
and R₅ is selected from the group consisting of
(a) —C₁₋₄alkyl optionally substituted with hydroxy,
(b) —S(O)₂C₁₋₄alkyl,
(c) —C(O)—NR₁₀R₁₁,
(d) HET₂, and
wherein choice (d) is optionally mono or di-substituted with substituents selected from:
(1) halo,
(2) —CN,
(3) —OH,
(4) —C₁₋₄alkyl optionally substituted with hydroxy, halo or cyano,
(5) —CF₃,
(6) —OC₁₋₄alkyl optionally substituted with hydroxyl or halo,
(7) —C(O)OH, and
(8) —C(O)O—C₁₋₃alkyl, and
(9) —C(O)—NR₁₉R₂₀,
wherein R₁₀, R₁₁, R₁₉ and R₂₀ are each independently selected from H and C₁₋₄alkyl, wherein C₁₋₄alkyl is optionally tritiated mono-, di-, or tri-substituted with halo, or
R₂ is selected from the group consisting of:
(1) hydrogen,
(2) —Cl_{—}6alkyl, and
(3) —C₃₋₆cycloalkyl,
wherein choice (2) and (3) are each optionally mono or di-substituted with substituents independently selected from the group consisting of
(a) halo,
(b) —CN,
(c) —OH,
(d) -hydroxy C₁₋₄alkyl,
(e) —CH₃,
(f) —CF₃, and
(g) —OCH₃;
R₃ is selected from the group consisting of
(1) phenyl,
(2) pyrimidinyl,
(3) pyridinyl,
wherein choices (1), (2) and (3) are each optionally mono or di-substituted with halo, haloC₁₋₄alkyl, or —OC₁₋₄alkyl optionally substituted with halo.

25. A method of treating a disease selected from acute pain, chronic pain, neurogenic pain, migraine; pain caused by inflammation, and neuropathic pain, anxiety, an eating disorder, obesity, elevated intraocular pressure, glaucoma, a cardiovascular disorder, depression, an inflammatory disorder, asthma, Crohn's disease, and inflammatory bowel disease, food allergy, asthma, skin inflammation, emesis, allodynia. hyperalgesia, headache, visceral pain, dental pain, pain associated with burns, menstrual pain, dysmenhorrea, primary dysmenorrhea, rheumatoid arthritis, juvenile rheumatoid arthritis, osteoarthritis, post operative pain, gynecologic surgery, abdominal surgery, incisions, oral surgery and back pain, epilepsy and epileptiform-iduced damage, exposure to excitotoxic neurotoxins, excitotoxicity, ischaemic brain damage, cerebral ischaema, traumatic injury, depression, anxiety, sleep disorders, Alzheimer's disease, Parkinson's disease, Huntington's disease, amyotropic lateral sclerosis, multiple sclerosis, tourette-s syndrome, schizophrenia, glaucoma, pain, addiction, inflammation, allergic responses, eating disorders, low blood pressure, hypertension, respiratory problems, cancer tumour growth, chemotherapy complications, asphyxia, attention deficit disorder, and gastrointestinal diseases, including nausea and vomiting, gastric ulcers, secretory diarrhea, paralytic ileus, inflammatory bowel disease, colon cancer, gastro-oesophageal reflux conditions, pruritus, fatty liver disease, and non-alcoholic steatohepatitis, and irritable bowel syndrome comprising: administration of a composition according to claim 1.
